(19) United States
(12) Patent Application Publication
(10) Pub. No.: US 2026/0221249 A1
(43) Pub. Date: Jul. 30, 2026

(54) SYSTEMS AND METHODS USING MULTIDIMENSIONAL LANGUAGE AND VISION MODELS AND MAPS TO CATEGORIZE, DESCRIBE, COORDINATE, AND TRACK ANATOMY AND HEALTH DATA

(71) Applicant: MoFaIP, LLC, Holland, OH (US)

(72) Inventor: Matthew A. Molenda, Monclova, OH (US)

(73) Assignee: MoFaIP, LLC, Holland, OH (US)

(21) Appl. No.: 19/572,360

(22) Filed: Mar. 19, 2026

Related U.S. Application Data
(63) Continuation of application No. 18/225,872, filed on Jul. 25, 2023.
Application No. 18/225,872 is a continuation-in-part of application No. PCT/IB2022/000814, filed on Dec. 12, 2022.
Application No. 18/225,872 is a continuation-in-part of application No. PCT/IB2022/000777, filed on Dec. 12, 2022.
Application No. 18/225,872 is a continuation-in-part of application No. PCT/IB2022/000793, filed on Dec. 12, 2022.
Application No. 18/225,872 is a continuation-in-part of application No. PCT/IB2022/000813, filed on Dec. 12, 2022.
Application No. 18/225,872 is a continuation-in-part of application No. PCT/IB2022/000824, filed on Dec. 12, 2022.
Application No. 18/225,872 is a continuation-in-part of application No. PCT/IB2022/000823, filed on Dec. 29, 2022.
Application No. 18/225,872 is a continuation-in-part of application No. PCT/US2023/066761, filed on May 9, 2023.
Application No. 18/225,872 is a continuation-in-part of application No. PCT/US2023/066763, filed on May 9, 2023.
Application No. 18/225,872 is a continuation-in-part of application No. PCT/US2023/067049, filed on May 16, 2023.
(60) Provisional application No. 63/265,216, filed on Dec. 10, 2021.
(60) Provisional application No. 63/265,216, filed on Dec. 10, 2021.
(60) Provisional application No. 63/265,216, filed on Dec. 10, 2021.
(60) Provisional application No. 63/265,216, filed on Dec. 10, 2021.
(60) Provisional application No. 63/265,216, filed on Dec. 10, 2021.
(60) Provisional application No. 63/294,653, filed on Dec. 29, 2021.
(60) Provisional application No. 63/294,653, filed on Dec. 29, 2021.
(60) Provisional application No. 63/267,269, filed on Jan. 28, 2022.
(60) Provisional application No. 63/267,269, filed on Jan. 28, 2022.
(60) Provisional application No. 63/315,289, filed on Mar. 1, 2022.
(60) Provisional application No. 63/315,289, filed on Mar. 1, 2022.
(60) Provisional application No. 63/315,289, filed on Mar. 1, 2022.
(60) Provisional application No. 63/315,289, filed on Mar. 1, 2022.
(60) Provisional application No. 63/315,289, filed on Mar. 1, 2022.
(60) Provisional application No. 63/269,516, filed on Mar. 17, 2022.
(60) Provisional application No. 63/269,516, filed on Mar. 17, 2022.
(60) Provisional application No. 63/269,516, filed on Mar. 17, 2022.
(60) Provisional application No. 63/269,516, filed on Mar. 17, 2022.
(60) Provisional application No. 63/269,516, filed on Mar. 17, 2022.
(60) Provisional application No. 63/269,516, filed on Mar. 17, 2022.
(60) Provisional application No. 63/362,791, filed on Apr. 11, 2022.
(60) Provisional application No. 63/362,791, filed on Apr. 11, 2022.
(60) Provisional application No. 63/362,791, filed on Apr. 11, 2022.
(60) Provisional application No. 63/362,791, filed on Apr. 11, 2022.
(60) Provisional application No. 63/362,791, filed on Apr. 11, 2022.
(60) Provisional application No. 63/362,791, filed on Apr. 11, 2022.
(60) Provisional application No. 63/364,393, filed on May 9, 2022.
(60) Provisional application No. 63/364,393, filed on May 9, 2022.
(60) Provisional application No. 63/364,393, filed on May 9, 2022.
(60) Provisional application No. 63/364,393, filed on May 9, 2022.
(60) Provisional application No. 63/364,393, filed on May 9, 2022.
(60) Provisional application No. 63/364,393, filed on May 9, 2022.
(60) Provisional application No. 63/364,764, filed on May 16, 2022.
(60) Provisional application No. 63/364,764, filed on May 16, 2022.
(60) Provisional application No. 63/364,764, filed on May 16, 2022.
(60) Provisional application No. 63/364,764, filed on May 16, 2022.
(60) Provisional application No. 63/364,764, filed on May 16, 2022.
(60) Provisional application No. 63/364,764, filed on May 16, 2022.
(60) Provisional application No. 63/364,764, filed on May 16, 2022.
(60) Provisional application No. 63/365,026, filed on May 20, 2022.
(60) Provisional application No. 63/365,026, filed on May 20, 2022.
(60) Provisional application No. 63/365,026, filed on May 20, 2022.
(60) Provisional application No. 63/365,026, filed on May 20, 2022.
(60) Provisional application No. 63/365,026, filed on May 20, 2022.
(60) Provisional application No. 63/365,373, filed on May 26, 2022.
(60) Provisional application No. 63/365,373, filed on May 26, 2022.
(60) Provisional application No. 63/365,373, filed on May 26, 2022.
(60) Provisional application No. 63/365,373, filed on May 26, 2022.
(60) Provisional application No. 63/365,373, filed on May 26, 2022.
(60) Provisional application No. 63/366,107, filed on Jun. 9, 2022.
(60) Provisional application No. 63/366,107, filed on Jun. 9, 2022.
(60) Provisional application No. 63/366,107, filed on Jun. 9, 2022.
(60) Provisional application No. 63/366,816, filed on Jun. 22, 2022.
(60) Provisional application No. 63/366,816, filed on Jun. 22, 2022.
(60) Provisional application No. 63/369,469, filed on Jul. 26, 2022.
(60) Provisional application No. 63/369,469, filed on Jul. 26, 2022.
(60) Provisional application No. 63/369,469, filed on Jul. 26, 2022.
(60) Provisional application No. 63/369,717, filed on Jul. 28, 2022.
(60) Provisional application No. 63/369,717, filed on Jul. 28, 2022.
(60) Provisional application No. 63/369,717, filed on Jul. 28, 2022.
(60) Provisional application No. 63/369,717, filed on Jul. 28, 2022.
(60) Provisional application No. 63/369,717, filed on Jul. 28, 2022.
(60) Provisional application No. 63/370,879, filed on Aug. 9, 2022.
(60) Provisional application No. 63/370,879, filed on Aug. 9, 2022.
(60) Provisional application No. 63/370,879, filed on Aug. 9, 2022.
(60) Provisional application No. 63/373,469, filed on Aug. 25, 2022.
(60) Provisional application No. 63/375,325, filed on Sep. 12, 2022.
(60) Provisional application No. 63/375,325, filed on Sep. 12, 2022.
(60) Provisional application No. 63/375,325, filed on Sep. 12, 2022.
(60) Provisional application No. 63/382,371, filed on Nov. 4, 2022.
(60) Provisional application No. 63/482,693, filed on Feb. 1, 2023.
(60) Provisional application No. 63/494,652, filed on Apr. 6, 2023.
(60) Provisional application No. 63/470,546, filed on Jun. 2, 2023.
(60) Provisional application No. 63/521,020, filed on Jun. 14, 2023.

(51) Int. Cl. G16H 15/00; G16H 10/60

(52) U.S. Cl. CPC G16H 15/00; G16H 10/60

(57) ABSTRACT

The disclosed embodiments relate to building and applying multidimensional language and vision models and maps to categorize, label and track anatomy and health and other data. Language models are used to accurately, precisely, and reproducibly describe and translate anatomy and health data into any coded, linguistic, or symbolic language. Vision-language models are used to describe, document, associate, categorize, diagnose, track, translate, map, and visualize anatomy and other health data such as morphology and symptoms and treatment recommendations. Language-vision models are used to describe, document, associate, categorize, diagnose, track, summarize, relate, translate, map, and visualize anatomy and other health data such as morphology and symptoms and treatment recommendations and regimens.

### CROSS REFERENCE TO RELATED APPLICATIONS

This is a continuation application claiming the benefit of U.S. application Ser. No. 18/225,872, filed on Jul. 25, 2023, a continuation-in-part application claiming priority to international application PCT/IB2022/000814, filed on Dec. 12, 2022, which claims the benefit of United States Provisional Application No. 63,265,216, filed on Dec. 10, 2021, U.S. Provisional Application No. 63/294,653, filed on Dec. 29, 2021, U.S. Provisional Application No. 63/267,269, filed on Jan. 28, 2022, U.S. Provisional Application No. 63/315,289, filed on Mar. 1, 2022, U.S. Provisional Application No. 63/269,516, filed on Mar. 17, 2022, U.S. Provisional Application No. 63/362,791, filed on Apr. 11, 2022, U.S. Provisional Application No. 63/364,393, filed on May 9, 2022, U.S. Provisional Application No. 63/364,764, filed on May 16, 2022, U.S. Provisional Application No. 63/365,026, filed on May 20, 2022, U.S. Provisional Application No. 63/365,373, filed on May 26, 2022, U.S. Provisional Application No. 63/366,107, filed on Jun. 9, 2022, U.S. Provisional Application No. 63/366,816, filed on Jun. 22, 2022, and U.S. Provisional Application No. 63/369,717, filed on Jul. 28, 2022, each of which is incorporated by reference in the entirety.

U.S. application Ser. No. 18/225,872 also claims priority to international application PCT/IB2022/000777, filed on Dec. 12, 2022, which claims the benefit of U.S. Provisional Application No. 63/265,216, filed on Dec. 10, 2021, U.S. Provisional Application No. 63/315,289, filed on Mar. 1, 2022, U.S. Provisional Application No. 63/269,516, filed on Mar. 17, 2022, U.S. Provisional Application No. 63/362,791, filed on Apr. 11, 2022, U.S. Provisional Application No. 63/364,393, filed on May 9, 2022, U.S. Provisional Application No. 63/364,764, filed on May 16, 2022, United States Provisional Application No. 63/365,026, filed on May 20, 2022, U.S. Provisional Application No. 63/365,373, filed on May 26, 2022, U.S. Provisional Application No. 63/366,107, filed on Jun. 9, 2022, U.S. Provisional Application No. 63/369,717, filed on Jul. 28, 2022, U.S. Provisional Application No. 63/370,879, filed on Aug. 9, 2022, and U.S. Provisional Application No. 63/375,325, filed on Sep. 12, 2022, each of which is incorporated by reference in the entirety.

U.S. application Ser. No. 18/225,872 also claims priority to international application PCT/IB2022/000793, filed on Dec. 12, 2022, which claims the benefit of United States Provisional Application No. 63,265,216, filed on Dec. 10, 2021, U.S. Provisional Application No. 63/315,289, filed on Mar. 1, 2022, U.S. Provisional Application No. 63/269,516, filed on Mar. 17, 2022, and U.S. Provisional Application No. 63/362,791, filed on Apr. 11, 2022, each of which is incorporated by reference in the entirety.

U.S. application Ser. No. 18/225,872 also claims priority to international application PCT/IB2022/000813, filed on Dec. 12, 2022, which claims the benefit of United States Provisional Application No. 63,265,216, filed on Dec. 10, 2021, U.S. Provisional Application No. 63/315,289, filed on Mar. 1, 2022, U.S. Provisional Application No. 63/269,516, filed on Mar. 17, 2022, U.S. Provisional Application No. 63/362,791, filed on Apr. 11, 2022, and U.S. Provisional Application No. 63/369,717, filed on Jul. 28, 2022, each of which is incorporated by reference in the entirety.

U.S. application Ser. No. 18/225,872 also claims priority to international application PCT/IB2022/000824, filed on Dec. 12, 2022, which claims the benefit of United States Provisional Application No. 63,265,216, filed on Dec. 10, 2021, U.S. Provisional Application No. 63/267,269, filed on Jan. 28, 2022, U.S. Provisional Application No. 63/315,289, filed on Mar. 1, 2022, U.S. Provisional Application No. 63/269,516, filed on Mar. 17, 2022, U.S. Provisional Application No. 63/362,791, filed on Apr. 11, 2022, U.S. Provisional Application No. 63/364,393, filed on May 9, 2022, U.S. Provisional Application No. 63/364,764, filed on May 16, 2022, U.S. Provisional Application No. 63/365,373, filed on May 26, 2022, U.S. Provisional Application No. 63/366,107, filed on Jun. 9, 2022, U.S. Provisional Application No. 63/369,469, filed on Jul. 26, 2022, U.S. Provisional Application No. 63/370,879, filed on Aug. 9, 2022, and U.S. Provisional Application No. 63/375,325, filed on Sep. 12, 2022, each of which is incorporated by reference in the entirety.

U.S. application Ser. No. 18/225,872 also claims priority to international application PCT/IB2022/000823, filed on Dec. 29, 2022, which claims the benefit of U.S. Provisional Application No. 63/294,653, filed on Dec. 29, 2021, U.S. Provisional Application No. 63/269,516, filed on Mar. 17, 2022, U.S. Provisional Application No. 63/362,791, filed on Apr. 11, 2022, U.S. Provisional Application No. 63/364,393, filed on May 9, 2022, U.S. Provisional Application No. 63/364,764, filed on May 16, 2022, U.S. Provisional Application No. 63/365,026, filed on May 20, 2022, U.S. Provisional Application No. 63/366,816, filed on Jun. 22, 2022, U.S. Provisional Application No. 63/369,469, filed on Jul. 26, 2022, and U.S. Provisional Application No. 63/369,717, filed on Jul. 28, 2022, each of which is incorporated by reference in the entirety.

U.S. application Ser. No. 18/225,872 also claims priority to international application PCT/US2023/066761, filed on May 9, 2023, which claims the benefit of U.S. Provisional Application No. 63/364,393, filed on May 9, 2022, U.S. Provisional Application No. 63/364,764, filed on May 16, 2022, U.S. Provisional Application No. 63/365,026, filed on May 20, 2022, and U.S. Provisional Application No. 63/365,373, filed on May 26, 2022, each of which is incorporated by reference in the entirety.

U.S. application Ser. No. 18/225,872 also claims priority to international application PCT/US2023/066763, filed on May 9, 2023, which claims the benefit of U.S. Provisional Application No. 63/364,393, filed on May 9, 2022, and U.S. Provisional Application No. 63/364,764, filed on May 16, 2022, each of which is incorporated by reference in the entirety.

U.S. application Ser. No. 18/225,872 also claims priority to international application PCT/US2023/067049, filed on May 16, 2023, which claims the benefit of U.S. Provisional Application No. 63/364,764, filed on May 16, 2022, U.S. Provisional Application No. 63/365,026, filed on May 20, 2022, and U.S. Provisional Application No. 63/365,373, filed on May 26, 2022, each of which is incorporated by reference in the entirety.

U.S. application Ser. No. 18/225,872 also claims priority to U.S. Provisional Application No. 63/369,469, filed on Jul. 26, 2022, U.S. Provisional Application No. 63/369,717, filed on Jul. 28, 2022, U.S. Provisional Application No. 63/370,879, filed on Aug. 9, 2022, U.S. Provisional Application No. 63/373,469, filed on Aug. 25, 2022, U.S. Provisional Application No. 63/375,325, filed on Sep. 12, 2022, U.S. Provisional Application No. 63/382,371, filed on Nov. 4, 2022, U.S. Provisional Application No. 63/482,693, filed on Feb. 1, 2023, U.S. Provisional Application No. 63/494,652, filed on Apr. 6, 2023, U.S. Provisional Application No. 63/470,546, filed on Jun. 2, 2023, and U.S. Provisional Application No. 63/521,020, filed on Jun. 14, 2023, each of which is incorporated by reference in the entirety.

### FIELD

This application relates to medical systems, and more particularly, to graphical generation of medical records.

### BRIEF SUMMARY OF SELECTED EXAMPLES

In one embodiment, a method for generating a medical record, comprises: rendering, on a display, an anatomic representation; receiving an input, from an input device, having health data, wherein the input is text-based, visual-based, audio-based, and/or based on an interaction with the anatomic representation; processing the input to generate processed health data, the processed health data including a procedure, a diagnosis, a name of an anatomic site, and/or a description of an anatomic site; rendering, on the display, a marked anatomic site on the anatomic representation that is based on the processed health data, or an isolated anatomic representation having a marked anatomic site that is based on the processed health data; selectively rendering, on the display, a mirror image of the anatomic representation with the marked anatomic site or the isolated anatomic representation with the marked anatomic site based on a user preference or context; selecting one of a plurality of templates, each of the templates having one or more fields; populating one of the fields with the anatomic representation with the marked anatomic site or the isolated anatomic representation with the marked anatomic site to generate a populated selected template; and generating a health record including the populated selected template.

In certain embodiments, the anatomic representation includes a plurality of predefined anatomic sites. In certain embodiments, one of the predefined anatomic sites is located on and associated with an anatomic region with one or more subregions. In certain embodiments, the interaction with the anatomic representation includes generating a preview of the anatomic region with the one or more subregions associated with the one of the predefined anatomic sites. In certain embodiments, the input is a scanned image of a physical print of the anatomic representation with markups, wherein the markups include the health data. In certain embodiments, the physical print of the anatomic representation with markups includes orientation markers and processing the input includes detecting orientation markers and normalizing the axis based on the orientation markers. In certain embodiments, processing the input is performed using a language model, a vision-language model, and/or a language-vision model. In certain embodiments, the marked anatomic site is associated with the processed data. In certain embodiments, the description of the anatomic site includes a relationship between the marked anatomic site and another anatomic site, wherein the relationship includes a distance between the marked anatomic site and the another anatomic site, a spatial relationship between the marked anatomic site and the another anatomic site, and/or a data-based relationship between the marked anatomic site and the another anatomic site. In certain embodiments, populating one of the fields includes populating one or more additional fields of the fields with the processed health data. In certain embodiments, processing the input includes detecting a nontechnical term for the processed health data and converting the nontechnical term into a technical term. In certain embodiments, processing the input includes detecting a plurality of languages and translating the plurality of languages into the processed health data. In certain embodiments, the input includes a coded input and processing the input includes decoding the coded input into the processed health data. In certain embodiments, generating the health record includes formatting the health record into a database record suitable for an electronic health record database.

In one embodiment, a system for generating a medical record, comprises: a processer; a medium in communication with the processor, wherein the medium is tangible, non-transitory, and computer readable; processer-executable instructions stored on the medium, the processor-executable instructions defining a mapping platform including a data processing module, a knowledge base module, and a generation module; a display in communication with the medium; and an input device in communication with the medium and display; wherein the mapping platform is configured to: render, using the processor, an anatomic representation of a human on the display, receive, from the input device, an input having health data, wherein the input is text-based, visual-based, audio-based, and/or based on an interaction with the anatomic representation, process, using the data processing module, the input to generate processed health data, the processed health data including a procedure, a diagnosis, a name of an anatomic site, and/or a description of an anatomic site, render, using the processor, on the display, a marked anatomic site on the anatomic representation that is based on the processed health data, or an isolated anatomic representation having a marked anatomic site that is based on the processed health data, selectively rendering, using the processer, a mirror image of the anatomic representation with the marked anatomic site or the isolated anatomic representation with the marked anatomic site on the display based on a user preference; selecting one of a plurality of templates, from the knowledge base module, each of the templates having one or more fields; populating one of the fields, using the generation module, with the anatomic representation with the marked anatomic site or the isolated anatomic representation with the marked anatomic site to generate a populated selected template; and generating, using the generation module, a health record including the populated selected template. In certain embodiments, the marked anatomic site can be a pin within an anatomic site path or path group, or a highlighted anatomic site path or path group.

In certain embodiments, the system further comprises a printer configured to print a physical health record. In certain embodiments, the input device includes an image capturing device configured to scan a physical representation of the anatomic representation with markups, wherein the markups include the health data. In certain embodiments, the processor and the medium are located on one or more servers. In certain embodiments, the processor, the medium, and the display are located on a mobile phone, a tablet, a laptop, a computer, a microphone, a speaker, a headset, goggles, glasses, a contact lens, and/or an electronic device as non-limiting examples.

In one embodiment, a tangible, non-transitory, and computer-readable medium having processer-executable instructions stored thereon that when executed by a processor causes a method for generating a medical record, comprises: rendering, on a display, an anatomic representation; receiving an input, from an input device, having health data, wherein the input is text-based, visual-based, audio-based, and/or based on an interaction with the anatomic representation; processing the input to generate processed health data, the processed health data including a procedure, a diagnosis, a name of an anatomic site, and/or a description of an anatomic site; rendering, on the display, a marked anatomic site on the anatomic representation that is based on the processed health data, or an isolated anatomic representation having a marked anatomic site that is based on the processed health data; selectively rendering, on the display, a mirror image of the anatomic representation with the marked anatomic site or the isolated anatomic representation with the marked anatomic site based on a user preference; selecting one of a plurality of templates, each of the templates having one or more fields; populating one of the fields with the anatomic representation with the marked anatomic site or the isolated anatomic representation with the marked anatomic site to generate a populated selected template; and generating a health record including the populated selected template. In certain embodiments, the marked anatomic site can be a pin within an anatomic site path or path group, or a highlighted anatomic site path or path group, that can be marked physically (e.g., on paper) or digitally through a physical input device (e.g., a touchscreen) as non-limiting examples.

### DESCRIPTION OF FIGURES

FIG. 1A illustrates an example embodiment of a system.

FIG. 1B illustrates a non-limiting example embodiment of a method enabled by the system.

FIG. 1C illustrates a non-limiting example embodiment of a method enabled by the system.

FIG. 1D illustrates an example path with custom axes, segmentation, and relational instructions, directional planes.

FIG. 1E illustrates an example path with associated data.

FIG. 1F illustrates an example embodiment of a system.

FIG. 1G is a screenshot of an example of hierarchical visualization overlay of one embodiment.

FIG. 1H is a screenshot of an example of hierarchical visualization underlay of one embodiment.

FIG. 1I is a cross-section diagram depicting an example representation of anatomic site paths in a single plane.

FIG. 1J is the example representation shown in FIG. 1I shows that custom axes are parallel and perpendicular to the cross-section of anatomic site paths in a single plane, axial mirroring, and auto-relation.

FIG. 1K is an example screenshot of a plane with a rotated bounding box and custom defined axes.

FIG. 1L is an example screenshot of a patient facial diagram displaying enhanced anatomic sites, hierarchical painting of anatomic sites, auto-relation of anatomic sites, hierarchical travel or hierarchical association of anatomic sites, and other capabilities.

FIG. 1M is an example screenshot of a patient facial diagram displaying a translation with quick zoom multidimensional targeting and hierarchical diagnostic distribution painting options.

FIG. 1N is an example screenshot showing map synchronization and pin-level data.

FIG. 1O is an example screenshot of a two-dimensional model with customized axes and defining enhanced detail regions and automatic pin relationships.

FIG. 1P is an example screenshot of a two-dimensional model with customized axes defining an enhanced detail plane, and customization of an axial boundary in the enhanced detail region.

FIG. 1Q is an example screenshot of a marked anatomic site, namely pins on an anatomic site that are automatically related to each other.

FIG. 1R is an example screenshot of a marked anatomic sites, namely hierarchical painting of morphologies with time point tracking.

FIG. 1S is an example screenshot of a marked anatomic sites, hierarchical painting with associated treatment recommendations.

FIG. 1T depicts an example printed output of targeted, isolated and combined visual previews with associated treatment recommendations.

FIG. 1U depicts an example digital regimen map with associated treatment recommendations on combined and isolated visual previews.

FIG. 1V depicts an example printed output with associated treatment recommendations with isolated visual previews and simplified, combined anatomic site descriptions.

FIG. 1W is an example screenshot of an anatomic site name builder associated with a specific pin along with an isolated visual preview.

FIG. 1X depicts an example progressive linguistic and visual sub-segmentation of a dynamic anatomic address.

FIG. 1Y is an example partial screenshot of dynamic anatomic addresses with select patient characteristics.

FIG. 1Z is an example partial screenshot of a dynamic anatomic addresses with alternative selected patient characteristics to dynamically filter the map.

FIG. 2A is an example flowchart of a method enabled by the system.

FIG. 2B is an example screenshot of an anatomic site code translator.

FIG. 2C is an example screenshot of an exemplar anatomic site name to code translator.

FIG. 2D is an example screenshot of another exemplar anatomic site name to code translator.

FIG. 3A depicts an example flowchart of an embodiment of a method.

FIG. 3B depicts an example pathology requisition form.

FIG. 3C depicts an example timeline of dynamic anatomic site history.

FIG. 3D depicts an example of a method enabled by the system for a collaborative logbook with different entities having different permissions.

FIG. 3E is an example screenshot of a collaborative logbook example.

FIG. 3F depicts an example automatically replotted anatomic site with associated diagnosis and diagnosis extension data blocks.

FIG. 3G depicts an example automatically generated Mohs map.

FIG. 4A is an example omnidirectional data model that illustrates the capabilities of the data block engine.

FIG. 4B is an example screenshot of FIG. 1N with portions translated to Chinese and added symbolically delimited and/or defined filename.

FIG. 4C is an example screenshot showing options to customize the anatomic site name sequence configuration and the data block within a file name builder.

FIG. 4D is an example screenshot of a modal view of a thumbnail image and its accompanying symbol delimited and symbol defined file name.

FIG. 4E is an example screenshot of a coordinated anatomy data in correspondence with a color-coded legend, and symbolic definitions for the anatomic site group.

FIG. 4F is an example legend of representative symbolic searches within a reSearch engine.

FIG. 4G is an example legend of representative application examples to re-create pins on regions of interest.

FIG. 4H is an example screenshot of an artificial intelligence collated patient history in an anatomic region of interest made possible by data blocks.

FIG. 5A is an example flowchart of a method enabled by the system.

FIG. 5B is an example representative annotated paper record with handwritten markings.

FIG. 5C illustrates an example digital interpretation of only the handwritten markings of FIG. 5B.

FIG. 5D is an example generated electronic version of the paper record in FIG. 5B.

FIG. 5E in an example anatomic representation in paper form, namely an anatomic map with markups, e.g., handwritten annotations.

FIG. 5.F illustrates an example digital interpretation of the handwritten markings overlayed on the digital photo of the paper form in FIG. 5E.

FIG. 5G is an example electronic record generated from the paper form in FIG. 5E with automatic documentation and mapping of correct procedures, diagnoses, marked anatomic sites, notes, patient demographics, and billing codes.

FIG. 5H is an example anatomic representation in paper form, namely an anatomic map in Chinese with hand-colored anatomic distributions.

FIG. 5I is an example generated electronic version of the paper record in FIG. 5H with detected color, area, intensity, and distribution in Chinese.

FIG. 5J is an example generated electronic version depicted in FIG. 5I translated to English.

FIG. 6A is an example flowchart illustrating information management by a method enabled by the system.

FIG. 6B is an example anatomic representation, namely a shadow chart.

FIG. 6C is an enlarged view of a portion of the example shadow chart in FIG. 6B.

FIG. 6D is an enlarged view of an alternate portion of the example shadow chart in FIG. 6B.

FIG. 6E is an example captured image of an annotated and marked up version of the example shadow chart in FIG. 6B.

FIG. 6F is an enlarged view of a portion of the example capture from FIG. 6E.

FIG. 6G is an enlarged view of an alternate portion of the example capture from FIG. 6E.

FIG. 6H is an example screenshot showing FIG. 6F converted to a digital record.

FIG. 6I is an example screenshot showing FIG. 6G converted to a digital record.

FIG. 6J is an example printed shadow chart showing color annotations.

FIG. 6K is an example screenshot showing FIG. 6J converted to a digital record with correlating diagnoses based on color detection.

FIG. 6L is an example screenshot showing FIG. 6J converted to a digital record with correlating diagnoses based on color detection automatically translated to English.

FIG. 6M is an example screenshot showing a visual alert on a shadow chart.

FIG. 7A. illustrates an example simplified block diagram of a method relevant to anatomy and morphology enabled by the system.

FIG. 7B shows example morphology detections of the example photo from FIG. 10D.

FIG. 7C shows an example combined anatomic map and summary of detections of the example photo from FIG. 10E, with English and Chinese correlates.

FIG. 7D illustrates one embodiment of an automatically encoded diagnosis and diagnosis extensions.

FIG. 7E is an example screenshot illustrating coded and symbolic translation of an anatomic site description.

FIG. 8A illustrates an example block diagram of an omnidirectional neural network for ranges, collections, and categorizations of data.

FIG. 8B shows a screenshot of one embodiment of interactive range categorizations and data collections used to describe and encode anatomy, diagnosis, and procedure.

FIG. 8C is an example of a diagnosis and diagnosis extensions shown in Spanish with full translations and encodings before natural language processing.

FIG. 8D shows a screenshot of an example of the visual ranges of anatomy under a given point or site.

FIG. 9A illustrates example marked anatomic sites or “areas of interest” represented by visible pins on an anatomic representation, namely an anatomic map.

FIG. 9B illustrates an example invisible area of interest on an anatomic map.

FIG. 9C illustrates the example areas of interest from FIG. 9A reproduced at a different point in time.

FIG. 9D illustrates an example area of interest in a void unmapped space.

FIG. 9E illustrates an example relocation of the unmapped area of interest in FIG. 9D to a mapped location.

FIG. 9F illustrates an example reordering of the areas of interest from FIG. 9A and shows a mirror view.

FIG. 9G illustrates a non-limiting example of a method.

FIG. 9H illustrates a non-limiting example of a method.

FIG. 9I illustrates a non-limiting example of a method.

FIG. 9J illustrates a non-limiting example of a method.

FIG. 9K illustrates a non-limiting example of a method.

FIG. 9L illustrates a non-limiting example of a method.

FIG. 9M illustrates a non-limiting example of a method.

FIG. 9N illustrates a non-limiting example of a method.

FIG. 9O illustrates a non-limiting example of a method.

FIG. 9P illustrates a non-limiting example of a method.

FIG. 9Q illustrates a non-limiting example of a method.

FIG. 9R illustrates a non-limiting example of a method.

FIG. 9S illustrates a non-limiting example of a method.

FIG. 9T illustrates a non-limiting example of a method.

FIG. 9U illustrates a non-limiting example of a method.

FIG. 9V illustrates a non-limiting example of a method.

FIG. 9W illustrates a non-limiting example of a method.

FIG. 9X illustrates a non-limiting example of a method.

FIG. 9Y illustrates a non-limiting example of a method.

FIG. 10A is an example anatomic representation, namely a three-dimensional model depicting a dynamic anatomic map with associated addresses.

FIG. 10B is an example anatomic representation, namely a three-dimensional avatar visualization depicting patient characteristics.

FIG. 10C is an example anatomic representation, namely a two-dimensional diagram of multiple anatomic perspectives depicting the same pin location.

FIG. 10D is an example patient photograph.

FIG. 10E is an example patient photograph overlayed with the accompanying hierarchical dynamic anatomic addressing method enabled by the system.

### DETAILED DESCRIPTION OF SELECTED EXAMPLES

The following detailed description and the appended drawings describe and illustrate various examples of systems, methods, embodiments, engines, calculations, models, information systems, and algorithms that are stored on tangible, non-transitory, and computer-readable medium having processor-executable instructions stored thereon. The description and illustration of these examples can enable one skilled in the art to make and use various examples of multidimensional labeling, coordinated language model type modeling, relational capabilities of the systems, artificial intelligence, and generative capabilities. A non-limiting list of other capabilities described herein include tracking, translation, reproducibility, transformation, mirroring, aligning, targeting, extracting, detecting, describing, enhancing, calculating, communicating, overlaying, underlaying, encoding, searching, modifying, processing, reproducing, collating, and summarizing to name a few. They do not limit the scope of the claims in any manner.

References in the specification to “embodiment” “one embodiment,” “certain embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. The terms “for example,” “e.g.,” “as one example”, “as an example”, “as one non-limiting example”, etc. indicate one or more non-limiting examples, even when the “non-limiting” term is not expressly written in the description. When one component of a system or method is listed with “e.g.,” or “e.g., enabled by”, this is intended to provide a non-limiting example or non-limiting examples, and it is contemplated that other components of the system may be substituted, added, subtracted, or otherwise modified by one skilled in the art. In the teachings herein, the system can form a method, and conversely the method can form a system. Also, an anatomic “visualization” can be a type of an anatomic “representation.” The terminology does not limit the scope of the claims in any manner.

Traditionally, there has been no way to use language models, language-vision models, vision-language models, and/or other data models, to precisely, accurately, and reproducibly describe, visualize, track, and target anatomic sites or other health findings. Numerous ontologies exist, but there is not a single model that unifies language, encodings, and vision across multidimensional space and through time for anatomy and health data. Disjointed record systems that use different languages or human generated language as free text often lack precision and reproducibility, and present numerous issues solved by the teachings herein. The relationships between two or more points or sites are established and described through a combination physical proximities or distances (such as coordinates, overlays, or underlays on an image, map, avatar, or illustration), data-based proximities (such as in a hierarchical or relational database stored on a server), semantic/linguistic comparisons (such as two anatomic sites that have the same or similar linguistic, coded, or symbolic name elements), customized axes (on individual or grouped paths in a map file, avatar, diagram, or image, for example), and directional planes (independent path or path groups that supply separate directional information and custom axes regardless of the customized axes on the anatomic site paths and path groups, as one example). The magnitude of distance between points or areas in one or more axes can also be described with linguistic, coded, and symbolic, and calculated (such as numerical measurements) language through a processor and plurality of logical comparisons from one or more databases stored in the physical medium. Path or path groups stored within or extrapolated from a multimedia file stored in the physical medium also contain segmentation instructions stored within their metadata, and those segmentation instructions communicate with the database and a processor in certain embodiments, to provide enhanced descriptions and visualization of paths or path groups with progressive segmentation (e.g., representation), regressive segmentation, progressive coordination, regressive coordination, and/or enhanced relational descriptions through any combination of language models, vision-language models, language-vision models, and/or Dimensionally Extended 9-Intersection Models enabled by a coordinated language model engine comprising of databases stored on physical storage mediums on physical servers that are in communication with physical processor, output devices, displays, paper or other printable media, and captured or stored multimedia files within a physical storage medium.

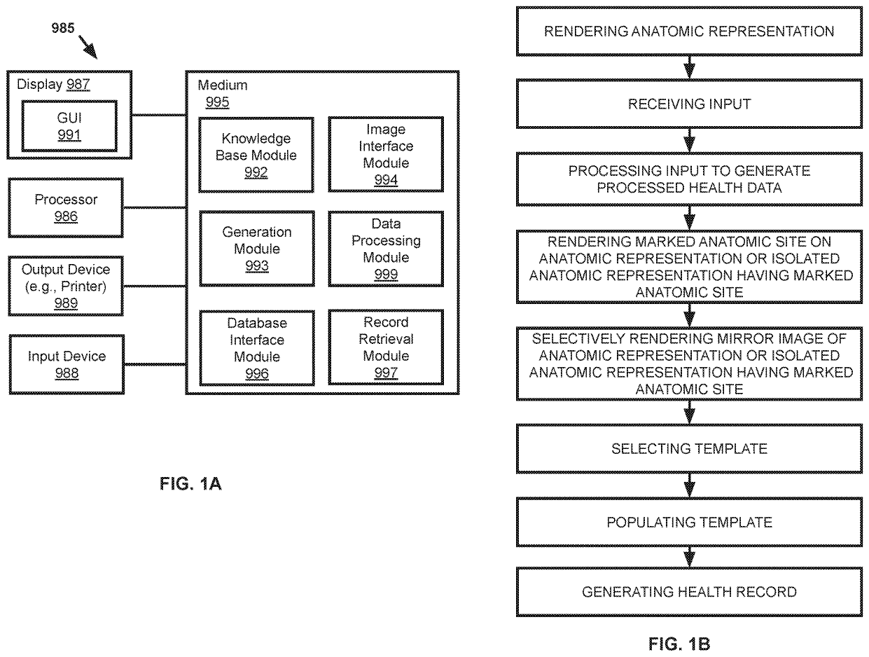

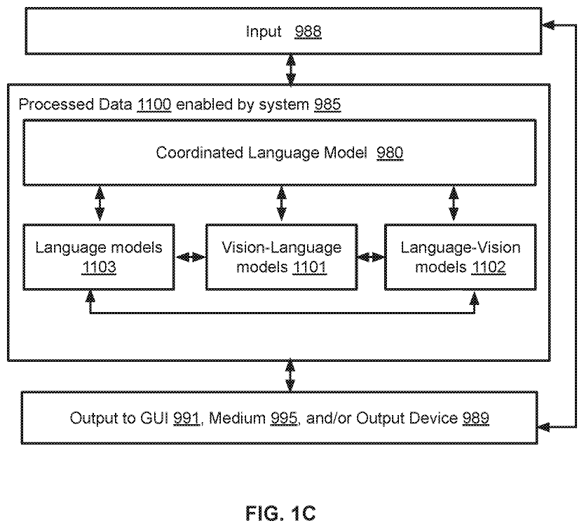

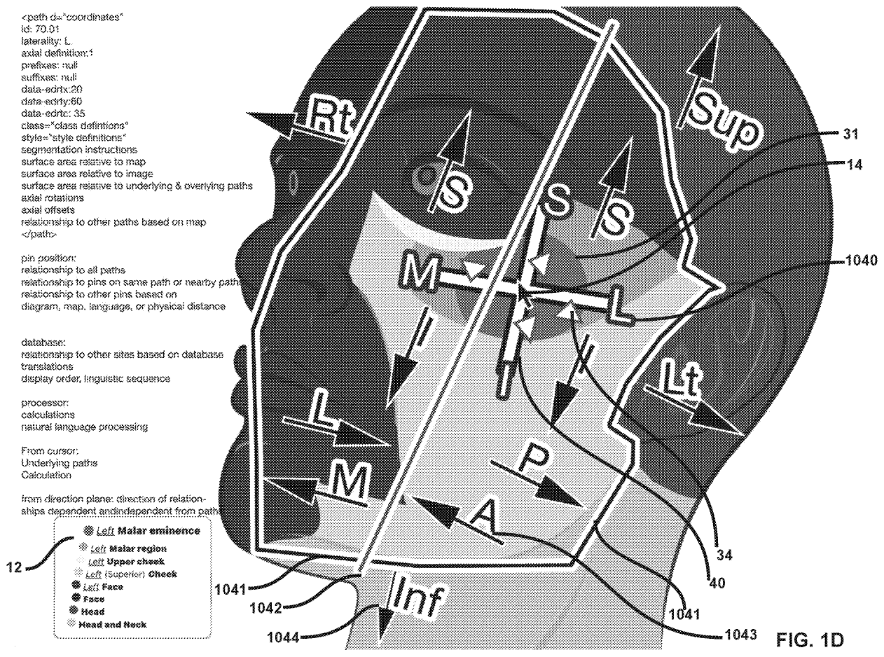

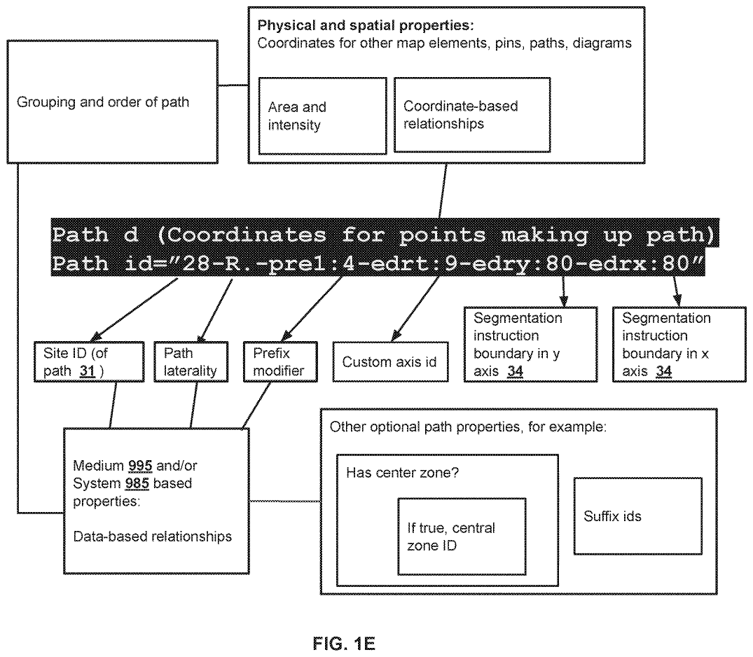

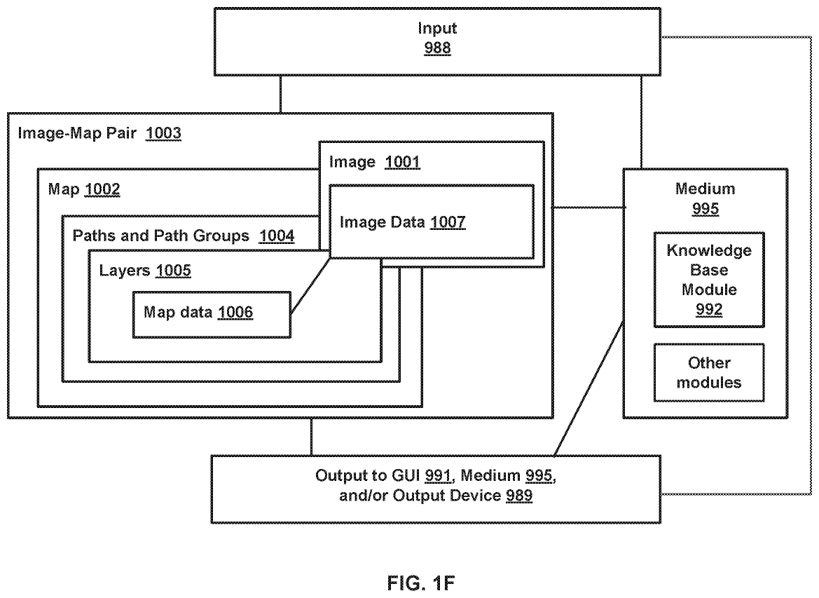

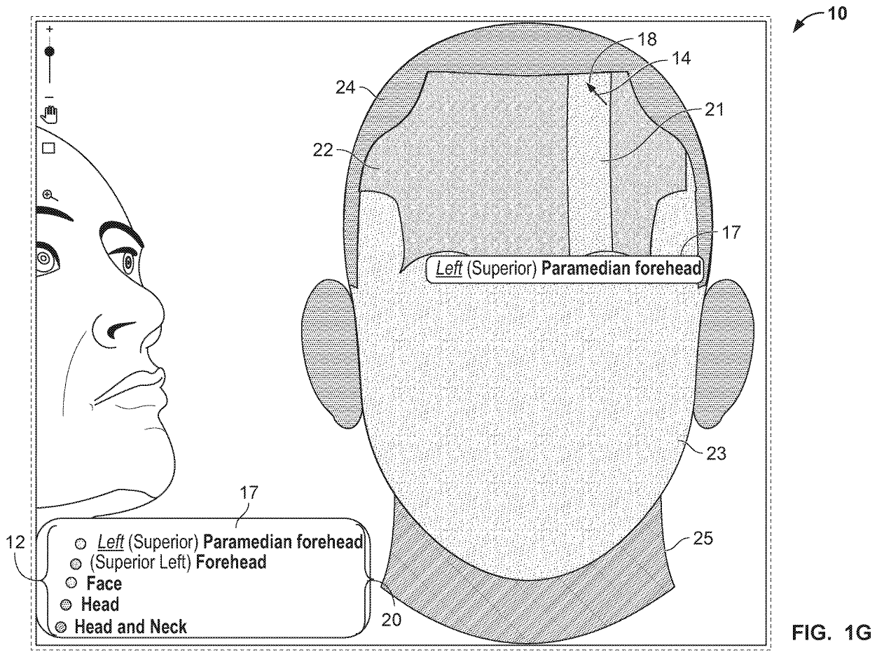

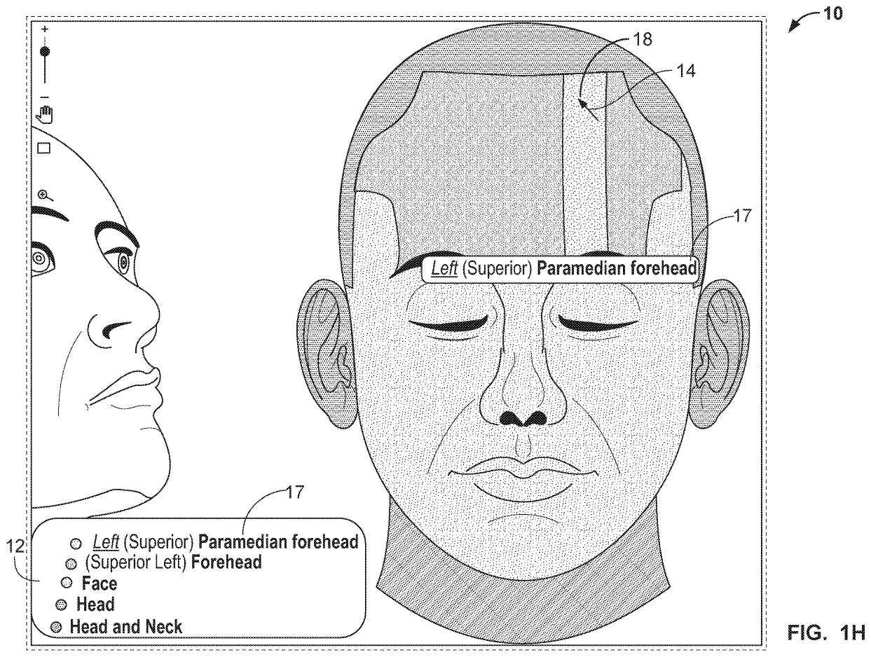

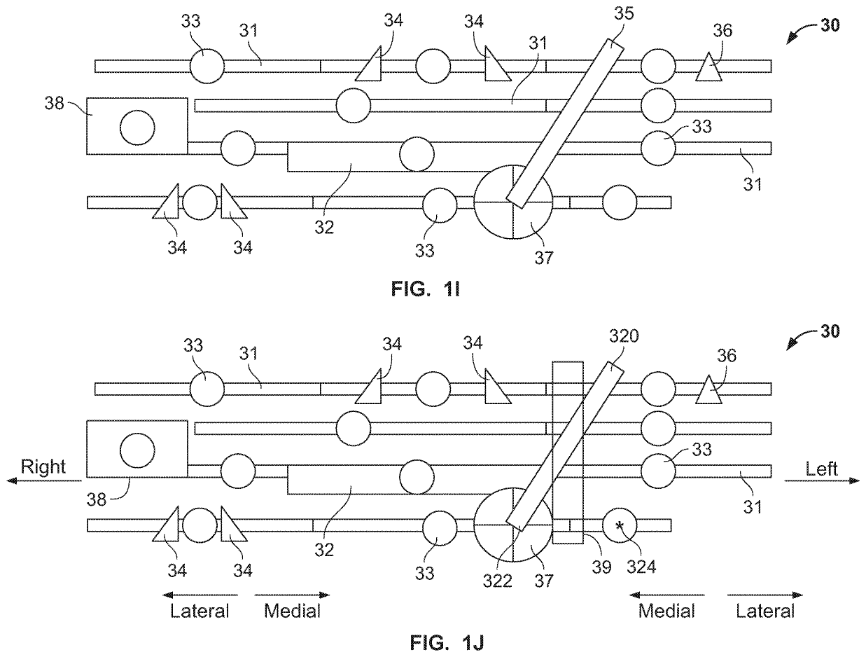

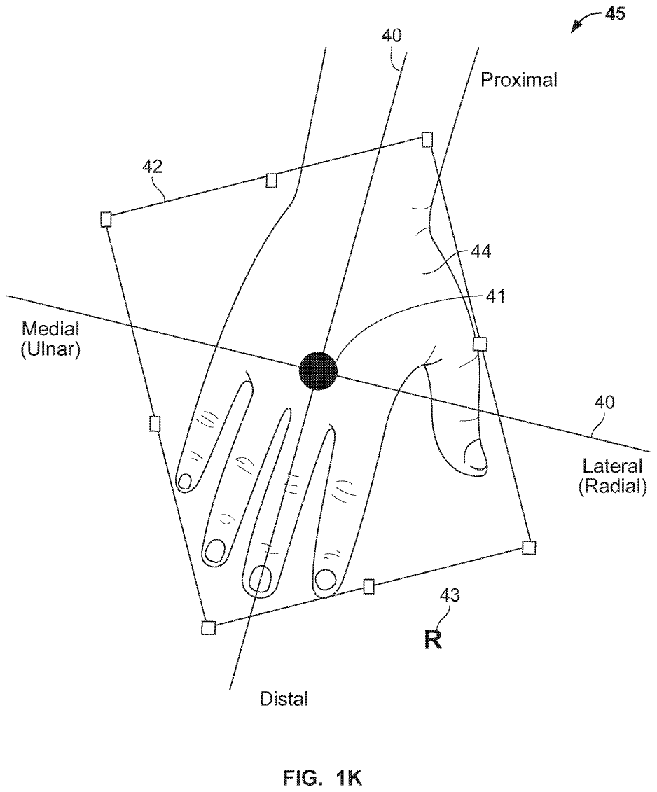

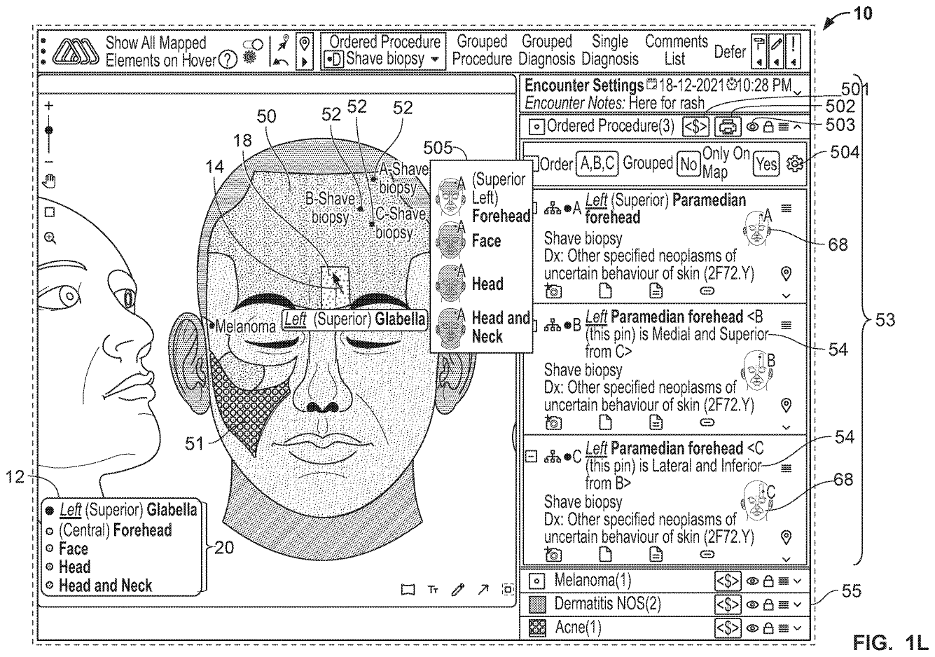

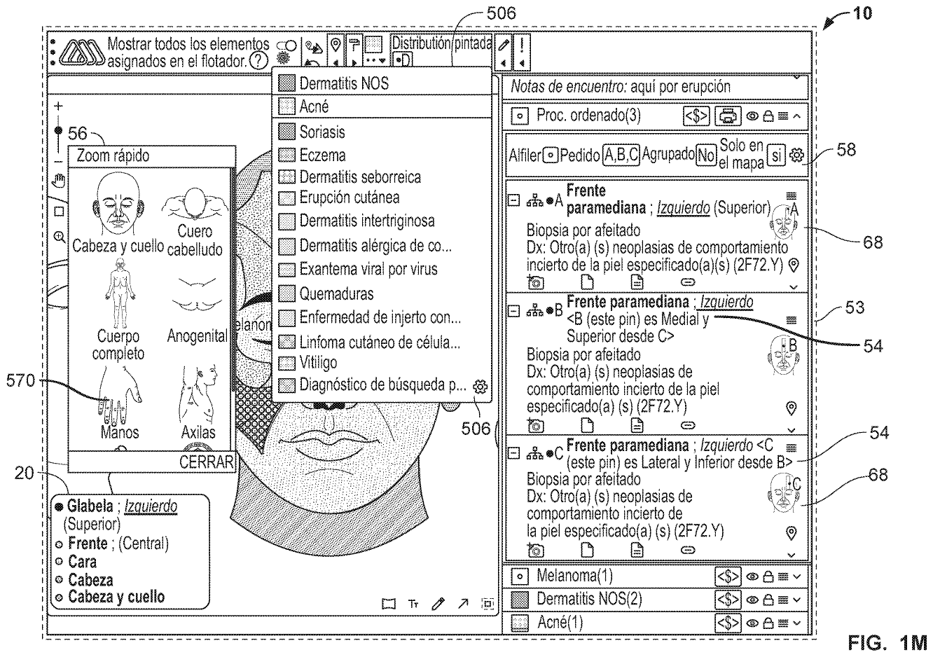

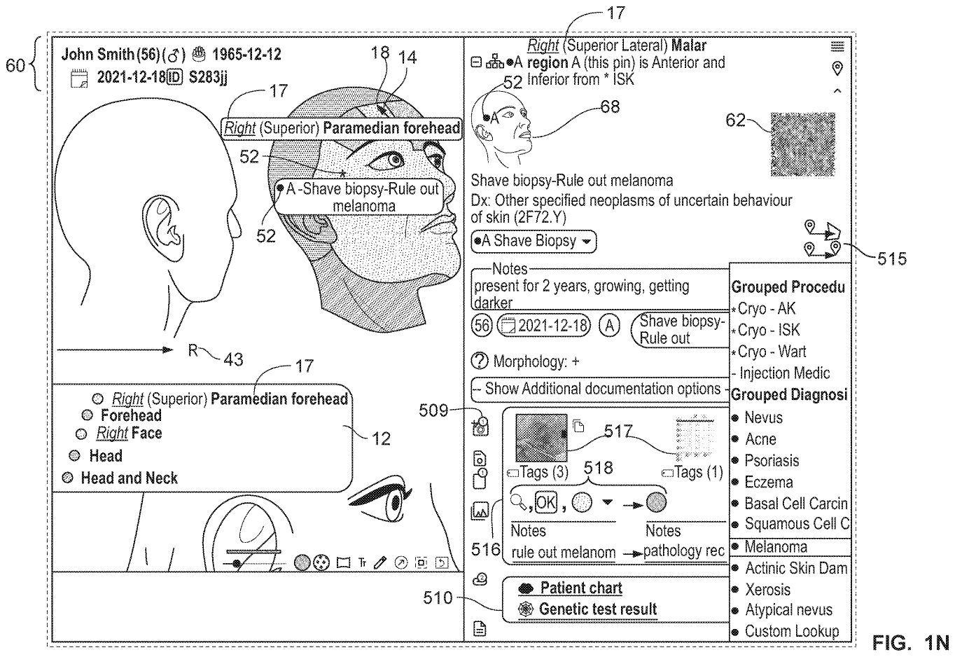

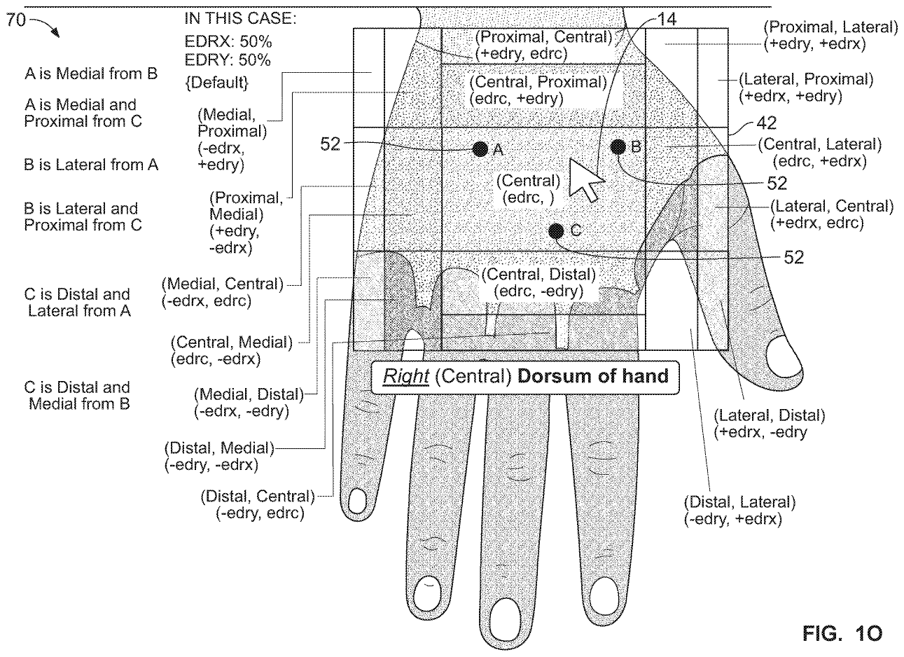

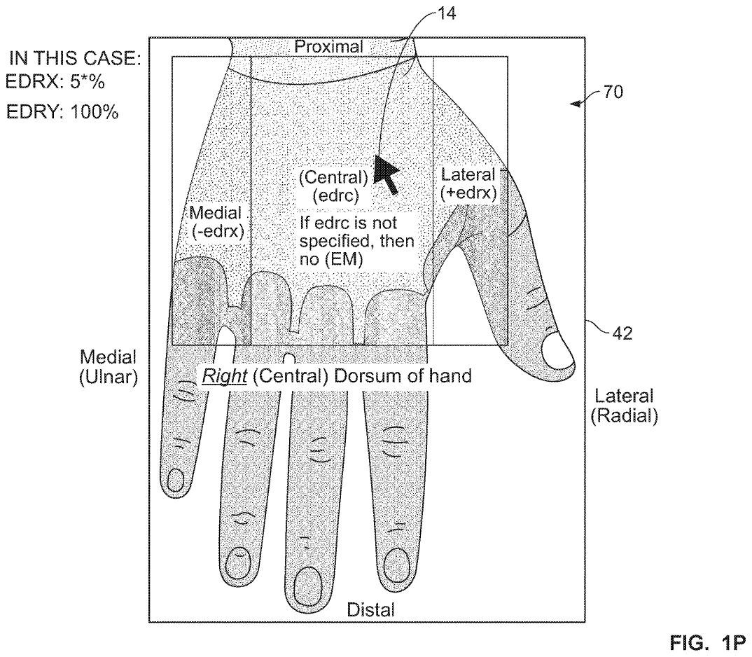

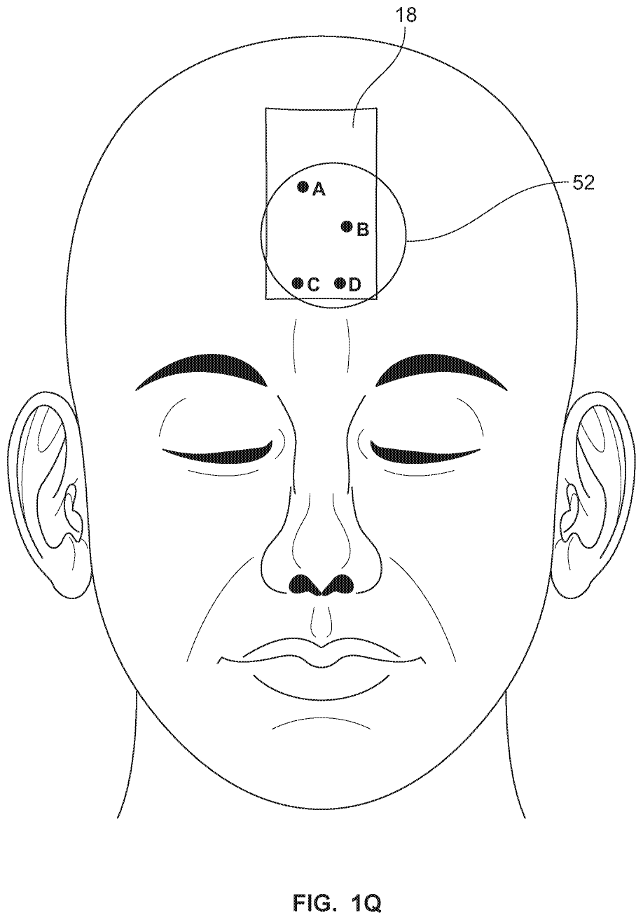

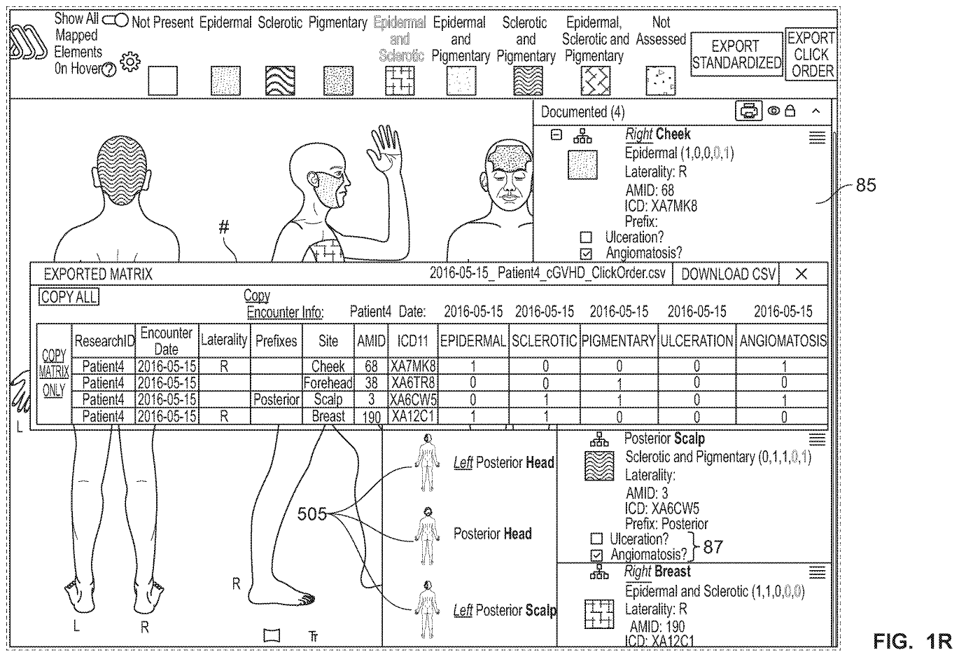

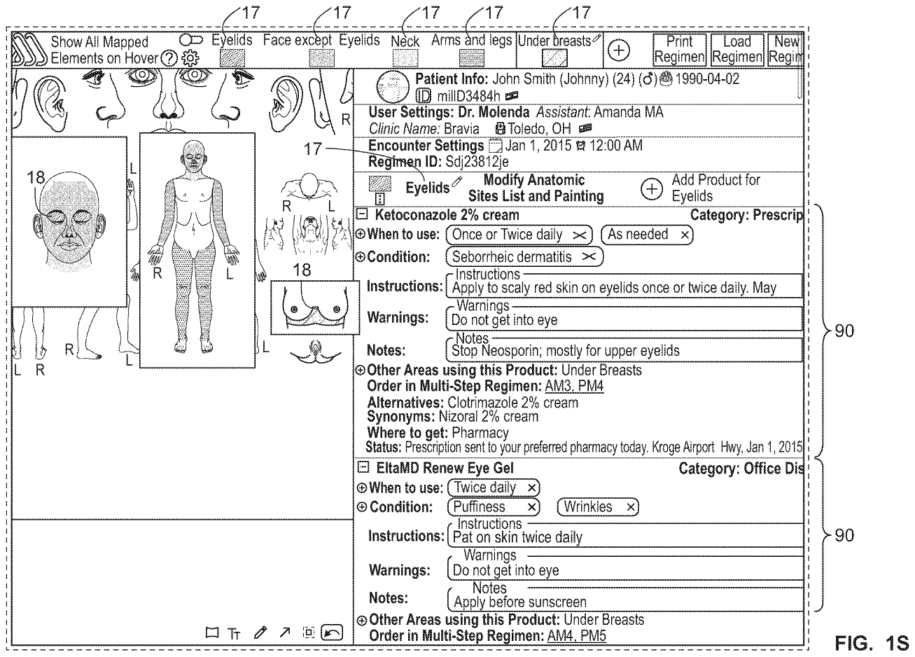

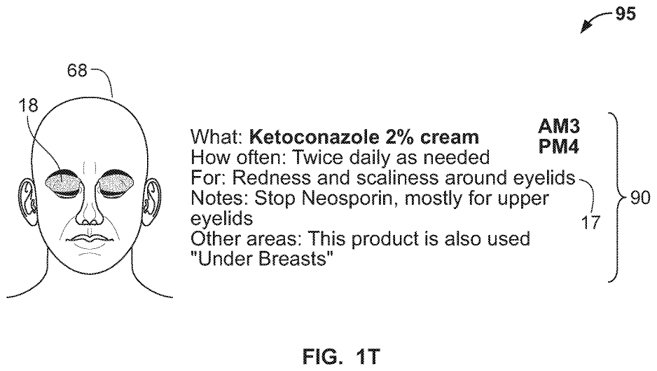

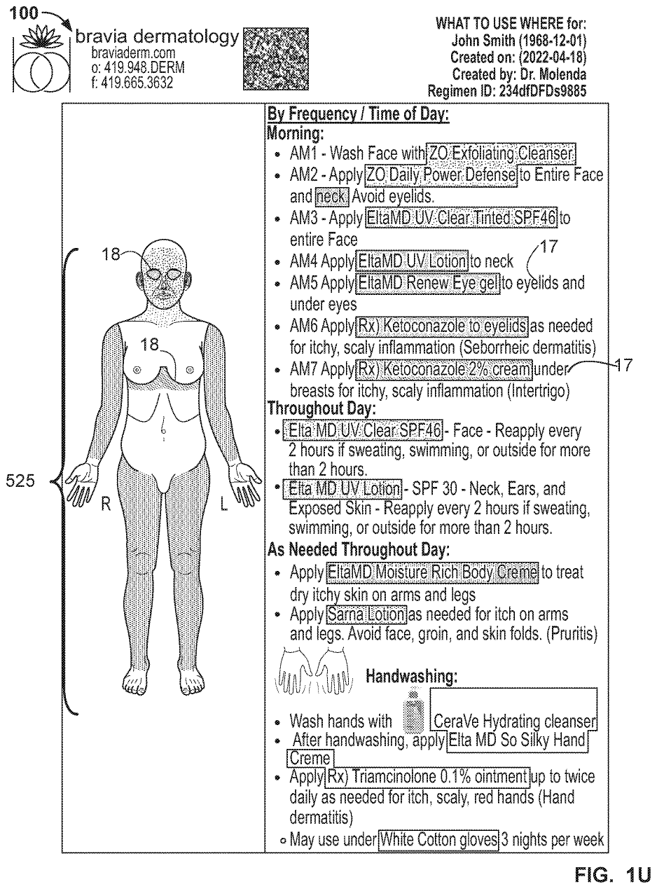

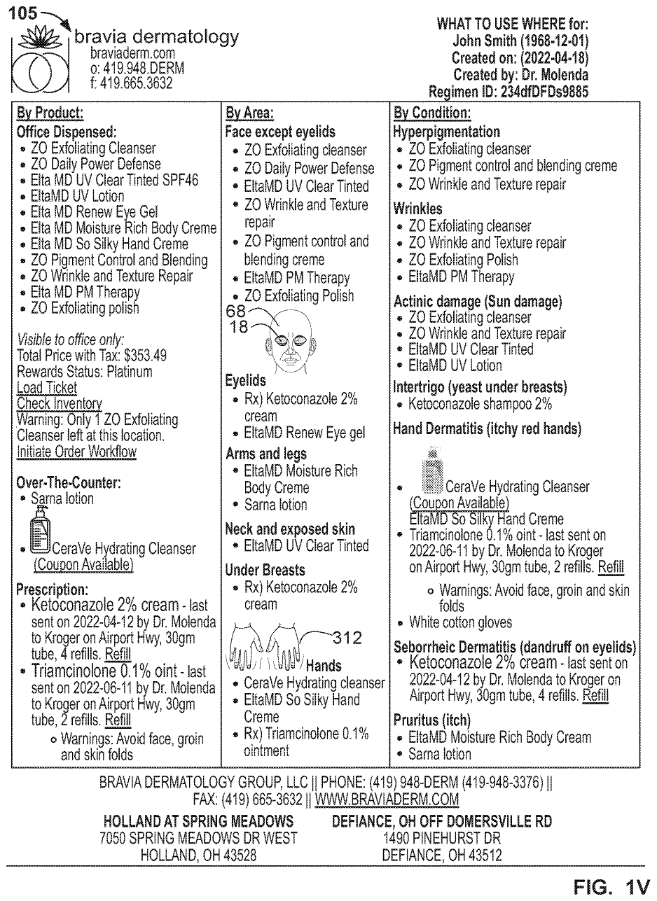

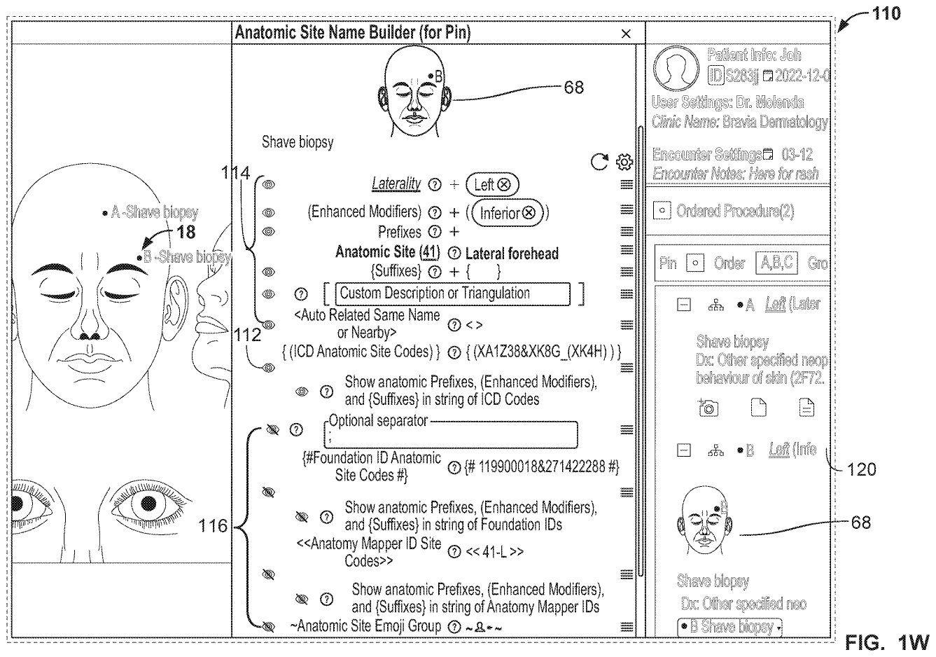

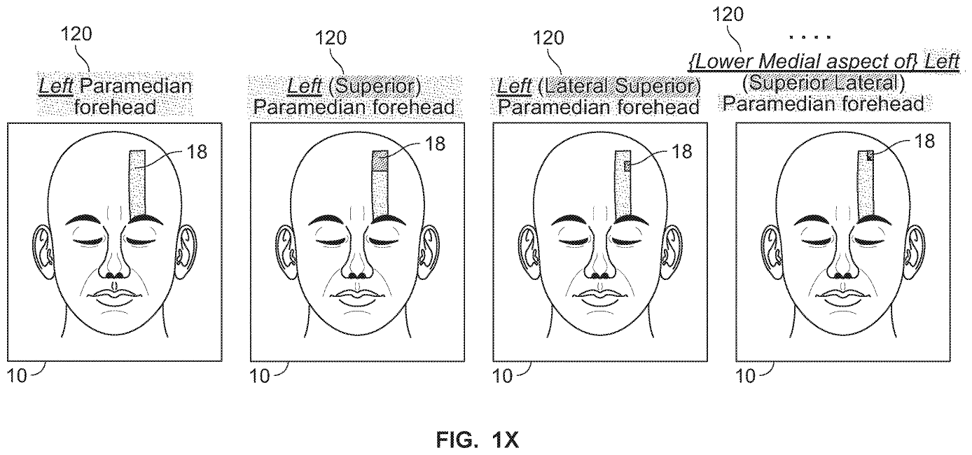

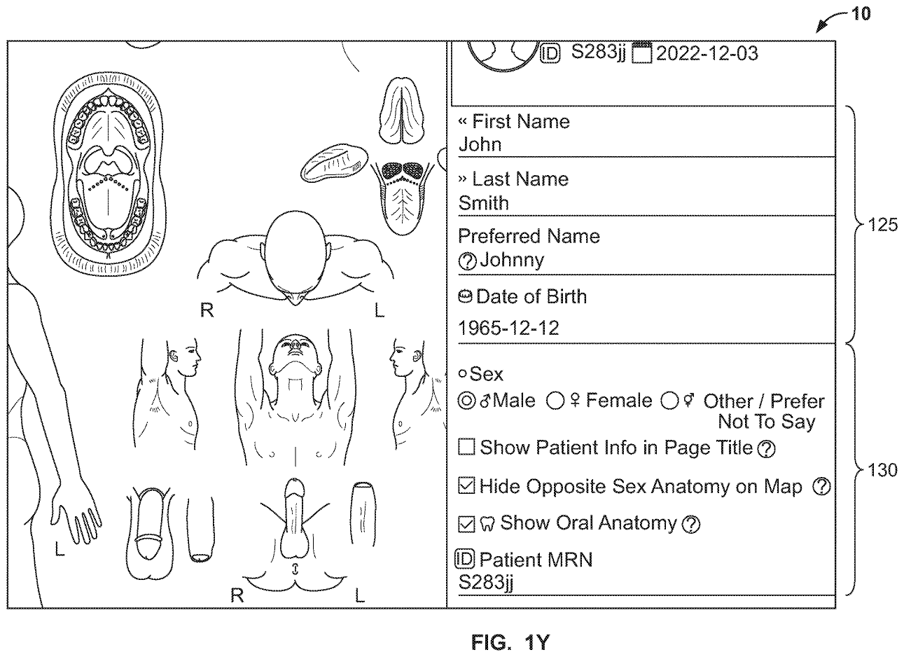

FIG. 1A illustrates an example embodiment of a system 985. The system 985 includes a processor 986, a medium 995, a display 987, an input device 988, and/or an output device 989.

The processor 986 can be in communication with the medium 995. The processor 986 can include any type of general or specific purpose processor. In certain embodiments, the processor 986 can include multiple processors. As non-limiting examples, the processor 986 can include one or more general-purpose computers, special purpose computers, microprocessors, digital signal processors (DSPs), field-programmable gate arrays (FPGAS), application-specific integrated circuits (ASICs), and processors based on a multi-core processor architecture. The processor and the memory can be located on the same device, e.g., a server, a mobile phone, a tablet, a laptop, a computer, a headset, an electronic device, etc.

The medium 995 is a tangible, physical, non-transitory computer readable medium 995 with processor-executable instructions stored thereon. The medium 995 can be located on the same device as the processor 986 or a separate device. In certain embodiments, the processor 986 and the medium 995 are located on one or more servers. The medium 995 can be one or more memories and of any type suitable to the local application environment and can be implemented using any suitable volatile or nonvolatile data storage technology such as a semiconductor-based memory device, a magnetic memory device and system, an optical memory device and system, fixed memory, and removable memory. For example, the medium 995 can comprise of any combination of random-access memory (RAM), read only memory (ROM), static storage such as a magnetic or optical disk, hard disk drive (HDD), solid state drive (SSD), or any other type of non-transitory machine or computer readable media. The tangible medium 995 also enables various network types, such as neural networks and/or artificial neural networks; and models such as data models, language models, language-vision models, vision-language models, coordinated language models, and/or other models; engines such as search engines, research engines, anatomy-data engines, non-anatomy data engines, generation engines, dissection and categorization engines, coordinated language model engines, encoding engines, summarization engines, data-blocking engines, analysis engines, record generation engines, translations engines, morphology mapping engines, form processing engines, mapping engines, coding engines, search engines, and/or visualization engines; and/or capabilities such as artificial intelligence, machine learning, augmented reality, virtual reality, spatial computing, and/or computer vision; when the medium 995 is in communication with the processor 986 and/or other components of the system 985, as non-limiting examples enabled by the teachings herein.

The processor-executable instructions define a mapping platform and when executed by the processor 986, can render a graphical user interface (GUI) 991 on the display 987 thereby enabling a user to interface with the mapping platform. The GUI 991 can include one or more interfaces, also called “screenshots” with interactable objects, e.g., anatomic representations, that will be further described in further detail below. In certain embodiments, the GUI 991 can also be combined with or part of other components of the system such as an output device 989, input device 988, and/or medium 995.

The mapping platform enabled by the system 985 includes a plurality of modules, with non-limiting examples comprising, a knowledge base module 992, a generation module 993, a database interface module 996, a record retrieval module 997, the image interface module 994, and/or a data processing module 999. Each of the plurality of modules can communicate with each other. It should be appreciated that these modules can be combined into fewer modules or separated into additional modules, as desired by one skilled in the art.

The knowledge base module 992 can be configured to receive and store templates, each comprising a set of fields, and in certain embodiments is in communication with the database interface module 996. The generation module 993 can be configured to correlate and select one of templates based on user input, e.g., a selected procedure, a location on an anatomic region, etc., and populate the set of fields of the selected template, e.g., text describing the selected procedure, text describing the location of the anatomic region, a multimedia item associated with the anatomical region of the patient, etc. The generation module 993 can be further configured to output health records, such as digital data to the GUI 991 and output data to output devices 989, e.g., a printer 989, to generate physical outputs, such as labels, images, forms, maps, and/or other health records. It should be appreciated that all examples included throughout these teachings herein are non-limiting example components that illustrate non-limiting embodiments of systems and methods enabled by the system 985 configured to execute instructions stored on the tangible medium 995 in communication with the physical processor 995 and other physical components of the system 985.

The database interface module 996 can be configured to generate a database record suitable for an electronic health based on the populated template. The database interface module 996 also stores, on physical storage medium 995, various different databases, with non-limiting examples including databases related to anatomy, modifiers of anatomy, anatomy visualizations (e.g., representations), health, patient, user, entity (e.g., a practice with multiple physical locations), permissions, and/or encounter data. The record retrieval module 997 can be configured to store a timeline of clinical activity, e.g., a timeline for a selected anatomical location, a particular diagnosis, etc. The image interface module 994 can be configured to process a received visualization for use within the mapping platform, e.g., processing a received image from an image capturing device as a non-limiting example of an input device 988. The image interface module 994 can be a submodule of the data processing module 999. Additional information regarding these modules, along with others, will be described in further detail below.

The data processing module 999 is configured to process and convert data received from the input device 988 or other modules into processed data, as shown in FIG. 1A, which can be used as an input or output for other modules. The data processing module 999 can include various language models, language-vision models, vision-language models, and/or other data and/or spatial models working separately or together, forming a coordinated language model engine as one non-limiting example. Various models can work omnidirectionally as shown in one example, with a language model, a vision-language model, and a language-vision model working together to form an example model that coordinates language, semantics, code, and vision. Non-limiting output examples include translations, coordinates, paths, maps, visualizations, pins, distributions, images, interactive language, encodings, relationships, diagnoses, morphologies, detections, categorizations, timelines, histories, forms, labels, descriptions, avatars, libraries, calculations, symbols, bookmarks, filenames, modified metadata, modified data, organized data, collated data, vertices, groups, axial changes such as mirroring, axial definitions, offsets, color coded legends, heatmaps, texture and pattern application, skin tone, skin type, procedures, tracking points, and/or dynamic anatomic addresses. It should be appreciated that the aforementioned functions of the modules are non-limiting examples, and other aspects will be discussed throughout the disclosure.

Non-limiting example language models, enabled by the system 985 and modules (e.g. the data processing module 999 as one non-limiting example) stored in the physical medium 995 and processed by the processor 986 include: a language model describing anatomy and relationships, morphology, and health data with healthcare language and synonyms in any linguistic language; a vision-language model using computer vision, augmented reality, virtual reality, or artificial intelligence to detect and describe anatomy which can also calculate coordinates for each detection; a language-vision model that receives language, code or symbols to generate visualizations of anatomy and other health findings related to anatomy, morphology, or procedures; and/or a language-vision model that uses language to plot points and distributions (e.g. coordinates) on visualizations that contain anatomy.

The display 987 can be in communication with the medium 995. The display 987 facilitates presentation of the GUI 991. The display 987 can be located on the same device as the processor 986, the medium 995, or on a separate device. Non-limiting examples of the display 987 can include different display technologies, such as cathode ray tubes (CRT), liquid crystal displays (LCD), Organic light emitting diodes (OLED), electronic paper (e-ink), etc. In certain embodiments, the processor 986, the medium 995, and the display 987 are located on a mobile phone, a tablet, a laptop, a computer, a headset, contact lenses, glasses, a speaker, a microphone, and/or other electronic devices.

The input device 988 can be in communication with the medium 995 and the display 987. The input device 988 enables a user to interact with the mapping platform, e.g., via the GUI 991. The input device 988 can be located on the same device as the display 987 or on a device separate from the display 987. Non-limiting examples of the input device 988 include tablets, smartphones, computers, headsets, cameras, scanners, goggles, sensors, speakers, microphones, other electronic devices, etc. In certain embodiments, the input device 988 includes an image capturing device configured to scan a physical representation with markups of the anatomic representation, as will be discussed in further detail below. The input device 988 can be configured to enable a user to transmit data to the mapping platform, for example, text, images, and video. The mapping platform can receive a multiple of different inputs via the input device 988. Non-limiting examples of inputs can include text, language, code, coordinates, images, illustrations, visualizations, handwriting, typed characters, QR codes, avatars, 3D models, videos, eye movements, gestures, markups, annotations, selection on the GUI 991, and/or voice as non-limiting examples. The input device 987 can also be paper, such as a map or visualization (e.g., representation) printed on paper, or an electronic device combined with paper, such as a pen that detects the position of the markup and/or annotation as a user marks up a paper, as other non-limiting examples.

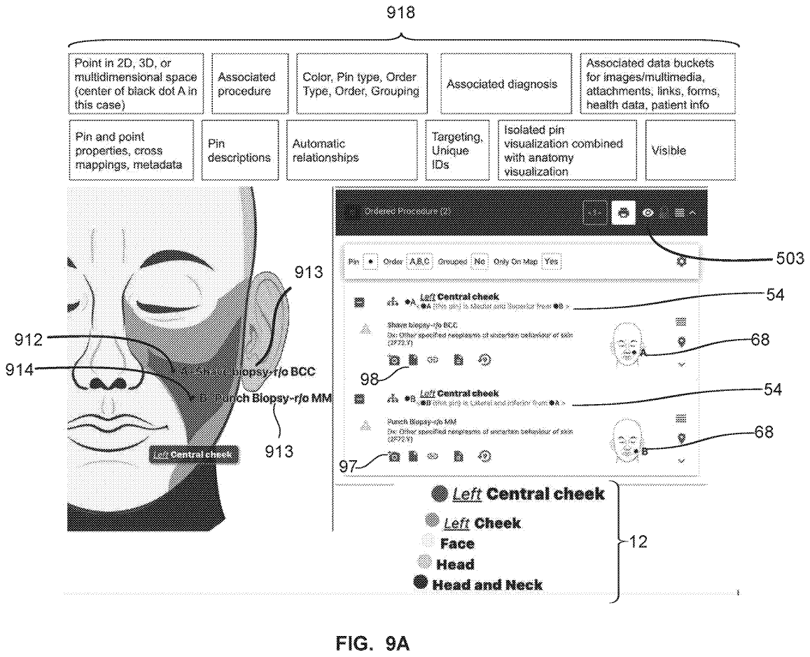

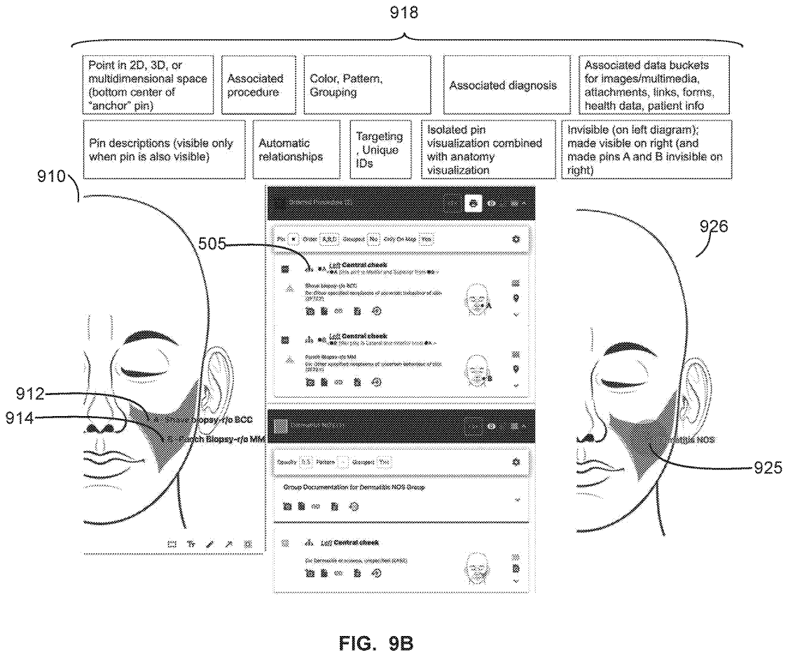

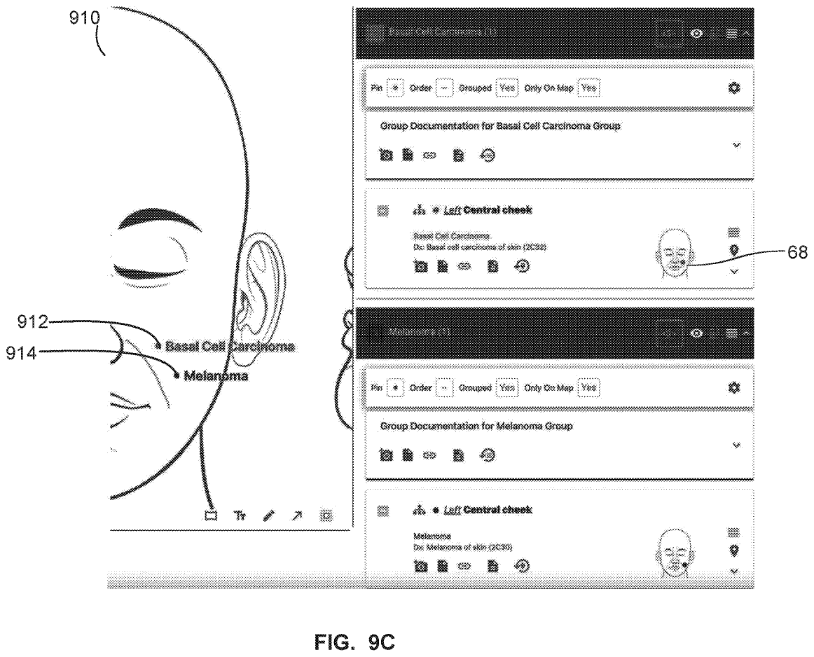

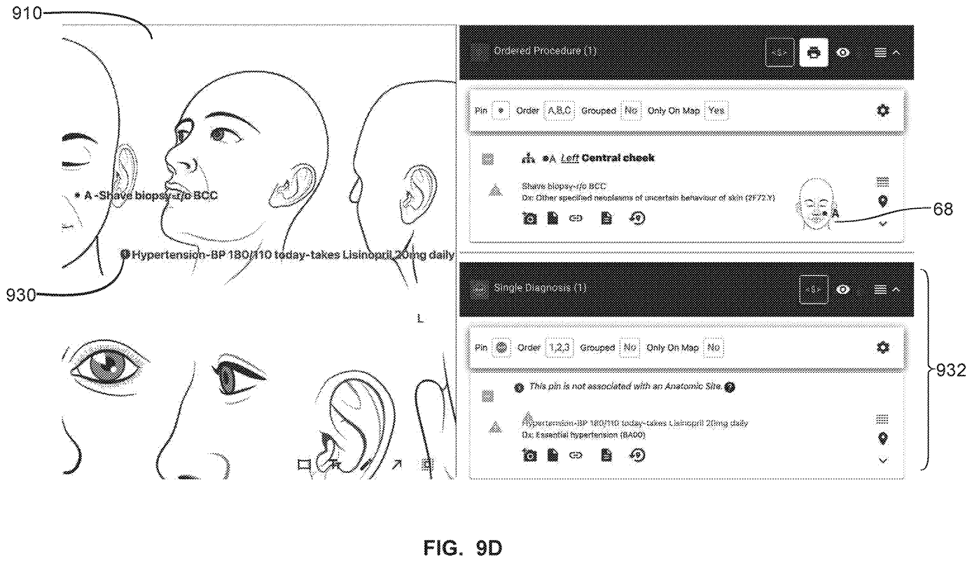

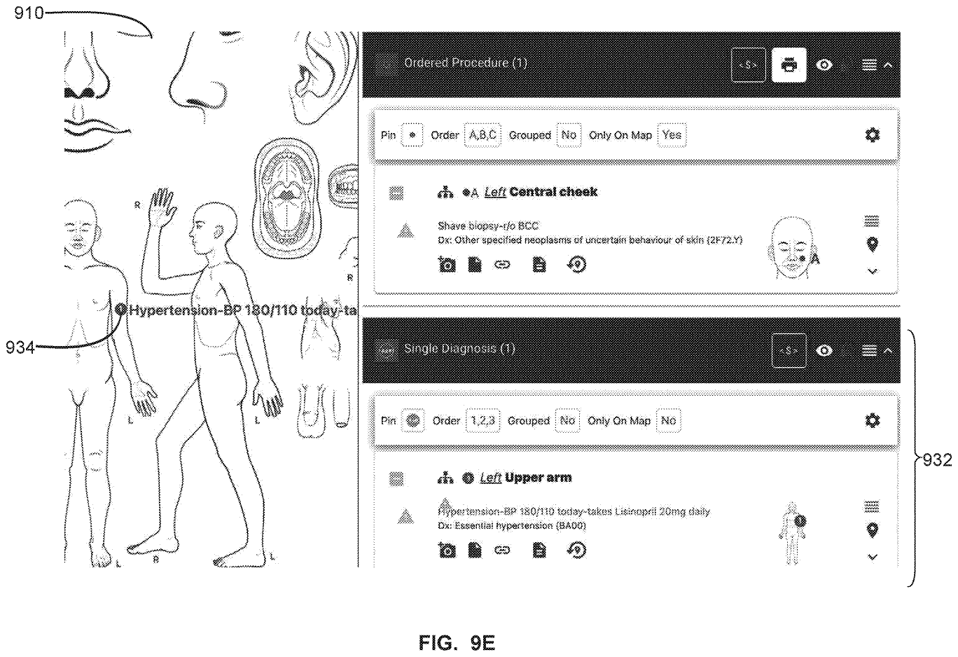

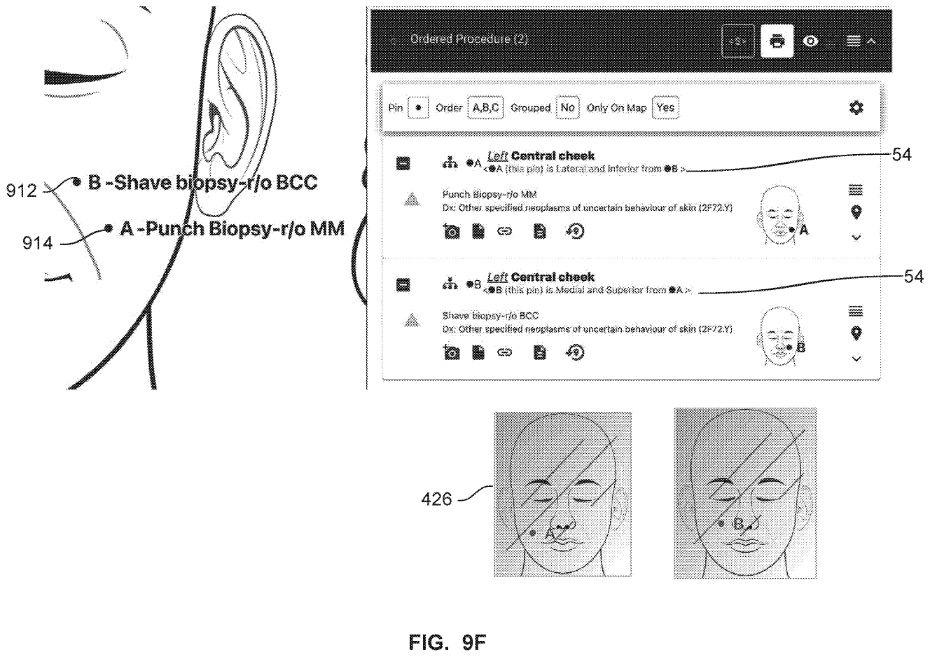

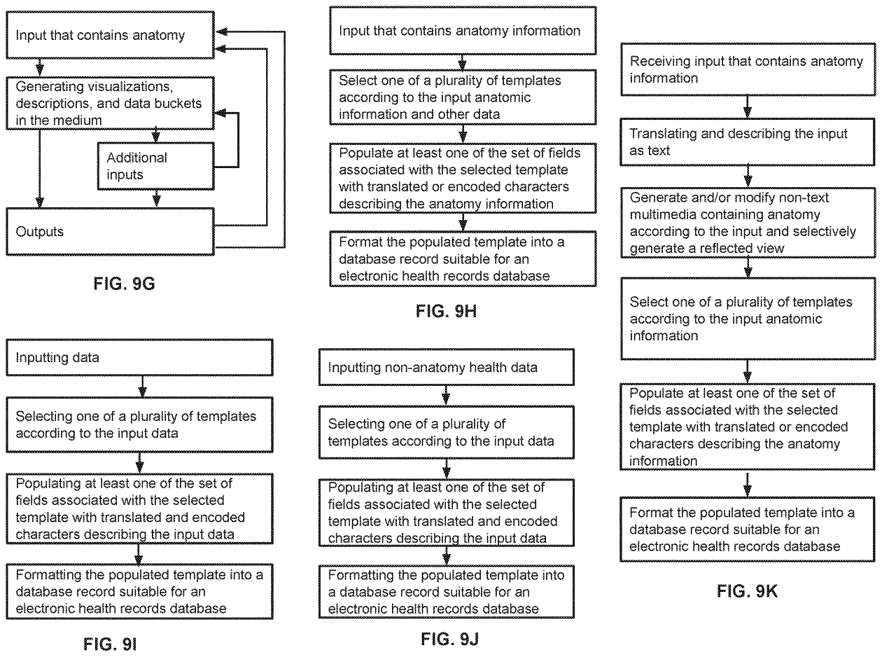

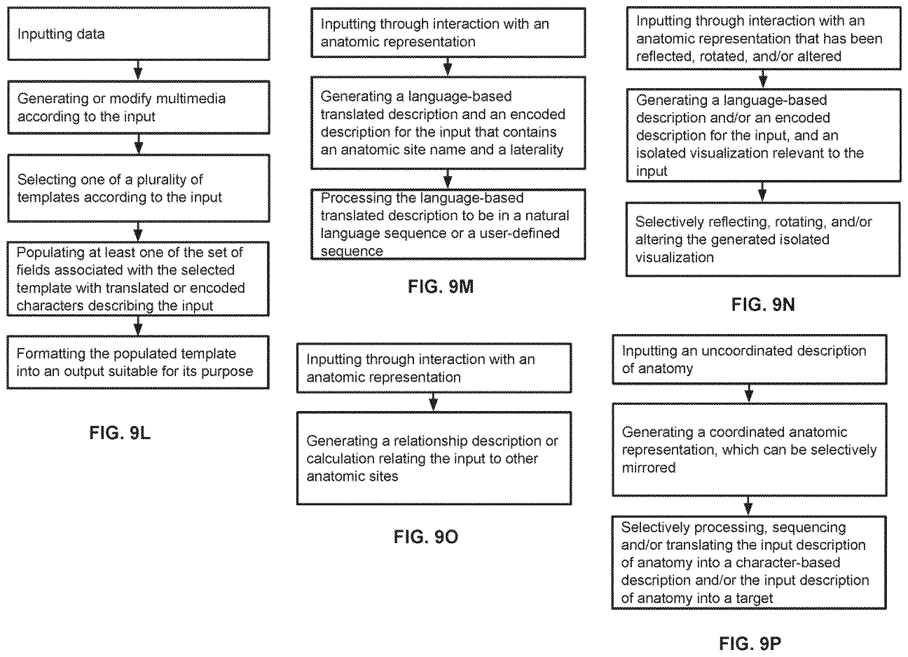

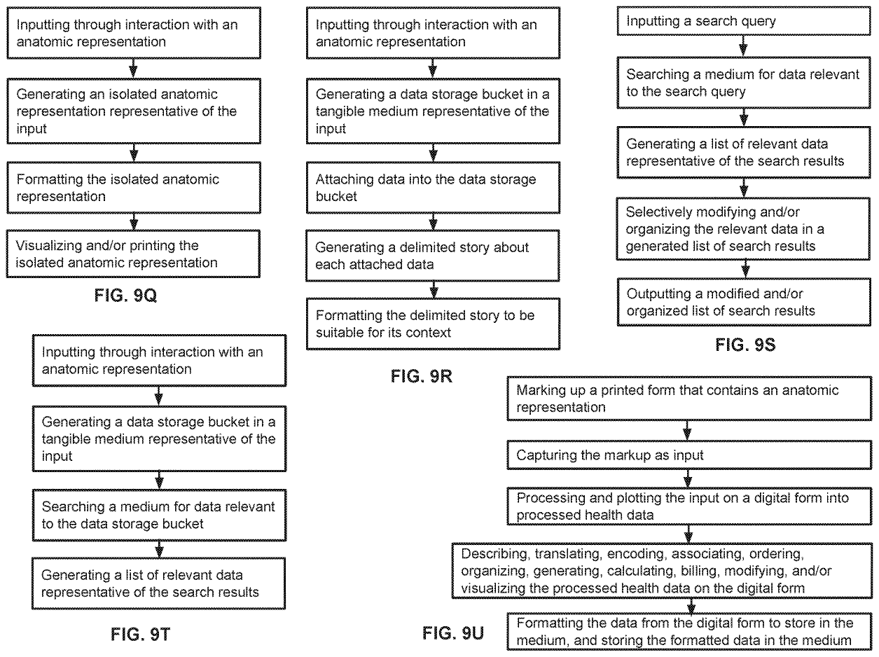

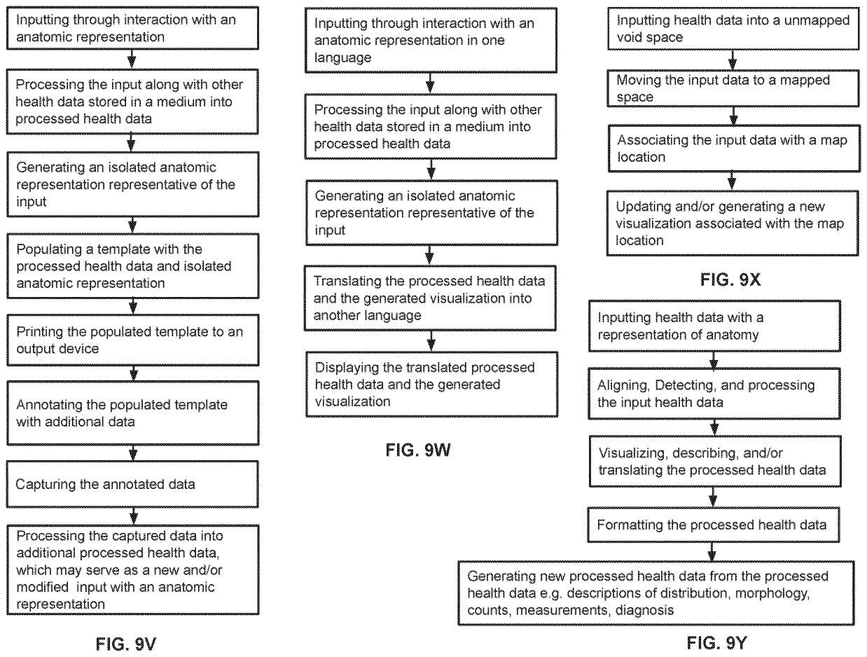

FIG. 1B illustrates a non-limiting example embodiment of a method enabled by the system 985. FIG. 9G through FIG. 9Y similarly illustrate non-limiting example embodiments of the system, systems, method, and/or methods enabled by the teachings herein. Each block of the non-limiting example embodiments in FIG. 1B, FIG. 9G through FIG. 9Y, and throughout the figures and specification of the teachings, is a non-limiting example step of a system and/or method that may be combined, omitted, skipped, re-ordered, expanded, reduced, or modified according to the teachings herein. For example, an embodiment such as FIG. 1B can include: Rendering anatomic representation (which may be an optional step in certain embodiments, e.g. when the anatomic representation is on physical subject such as a human, mannequin, object, or animal as non-limiting examples), receiving input, processing input to generate processed health data, rendering marked anatomic site on anatomic representation or isolated anatomic representation having marked anatomic site, selectively rendering mirror image of anatomic representation or isolated anatomic representation having marked anatomic site, selecting template, populating template, and generating health record. Other non-limiting examples are also included in FIG. 1B and FIGS. 9G through FIG. 9Y, such as in FIG. 9G which can include, for non-limiting example: Accepting input that contains anatomy through the input device 988; Generating visualizations (e.g. representations), descriptions, and data buckets in the tangible medium and/or displaying them on the display 987 with the GUI 991; Selectively accepting additional inputs (e.g. in the data buckets stored in the medium 995 in the database interface module 996); Generating outputs (e.g. with the generation module 993) related to the generated visualizations, descriptions, data buckets, and additional inputs; Selectively recycling the generated outputs as additional inputs (e.g. with the data processing module 999) into the system.

FIG. 9H includes non-limiting example steps such as: Input that contains anatomy information; Selecting one of a plurality of templates according to the input anatomic information and other data; Populating at least one of the set of fields associated with the selected template with translated or encoded characters describing the anatomy information; Formatting the populated template into a database record suitable for an electronic health records database.

FIG. 9I includes non-limiting example steps such as: Inputting data; Selecting one of a plurality of templates according to the input data; Populating at least one of the set of fields associated with the selected template with translated and encoded characters describing the input data; Formatting the populated template into a database record suitable for an electronic health records database.

FIG. 9J includes non-limiting example steps such as: Inputting non-anatomy health data; Selecting one of a plurality of templates according to the input data; Populating at least one of the set of fields associated with the selected template with translated and encoded characters describing the input data; Formatting the populated template into a database record suitable for an electronic health records database.

FIG. 9K includes non-limiting example steps such as: Receiving input that contains anatomy information; Translating and describing the input as text; Generating and/or modifying non-text multimedia containing anatomy according to the input and selectively generate a reflected view; Selecting one of a plurality of templates according to the input anatomic information; Populating at least one of the set of fields associated with the selected template with translated or encoded characters describing the anatomy information; Formatting the populated template into a database record suitable for an electronic health records database.

FIG. 9L includes non-limiting example steps such as: Inputting data; Generating or modify multimedia according to the input; Selecting one of a plurality of templates according to the input; Populating at least one of the set of fields associated with the selected template with translated or encoded characters describing the input; Formatting the populated template into an output suitable for its purpose.

FIG. 9M includes non-limiting example steps such as: Inputting through interaction with an anatomic representation; Generating a language-based translated description and an encoded description for the input that contains an anatomic site name and a laterality; Processing the language-based translated description to be in a natural language sequence or a user-defined sequence.

FIG. 9N includes non-limiting example steps such as: Inputting through interaction with an anatomic representation that has been reflected, rotated, and/or altered; Generating a language-based description and/or an encoded description for the input, and an isolated visualization relevant to the input; Selectively reflecting, rotating, and/or altering the generated isolated visualization.

FIG. 9O includes non-limiting example steps such as: Inputting through interaction with an anatomic representation; Generating a relationship description or calculation relating the input to other anatomic sites.

FIG. 9P includes non-limiting example steps such as: Inputting an uncoordinated description of anatomy; Generating a coordinated anatomic representation, which can be selectively mirrored; Selectively processing, sequencing and/or translating the input description of anatomy into a character-based description and/or the input description of anatomy into a target.

FIG. 9Q includes non-limiting example steps such as: Inputting through interaction with an anatomic representation; Generating an isolated anatomic representation representative of the input; Formatting the isolated anatomic representation; Visualizing and/or printing the isolated anatomic representation.

FIG. 9R includes non-limiting example steps such as: Inputting through interaction with an anatomic representation; Generating a data storage bucket in a tangible medium representative of the input; Attaching data into the data storage bucket; Generating a delimited story (e.g. such as with symbolic delimiters and symbolic definitions) about each attached data; Formatting the delimited story to be suitable for its context (e.g. such as by de-identifying the data and renaming the files without the identifiable patient information).

FIG. 9S includes non-limiting example steps such as: Inputting a search query; Searching a medium for data relevant to the search query; Generating a list of relevant data representative of the search results; Selectively modifying and/or organizing the relevant data in a generated list of search results; Outputting a modified and/or organized list of search results to a physical display and/or file stored in the medium.

FIG. 9T includes non-limiting example steps such as: Inputting through interaction with an anatomic representation; Generating a data storage bucket in a tangible medium representative of the input; Searching a medium for data relevant to the data storage bucket (e.g. a timeline of past, present, or future records associated with an anatomic site or region), Generating a list of relevant data representative of the search results for display and/or interaction to the output device and/or storage in the tangible medium.

FIG. 9U includes non-limiting example steps such as: Marking up a printed (e.g. on paper and/or a digital display) form that contains an anatomic representation; Capturing the markup as input (e.g. as pen on paper and/or with a digital pen that tracks the position on paper); Processing and plotting the input on a digital form into processed health data; Describing, translating, encoding, associating, ordering, organizing, generating, calculating, billing, modifying, and/or visualizing (other non-limiting e.g. diagnosing, categorizing, measuring, relating, etc.) the processed health data on the digital form; Formatting the data from the digital form to store in the medium, and storing the formatted data in the medium.

FIG. 9V includes non-limiting example steps such as: Inputting through interaction with an anatomic representation; Processing the input along with other health data stored in a medium into processed health data; Generating an isolated anatomic representation representative of the input; Populating a template with the processed health data and isolated anatomic representation; Printing the populated template to the output device; Annotating the populated template with additional data; Capturing the annotated data; Processing the captured data into additional processed health data, which may serve as a new and/or modified input with an anatomic representation.

FIG. 9W includes non-limiting example steps such as: Inputting through interaction with an anatomic representation in one language; Processing the input along with other health data stored in a medium into processed health data; Generating an isolated anatomic representation representative of the input; Translating the processed health data and the generated visualization into another language; Displaying the translated processed health data and the generated visualization.

FIG. 9X includes non-limiting example steps such as: Inputting health data into an unmapped void space; Moving the input data to a mapped space; Associating the input data with a map location; Updating and/or generating a new visualization associated with the map location.

FIG. 9Y includes non-limiting example steps such as: Inputting health data with a representation of anatomy; Aligning, Detecting, and/or processing the input health data; Visualizing, describing, and/or translating the processed health data; Formatting the processed health data; Generating new processed health data from the processed health data e.g., descriptions of distribution, morphology, counts, measurements, diagnosis, etc.

FIG. 1B Inputting through interaction with an anatomic representation, Generating a language-based translated description and an encoded description for the input that contains an anatomic site name and a laterality, Processing the language-based translated description to be in a natural language sequence or a user-defined sequence; Inputting through interaction with an anatomic representation, Generating a relationship description or calculation relating the input to other anatomic sites; Inputting through interaction with an anatomic representation that has been reflected, rotated, and/or altered, Generating a language-based description and/or an encoded description for the input, and an isolated visualization relevant to the input, Generating a language-based description and/or an encoded description for the input, and an isolated visualization relevant to the input, Selectively reflecting, rotating, and/or altering the generated isolated visualization; Inputting an uncoordinated description of anatomy, Generating a coordinated anatomic representation, which can be selectively mirrored (e.g. reflected) and/or otherwise manipulated and/or transformed. It is contemplated that a plurality of permutations could result from these non-limiting example teachings.

FIG. 1C illustrates an example embodiment of a method, system, and/or process enabled by the system 985, which uses the data processing module 999 stored on the tangible medium 995 in communication with the processor 986 to generate processed data 1100 and/or processed health data, enabled by the system 985. Omnidirectional communication occurs within a system comprised of language models 1103, vision-language models 1101, language-vision models 1102, and/or other models to communicate in a coordinated language model type model 980 as one example. The illustrated example embodiment processes Input 988 received through an input device into processed data 1100 through communication with the processor 986 to generate and/or communicate output to the GUI 991, Medium 995, and/or output device 989. It is contemplated that the output can be recycled back into the system as input 988, and/or that the input could be shown as unprocessed output through communication with the GUI 991, Medium 995, and/or Output device 989.

FIG. 1D illustrates an example view on the GUI 991 on a physical display 987 of a path with custom axes, segmentation, and relational instructions, directional planes. A path 31 is shown in red in one non-limiting embodiment, with additive color coding shown in grayscale shading for this non-limiting example embodiment. The path is enabled by coordinates in a file stored on the medium 995 relative to coordinates of an image 1001; and relative to coordinates for a diagram within the image 1001. Paths 31 are coordinated, grouped (e.g., in path and path groups 1004), ordered, and layered with unique identifiers such as those for the anatomic site name, translation or code. Each path 31 highlighted may have custom axes 40 (shown in solid white lines outlined in black in this embodiment), axial definitions 1040 (with each custom axis shown as white letter outlined in black in this embodiment, with “S” meaning Superior, “L” meaning Lateral, “I” meaning Inferior, and “M” meaning Medial in the current non-limiting example), angles (as illustrated by the non-right angles of the axes 40 in this embodiment), segmentation instructions 34 (to describe segmentation in a path through language, code, symbols, highlighting, pattern application, and visually) stored on the medium 995 and processed with the processor 986 as illustrated by triangles pointed at a custom position on the custom axis and defined by a percentage, proximity, or deviation from center or custom offset; a color, pattern, texture, pin, multiple pins, multiple patterns or textures, area of self, area relative to group, area relative to diagram, area relative to multiple diagrams, area relative to multiple groups, opacity, intensity, or other metadata associated with it. Each path 31 may also be within one or more direction planes 1041 (and 1042 in an example embodiment), with two direction planes illustrated in this embodiment shown as solid black lines outlined in white that are split 1042 by a gray line outlined in white. The direction planes can be independent of the paths and transect them, as shown in the embodiment crossing multiple paths with a different angle from the custom axes 40 of the paths 31. Direction planes 1041 (and 1042) can be square, rectangular, or uniquely shaped and curved as shown in the embodiment. Direction planes in the example are shown as black arrows with white outlines connected to single black letters with white outlines in the embodiment. In this example for the direction planes, “A” means anterior 1043, “S” means Superior, “I” means Inferior, “P” means Posterior, “L” means Lateral, “M” means Medial. Two or more direction planes can belong to a direction group 1044, which also has its own set of directional instructions, as illustrated in the embodiment. The directions for a direction group in the example are shown as black arrows with white outlines connected to black letters with white outlines in the embodiment, with “Sup” meaning Superior, “Lt” meaning Left, “Rt” meaning Right, and “Inf” 1044 meaning inferior in the current example. A direction group 1044 can also be formed by a path group, such as all of the paths 31 illustrated on the left face in the embodiment and rendered in a color-coded legend 12 on the GUI 991 on a display 987. It is contemplated that each path 31, path group, direction plane 1041, and direction group 1044 can have its own custom ordering, properties, rotation, axial definition, order, topography, pattern, color, opacity, intensity, or texture. Layering and ordering are taught by this embodiment by the color coded legend 12 correlating with the tip of the cursor 14 when physically shown on the GUI 991. Each path 31, path group, direction plane 1041, or direction group 1044 may have metadata such as identifiers, axial definitions 1040, segmentation instructions 34, central definitions (e.g., how do we define the center of the path? With a term “central” or “mid” or “midline” as some examples), prefixes and suffixes, laterality such as left, right, or bilateral. It is also contemplated that paths do not have to be contiguous as one skilled in the art would know (such as a compound path in vector graphics). The metadata derived from a map position, such as the cursor in this embodiment, communicates with the processor 986 to transform, translate, relate, calculate, and correlate the data delivered from the database interface module 996 with a module such as the data processing module 999, which is stored on local-, network-, or cloud-based storage on the tangible medium 995. When two or more points of interest exist on the same path and same dimension, they are related to one another with natural and coded language by calculating their positions relative to the custom axial definitions through communication with the medium 995, the processor 986, and/or other components of the system 985. Since paths 31 do not need to have custom axial definitions, and since it may be desirable to describe relationships between 2 pins on different paths, the axial definitions of a path group, a direction plane 1041, or a direction group 1044 act as relationship calculator substitutes to relate the points with natural and coded, machine readable language through interaction between the medium 995, the processor 986, and/or other components of the system 985. When points of interest are to be related to one another, the magnitude based on the deviation from center of the custom axis of the path, path group, direction plane, and/or direction group as one example; and/or the magnitude of physical distance between the points of interest (e.g. pixels in any direction as one example) is used to apply “magnitude modifiers” such as “barely” and “very” to describe relationships and/or to omit directional semantic descriptions that may be confusing to human interpretation (for example, if a point is directly above another point (+y on y axis) and minimally changed on the (x axis), the x axis description can be omitted by the data processing module 999 stored on the medium 995 to minimize human confusion in the interpretation of the relational description as one example.

FIG. 1E illustrates an example path with associated data, which may be described as the “anatomy of the stored data in a single example path.” In certain embodiments, each path 31 is comprised of a site ID and other components such as the non-limiting examples in the illustration of a path laterality, modifiers such as prefixes and suffixes, a custom axis id, and custom segmentation instructions 34. Paths 31 may belong to path and path groups 1004 and overlaid and or underlaid relative to an image 1001 when displayed on the GUI 991. Paths 31 may have physical and/or spatial properties, such as coordinates for other map elements, pins, paths, and/or diagrams. The physical and/or spatial properties can include an area and intensity, such as a surface area, calculated, and/or may have coordinate-based relationships defined and/or processed in the medium 995 by the processor 986 in the system 985, and/or output to the GUI 991 in a display 987 or to an output device 989. Similarly, the path 31 components may interact with the tangible medium 995 and/or the processor 986 and/or components of the system 985 to deliver outputs from data-based relationships. Other non-limiting examples of the illustrated embodiment include other optional properties, such as a central zone definition defined by Boolean logic stored in the medium 995 and processed through the processor 986 to deliver a translated description of a central zone by ID number. Symbolic, linguistic, or coded suffix components are other non-limiting examples of path 31 data stored in the tangible medium 995.

FIG. 1F illustrates one embodiment of the system 985 that contains an example interaction of an image 1001 and map 1002. Input 988 as non-limiting examples of input enabled by physical input devices 988 can comprise of points/pins, hover, touch, selections, detections, distributions, descriptions, alignments, scans/images, coordinates, translations, targets, eye trackings, typed, or verbal input. Image 1001 and Map 1002 are paired together creating an Image-Map Pair 1003 with alignment defined by the map or avatar itself or by computer vision, machine learning, artificial intelligence, or even by manual alignment and refinement enabled by the processor 986 in communication with one or more modules and/or submodules in the tangible medium 995 and/or other components of the system 985. While the embodiment illustrates the Image data 1007 over the Map data 1006 this can be reversed to have the map data 1006 over the image data 1007. Image data 1007 can include but is not limited to isolated images, image segments, image groups, avatars, coordinates, landmarks, specific properties, alignments, segmentation instructions, bleed zones, pixel groups, printing instructions, and pixel collections. Images can be any form of multimedia including but not limited to illustrations, photos, videos, digital images, and/or 3D scans. The map has paths and path groups 1004 in a non-limiting two-dimensional vector embodiment, or may have vertices and vertex groups/collections in a non-limiting three-dimensional embodiment. The paths and path groups 1004 comprising the map data may include any of the following examples in this non-limiting list: identifiers, lateralities, prefixes, suffixes, custom coordinates, axes, axial definitions, layers, layer order, specific properties (like male versus female), calculations like area, path segmentation instructions, rotations, scales, transformations, visibility settings, patterns, textures, and relationships. Databases, neural networks, and models are non-limiting examples of modules stored in the tangible medium 995 and communicate through its various modules and submodules process and serve data through Application Programming Interfaces (APIs) and other data processing modules 999 to the libraries and knowledge bases 992 within the knowledge base module 992 and to the Image-Map Pair 1003 and the data can be displayed on a display 987 in communication with the GUI 991. Databases, neural networks, and models may be stored locally or in the “cloud” off premises location on a hard drive, in cache, local storage, random access memory as non-limiting examples of components of the system 985. Some examples of Databases, neural networks, and models, which may be component or submodules of the data processing module 999 and/or a database interface module 996 as non-limiting examples, include but are not limited to: language and translation database, language models 1103, language-vision models 1102, vision-language models 1101, coordinated language model 980 engines, patient database, encounter/schedule database, encoding database, symbolic database, cross-mapping database, relational database, coordinates database, synonyms database, engines, settings, language rules for natural language processing, morphology database, diagnosis database and API like ICD-11, other databases, and training databases for machine learning, AI, and computer vision. The Databases, neural networks, models, and other modules on the medium 995 enabled by the processor 986 communicate with the input 988, image-map pair 1003 together or with one of its components, such as the knowledge base module 992 and with a plurality of outputs such as the GUI 986, the tangible medium 995, and/or an output device 989. Some non-limiting examples of knowledge base modules 992 and other modules in the tangible medium 995 include image-map pair library, standard outputs, language synonyms to code and vision database and engine, reference data/source of truth, training data, coordinates, template library, forms library, and forms building database. Input interaction with any component or combination of components of the Image-Map pair 1003, databases, neural networks, and models enabled by the tangible medium 995, or libraries and knowledge base modules 992 will generate output. Output may include but is not limited to Visualization, Description, Language, Encodings, Relationships, Segmentations, Symbols, Summaries, Proximities, Calculations, Deviations, Transformations, Translations, Physical forms, interactive forms, tracking IDs, distributions, printable forms, shadow charts, etc, and output may be to the GUI 991, an output device 989, the medium 995, and/or other components of the system 985. API interaction with the processor 986 and/or tangible medium 995 can occur at any step but is not a requirement for any step, and may be enabled by modules, submodules, and module combinations on the medium 995.

(APP01) Traditionally, mapping and labeling of specific anatomic sites within medical records is achievable with electronic health records that include two-dimensional or three-dimensional avatars and mapped anatomic sites. Some even have the ability to drop a pin and output an anatomic site description. However, none of the traditional methods are capable of combined real-time site description enhancement, translation, encoding, relational descriptions, site categorization, cross-mapping, axial reflection and/or mirroring, and color coding through all levels of an anatomic hierarchy as enabled by the system 985 with the medium 995 and processor 986 configured to process a combination of coordinated data and/or uncoordinated data receive through communication with an input device 988 and/or a component of the tangible medium 995. The embodiments illustrated operate in the system 985 on real-time axis switching in path defined different anatomic sites that have different levels, layers, centers, offsets, or rotations and simultaneous labeling and relational descriptions enabled by these different axes. This dramatically improves upon existing anatomical mapping of medical records by enabling multifunctional and multidimensional mapping simultaneously through all levels of a patient's anatomic hierarchy in real-time (or on-demand) creating a more comprehensive patient electronic health record that can track features associated with anatomic sites and regions over time.

Methods and systems are described herein for specific, enhanced, reproducible, translated, visualized, dynamic, descriptive, and encoded anatomic sites facilitate improved communication, documentation, tracking, understanding, and descriptions of anatomic sites or regions affected by diseases, treatments, symptoms, and morphologies across different systems and languages. For example, for an English-speaking physician to communicate with a patient who does not know English, the two would traditionally have to rely on a translator or translation device to communicate on the patient's diagnosis and treatment. Furthermore, traditional labeling and encoding of specific anatomic sites within medical records and associating that anatomy labeling with diagnoses, images, records, billing codes, anatomic site codes, tags, external links, and translations, and visualizations is a manual, multi-step and time-consuming process that requires medical knowledge and human cognitive workload. Additionally, labeling different sections within a defined anatomic region has been a manual process, and within a single layer, axis, or dimension at a time. However, by creating a system that incorporates multiple coded, linguistic, or symbolic languages and in real-time (or on demand) allows for dynamic visualizations of defined different anatomic sites the patient and physician can simultaneously view the anatomic site of interest, the associated site information, and any updates in their own preferred language, thus improving communication between the two, eliminating the need for extensive medical knowledge of anatomical names, and improving tracking of the patient's conditions and treatment.

For example, with an embodied example system the non-English speaking patient can stamp or hierarchically paint the anatomic locations or regions of their concerns (e.g., symptoms, lesions, rashes) on avatars in a mirrored (reflected, and/or selectively rotated or transformed based on the detected context (e.g. camera is in “selfie-mode” in the image interface module 994), view in their preferred language on the GUI 991, simultaneously and in real-time the English-speaking physician can visualize the GUI 991 on a separate display 987 the patient's report in English from an outside-observer view. In certain embodiments, the patient can even attach their own photos of lesions they are concerned about directly to the anatomic site on an input device 988 in their preferred language, and the physician can add treatment recommendations, observations, diagnoses, and other data in English, which the patient will see in their preferred language on the GUI 991. Further, if treatment is ongoing, for example for a rash, if different anatomic regions of the rash are responding differently to treatment, the patient can report on the reproduced distribution in a visual and descriptive workflow on the GUI 987 in communication with the medium 995 as components of the system 985, without actually knowing anatomy names, and thus creating tracking points for disease response by time, by site, and by treatment automatically. In certain embodiments, this is enabled by a coordinated language model 980 engine enabled by the data processing module 999, combining language models 1103, language-vision models 1102, vision-language models 1101, and/or other models stored on the tangible medium 995.

Conventional solutions are unable to allow for simultaneous, reproducible, and automatically enhanced translations and visualizations of anatomy nor do they offer translated and visualized travel through an anatomic hierarchy shown on a physical display 987 with the GUI 991. The embodiments illustrated generate, through a generation module 993, automatically enhanced translations and visualizations to multiple mapping workflows to facilitate communication, documentation, understanding, and tracking. To overcome limitations in conventional methods of anatomic mapping, the system generates a dynamic anatomic address through a generation module 993 for every anatomic point and region on the body, with each address serving as a multidimensional data tracking and collation point. Thus, data relevant to anatomic site and distribution can be tracked and collated for different time points in different patients, and for populations. In certain embodiments, a timeline can be generated by the generation module 993 in communication with the data processing module 999 and/or the record retrieval module 997, and shown in the translated and context-aware GUI 991.

Dynamic anatomic addresses apply to points of interest, which may be represented as pinpoints, segments of anatomic sites, groups of anatomic site segments, vertices, vertice groups, vertice collections, complete anatomic sites, groups of anatomic sites, different levels of hierarchy, different groups, and different organ systems simultaneously. Visual definitions and dynamic custom coordinates of anatomic addresses can be looked up by anatomic site name in any combination of language or code, and progressively (and/or regressively) sub-segmented with directional and magnitude modifier terms with mixed code and language order (on which the data-processing module 999 applies coded and linguistic dissection to provide the visualizations and translations from the knowledge base module 992 and/or the generation module 993 in certain embodiments); and those definitions can be detected and visualized in different views and images and multimedia automatically. The system 985 may use hierarchical painting of anatomy to automatically capture distributions, body surface area calculations, and intensity (such as first-degree, second-degree, third-degree burns; also important in determining Psoriasis and Severity Index (PASI) scores for psoriasis and EASI scores for atopic dermatitis affecting insurance coverage/prior authorization documentation in the United States) and simultaneously provide a standardized description of each distribution segment, an isolated visual preview of each distribution segment, and a combined visual preview of the different distribution segments, in both outside observer and alternate (e.g. mirror) views. The system 985 can be configured to provide a data matrix that tracks disease distribution, intensity, and surface area automatically with linguistic descriptions, machine readable descriptions, and visualizations, more effectively tracking a patient's conditions. Other contemplated examples of automatic calculation of scoring that is enabled by anatomic sites, groups of anatomy, anatomic distribution, and health metadata include cancer staging such as automatically using the engines, databases, health data, anatomy data stored in the medium 995 and processors 986 to calculate tumor staging, nodal staging, and metastatic staging (TNM method) used by the NCCN Guidelines (National Comprehensive Cancer Network) as one example, or calculating a Mohs Appropriate Use Criteria (AUC) score based on anatomic site and other health data features found in the patient history, pathology report, photographs/images, measurements, and other health data. One example of a module in the medium 995 that could store the data on the aforementioned non-limiting examples is the knowledge base module 992.

The dynamic anatomic addresses along with standardized anatomy codes, names, and symbols, patient data, diagnosis data, encounter data, tags, and other data is used to generate language-agnostic file naming, grouping, and exporting function (as outputs to the medium 995, as one example) with optional universal symbolic low-character-count delimiters to automatically write a language agnostic story about an anatomic site. The data is combined into a string and separated with meaningful delimiters into an order-independent, structureless, meaningful story by, for example, the data processing module 999 and/or the generation module 993. This story does not have to fit into an electronic health record (EHR) or other defined data structure, and it can be stored in an unstructured file system as a submodule of the database interface module 996 as one example. This creates a data block that can be truncated in a file name, encrypted into static or evolving QR codes (or other with or without encryption), stored in exported file metadata in the medium 995, stored as a digital and targetable “bookmark,” exported to a database or file wrapper (such as a Digital Imaging and Communications in Medicine (DICOM) wrapper), filtered, searched, de-identified, encoded, and tagged. The data stored in the tangible medium can be in communication with the processor 986 and/or a module, submodule, and/or module combination in the medium 995.

The anatomic site that the dynamic anatomic address correlates to is visually depicted on a display 987 for the system 985 user on anatomic maps and/or visualizations and/or representations, including actual patient multimedia that includes photographs, video, live views through camera previews or augmented reality as non-limiting input device 988 examples, two dimensional views, three-dimensional views, four-dimensional views (over time), multi-dimensional views, medical imaging (such as X-rays, CT scans, MRIs, or Ultrasounds), virtual reality, and mixed reality. These visualizations can be two- or three-dimensional and may accept input from an input device 988. Each anatomic map is comprised of layers of paths, shapes, and compound paths stacked in two-dimensional or three-dimensional space and stored on the medium 995. A fourth dimension is added when the maps are compared over time, such as for tracking points, anatomic sites, or groups of anatomic sites, as enabled by the data processing module 999, the record retrieval module 997, and/or the generation module 993 as an example. Multiple, unlimited dimensions are added when each path or layer has its own set of axes, centers, offsets, angles, medians, and rotations, as does each group of paths or layers, or group of groups. Each path 31 or layer could also be known as a vertice group or vertice collection such as in three-dimensional modeling in alternate embodiments. Each path or layer may function alone, and may have its own center, offset, rotation, elevation, depression, topography, surface area, hierarchy level, laterality, prefix, suffix, name, axis definition, and metadata within the system 985 and/or the medium 995. Each path 31 or layer can independently scale, align, rotate, move, remain static, or be shown, hidden, colored, patterned, or highlighted. Each path 31 or layer can also dependently scale, align, rotate, move, remain static, or be shown, hidden, colored, patterned, or highlighted-depending on its membership in a group or on patient characteristics as defined in the medium 995 of the system 985. In certain embodiments, group membership might be the paths or layers that are part of the left eye group on a facial diagram group, which represents a group (left eye) of groups (facial). In certain embodiments, patient characteristics may be selectively hidden or filtered out when those characteristics are immaterial to the patient, such as the multidimensional maps for deciduous dentition (children's teeth) map groups for an adult patient or male genitalia map groups for a female patient. Continuing this example, selective filtering enabled by the data processing module, enables certain embodiments of a paper anatomic map to serve as a consistent input device 988 whereas the digital twin map may show irrelevant characteristics as whitespace on the GUI 991 in certain embodiments. A single path 31 or layer of the patient's anatomic map may belong to multiple groups of the patient's anatomic map simultaneously as defined in the medium 995 in one example. Further, paths or layers can be selectively targeted and automatically related to one another based on their custom axial definitions or group axial definitions. Groups have the same dependent and independent properties as layers or paths 31. Paths 31 can be further defined by pixel groups, coordinate groups, vertex/vertice groups, or coordinates and may be targeted through coordinates or uncoordinated inputs, such as detected language, code, symbols, or image-based detections by the system 985 where the processor 986 is in communication with the medium 985.

On a computing device equipped with a graphical user interface (GUI) 991 and processor 986, multidimensional maps may be overlaid or underlaid, and separately aligned in different dimensions, on anatomic images, avatars, and diagrams. Embodied example systems and methods are enabled by combinations of the GUI 991, the medium 995 that can include non-limiting examples such as structured databases, unstructured databases, tables, application programming interfaces (APIs), neural networks (as an example of a communication between the knowledge base module 992, the data-processing module 999, the database interface module 996, and/or the other modules in the medium 995), hard drives, caches, natural language processors (NLP, as an example of the data processing module 999), graphical processing unit (GPU), cloud storage, random access memory (RAM), engines, and user input such as through computer mouse; touch-screen display; microphone to accept voice or audio input; speaker and microphone to facilitate bidirectional communication between human and machine; eye-tracking; gestures; handwriting; hand-drawn markup or annotation; and/or graphical processing units (GPU). A map visualization system in certain embodiments is used to selectively filter or show, on the GUI 991, all levels of an anatomic hierarchy under a point, such as a cursor or a placed pin, while simultaneously showing the points relationship to each level of hierarchy, and enhanced descriptions and translations of the anatomic site at that particular point. In certain embodiments, an additive color sequence is used to visualize, on the GUI 991, all levels of an anatomic hierarchy simultaneously at a point, with a real-time, color-coded legend that displays on the GUI 991 enhanced, translated, and encoded anatomic descriptions based on the multidimensional custom axes of the system 985 stored on the medium 995 in communication with the processor 986.

Furthermore, with a vector-based approach in certain embodiments that uses points, lines, and polygons in both two-dimensional (2D) and three-dimensional (3D) models for visual definitions enabled by a system and/or method described, and through use of a Dimensionally Extended 9-Intersection Model (DE-9IM) stored in the medium 995, the coordinated language model 980 engines are expanded to be capable of describing relationships with semantics such as “equals”; “within”; “intersects with”; “crosses with”; “overlaps with”; and “touches” properties. Custom axes stored in the medium 995 can also refine those descriptions with appropriate directional modifiers (e.g., the inferior aspect of A touches the superior aspect of B; and vice versa) when processed in communication with the processor 986. The DE-9IM model complements the ontologic data-based models by connecting spatial relationships with text-based hierarchical and cross-linked relationships. Customized axes allow for automatic human readable prose definitions, relationships, and machine-readable definitions for different path segments, complete paths, and path groups enabled by communication between the processor 986 and components in the medium 995. For example, the system 985 and/or method of description can automatically describe the origin and insertion points of muscles to bones or other muscles with more precision than is traditionally available even in anatomic atlases. Angles, measurements, distances, shapes, surface areas, rotations, and other data become automatically describable and relatable through interaction with the processor 986 and data stored in the medium 995. For clarity, in the examples, methods, and systems, paths may also be defined as vertices or vertices groups or collections of coordinates. Relationships can also be derived from text-based hierarchies and cross-mappings and ontologies; even linking different body systems, organ systems, or functional systems, or other mappings (e.g., dermatomes).

When there are two or more points of interest, the custom axes of the points or the custom axes of a group membership stored in the medium 995 are used in the knowledge base module 992 in communication with the data processing module 999 and/or other modules to generate artificial neural networks, such as through the generation module 993, and other information systems to automatically describe their directional relationships to one another, in human readable and machine-readable language. Encoding, using an autoencoder type neural network in the medium 995 as one example, and cross-mapping of each component of the enhanced anatomic site descriptions (in communication with a database interface module 996 as one example) shown on the map also occurs simultaneously and in real-time in certain embodiments. In one iteration, encoding is converting points on an anatomic image to a code string containing laterality and anatomic site name from a dictionary (e.g., stored on a database interface module 996 or other module of the medium 995), such as the International Classification of Diseases (“ICD-11”), a globally used diagnostic tool for epidemiology, health management and clinical purposes. In another iteration, encoding simultaneously occurs for the prefixes, suffixes, and enhanced modifier description components of the enhanced and translated anatomic site name, with natural linguistic sequencing applied through a natural language processing through communication with the data processing module 999 as one example. In another embodiment, cross-mapping would automatically show all codes or names from other anatomic lexicons, such as the New York University numbering system (“NYU numbers”), which allows clinicians to easily identify areas of the body with standardized non-hierarchical, two-dimensional numbered diagrams and may be stored in the knowledge base module 992 and/or other module of the medium 995. Cross-mapping and automatic correlation to multidimensional maps enables, through the processor 986 communicating with the medium 995 and/or its components, lesion tracking over time, for all levels of an anatomic hierarchy, at any point on the diagram, at any cross-mapping point.

In certain embodiments, the anatomy mapping and labeling engine of the system 985 is configured to document enhanced, standardized, optionally segmented, and multidimensional locations and descriptions of procedures, diagnoses, treatments, symptoms, morphologies, or other patient characteristics. Hierarchical selectors in the embodiments illustrated and shown on the GUI 991 allow for documented pins and distribution segments to travel through different levels of the anatomic hierarchy and dimensions of the anatomic map, with real-time visualization, translation, and site descriptions for each anatomic site in the hierarchy based on input received from an input device 988. The embodiments illustrated also include a system and/or method of hierarchical painting of anatomic sites and site segments, to simultaneously visualize and label, through an input device 988, distributions and intensities of properties like diseases, morphologies, symptoms, and patient education on anatomic maps. Additionally, these anatomic maps can allow for visualization from both outside observer view and/or alternative views such as a mirrored “selfie” view 426 shown on the GUI 991 on a context-aware display 987. Expanding this example, perspective defaults can be mirrored 426, allowing for documentation, translation, labeling, mapping, and visualization in both outside observer and selfie views 426.

Certain embodiments of the system 985 dramatically improve existing anatomical mapping of medical records. Multifunctional and multidimensional mapping is enabled by the system 985 with the processor 986 in communication with the medium 995 that can be configured to receive input from an input device 988 and simultaneously output to an output device 989, the GUI 991, and/or the medium 995 all levels of a patient's anatomic hierarchy in real-time (or on demand), creating a more comprehensive patient electronic health record capable of tracking features associated with anatomic sites and regions over time, such as when processed by the record retrieval module 997 in the medium 995.

Again, the examples illustrated herein for embodied application system 985 examples and functions and/or modules enabled by them such as engines, calculations, models, information systems, algorithms, and other embodiments of methods and systems enabled by communication between displays 987, stored data in the medium 995 such as on hard drives and cache, stored databases, printed and physical data such as manual markup received as input devices 988 for example, engines, map files, stored files and multimedia, and other electronic hardware in the medium 995 configured to map and label specific anatomic sites configured to provide enhanced directional modifiers labeled simultaneously by the system 985 in unlimited layers, hierarchies, tissues, organ systems, and groups with simultaneous categorization and translation into any coded, linguistic, or symbolic language in real-time and multidimensional space. The example systems and methods enabled by the system 985 herein provide greater functionality than existing electronic anatomic mapping such that human and machine readable, translated and enhanced real-time descriptions with or without magnitude modifiers to automatically describe relationships, distances, surface areas, volumes, and findings between two or more anatomic sites, regions, or pins. Distance from center can be detected as well as proximities to center, angles, rotations, medians, and offsets, and these detections are used to generate more meaningful and improved anatomic site names 17, descriptions, relationships, borders, and visualizations through the generation module 993 as one non-limiting example. The magnitude of axial deviations can automatically and optionally reorder the directional modifier descriptors to make the combined descriptions and visualizations more meaningful, precise, and accurate. Linguistic and visual sub-segmentation are also configured by modules in the medium 995, such as the data processing module 999 and/or the generation module 993 as non-limiting examples, to generate enhanced descriptions with corresponding visual previews (in isolation or related to groups), that both change and realign dynamically with the patient when needed and remain static in terms of standardized language and code sequences.

Referring to the drawings, FIG. 1G is a non-limiting example screenshot of the GUI 991 showing an anatomic representation with anatomic sites associated with an anatomic region with one or more subregions to show the hierarchical visualization overlay. The embodiment depicts an anatomic visualization 10 with five hierarchically labeled, color-coded, enhanced, and translated anatomic site descriptions 20 correlating with the point of the cursor 14 over color-synced anatomic site regions in this example. The multidimensional anatomic map stored on the medium 995 that has been configured for this embodiment to simultaneously display all hierarchical levels overlaid on an anatomic visualization 10, thus obscuring it on the GUI 991 in this example. The five enhanced, translated anatomy labels 20 corresponding with an anatomic site at a cursor point 18 in this embodiment are listed in the color-coded legend 12 with a portion of the dynamic anatomic address for this point. A dynamic anatomic address in the system 985 is associated with an anatomic site stored in the medium 995 and acts as a data bucket, in a module of the tangible medium 995 such as a database interface module 996, in which to store data associated with the anatomic site but is dynamic in that it simultaneously stores the data in relative buckets across its lineage as well as proximately located buckets and is capable of moving based on patient anatomy changes through communication with the processor 986 and/or modules in the tangible medium 995. For example, as a patient naturally ages, the body grows and stretches as well as sustain trauma. Therefore, a dynamic anatomic address on a twenty-year-old may move multiple times as the patient ages and experiences things like weight gain or childbirth or injury or other such event as one skilled in the art would understand. The dynamic anatomic address must include an anatomic site name, encoding, index, or symbol and may include any combination of additional applicable descriptive elements, including but not limited to, laterality, prefixes, suffixes, enhanced modifiers, custom descriptions, triangulations, measurements, automatic relationship descriptions to other anatomic sites or pins, translations, synonyms, cross-mappings, categorizations, coordinates, paths, axes, rotations, angles, topographies, depressions, elevations, visual definitions, segments, polyhierarchical membership, patterns, colors, intensities, surface areas, linked metadata, angles, centers, offsets, calculations, deviations, proximities, rotations, pin IDs, test IDs, historical information, visual definitions, visual previews, dates, notes, identifiers, groups, lineage (hierarchical parents, siblings, children, cousins, relatives, neighbors, etc.), zoning, visualization based on lineage, natural linguistic sequencing, attachments, language, multimedia, detections, symptoms, symbolic definitions, morphologies, skin tone, skin type, sex/gender variability, race/ethnicity variability, time-points, outside-observer view, mirror/selfie view 426, positions, coordinates, site segments. The aforementioned properties may be stored in a module of the medium 995 such as in a database interface module 996 as one non-limiting example. Further, a dynamic anatomic address can overlay or underlay with respect to images shown on a display 987 and be configured to communicate with the processor 986 and/or the medium 995 to dynamically recalibrate to images such as diagrams, photos, videos, avatars, partial images regardless of the image size or segments in view, while simultaneously providing visualization of self, relatives and lineage and providing correct anatomic site names and categorizations in any coded, linguistic, or symbolic language. It is contemplated that overlay of a hierarchical map could still be used in other non-limiting embodiments by keeping the entire hierarchical map in a separate group, and changing the opacity of the group, thereby solving unexpected color blending on the GUI 991 that could cause user confusion.

The illustrated embodiment depicts the following regions: left superior paramedian forehead 21, superior left forehead 22, face 23, head 24, and head and neck 25. In the illustrated embodiment, the user has placed the cursor 14 over path-defined anatomic sites 18 with custom axes as a temporary site selection in the anatomic visualization 10. An enhanced description 16 is displayed as a result of the cursor 14 hovering over the anatomic site 18 as enabled by an input device 988 in communication with the GUI 991 in a display 987. The color-coded legend 12 additionally tells the user the hierarchy of the anatomic site 18 that the cursor 14 is within, over, and/or under with a portion of the dynamic anatomic address for the anatomic site 18 visible in multiple hierarchical levels based on the custom axes of each level. In the illustrated embodiment, the color-coded legend 12 is indented by the relative hierarchical level in the GUI 991 to enhance human comprehension. It is contemplated that the coded legend 12 could be any visually distinct code such as a ROYGBIV (red, orange, yellow, green, blue, indigo, violet) scheme or any dynamic color or pattern sequence as defined by a component of medium 995 or input device 988 in communication with the processor 986 as one example. With opacity adjustments as an alternative for overlaying, color blending occurs creating difficulties in hierarchical visualization, but these can be accounted for programmatically through site segmentation, addition, subtraction, multiplication, and other methods when overlays are needed other avatars, photos, and other images. It is also contemplated that dynamically changing overlay and underlay rules and functions generates, through the generation module 993 and/or other modules as one example, relevant anatomic visualizations 10 to the user and targets the dynamic anatomic address 18 with additional visualization steps depending on their underlay or overlay status defined in the medium 995.

FIG. 1H shows the same anatomic visualization 10 as FIG. 1G with the multidimensional hierarchical anatomic map underlaid under an anatomic representation rather than overlaid to prevent obscuring the borders of the anatomic visualization 10. The cursor 14 is again placed, through an input device 988, over an anatomic site 18 as a temporary site selection in the system 985. It is contemplated that the specific anatomic site 18 could be selected with an input device 988 and saved with a pin or anchor or other such tagging mechanism in the tangible medium 995 as one skilled in the art would know. The method of this embodiment applies such pin or anchor simultaneously in all layers, levels, dimensions, custom axes, and anatomic sites, at any angle. The enhanced anatomic site description 17 is shown for the point of the cursor 14, with the color coded legend 12 also shown corresponding with the underlaid mapping in the GUI 991.

FIG. 1I depicts a diagram of a cross-section 30 representative of a single plane for an anatomic site or site segment and the paths 31, centers 33, segmentation boundaries 34 at different deviations from center, and approach angle 35 to a point of interest 37 related to that anatomic site or site segment. The non-limiting components illustrated in this figure are stored in the tangible medium 995 and may communicate with the processor 986, a modular component of the medium 995, and/or other components in the system 985. In this example, the cross-section 30 indicates the offset from center 36 for a single path and show multiple paths 31, including a path that span two levels 38 and a path of alternate thickness 32. Each path has custom axial definitions, which may differ from one to another, and a plurality of other properties such as: anatomic site name, identifiers, laterality, prefixes, suffixes, axial definitions (for axes), axial customizations, coordinates, rotations, scales, offsets, centers, thicknesses, bounding boxes, color, pattern, opacity, stroke, visual attributes, animations, group memberships, group axial definitions, curves, zones, linguistic segmentation instructions, visual segmentation instructions, relationships, position, level, mathematical translations, linguistic translations, transformations, links, order, perspective, view, and other metadata. It is contemplated that each path 31 may touch paths in the same level, may span multiple levels, may touch, overlap, or be separated from other paths (leaving gaps in the level). Gaps may be automatically filled by underlying, overlying, or neighboring paths, passed through, or ignored. It is further contemplated that paths may have different thicknesses in different planes and can belong to multiple groups and simultaneously have proximity based physical neighbors and relatives defined by the path positions, and data-based neighbors and relatives, defined by data sets such as a text-based anatomic hierarchy. It is contemplated that the system 985 component or components within the system 985 can form artificial neural networks to process health data to describe, translate, relate, encode, reflect and visualize physical and data-based relationships simultaneously.

FIG. 1J depicts the same cross-section as FIG. 1K, except a median axis 39 has been added to the instructions stored on the medium 995 which are executable by the processor 986. The median in the illustrated non-limiting embodiment is perpendicular to the cross-section paths 31 and off center. The custom axes for the paths reflect independently from the custom axes of the cross-section which enables multidimensional mirroring, reflecting, and/or altering of customized axes even when an anatomic map is off-center, while the cross-section can still reflect along the defined median in this embodiments and/or in different embodiments. Describing the paths 31 for the angled insertion point 320 and termination point 322 in the illustrated embodiment: insertion point 320 enters on a left medial path, crosses through the median 39, and the termination point 322 is on the right medial path. A second point of interest 324 can be described as on the left central path. In the illustrated embodiment the second point of interest 324 is auto-related to the termination point 322; however, since they have different path defined axes, a secondary axis is used to auto-related the two points, in this non-limiting case, the cross-sectional axis. The relationships between the points are described as the second point of interest 324 is left from the termination point 322 and the termination point 322 is right from the second point of interest 324 automatically applying the correct custom axial information. The cross-section axes are reflected in mirror view, even when mirror view does not contain a midline or median. Therefore, the path axes do not require an overall midline, and can be mirrored, reflected, and/or otherwise altered independently of the midline in certain embodiments. There can be midline dependent axes, exemplified in this non-limiting figure as “Right” and “Left” and mirror, reflect, and or otherwise transform and/or alter along the median. “Right” is shown on the left of this figure because that is how it would appear on the frontal view of human anatomy from an outside observer perspective. In a mirror, reflected, and/or otherwise altered view, “Right” could be on the right, and “Left” could be on the left, resulting in an alternate view of the outside observer perspective aka a “selfie view” in certain embodiments. It is contemplated that paths, path segments, path groups, maps, map groups, and relationships can have multiple midlines and medians, for example the trunk of the body has a median and midline, but so does each extremity (arm or leg) in relation to itself and to the trunk in certain embodiments stored in the medium 995 of the system 985. Another example of multiple medians is when more than one map group and diagram group appears on the same anatomic map set, such as a map set that contains multiple visualizations simultaneously stored on the medium 995, like multiple views of the face at different angles shown on the GUI 991 in a display 987 processed through a module such as the data processing module 999 as one non-limiting example.

Proximity-based and data-based grouping also occurs simultaneously, with certain embodiments applying artificial neural networks enabled by modules in the medium 995, such as the data processing module 999 as one non-limiting example, in communication with the processor 986. Data-based relationships may be defined within the map structure such as in the knowledge base module 992 as one non-limiting example, and/or from an external source, such as a database in a database interface module 996 as another non-limiting example. In one example of a structure-based map relationship, both right and left ears together are one group entity on a single diagram, even though they are not touching each other. In an example of an external data-based relationship that can include the knowledge base module 992 as one example, ICD-11 defines the ear (pinna) as a singular concept further defining laterality or bilaterality by separate concepts in a separate code group. As each point of interest travels through each path, perpendicular to the plane or at an angle, the axes of interpretation are switched in real-time, enabled by the system 985 with the processor 986, enabling descriptions of angles and measurements needed to reach to a terminal point of interest.

FIG. 1K depicts an anatomic plane 45 of a right hand 44 with custom defined axes 40 at a particular rotation and angle as defined, stored, and processed in the medium 995 of the system 985 that communicates with the processor 986. The custom defined axes 40 in this two-dimensional plane 45 intersect at a slight offset from the center for fine tuning and calibration of axial positions. A bounding box 42 is illustrated over the hand 44 with a center point 41. In this example, the figure also depicts the laterality label 43 of the anatomic plane. As one skilled in the art would know, the label for points within the paths, path groups, and segments within this example custom axes could be labeled as proximal, lateral (radial), distal, medial (ulnar), or with a combination of the terms, depending on the point position within the custom axes.

Also embodied in FIG. 1L is a screenshot of a non-limiting embodiment of an anatomic visualization 10 that depicts, on the GUI 991 in the system 985, simultaneous, hierarchical, multidimensional, real-time coloring and labeling of anatomic sites under a cursor position with a color-coded legend representing the enhanced, translated anatomic site descriptions for the colored anatomic sites 50. Also depicted are hierarchically painted anatomy distribution segments with color-coding and patterned, non-patterned, and intensity visualization 51, auto-relation descriptions 54 of the relationships between pins B and C in the pin list 53 (with this embodiment relating pins B and C 52, the pin list 53 describes that “B (this pin) is medial and superior from C” and “C (this pin) is lateral and inferior from B”) (e.g. auto-relation can be calculated by communication between the data processing module 999, the database interface module 996, and/or other modules in the medium 995), simultaneous hierarchical selection, relative visualization (to borders of each anatomic site), translation (with the illustrated embodiment describing pin A 52 simultaneously as the “left (superior) paramedian forehead” in the pin list 53, and as “(superior left) forehead”, “face”, “head” and “head and neck” in the hierarchical selector 505 that also shows pin A 52 isolated and related to the borders of each anatomic site in the anatomic hierarchy), and description of dynamic anatomic address components for the first list item in the example pin list 53, and other functions. Placed pins 52 on the anatomic visualization 10 indicate locations where patient medical events have occurred and are stored in the system 985 with the medium 995 that has modules in communication with the processor 986 as one example. In the illustrated embodiment, dynamic pin descriptions indicate shave biopsies were performed on various locations on the forehead. The pin list 53 shows an isolated visual preview including information associated with each pin 52, including pin order, and pin position relative to the selected anatomic site component of the dynamic anatomic address, automatically populated diagnosis with a description and code (for non-limiting example by the data processing module 999 in communication with the knowledge base module 992), and the dynamic pin description in the same color as the pin, allowing for synchronization to the map and other outputs. Below the pin list 53 there are diagnoses 55 listed in collapsed lists correlating with other pins (melanoma) and painted distribution segments (dermatitis NOS and acne) on the layered anatomic map 50 shown on the GUI 991. In the illustrated embodiment, despite pins 52 and hierarchically painted distribution segments with colors, patterns, and opacities occupying anatomic site segments, the color-coded legend to display the multidimensional site visualizations and translated descriptions relative to the cursor 14 is still visualized on the GUI 991 without interruption because of its underlayment. Hierarchical overlays again would cause real-time visualization obscurement issues. FIG. 1L is a representative screenshot of a non-limiting embodiment of an anatomic visualization 10 that depicts simultaneous, hierarchical, multidimensional, real-time coloring 50 and labeling of anatomic sites under a cursor position (e.g., determined by an input device 988 such as a mouse) with a color-coded legend representing the enhanced, translated anatomic site descriptions for the colored anatomic sites. Also depicted are hierarchically painted anatomy distribution segments 51 with color coding and patterned, non-patterned, and intensity visualization, auto-relation describing the relationships between pins 52 B and C in the pin list 53 (with this embodiment relating pins B and C 52, the pin list 53 describes that “B (this pin) is medial and superior from C” and “C (this pin) is lateral and inferior from B”), simultaneous hierarchical selection, relative visualization (to borders of each anatomic site), translation (with the illustrated embodiment describing pin A 52 simultaneously as the “left (superior) paramedian forehead” in the pin list 53, and as “(superior left) forehead”, “face”, “head” and “head and neck” in the hierarchical selector 505 that also shows pin A 52 isolated and related to the borders of each targeted anatomic site in the anatomic hierarchy), and description of dynamic anatomic address (a reproducible location within the dynamic anatomy library in a medium 995) components, and other functions. Placed pins 52 on the anatomic visualization 10 displayed with the GUI 991 indicate locations where patient medical events have occurred. In this example, dynamic pin descriptions indicate shave biopsies were performed on various locations on the forehead, and the inputs from an input device 988 can be stored in the tangible medium 995 of the system 985. The pin list 53 shows an isolated visual preview 68 including information associated with each pin 52, including pin order, and pin position relative to the selected anatomic site component of the dynamic anatomic address, automatically populated diagnosis with a description and code, and the dynamic pin description in the same color as the pin, allowing for synchronization to the map and other outputs (e.g. enabled by components of the system 985 such as the data processing module 999). Below the pin list 53, there are diagnoses listed in collapsed lists correlating with other pins (melanoma) and painted distribution segments 51 (dermatitis NOS and acne) on the layered anatomic map 50. Despite pins 52 and hierarchically painted distribution segments 51 with colors, patterns and opacities (as determined by the input device 988 in certain embodiments) occupying anatomic site segments, the color-coded legend to display the multidimensional site visualizations and translated descriptions relative to the cursor point is still visualized with the GUI 991 without interruption because of its underlayment. Hierarchical overlays again could cause real-time visualization obscurement issues as illustrated in FIG. 1G, which is solved by the system 985 when generating the visualization with the generation module 993 as one example.

FIG. 1M depicts the same anatomic visualization 10 in a Spanish translation as the previous English figure. The translation occurs in real-time and simultaneously when instructions stored in a module in the medium 995 are in communication with the processor 986, allowing for multiple users to use the same tools and visualizations in a shared, real-time session but in different languages (for example through communication with the data processing module 999, the knowledge base module 992, and/or other modules in the medium 995 in the system 985 to display a translated GUI 991 on different physical displays 987 as non-limiting examples). It is contemplated that the embodied application is capable of translating diagnosis extensions and extended descriptions and all displayed text, including automatic enhanced descriptions of the multidimensional anatomic maps, points, distribution segments, and visualizations, in any coded, linguistic, or symbolic language through a plurality of neural networks and engines enabled by the system 985. Also depicted on the GUI 991 is a quick zoom functionality 56 that has translated isolated diagram visualizations to selectively target, find, and zoom in on multidimensional anatomic areas of interest. Also depicted are options in a hierarchical painting method 57 to paint diagnoses with different colors, patterns, intensities, opacities, and properties to multidimensional anatomic maps or avatars which are derived from and/or updated in modules in the tangible medium 995. It is contemplated that surface areas and intensity of involvement are also automatically calculated based on anatomic distribution, such as with the knowledge base module 992 and/or the data processing module 997 as a non-limiting example.

It is contemplated to have automatically linked data buckets to each dynamic anatomic address (e.g. in the system 985 with the medium 995 in communication with the processor 986) that accepts, segregates, collates, orders, tags, annotates, extracts, labels, error corrects, and analyzes pin-level photos, attachments, and links, and generates and stores dynamic forms (with or without encryption) for printing, signing, sharing, and saving. The forms and files are labeled automatically with dynamic anatomic address metadata, in any combination of the filename, file metadata, or file contents. It is contemplated that the system 985 with the tangible medium 995 in communication with the processor 986 enables artificial intelligence and machine learning processes that can extract, modify, analyze, and categorize different components of the dynamic anatomic address and the contents of their data buckets stored in the medium 995. Additionally certain embodiments enabled by the system 985 described herein is the ability to visualize an anatomic site preview, such as an isolated visual preview on the GUI 991, during transfer, copy, or merging of data in the system 985. This example provides a visual aid to the user on precisely where the data is going to go rather than simply relying on text. As one example, the visualizations reduce the risk of associating a photo with the wrong anatomic site or wrong bucket.

FIG. 1M also depicts the same anatomic visualization 10 and color-coded legend 12 of FIG. 1L in a Spanish translation on the GUI 991. The translation occurs in real-time and simultaneously, e.g., through the data processing module 999, allowing for multiple users to use the same tools and visualizations in a shared, real-time session but in different languages. It is contemplated that the embodied application system is capable of translating diagnosis extensions and extended descriptions and all displayed text, including automatic enhanced descriptions of the multidimensional anatomic maps, points, distribution segments, and visualizations, in any coded, linguistic, or symbolic language. Also depicted on the GUI 991 is a quick zoom 56 functionality that has translated isolated diagram visualizations 570 to selectively target, find, and zoom in on multidimensional anatomic areas of interest. Also depicted on the GUI 991 is a selector for hierarchical painting colors and patterns 506 with options for a hierarchical painting method to paint diagnoses with different colors, patterns, intensities, opacities, and properties to multidimensional anatomic maps or avatars. It is contemplated that surface areas and intensity of involvement are also automatically calculated based on anatomic distribution, e.g., as enabled by the system 985 with the processor 986 in communication with a module or the modules in the medium 995.

It is further contemplated that forms (e.g. such as those printed with the output device 989 and/or those shown on the GUI 991) will have relevant visual previews and isolated or grouped diagram segments, and photographs, thumbnails, and other multimedia and can accept electronic signatures and other digital inputs, while saving back to the relevant buckets including those in the dynamic anatomic address such that each bucket will show a dynamic count of relevant photos, attachments, links, and forms contained in it as depicted in FIG. 1N.

Functionality demonstrated by the non-limiting example embodiments allows for the user to attach a variety of patient data, including but not limited to photos, attachments, links, procedure type, procedure measurements, procedure counts, procedure weight/value, diagnosis, diagnosis category, insurance status, fee schedule, units, country-specific billing rules, region-specific billing rules, deductible status, copay status, coinsurance status, account balance status, discount status, and other billing associated metadata, as well as comments, and other patient data associated with a particular anatomic site. Such attachments may be stored in a database interface module 996 in the medium 995 as one non-limiting examples. It is contemplated that artificial intelligence and machine learning processes in the system 985 that may have modules in the medium 995 in communication with the processor 986 can apply neural networks and computer vision (e.g. that processes inputs received by the input device 988) to extract, modify, analyze, segment, combine, and categorize different components of non-anatomy data, the dynamic anatomic address and the contents of their buckets (e.g. with the data processing module 999). It is further contemplated that one skilled in the art would understand that any additional relevant data could additionally be incorporated into the patient record, by generating a formatted record with the generation module 993 and storing the record with a database interface module 993.

This embodiment further depicts functionality enabled by the system 985 including printing capability (e.g., to an output device 988), visibility toggle (e.g., to affect the GUI 991), label reordering capability (based on user interaction with an input device 988), customization of pins, and different types of pins (based on settings in the knowledge base module 992). The aforementioned and further mentioned examples are intended to be non-limiting illustrations of the system 985. It is contemplated that printing could allow a user to print a form that includes dynamic anatomic address, optionally enhanced anatomic site descriptions, informational codes such as QR codes, visual previews, physical labels with isolated or grouped visual previews in any label format, and all relevant diagnosis, encounter, patient, healthcare provider/physician, and clinic information. It is contemplated that a visibility toggle would hide and show relevant information on the display 987 screen in the GUI 991. The embodied example functionality would allow a user to reorder the pins through interaction with an input device 988. It is contemplated that when reordering occurs the corresponding list items, visualizations, map, and dynamic anatomic addresses are also dynamically reordered in the list and synchronized to the map. In the illustrated embodiment, a pin list sub-toolbar 58 allows the user to change additional pin-list options, such as pin-type, order type (from “A, B, C” to “1, 2, 3” in English or equivalent change in alternate languages), grouping, and whether the pin can be placed off the map in the whitespace (e.g. where it is not associated with an anatomic site). It is further contemplated that visual previews could show each pin and distribution segment, along with pin order and position relative to the anatomic site. It is contemplated that additional types of pins could include found pins that find and identifies the dynamic anatomic address in any level of hierarchy, in any language, in any dimension, on any image or an expanded pin allowing further documentation and tagging options, in any language, that are specific to dynamic anatomic address, the country the application is being used in, diagnosis, procedure, form, or other metadata (e.g. as enabled by the data processing module 999 of the system 985).

FIG. 1N further shows a screenshot with the real-time patient data 60 synchronized to an anatomic map that includes a mix of alpha-numeric and symbolic translations and delimiters (e.g., with translation performed by the data processing module 999 in communication with other modules of the medium 995 such as the knowledge base module 992). In this example, the patient's name is followed by a numeric age and symbolic translation indicating patient sex, a “birthday cake emoji” precedes the patient date of birth, and a “calendar emoji” precedes the encounter date. Symbolic emoji tags are shown for brevity and also translated with linguistic parallels in any coded or linguistic language, so either or both may be interacted with in the medium 995. The cursor 14 (e.g., with position determined by the input device 988) is shown over the right superior paramedian forehead, as indicated in the color-coded legend 12, and the laterality label 43 indicates “R” for “right” representing an outside-observer view of the anatomic visualization 10. In mirror or selfie view, which can be selectively shown on the GUI 991, the laterality label 43 would transform to an “L” representing “left”. Even though the diagrams and maps shown in this diagram do not have the midline of the body centrally located, mirroring or otherwise altering the axes (e.g., through the data processing module 999) still allows for multidimensional labeling, visualization, and translation because of the custom axes for each path. Also depicted is an example of the GUI 991 with functions to convert a pin to a different pin type 515 and a pin to distribution segment 515 and therefore an invisible pin that alters the display properties of a dimension and coordinate set of a map. Data buckets 516 (e.g., storage in the tangible medium 995 enabled by the database interface module 996) associated with the patient, the pin or distribution group, the pin or distribution list, the or the encounter may contain photos and a count of photos 509 associated with them or other data, such as links 510, as illustrated in the current embodiment. Also depicted in this embodiment are thumbnails for images and attachments 517 in the data bucket for this pin 52, and the images and attachments have associated symbolic tags 518 represented by emojis (e.g., enabled by the data processing module 999).

Two pins 52 are depicted. The first as an asterisk (*) representing a cryosurgery procedure to a diagnosis of an inflamed seborrheic keratosis. The second as “.A” representing a shave biopsy procedure on the diagnosis of a neoplasm of uncertain behavior of skin (2F72.Y). Further, the diagnosis component and other components like the pin description can change dynamically in the dynamic anatomic address, but the anatomic site information and visualization of the location can remain static (e.g., through targeted application of the system 985). This is especially helpful at different time points, since diagnoses can change with additional information, like a pathology report from the biopsy. In this example, a user could easily change from “.A” representing the shave biopsy procedure and the diagnosis of “neoplasm of uncertain behavior of skin (2F72.Y)” to a diagnosis of “melanoma” using the pin-to-pin transformation method as shown. Pins can also be transformed to distribution segments with a pin-to-distribution segment method, and vice versa, e.g., through interaction with the GUI 991 with the input device 988.

Additional information shown on the GUI 991 example could include site name preview, auto-relation, hierarchical selector 505, find pin, isolated visual preview, dynamic pin description, list subtype selector, QR code linked to dynamic anatomic address information, such as patient data, re-creation data, anatomic site data, and other health data. Links are also categorized with symbolic delimiters and tagged with language agnostic symbolic and translatable text tags, which can serve as symbolic definitions in the tangible medium 995. As one skilled in the art would know, information could be manually entered by a user or inserted automatically depending on the automatically translatable features of the embodied application system. In the present example, the procedure name (e.g., “shave biopsy”) could automatically be inserted into the notes box by automatically translatable text blocks, e.g., as enabled by the data processing module 999.

FIG. 1N also depicts a screenshot with dynamic patient and encounter synchronization of data to map. Real-time synchronized map data 60 includes text like patient name, symbolic translations like “male symbol emoji” for male sex as a symbolic definition, and symbolic delimiters like the “birthday cake emoji” to represent date of birth and the “calendar emoji” to represent encounter date, and is enabled in the system 985 through the medium 995 and/or modules such as the data processing module 999. It is contemplated that some symbols such as those for patient sex stand on their own as symbolic definitions, and do not need additional data, while others like the “birthday cake emoji” would typically be tied to other data like a date, and thus the date of birth would be delimited by the “birthday cake emoji” in an order independent and structureless string of data blocks. The cursor 14 (e.g., position on the GUI 991 determined by the input device 988) is shown over the right (superior) paramedian forehead with a corresponding color-coded legend 12 to the bottom left of the figure. On that same diagram, two pins are dropped, with a brown “*” representing cryosurgery procedure to a diagnosis of an inflamed seborrheic keratosis, and a red “. A” representing a shave biopsy procedure on the diagnosis of a neoplasm of uncertain behavior of skin (2F72.Y), with the “.” representing the precise point of the biopsy procedure. The QR code 62 that contains dynamic anatomic address (a multidimensional and reproducible anatomic site that is trackable at different time points) information is partially redacted, but it contains patient data, re-creation data, anatomic site data, and other data blocks, e.g., that can be in communication with the medium 995. This embodiment also depicts the icons for changing procedures, diagnoses, list memberships, and pins to distribution segments, along with a dropdown that shows the pin preview and description of the pin should the user wish to automatically change multiple components of the dynamic anatomic address but keep the selected anatomic site and site descriptions constant. In this embodiment, the user could easily change from “. A” representing a shave biopsy procedure on the diagnosis of a neoplasm of uncertain behavior of skin (2F72.Y) to a diagnosis of melanoma, thus dynamically changing some of the data blocks related to this pin and the other data attached to the pin, such as the file name diagnosis, and updating the medium 995. In other words, the diagnosis component and other components like the pin description change dynamically in the dynamic anatomic address, but the anatomic site information and visualization of the location remains static.

It is contemplated that files named with symbolic delimiters and symbolic definitions could easily name photographs and attachments with a meaningful written story or novel about that photo or attachment, automatically, e.g., enabled by the data processing module 999 in communication with the knowledge base module 993 and/or the generation module 993 as one non-limiting example. Dynamic changes in portions of the data blocks are especially helpful at different time points, since diagnoses can change with additional information, like a pathology report from the biopsy, but anatomic site data blocks will remain the same. The notes section was typed with the input device 988 in with free text in the example embodiment, and it is not currently tagged for translation. There are digital “chips” of data blocks accessible in the medium 995 relevant to the encounter that can be used to insert automatic, automatically translatable text blocks into the notes box, such as the procedure name (“shave biopsy” in this example). There is also a morphology selector and a symptom selector (not shown, but contemplated to be selected with the input device 988 as one example) in which the user can enter these automatically translatable features. It is contemplated that the user is a computer, and for example, the morphology data blocks can be detected from the images and added automatically e.g., through communication between the processor 986 and the medium 995. It is contemplated that other features could be added into different parts of the application from automatic image detection, such as skin type and skin tone in other embodiments. It is contemplated that skin subtyping and sub-toning can also be enabled in other embodiments of the system 985, such as a customized skin type and tone that has a much more granular scale and based on dynamic anatomic address average calculations and confidence intervals, and other features such as age. For example, sun exposed skin on the face may be more darkly pigmented in older patients who spent a lot of time in the sun, and the skin on their inner arm or buttock where there is typically less sun exposure would provide a more accurate skin type and tone measurement for the overall patient, and is therefore weighted more heavily in the skin subtype and subtone calculations from the different skin tone and skin type data blocks based on anatomic locations.

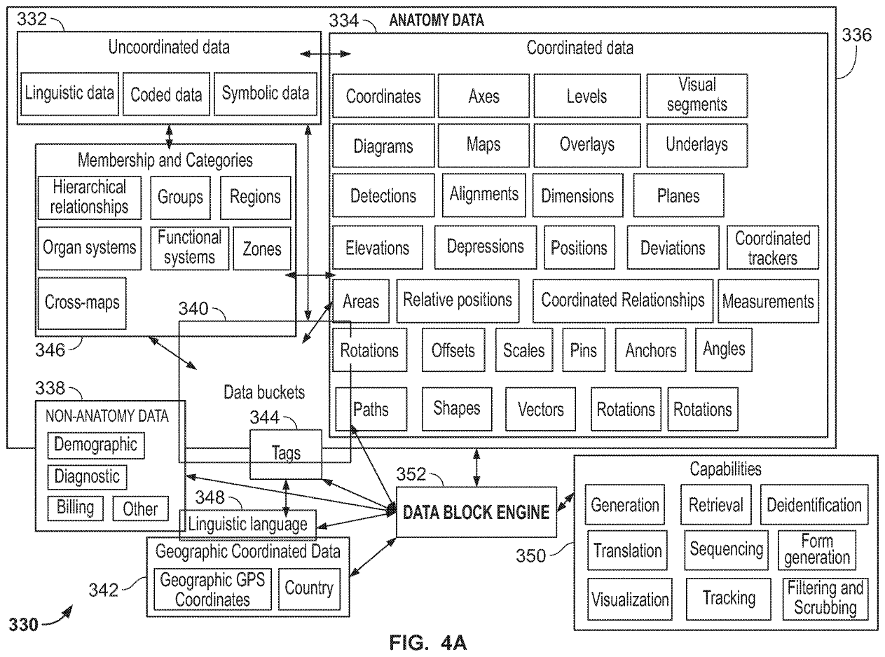

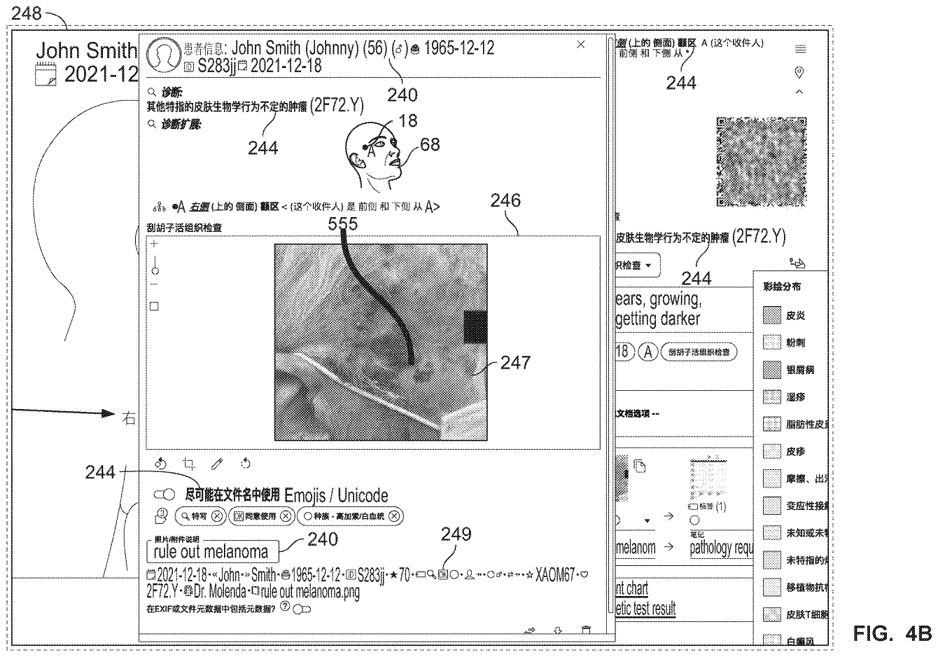

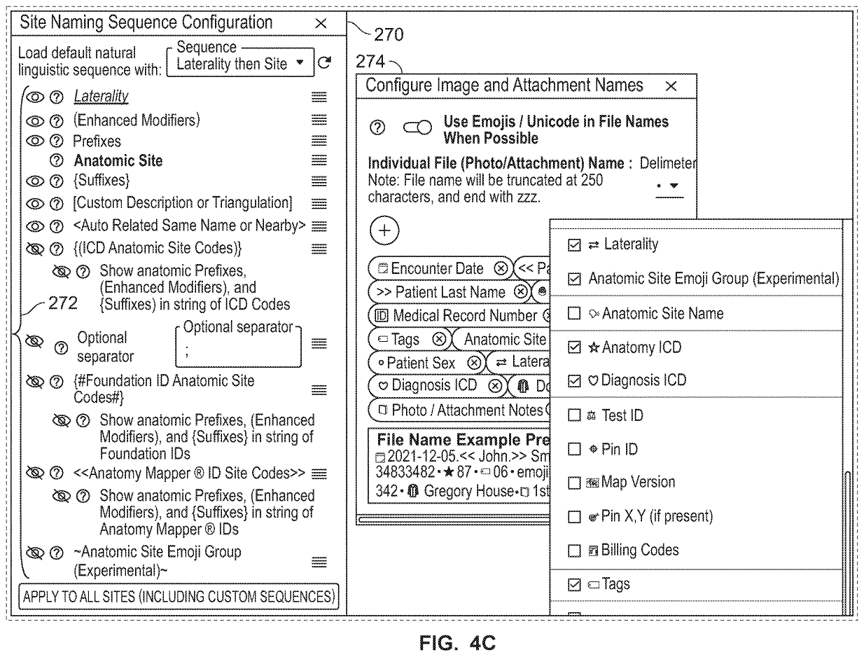

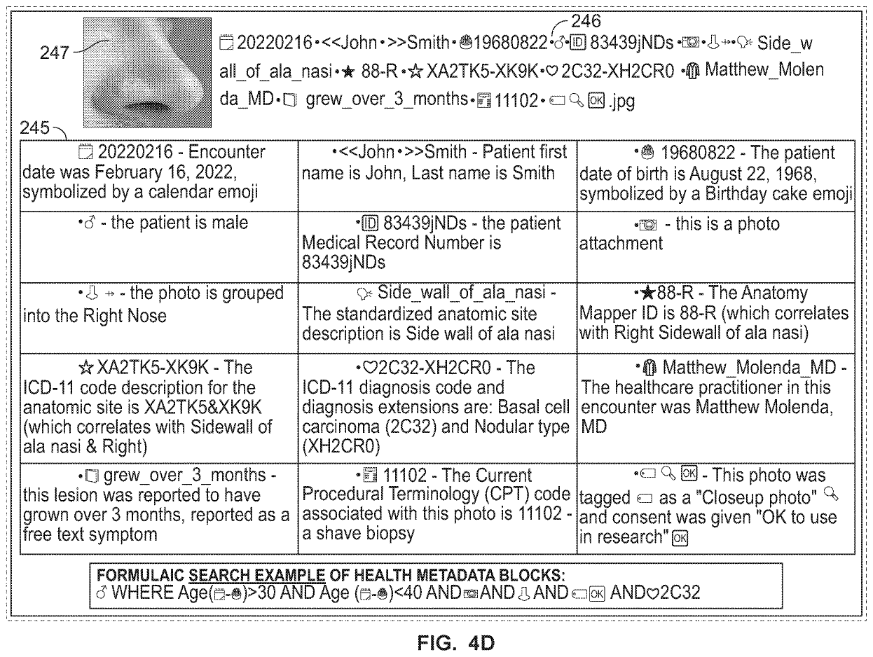

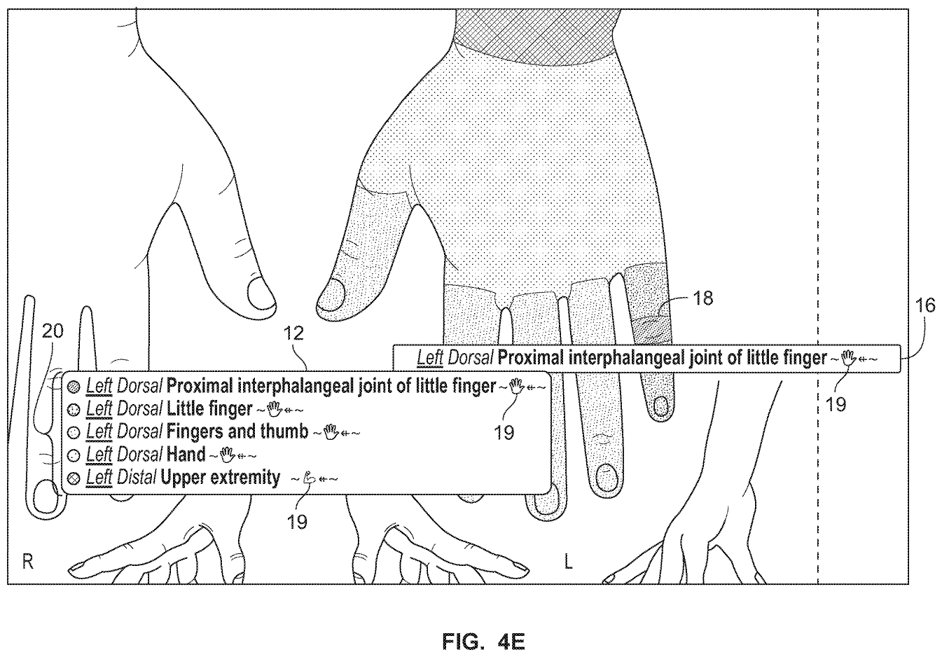

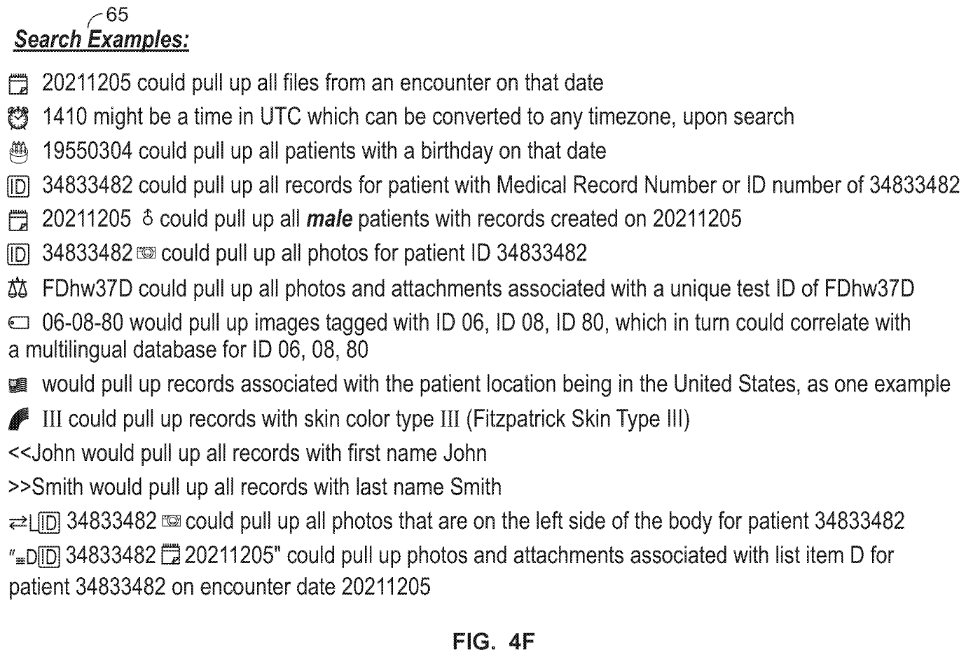

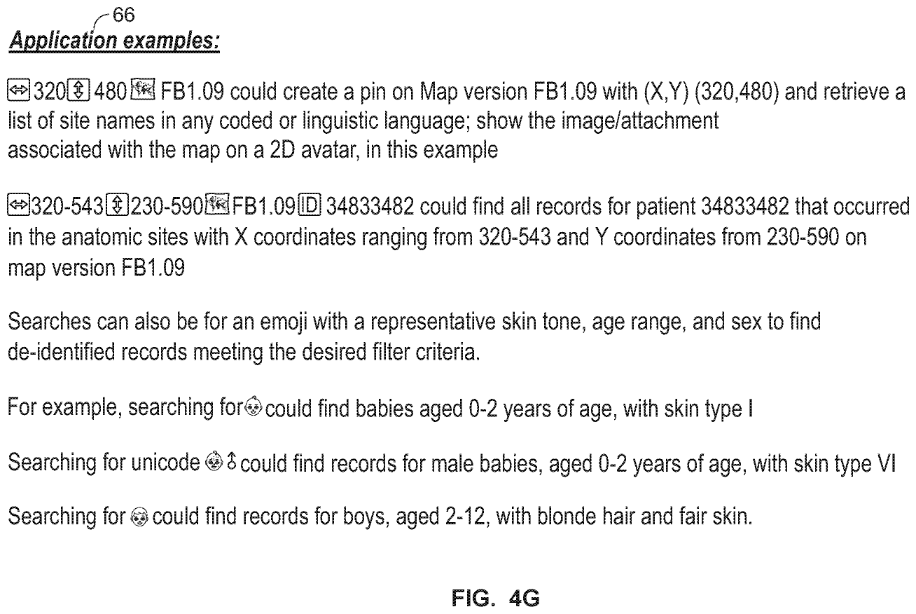

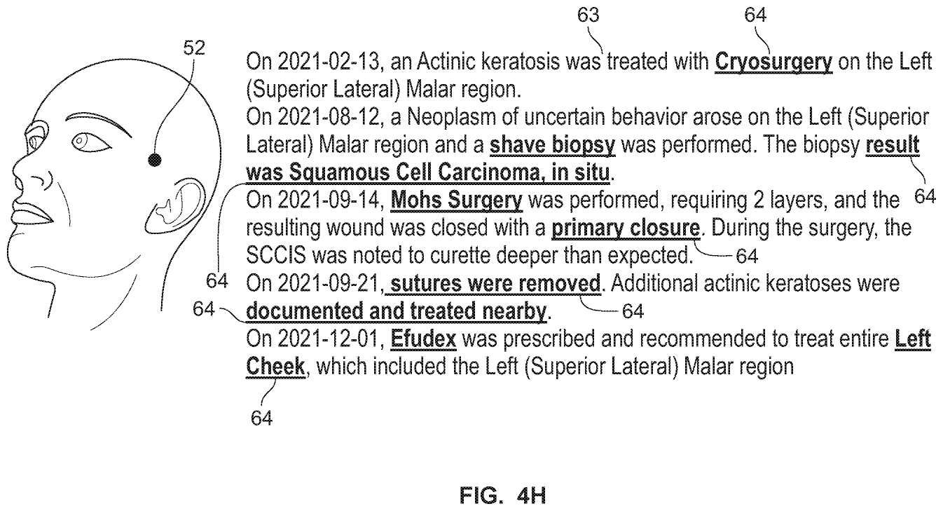

It is contemplated that lighting features captured by geographic GPS coordinates, weather, indoor/outdoor settings, lighting, and camera settings, and other EXIF metadata from the input device 988 can also be data blocked and analyzed (e.g., by the data processing module 999) for more accurate readings. It is further contemplated that standardized color wheels (e.g., as the input device 988) can also be used along with camera capture. In the depicted embodiment, each photo and attachment are joined into a collated bucket (e.g., with the database interface module 996) for this pin and dynamic anatomic address which has a symbolic definition, and are tagged and notated with other symbolic tags, definitions, and delimiters. It is contemplated that a symbolic definition can also serve as a symbolic delimiter, with the “tag emoji” being an example of this. The “tag emoji” symbolically delimits other symbolic defined tags, such as a “magnifying glass emoji” to tag a photo as a closeup, or an “OK emoji” tagging the image as approved for use in research. Thumbnails are shown for each photo and attachment in the currently joined bucket and clicking on it will bring up a modal in the GUI 991 to view the thumbnails as navigable and editable images in larger size (shown in FIG. 4B). Symbolic emoji tags are shown for brevity, but these tags are also translated with linguistic parallels in any coded or linguistic language using a neural network (e.g., as enabled by the modules in the medium 995). Photo notes and link descriptions are only translated based on user preference in certain embodiments (e.g., as enabled by the medium 995 such as with the knowledge base module 992). Links are also categorized with symbolic delimiters and tagged with language agnostic symbolic and translatable text tags.

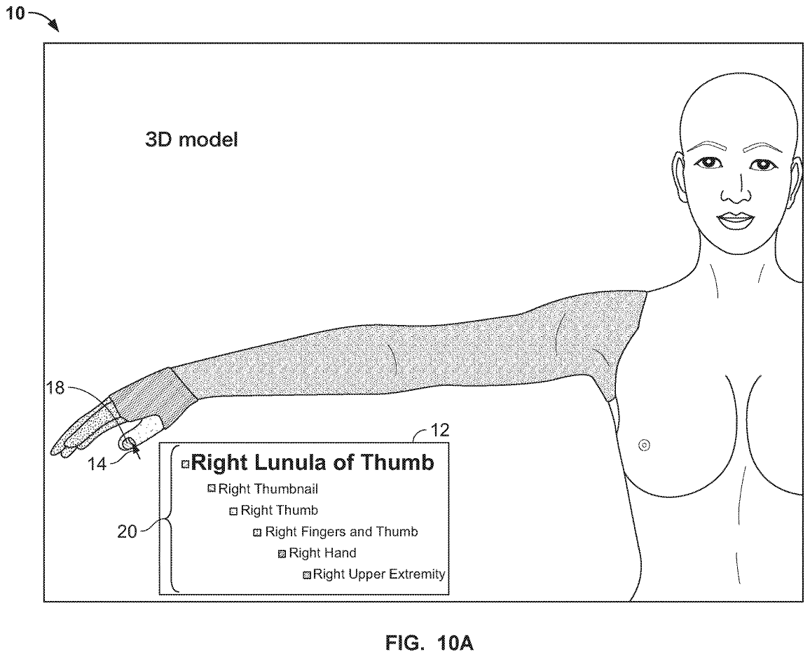

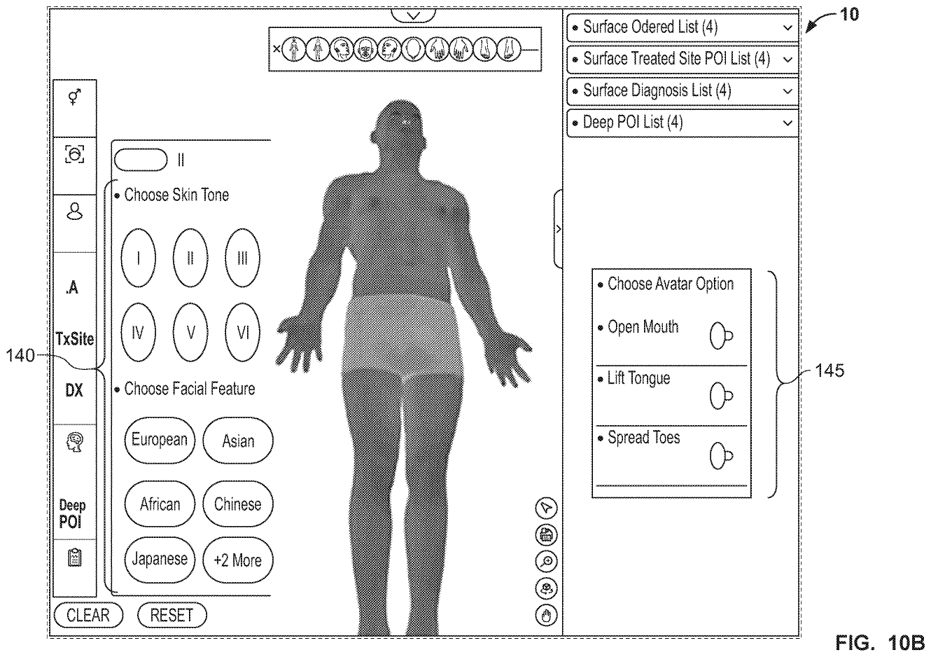

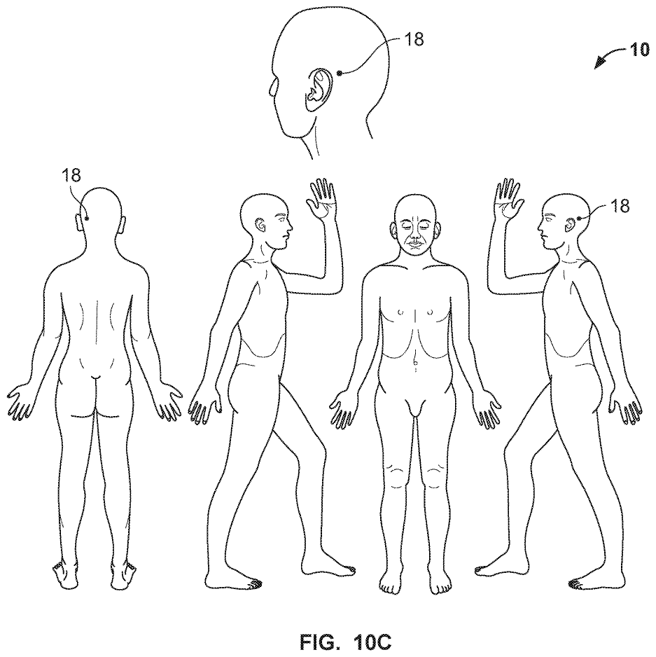

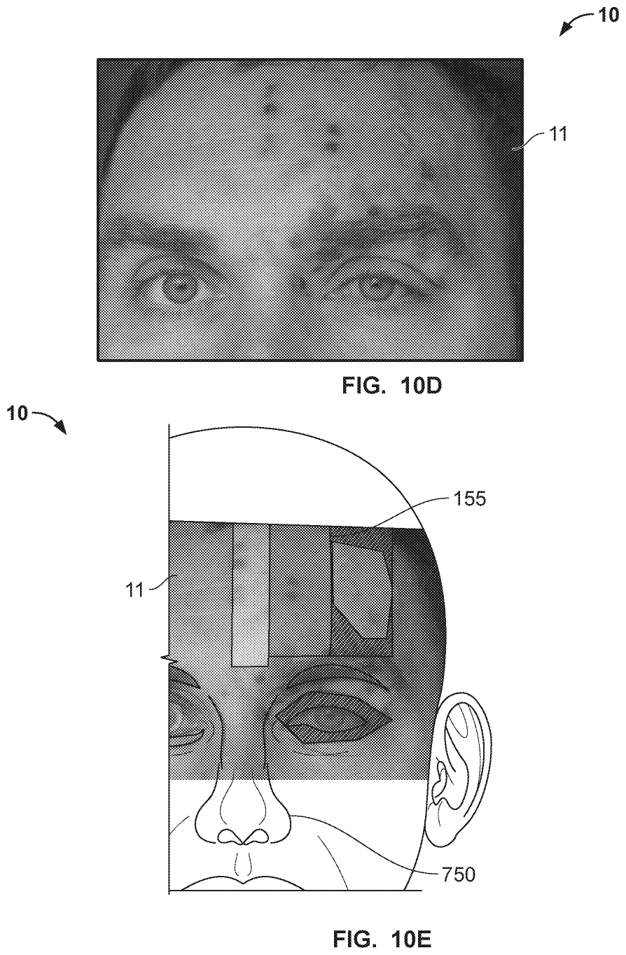

FIGS. 10 and 1P depict exemplar two-dimensional models of a right dorsum of hand 70. FIG. 1O shows a bounding box 42 segmented into customized Enhanced Detail Region (EDRs) on the GUI 991. It is contemplated that an EDR can be a custom shape, path, vector, or compound path, with custom offsets and angles stored and/or processed in communication with the medium 995. It is also contemplated that each EDR can determine proximity to and from center, angle to and from center, and magnitude of distance and angle from center, and each EDR can be broken up by an unlimited number of sub-segmentation boundaries. There are seventeen different sub-segmentation boundaries shown for this single example EDR. Three pins 52 (A, B, and C) are shown in the central zone, one of the seventeen example sub-segmented zones. The EDR custom axes (regardless of their defined thresholds for sub-segmentation boundaries) can be used to automatically relate the pins 52 to one another (e.g. in communication with the data processing module 999), showing that A is medial from B, A is medial and proximal from C, B is lateral from A, B is lateral and proximal from C, C is distal and lateral from A, and C is distal and medial from B. It is contemplated that magnitude sensitivity may also be applied to enhanced anatomic site descriptions, such that for example (distal, medial) is different than (medial, distal), sequenced based on the magnitude of the deviation from center and the custom sub-segmentation boundary thresholds (e.g., as enabled by the data processing module 999).

In addition to EDR, there are defined Enhanced Detail Planes (EDPs) that serve as backup relational planes, axes, and dimensions for relating two or more dynamic anatomic addresses, paths, or groups of paths that can have different and/or undefined EDRs. EDPs are also referred to as “Direction Planes” in the teachings herein. For example, if two pins are close to each other but cross the midline of the body, and auto-relation is desired, the medial and lateral dimensions would not make sense, so a different directional plane and axis like an EDP is used to describe that relationship (e.g. as enabled by the data processing module 999 in communication with the generation module 993 as one non-limiting example). FIG. 1P shows a non-limiting example representative EDP, defined by a bounding box 42 with axial synonym labels. The EDR in this embodiment also shows different sub-segmentation boundary definitions in the y-axis as compared to the FIG. 1O, omitting the automatic addition of the y-axis terms for this EDR only. It is also contemplated that EDR and EDP functions in the medium 995 also work in three dimensions by adding a z-axis to each customized coordinate set, and custom offsets, rotations, scales, measurements, selective alignments, vertices, and topographies (including depressions and elevations) are also added for increased accuracy and precision. Because the axes applied to paths are customized in the medium 995, even three-dimensional views can take advantage of multidimensional hierarchical labeling without having an avatar or avatar component center in view or containing a midline. It is also contemplated that dynamic anatomic addresses can be recreated, enhanced, partially changed, and auto-related at different time points, thus in the fourth dimension of time, and moved, repositioned, or merged into other dynamic anatomic addresses with patient changes such as surgical changes, weight gain or loss, and growth, e.g. through the data processing module 999 in communication with the record retrieval module 997 as one example. It is also contemplated that there are multiple medians and midlines on maps, images, and avatars representing anatomy in the tangible medium 995. For example, body midline and median are different then the arm midline and median. Such midlines can also change based on the rotation, view, and area of the visualized anatomy.

FIG. 1Q depicts four pins 52 on the same anatomic site 18, in this example the “central forehead” site that are automatically related to one another (e.g., enabled by the data processing module 999 and/or generation module 993). Descriptions of all pins on same anatomic site 18 are automatically related, even though, in this case, some pins cross or approach the body midline. Additionally, linguistic descriptions to describe the magnitude of axial deviations from each other and from center are called “magnitude modifiers” and may also be applied when appropriate. Example automatic relationships with magnitude modifiers (e.g. also can be enabled by the generation module 993 in communication with the data processing module 999 as one example) for the pins 52 in FIG. 1Q include: A is superior and right from B; A is superior from C; A is very superior and barely right from D; B is inferior and left from A; B is superior and left from C; B is superior from D; C is inferior from A; C is inferior and right from B; C is barely right from D; D is very inferior and barely left from A; D is inferior from B; and D is barely left from C.

It is contemplated that all descriptions are and/or can be translated automatically into any coded, linguistic, or symbolic language and directional arrows may also be depicted relating pins to one another in other embodiments. It is further contemplated that automatic directional omissions will be implemented when necessary to help avoid human user confusion when interpreting the automatic linguistic descriptions of the pin relationships (e.g., as enabled by the data processing module 999). For example, “A is Superior from C” automatically omits x-axis descriptions. Reordering or deleting a pin, such as deleting “C” in this sequence, automatically relabels “D” as “C” and updates all auto-relation calculations and descriptions that contain the new “C”.

It is contemplated that the non-limiting embodiment of the system 985 can depict painted hierarchical anatomic sites with defined but customizable morphologies. FIG. 1R depicts a screenshot of an example of hierarchical painting of morphologies related to a disease, rather than diseases as shown in a prior figure. The customizable morphologies (e.g., stored and interacted with in the database interface module 996) are painted to selected and segmented hierarchical components with dynamic anatomic addresses. Each morphology or morphological combination has any combination of color, opacity, pattern, and intensity. Body surface area is automatically calculated for each morphology in other embodiments at its selected dynamic anatomic address component. Each mapped morphology has a corresponding visualization, and hierarchical selectors 505 on the GUI 991 allow for different selections by the input device 988. An exported click-order matrix 85 that outputs the morphological findings into a tracking matrix (e.g., with the generation module 993), so that disease progression can occur by comparing different time points (e.g., with the record retrieval module 997 in communication with the data processing module 999 as one example). It is contemplated that output may also be exported to a standardized matrix for easier data tracking and analysis. It is further contemplated that data overflow and duplicates may be shown and accounted for in the standardized matrix exports. It is further contemplated that inputs and outputs contain trackable secondary features 87, shown as ulceration and angiomatosis in this non-limiting example, which are output into the tracking matrix. It is further contemplated that when the same site is selected on both lateralities (right and left), they can be optionally combined into a single (bilateral) visualization and description or kept separate. It is further contemplated that this hierarchical painting example documents morphologies on a single patient in a single point in time, which was done retrospectively in this non-limiting example, but can hierarchically paint on a combination of different patients and different time points, thus the system 985 enabling tracking of disease progression, resolution, or change in individual patients over time, or in populations.

The system allows for tracking through time as well. FIG. 1R is a screenshot of an embodiment illustrating morphologies of a disease mapped to anatomic visualizations at different points in time. Affected anatomic sites are highlighted in different colors, intensities, patterns, opacities, and hierarchical levels to provide both visual and descriptive characteristics of the morphology. This embodiment depicts single and combination morphologies. It is contemplated that surface areas and secondary features like ulceration and angiomatosis, and features such as intensities, counts, measurements are also mappable and trackable in the system 985. Disease progression is tracked by outputting a matrix as embodied as one example. In certain embodiments, the output matrix is presented at a single point in time based on click-order and cross-maps the anatomy to ICD-11 encoding. It is contemplated that the table could be arranged in alternate outputs, e.g., enabled by the generation module 993. This allows for tracking of morphologies over time and anatomic sites. It is also contemplated that surface areas and secondary features like ulceration and angiomatosis, and features such as intensities, counts, measurements are also mappable and trackable, e.g., with calculations enabled by the data processing module 999 as one example. It is contemplated that one skilled in the art would know that this is just one example for illustrative purposes, and there are unlimited examples of encodings, links, morphology descriptions, morphology combinations, other findings, and cross-maps that will evolve over time. As one non-limiting example, the morphology data is an example of the “non-anatomy health data” described in FIG. 9J.

It is contemplated that the morphologies generated by the system 985 (e.g., by the generation module 993) could be reintroduced to the system 985 and used as a training model and neural network (e.g., stored in the medium 995 with modules interacting with the processor 986) for machine learning, artificial intelligence, and deep learning. Data points generated by computer vision (e.g., by the generation module 993) for the training model can be vetted, approved, and modified by experts for accuracy and quality control.

FIG. 1S further depicts hierarchical painting that includes treatment recommendations 90 associated with the specific anatomic sites 18. Patients often receive multiple recommendations, sometimes with ten or more products listed, and can understandably get confused on what to use, where, and when. Visual hierarchical painting through mapping with anatomic site or site group descriptions (e.g., with the input device 988) can help to color code this information in an easily digestible format for the patient, in any language and creates a visual map of recommendations. It is contemplated that recommendations could build upon each other in areas of overlap. It is further contemplated that clinicians can stamp (e.g., with the input device 988) their recommendations or highlight areas (e.g., on the GUI 991) for where to apply their recommendations on standardized maps or directly on patient images creating a personalized visual map of recommendations with simultaneous condensed and simplified translated text descriptions of “what to use where” on their body. The visual map of recommendations can be printed to the output device 989, and if a recommendation is unilateral, the physical output labels as shown in FIG. 1T could be mirrored and/or otherwise altered by the image interface module 994, as one non-limiting example.

FIG. 1T depicts an exemplar output 90 (e.g., to the output device 989) with the treatment recommendation 90 for the anatomic sites 18 shown in a combined visualization in this non-limiting embodiment 525. It is contemplated that the output can be printed, digital, or both, with relevant isolated or combined visual previews as well. It is contemplated that regimen mapping can be applied to treatment mapping as well, such as documenting different types of cosmetic procedures or settings, in other embodiments. It is further contemplated that printed outputs to the output device 989 could be made into labels and then affixed directly to the container for products, including over the counter products, to better instruct the patient how and where each product should be used to minimize confusion particularly when there are multiple products being used at different times of day. Printed physical labels inform a patient about the product regimen such as, how to use it, where to use it (which anatomic locations 17), frequency, warnings, and more. It is contemplated that emoji labels can also be utilized to communicate how the patient should use the medication. For example, a pill could show a mouth with a cup of water to indicate it should be taken orally, a “pill emoji” and a “food emoji” could indicate it should be taken with food, or a pill with a “no sign emoji” and a “cheese emoji” could indicate to avoid dairy when taking. If the current exemplar were unilateral, for example directing the use on one eyelid, the visualization could be shown in mirror view (e.g. by the image interface module 994 and/or the generation module 993 as non-limiting examples) since this exemplar is representative of a physical label the patient could put directly on their product bottle, and the patient would likely be applying the product in front of a mirror. Such a label as described (unilateral, in mirror view) would serve to minimize patient confusion.

It is contemplated that the workflow of associating anatomic sites and regimens through the system 985 can flow in either direction. A user can assign (e.g., with the input device 988) a color to an anatomic site, then add products, treatments, and recommendations to the painted anatomic sites. Conversely, products could be assigned a color and then the products could be painted onto the avatar. Anatomic areas are defined by area names and colors (e.g., on the GUI 991). It is contemplated that when recommendations are asymmetric, the visualizations and sites can be plotted from the outside observer perspective by the physician and viewed in a mirror perspective by the patient to enhance patient understanding.

FIGS. 1U and 1V depict an electronic regimen map 100 on the GUI 991 with printable labels 102 to the output device 989 and affiliated educational instructions 105, respectively. In the embodiment, the educational instructions 105 provide visualizations by product, by area, and by condition (e.g., to the GUI 991 and/or to the output device 988). While a paper workflow generates a digestible report that can be handed to the patient; the electronic regimen can be saved into the patient's chart for tracking the regimen over time and initiation of other workflows. The electronic regimen can evolve over time, with input from the patient and the professional (e.g., on separate and/or the same input devices 988 as one example), in their preferred language (e.g., on the GUI 991 enabled by the knowledge base module 992 and/or data processing module 999 as one example). It is contemplated, in an electronic evolving regimen, the patient could give feedback on a product, report a side effect from a product, initiate a refill request for a product, find up to date manufacturer's coupons for a product, view product recall information, ask their professional about the product, report stopping the product, report starting a new product, or other electronic tasks (e.g. enabled by the input device 988 in communication with the system 985). It is further contemplated that automatic alerts could be sent to the patient and physician if there is a product recall and automatic reminders could be sent to the patient if they are expected to be running low. From the electronic regimen, when there are office-dispensed products recommended, a single link could add all the office-dispensed products that are in stock to a ticket system with real-time pricing update for the patient to initiate a purchase and the patient could also purchase remotely for products from office/retail facility (e.g. by populating a shopping cart on the GUI 991 with the input device 988).

In certain embodiments, products can be registered (e.g. with the database interface module 996 in the system 985) including information including but not limited to: photo, product name, product ingredients, product SKU, product category in user country (Rx, office dispensed, OTC), product vehicle, product warnings, product directions, product suggested frequency, product side effects, product anatomic sites to avoid, product recommended anatomic sites, product substitutes, product key recommended ingredients, product active ingredients, product availability in user country, product synonyms, and other product metadata. A patient can then select a product that was already recommended, from the global product library, from the localized product library (e.g., based on country), or from their custom/favorite library of products (e.g., with libraries stored and communicated within the tangible medium 995). Automatic product recommendations and warnings will populate (e.g., enabled by the data processing module 999) based on user preferences, patient condition, user frequency of selection, patient allergies or adverse reactions, patient interactions with other medications or conditions, patient has tried product already and failed, product availability (backordered, discontinued, banned in this country/region, etc.), substitutions, and other metadata. The patient can then print a physical label (e.g., to the output device 989) with a product image/thumbnail and the regimen for easy correlation and visual recognition by the patient. A product thumbnail can be expanded to show other metadata about the product on the electronic regimen on the GUI 991.

The ability to dissect, sequence, and visualize anatomic site name components within the embodied system 985 is depicted in FIG. 1W. The screenshot shows the Anatomic Site Name Builder 110 which allows a user to reorder components (e.g., through the input device 988 in communication with the GUI 991) of an anatomic site's name or description. The name components are linguistically dissected and automatically placed into digital “chips” (which themselves can be deleted and reordered) in the appropriate category (e.g., by the data processing module 999). In the illustrated embodiment, neural networks enabled by the system 985 (e.g., through modules in the medium 995 in communication with the processor 986) output the automatic coded translations and symbolic groupings in the GUI 991 which are showing for ICD-11, Foundation IDs, AnatomyMapper ID code strings, and for symbolic emoji grouping of this selected anatomic site component, and laterality in this non-limiting embodiment. The visibility toggle 112 has been toggled for the ICD-11 codes (e.g., with the input device 988) in this example, the non-limiting enhanced code string displayed being “XA1Z38&XK8G_(XK4H)” on the GUI 991.

Embodied as a non-limiting example in FIG. 1W is the system 985 delivering an enhanced anatomic site description or name with natural linguistic sequencing (or natural semantic sequencing) automatically separates the anatomic site description or name components into laterality, prefixes, enhanced modifiers, suffixes, anatomic sites, anatomic distributions, distribution segments, custom descriptions, codes (which can be alphanumeric, numeric, or other codes), and symbolic groupings (such as emoji groups). The visible name components 114, include but are not limited to laterality, enhanced modifiers, prefixes, suffixes, automatic coded translations, and symbolic groupings (here depicted showing for ICD-11 anatomy codes and Foundation ID codes and Anatomy Mapper IDs as enabled by the knowledge base module 992). The visibility toggle 112 of this example has been toggled for the ICD-11 codes in this non-limiting example, the code displayed being “XA1Z38&XK8G_(XK4H)”. In the illustrated embodiment, select name components and cross-mappings have been hidden with the visibility toggle 112. These hidden name components and cross-mappings 116 can be made visible again at the user discretion. The anatomic site name builder 110 displays linguistically dissected, reorderable, uncoordinated components of the selected hierarchy level's site name delivered by the mapping engine from a visual input selecting an anatomic site 18 on a multidimensional anatomic map. Name components are automatically placed into digital “chips” enabled by the system 985 in the appropriate category. The label reordering capability of the system allows for both reordering and deletion of dissected name components (e.g., with the input device 988). In certain embodiments, custom triangulation can be added. Also, in FIG. 1W, the user interface (e.g., on the GUI 991) behind the name builder is embodied in the example, which has an isolated visual preview of the selected pin in the list.

FIG. 1X shows the capability of the embodied system 985 to enable progressive linguistic subsegmentation (also can be described as semantic subsegmentation, which can be progressive (to more specificity) or regressive (to less specificity)) and visual sub-segmentation simultaneously to achieve pinpoint precision in defining anatomic sites 18 on an anatomic visualization 10 (e.g., as shown on the GUI 991 and enabled by modules in the tangible medium 995). Each anatomic visualization 10 (moving from left to right) progressively sub-segments from the previous until the right most visualization achieves pinpoint precision for the progressively described (e.g., through semantic language) dynamic anatomic address. Above each visualization 10 are the English enhanced linguistic anatomic site descriptions 120 of the progressively sub-segmented anatomic sites. Each subsequent diagram adds enhanced modification language and simultaneous visualization. In the illustrated embodiment, an enhanced modifier for sequence sensitivity is turned on in the system 985 causing the term “lateral” to be shown before “superior” in the anatomic site descriptions 120. Sequence insensitivity, another option in the system 985, would visualize the entire upper right aspect of the highlighted area (combining the two right-most visualizations 10 in this figure). Keeping sequence sensitivity on, visualization of the right-most diagram color-codes the description for the site description 120 to near pin-point precision (e.g., enabled by the data processing module 999 and/or generation module 993). In this embodiment, since “superior” is listed before “lateral” and sequence sensitivity is on for the modifier terms, the upper right of the highlighted area is more accurately and precisely targeted. It is contemplated that the same sub-segmentation could be applied to the generated diagrams (e.g., through the generation module 993) as shown or to other inputs (e.g., such as those from the input device 988) such as a patient photo, avatar, video, live camera for augmented reality, virtual reality avatar, or other multimedia. It is further contemplated that the anatomic site descriptions 120 could be linguistic, symbolic, coded, mathematical or some combination of descriptors. The embodiment exemplifies language transforming into precise coordinates enabled by the system 985 and is also describable as a linguistic (and/or semantic) global positioning system, and/or a semantic coordination system, etc.

FIGS. 1Y and 1Z depict the capabilities of the embodied examples of the system 985 to customize the anatomic visualization 10 generation (e.g., through the generation module 993) based on patient characteristics, as well as translation of those customizations (e.g., through the data processing module 999). The anatomic visualization 10 is representative of the patient identified by the identifying data 125. The customizable interface aspects 130 on the GUI 991 can adjust for properties like patient sex, language, visibility, and filtering of certain anatomies, etc. In the non-limiting embodiment depicted in FIG. 1Y, the text appears in English and symbolic language, the selected sex is male, show oral anatomy is selected and these customizations are reflected in the anatomic visualizations and maps 10. In comparison, in the non-limiting embodiment in FIG. 1.Z, the text appears as Chinese characters and symbolic language, the selected sex is female, the oral anatomy dynamic anatomic addresses are hidden, and these customizations are reflected in the anatomic visualizations 10. The embodied non-limiting system 985 allows for dynamic anatomic addresses that are not relevant to the patient to be filtered out or hidden automatically or with visibility toggling or by user preference (e.g., with the input device 988). The user interface GUI 991 with the anatomic visualization 10 can also be zoomed in or out. Vector and mathematical algorithms and models (e.g., stored and/or interacted with in the medium 995 in communication with the processor 986) allow for infinite and seamless scalability of each dynamic anatomic address, where neighbors and relatives are either affected or unaffected based on detected changes.

FIG. 10A depicts an anatomic visualization 10 with dynamic anatomic addressing on a non-limiting three-dimensional model visualizing the anatomic hierarchy over the right lunula of thumb with a corresponding color-coded legend 12 on the GUI 991. As with the two-dimensional anatomic visualizations, the cursor 14 (e.g., representing the position of the input device 988) hovers over an anatomic site 18 and a color-coded legend associates the linguistic descriptor with the appropriate color to define the five hierarchical regions 20 in this embodiment, which are: right upper extremity, right hand, right fingers and thumb, right thumb, right thumbnail, and right lunula of thumb. The site colors are underlaid and blended into the avatar, so as not to obscure the avatar or cause unexpected color blending caused by overlaying semi-transparent colors.

FIG. 10B depicts the customizability of a patient representative avatar to accurately represent patient characteristics 140 of a specific patient, including but not limited to adjusting skin tone, facial features, body features, body shape, and body size (e.g., through communication with the input device 988 and the GUI 991, or the data processing module 999 and/or generation module 993, as non-limiting examples). It is contemplated that textural maps could further enhance the avatar. In certain embodiments, a three-dimensional total body photograph can adjust all dynamic anatomic addresses and describe, categorize, and relate all areas of the body, in any coded, linguistic, or symbolic language. In another embodiment, high resolution three-dimensional captures detect, count, categorize, and assign gross and dermatoscopic-level features of lesions such as dermatoscopic-level morphologies and measurements. It is further contemplated that the GUI 991 allows for avatar manipulation 145 (e.g., with the input device 988), such as opening the mouth and sticking the tongue out to document on oral dynamic anatomic addresses or removing underwear to document anogenital dynamic anatomic addresses.

FIG. 10C is representative of the standardized anatomic mapping at consistent dynamic anatomic addresses. When plotting and markup occurs in three-dimensional anatomic visualizations (e.g., with the input device 988), corresponding plots and markups are simultaneously added to the anatomic sites 18 on the two-dimensional anatomic visualizations 10 (e.g., with the generation module 993) in one non-limiting embodiment. Likewise, this corresponding mapping occurs on the three-dimensional maps when plotting and markup occurs on the two-dimensional maps to ensure accuracy and consistency amongst all of the patient's anatomic visualizations. Likewise, when the same anatomic site is present on multiple two-dimensional map images, markup can selectively be shown or hidden on all views containing the same anatomic site. FIG. 10C further depicts that regions of interest can be simultaneously shown on all views of anatomic visualizations 10. In this embodiment, the anatomic site 18 or dynamic anatomic address can be seen in select perspective views of the anatomic visualization 10 on the GUI 991.

Mapping capabilities of the embodied system 985 can be used in conjunction with each other through neural networks, computer vision, and artificial intelligence enabled by the system 985 (e.g., through modules in the medium 995 in communication with the processor 986). FIGS. 10D and 10E depict anatomic visualizations 10 with such a scenario. FIG. 10D is a patient photograph 11 image added to the patient's record (e.g., by the input device 989). FIG. 10E depicts the multidimensional and hierarchical detection and visualization of anatomic sites 155 on the GUI 991 applied to the patient photograph 11 along with the alignment of the hierarchical visualization 750. It is contemplated that computer vision enabled by the system 985 can automatically count, categorize, characterize, and relate detected abnormalities, such as lesions or redness, in different anatomic sites and groups of anatomic sites. It is further contemplated that such detections will provide automatic diagnostic capabilities through machine learning and artificial intelligence (e.g., enabled by the data processing module 999 in communication with other components of the system 985, such as the knowledge base module 992 as one example), made possible by the distribution detection determined from the dynamic anatomic addresses of each detection.

The illustrated embodiments of the system 985 and/or method are a superior system to supply specific, relevant, enhanced, and translated anatomic site descriptions, coordinated visualizations, maps, and records based upon uncoordinated and mixed linguistic, coded, and symbolic anatomic site data. The system 985 generates (e.g., through the generation module 993) automatic, real-time, enhanced visualizations, coordinate assignments, avatars, maps, record delivery, and translations of uncoordinated and mixed linguistic, coded, and symbolic anatomy data inputs. It is contemplated that in a preferred embodiment the components of an anatomic site description or name are capable of being reordered.

In one example embodiment, a method for mapping, visualizing, tracking, translating, encoding, describing, and/or labeling anatomic sites, the method comprising: selecting an anatomic site on an anatomic map wherein the anatomic map is defined by two or more defined paths of different sizes wherein the paths are automatically relatable to one another or automatically enhanceable through descriptions with directional modifiers using custom axes or custom planes; providing an output wherein the output is at least one of the following: optionally associating at least one component of anatomic site data with the anatomic site wherein the site data is metadata relevant to the anatomic site; optionally creating a dynamic anatomic address that can be tracked spatially and through time wherein the address accepts images, attachments, records, links, and other data; optionally visualizing a point of interest relative to path and the point of interest relative to all underlying and overlying and neighboring paths, in any hierarchical level or group, wherein all paths are described, sequenced, and categorized in any coded, linguistic, or symbolic language; wherein the visualization is overlaid, underlaid, or aligned to other multimedia containing anatomy to apply multidimensional and optionally mirrored anatomic mapping, descriptions, and visualization; wherein the point of interest is movable such that when moved it travels to different positions within all the paths above, and below, and near it and such that the point of interest can optionally be moved to different maps, diagrams, organ systems, or path collections; optionally generating multidimensional descriptions, visualizations, and map positionings in while the point of interest is moving; optionally grouping paths or path segments with other paths or path segments to create a distribution list, and a hierarchical painting method where the hierarchical painting method allows for targeted, visualized hierarchical path selection of overlying and underlying paths, with translated visualizations, and descriptions, and calculations; wherein the distribution list includes surface area, intensity, and detected data, such as counts, morphology, treatment recommendations, or other metadata, for each group component, which can be output into a plurality of templates; optionally linking multidimensional anatomic points and distribution segments to points in time wherein the linked points track progression, resolution, and change in diseases, diagnoses, morphologies, treatment regimens, and patient data with visualizations, human readable outputs and categorizations, matrices, machine readable outputs, calculated outputs, and other outputs; optionally outputting translated, enhanced anatomic descriptions, cross-mappings, and encodings, and/or isolated visualizations for all defined paths under and over a point of interest or targeted defined path.

In certain embodiments, the anatomic site is represented by a pinpoint, segment of anatomic sites, groups of anatomic site segments, complete anatomic sites, groups of anatomic sites, different levels of hierarchy, different groups, and different organ systems simultaneously. In certain embodiments, the defined paths are polygons, vertice groups, compound paths, lines, and/or shapes. In certain embodiments, the defined paths are dependently or independently scaled, rotated, excluded, included, aligned, and moved based on path or path group memberships, filters, patient characteristics, or detections. In certain embodiments, the metadata is an anatomic site name, identifiers, laterality, prefixes, suffixes, axial definitions, axial customizations, orientation, synonyms, coordinates, rotations, scales, offsets, centers, thicknesses, bounding boxes, color, pattern, opacity, stroke, visual attributes, animations, group memberships, group axial definitions, curves, zones, linguistic segmentation instructions, visual segmentation instructions, relationships, position, areas, surface areas, intensity, level, mathematical translations, linguistic translations, transformations, links, order, perspective, view, and/or mirrored axis. In certain embodiments, the custom axes label, describe, and relate points of interest or paths of interest in multiple map dimensions and hierarchical levels. In certain embodiments, the custom axes are reversible to apply mapping, descriptions, labeling, translations, and visualization in an opposite perspective. In certain embodiments, the laterality labels on anatomic maps, avatars, and visualizations automatically adjust for axes in view in the correct context, view, rotation, and/or language. In certain embodiments, the visualization intensity is user assigned. In certain embodiments, the visualization, description, visualization intensity and cursor, pin, and/or other targeted position is automatically calculated with variables from path intersections, overlays, and underlays related to the other paths variables, including but not limited to opacities, color blending, pattern blending, addition, division, multiplication, and subtraction. In certain embodiments, the visualizations are automatically categorized, grouped, described, and labeled into human and machine-readable labels and color coding. In certain embodiments, the custom axes enable calculation of angle, distance, rotation to describe relationships between at least two points including an insertion point and termination point. In certain embodiments, the custom axes enable relational descriptions between two or more points, with optional description modifications describing the magnitude of such relationships. In certain embodiments, the custom axes describe the positions of points of interest within anatomic sites with sequence sensitivity, such that magnitude of deviation affects the order and/or presence of the directional modifiers; In certain embodiments, the magnitude dynamically changes linguistic descriptions, coded descriptions, and calculations based on the positions or travel of points of interest; In certain embodiments, the visualizations, descriptions, and associated data are applied and aligned to images, videos, avatars, and other multimedia in paper or electronic workflows, wherein the electronic workflows may be virtual reality, mixed reality, and/or augmented reality and wherein the applied and aligned data can represent different time points such as a timeline. In certain embodiments, the visualizations, descriptions, and associated data are applied and aligned to images, videos, avatars, and other multimedia in paper or electronic workflows, wherein the electronic workflows apply artificial intelligence, machine learning, and computer vision.

In another embodiment, a method for hierarchical painting and visualization of anatomic sites, the method compromising: selecting an anatomic site or site segment on a hierarchical anatomic map wherein the anatomic map is comprised of paths or path segments of different sizes and the path or path segments define the anatomic sites and site segments; optionally applying a color and/or pattern and/or intensity to an anatomic site or site segment wherein the application is associated with health data such as diagnoses, symptoms, morphologies, and treatment recommendations; optionally traveling through the hierarchical anatomic map with hierarchical selectors that visualize the anatomic sites and translated descriptions wherein the travel applies the color and/or pattern and/or intensity to the destination anatomic site; optionally outputting a distribution list or tracking matrix that contains the anatomic sites lists; wherein the distribution list includes surface area, intensity, and detected data, such as counts, morphology, treatment recommendations, or other metadata, for each group component, which can be output into a plurality of templates; optionally linking multidimensional anatomic points and distribution segments to points in time wherein the linked points track progression, resolution, and change in diseases, diagnoses, morphologies, treatment regimens, and patient data with visualizations, human readable outputs and categorizations, matrices, machine readable outputs, calculated outputs, and other outputs; optionally associating at least one component piece of anatomic site data with the anatomic site wherein the site data is metadata relevant to the anatomic site; optionally creating a dynamic anatomic address that can be tracked spatially and through time wherein the address accepts images, attachments, records, and other data; optionally visualizing a point of interest relative to path and the point of interest simultaneously relative to all underlying and overlying and neighboring paths, combined and/or in isolation, in any hierarchical level or group, wherein all paths are described, sequenced, and categorized in any coded, linguistic, or symbolic language; wherein the visualization is overlaid, underlaid, or aligned to other multimedia containing anatomy to apply multidimensional and optionally mirrored anatomic mapping, descriptions labeling, and visualization; wherein the point of interest is movable such that when moved it travels to different positions within all the paths above and below it; simultaneously generating multidimensional descriptions labeling, visualization, and map positioning in real-time while the point of interest is moving; optionally grouping paths or path segments with other paths or path segments to create a distribution list, and a hierarchical painting method where the hierarchical painting method allows for targeted, visualized hierarchical path selection of overlying and underlying paths and neighboring, with translated real-time visualizations and descriptions; wherein the distribution list includes surface area, intensity, and detected data, such as counts, morphology, treatment recommendations, or other metadata, for each group component, which can be output into a plurality of templates; linking multidimensional anatomic points and distribution segments to points in time wherein the linked points track progression, resolution, and change in diseases, diagnoses, morphologies, treatment regimens, and patient data with visualizations, human readable outputs and categorizations, matrices, machine readable outputs, calculated outputs, and other outputs; optionally outputting translated, enhanced anatomic descriptions, cross-mappings, and encodings, and/or isolated visualizations for all defined paths under and over a point of interest or targeted defined path.

In certain embodiments, the hierarchical painting is visualized, described, translated, measured, enhanced, related, and/or encoded in multiple hierarchical levels simultaneously with a corresponding color coded legend.

In another embodiment, a method for translating a point within a path, a path segment, a path name, or a path group on an anatomic visualization into any coded, linguistic, or symbolic language; wherein that paths are optionally mirrored, reflected, transformed, rotated, scaled, or manipulated.

In another embodiment, a system for translating a point within a path, a path segment, a path name, or a path group on an anatomic visualization into any coded, linguistic, or symbolic language; wherein that paths are optionally mirrored, reflected, transformed, rotated, scaled, or manipulated.

In another embodiment, a method for painting or marking up paths and path segments on anatomic maps, the method comprising: describing the painted or marked up areas of anatomy in any coded, symbolic, or linguistic language; optionally calculating the surface area of the painted or marked up areas relative to the map.

In another embodiment, a system for painting or marking up paths and path segments on anatomic maps, the method comprising: describing the painted or marked up areas of anatomy in any coded, symbolic, or linguistic language; optionally calculating the surface area of the painted or marked up areas relative to the map.

In another embodiment, a method for generating a description of a point, path, path segment, or path group on an anatomic map, image, or avatar in any coded, linguistic, or symbolic language and optionally automatically encoding the anatomic site and modifiers of the anatomic site, optionally including laterality, prefixes, suffixes, synonyms, and enhanced directional descriptors, optionally simultaneously through multiple levels of hierarchy and relationships; optionally associating the anatomy data with any combination diagnosis data, patient data, procedure data, calculated data, measured data, morphology data, relational data, billing data, synonyms, or other health data and optionally automatically encoding and translating the associated data; optionally visualizing a point, path, path segment, or path group in relation to underlying, overlying and nearby anatomic sites and points, paths, path segments, or path groups.

In another embodiment, a system for generating a description of a point, path, path segment, or path group on an anatomic map, image, or avatar in any coded, linguistic, or symbolic language and automatically encoding the anatomic site and modifiers of the anatomic site, optionally including laterality, prefixes, suffixes, synonyms, and enhanced directional descriptors, optionally simultaneously through multiple levels of hierarchy and relationships; optionally associating the anatomy data with any combination diagnosis data, patient data, procedure data, calculated data, measured data, morphology data, relational data, billing data, synonyms, or other health data and optionally automatically encoding and translating the associated data; optionally visualizing a point, path, path segment, or path group in relation to underlying, overlying and nearby anatomic sites and points, paths, path segments, or path groups.

In certain embodiments, the same or multiple users can interact with the same map, annotations, markup and health data with different languages, preferences, or templates simultaneously through concurrent and collaborative sessions.

In another embodiment, a method for healthcare documentation, communication, annotation, markup, and/or collaboration wherein visual and linguistic inputs are translated in real-time in any linguistic, coded, or symbolic language.

In another embodiment, a system for healthcare documentation, communication, annotation, markup, and/or collaboration wherein visual and linguistic inputs are translated in real-time in any linguistic, coded, or symbolic language.

In certain embodiments, the translated documentation can be used to further dissect and encode data into billing codes, diagnosis codes, anatomy codes, extension codes, and/or cross-mapped codes in a manner that is specific to the patient and user data.

In certain embodiments, the data specific to the patient and user includes photos, attachments, links, procedure type, procedure measurements, procedure counts, procedure weight/value, diagnosis, diagnosis category, insurance status, fee schedule, units, country-specific billing rules, region-specific billing rules, deductible status, copay status, coinsurance status, account balance status, discount status, and other billing associated metadata, as well as comments, and other patient data associated with a particular anatomic site.

In another embodiment, a system for anatomic site visualization and description that highlights and describes anatomic sites, anatomic site segments, anatomic distributions, anatomic distribution segments, and/or anatomic relationships in natural language in any coded, linguistic, or symbolic language, the system comprising: linguistic elements of anatomy broken down into components in any combination or order of laterality, prefixes, suffixes, enhanced descriptions, relationships, synonyms, name, measurement, triangulation, calculation, cross-mapping, or language-based description; optional encoded elements of anatomy broken down into components in any combination or order of laterality, prefixes, suffixes, enhanced descriptions, relationships, synonyms, name, measurement, triangulation, calculation, cross-mapping, or code-based description; optional symbolic elements of anatomy broken down into components in any combination or order of laterality, prefixes, suffixes, enhanced descriptions, relationships, synonyms, name, measurement, triangulation, calculation, cross-mapping, or symbol-based description; Visualization elements that highlight and optionally color-code the anatomy correlating with any combination of linguistic elements, encoded elements, or symbolic elements on visualizations that contain anatomy; alternative visualization options in mirrored, scaled, aligned, rotated, or transformed axes.

(APP02) Standardized lexicons for anatomy are usually limited to a single language, or less than a handful of languages at best, and recent publicly available lexicons like the International Classification of Diseases (“ICD-11”) anatomy chapter simply lists terms without visualizations. Existing anatomic references, including digital references, are unable to detect and translate mixed inputs automatically and in real-time. Furthermore, existing anatomic references do not assign coordinates to anatomic sites derived from mixed inputs and are unable to apply progressive sub-segmentation to enhance visualization to near pinpoint precision, are unable to mirror or otherwise alter the described visualizations, and furthermore do not supply anatomic maps relevant to the anatomy lookup. A practical application of the embodiments illustrated in the teachings herein would be to lookup and apply precise visualizations, coordinated targets, and multidimensional anatomic maps to coded descriptions of anatomy.

In the embodiments of the system 985 illustrated, visual definitions, relevant anatomic maps, visualizations, photos, and records relevant to a patient and dynamic custom coordinates of anatomic addresses can be looked up by anatomic site name and components (such as laterality, prefixes, and suffixes) in any combination of linguistic language or code or symbols (e.g. stored and interacted with in a medium 995 in communication with a processor 986), and progressively sub-segmented with directional and magnitude modifier terms with mixed code and language order (on which the embodiments illustrated applies coded and linguistic dissection to provide precise visualizations and translations). Those definitions can be detected and visualized in different views, images, and multimedia automatically. Additionally, the mixed input generates relevant multidimensional anatomic maps and avatars, with automatically targeted, enhanced, optionally progressively sub-segmented, and color-coded visualization (e.g., with the generation module 993). Furthermore, descriptions of anatomy extracted from medical records (e.g., with the record retrieval module 997), in linguistic, coded, or symbolic form, can be used as mixed inputs to collate patient records for an anatomic area of interest while simultaneously visualizing the anatomic area of interest.

The embodiments illustrated include a method for detecting and translating uncoordinated, mixed linguistic, coded, and symbolic anatomic site data into coordinates, axes, visualizations, maps, avatars, targets, record results, and sequenced data. The mixed inputs (e.g., through the input device 988) may be text terms or verbal inputs in any linguistic language, or coded inputs such as ICD-11 codes for anatomy, or numerical codes corresponding to an anatomic site, region or other descriptive term, or symbolic inputs such as emojis. Semantic coordination and/or linguistic coordination are phrases that can be used to describe some of the practical applications of the system 985. In an exemplar of symbolic code, a “nose emoji” categorizes nasal sites. Another symbolic input would include an image, illustration, photograph, video, avatar, or other multimedia that contains a nose, with the nose being the area of interest defined by the user or the system or method. The mixed inputs are automatically detected, categorized, and organized into fully sequenced translations, including synonyms, and into visualizations and coordinates relative to anatomic sites (e.g., with the generation module 993 in communication with the data processing module 999 as one example). Linguistic inputs that are categorized can include identifiers, lateralities, prefixes, suffixes, anatomic site names, categories, modifier terms such as directional modifiers, custom descriptors, anatomic distributions, distribution segments, and synonyms (including semantic, linguistic and symbolic slang synonyms such as “noggin” for head, or “peach emoji” or buttock) for each of the preceding inputs, as non-limiting examples. The embodiments illustrated delivers language translations in a natural linguistic sequence for the language (e.g., through the modules in the medium 995 in communication with the GUI 991 or other output device 989), with corresponding generated visualizations, including anatomic maps and avatars which themselves can include multidimensional, custom axes defined coordinate systems, and records related to a patient that have anatomic descriptions or images associated with them. The delivered anatomic maps and avatars can be overlaid or underlaid and aligned to other images that contain anatomy by the system 985. Additionally, the embodiments can include progressive sub-segmentation of the mixed and uncoordinated inputs to deliver human readable, accurate, and precise descriptions with corresponding visualizations and coordinates up to a pinpoint level, as well as coded descriptions. The embodiments illustrated also include axial mirroring (e.g., reflection) and/or other axial alterations to display (e.g., on the Display 987) the mixed-input-derived visualizations and anatomic maps, to show and interact with the visualizations and maps in both outside-observer view and in selfie-view. In certain embodiments, one or more of the reflection axes can deviate from the standard reflection axis of 180 degrees, so the reflection axes and angles are therefore uniquely targetable and modifiable.

The system 985 also interacts with the display 987 and the GUI 991, which can serve as an input device 988 and an output device 989 such as a digital whiteboard, through artificial intelligence and language models 1103, has an interactive conversation with a surgeon, in certain embodiments, to double check all documentation. The whiteboard presents an interactive conversational verbal summary of the encounter, with known details and details that need to be filled in. A non-limiting example of such a conversation and summary is as follows, with logical steps taken where the AI understands which details to present verbally (e.g. through the output device 988 speaker) and which details are already known by presenting verbally in a context aware manner, and which details are still needed by filling in the blanks represented as more than 2 underscores (___) and example language in parentheses ( ) in a sample conversation regarding a “Mohs Surgery” between the electronic device (known as “WHITEBOARD” in the exemplar conversation) and the surgeon (known as “PHYSICIAN” in the following exemplar conversation) that can occur while the surgery is taking place, in the present exemplar. Also in the example, the physician is Dr. Molenda, and the patient is Mr. Smith:

WHITEBOARD: “Today's______(e.g. basal cell carcinoma on the left nasal ala) has been documented in the Interval Cancer History, and on the top line of the Patient Clipboard in the electronic record for______(e.g. basal cell carcinoma) in the following format: (e.g. 2021 Jun. 17 BCC left nasal ala, treated by Molenda with Mohs with Bilobed flap). The closure type is documented in the electronic record as______closure type (e.g., Bilobed flap). Dr. Molenda, can you please provide me with a secondary defect measurement?”

PHYSICIAN: “the secondary defect is 2.3 by 4.5”

WHITEBOARD: “Thanks you, the secondary defect has been documented as 2.3×4.5, creating a total repair size of 14.35 square centimeters when combining the primary and secondary defects. The billing code for the repair that resulted based on the measurements and the anatomic location on the nose is “14061.” Due the proximity to the nostril, I have also documented the risk to bodily function of breathing in the patient record. I also categorized the repair as a “major repair” based on the CPT code, and added the correct-57 modifier to today's evaluation and management code of 99215. Since you decided to perform the major surgery today, I also documented the decision making process and conversation of risks, benefits, and alternatives I heard you have with Mr. Smith. The patient has an allergy to Penicillin, and has 2 interactions with Doxycycline. I know you typically prefer sending Keflex, but due to this allergy, and the minimal severity of the interactions with Doxycycline, would you like to send the Doxycycline 100 mg by mouth twice daily?” PHYSICIAN: “Can you tell me more about the interactions?”

WHITEBOARD: “There is an interaction with the magnesium and multivitamin reported by the patient”

PHYSICIAN: “Please send the doxycycline and have patient hold the magnesium and multivitamin for 1 week”

WHITEBOARD: “The Doxycycline has been sent for 1 week to the patient's preferred pharmacy, CVS at 1249 Lane Ave in Toledo”

As one skilled in the art would know, the above example conversation has an innumerable number of permutations. Other non-limiting examples where a conversation can be initiated or continued with the interactive digital whiteboard include: “Hey Whiteboard, show me the pathology report”, “Hey Whiteboard, show me the digital slide”, “Hey Whiteboard, show me the pre-biopsy photo”, “Hey whiteboard, show me the future spots that need treatment”, “Hey whiteboard, show me the pre-operative photos.”, “Hey whiteboard, show me the shadow chart”, “Hey whiteboard, show me the consent form for today's procedure”, “Hey whiteboard, overlay the shadow chart on this patient on my augmented reality goggles.” Such non-limiting examples can generate output, e.g., with the generation module 993 and display the output on the GUI 991 and/or other output devices 989.

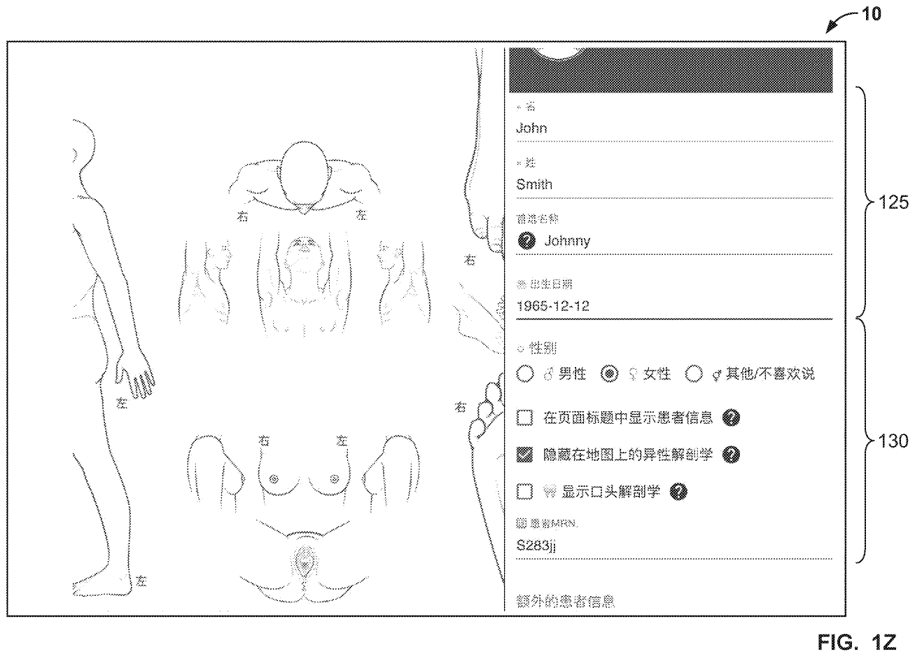

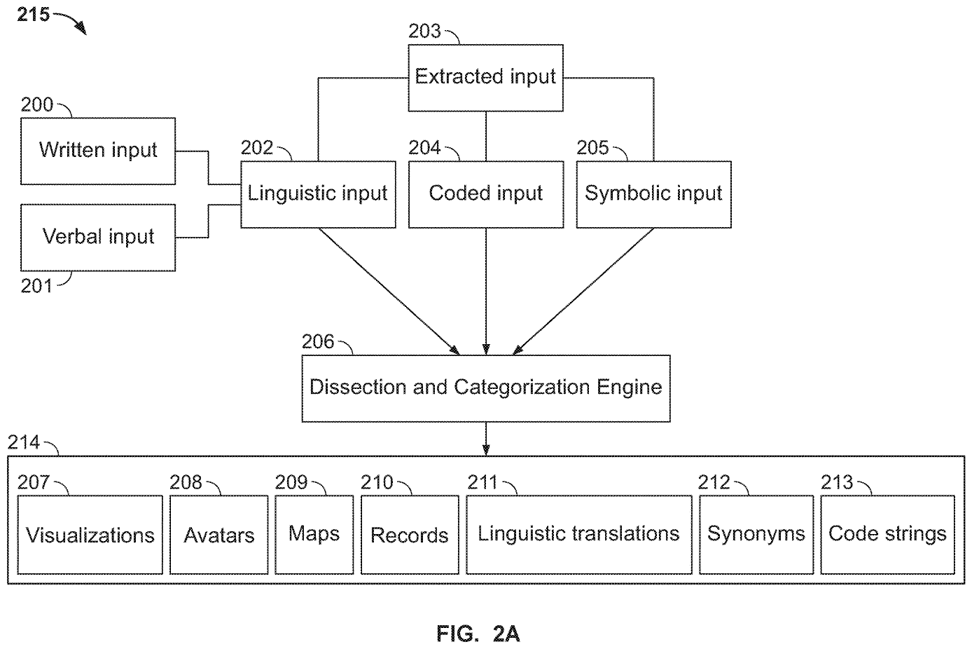

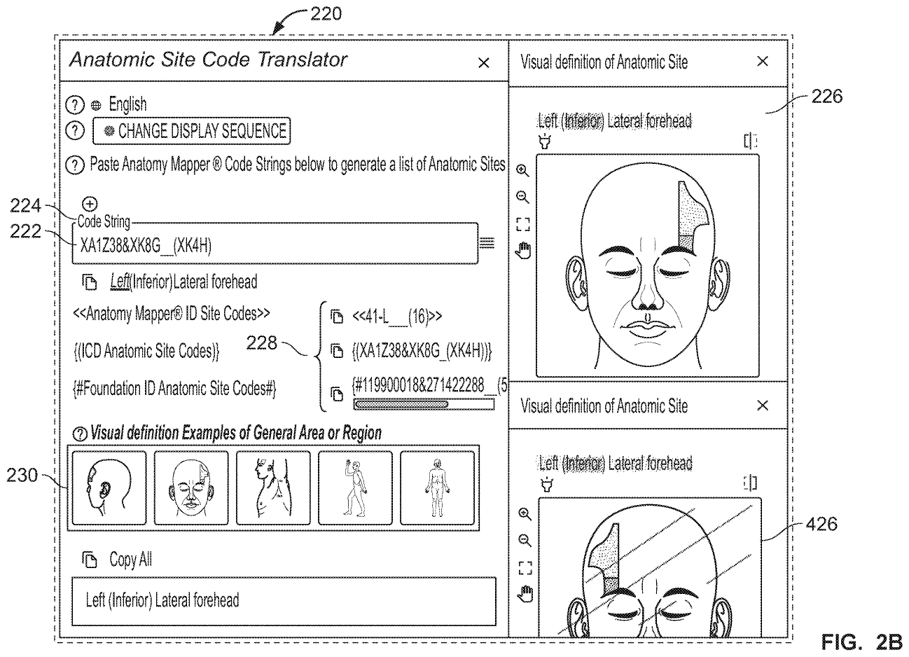

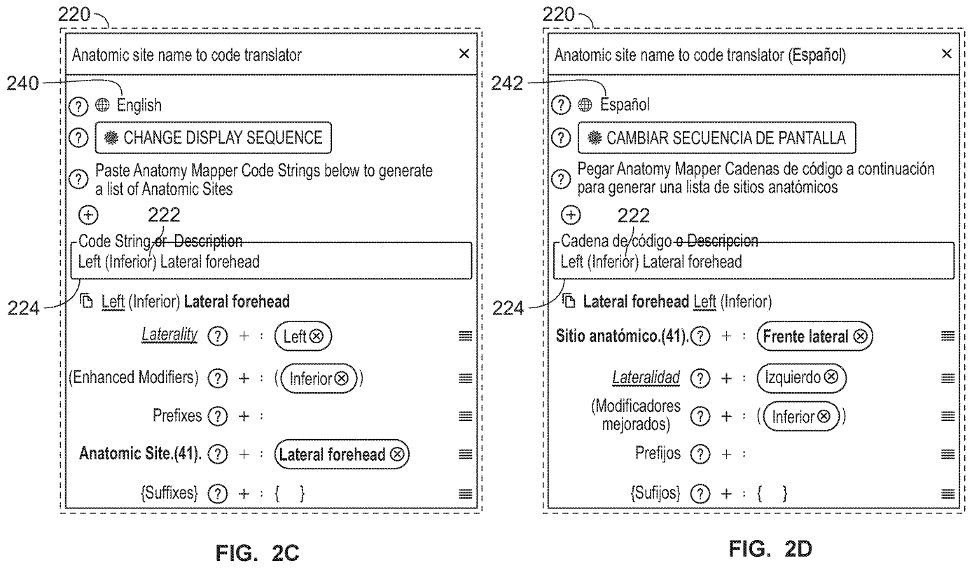

FIG. 2A illustrates a method and/or process 215 enabled by the system 985 that incorporates mixed inputs (e.g., from the input device 988) to various outputs 214 (e.g., to the output device 988). Inputs may be input by a user as either written inputs 200 or verbal inputs 201 or extracted inputs 203 from an existing record, such as a detected text description that contains an anatomic site name synonym and a laterality. Extracted inputs may include linguistic input 202, coded input 204 or symbolic input 205. Linguistic input 202 may include linguistic descriptions of anatomic site that include any of the following non-limiting examples: identifiers, lateralities, prefixes, suffixes, anatomic site names, categories, modifier terms such as directional modifiers, custom descriptors, anatomic distributions, distribution segments, and synonyms each of the preceding inputs. Linguistic input 202 is a mixed input category in that it may comprise written 200 or verbal 201 input (e.g., through the input device 988). The linguistic input 202 is language agnostic and therefore can also be in a mixed language, such as a mixture of English, Chinese, and Spanish to describe different components of the anatomic site. Written input 200 is derived from text-based linguistic descriptions of anatomic site, as defined above, including the synonyms, which is typed, pasted, or extracted from handwriting or optical character recognition (e.g., with the input device 988 and/or data processing module 999). Verbal input 201 is spoken live or via a recording, in any language. In certain embodiments, linguistic input 202 is “ear” (anatomic site) or “left” (laterality). Such extractions may be in any linguistic language. Other mixed inputs include coded input 204 and symbolic input 205. Coded input 204 is numeric or alphanumeric in most cases but includes other unicode characters. In certain embodiments, coded input 204 is the ICD code for a “left arrow emoji” laterality, “XK8G.” Symbolic input 205 is a drawing, character, icon, or image, emoji, or unicode character, and/or a string of these symbols as non-limiting examples. In certain embodiments, symbolic input 205 for ear is the “ear emoji” or a picture or diagram of an ear. Each of these mixed inputs are detected to be descriptors of anatomy, modifiers of anatomy, or laterality and are organized into digital “chips” or blocks of data by a dissection and categorization engine 206 in the method 215 enabled by the system 985 (e.g., enabled by the data processing module 999). The method 215 enabled by the system 985 uses the dissected and categorized inputs in a neural network to generate (e.g. with the generation module 993) outputs 214, including visualizations 207, avatars 208, maps 209, records (e.g., medical records that describe the left ear) 210, linguistic translations (e.g., left posterior surface of pinna) 211, synonyms (e.g., left back of ear) 212, and code strings (e.g., XA3S47&XK8G) 213.

The embodiments demonstrate further capability of automatically converting coded input by the system 985. FIG. 2B depicts a screenshot of an anatomic site code translator 220. In the illustrated embodiment the code string 222 identified is the ICD-11 code string “XA1Z38&XK8G_(XK4H), which corresponds with the “left (inferior) lateral forehead.” Inputting the code string 222 into the input box 224 (e.g., with the input device 988) returns all relevant visualizations 226 related to the patient, maps, and avatars, including highlighted, targeted, enhanced, and color-coded locations on multidimensional maps (e.g., with the record retrieval module 997 and/or the generation module 993 as non-limiting examples). In other embodiments, the code string 222 could be “lateral forehead izquierdo (XK4H)” or any other combination of uncoordinated and mixed inputs to reach the same result. Translations 228 into other coded, linguistic, and symbolic languages are delivered automatically and simultaneously based on user preference (e.g., in the knowledge base module), the depicted embodiment showing three different code string translations. Visual definitions 230 are also generated with enhanced modification visualized through automatic color-coding (e.g., with the generation module 993). In certain embodiments, the anatomic site and laterality components of the anatomic site could be visualized in red, and the enhanced modifier showing the possible zoned area of interest within the anatomic site could be shown in blue (on the GUI 991). In the illustrated embodiment, the outside observer view is shown. In other embodiments, the selfie/mirror view can be depicted as well as depicted with a silver gradient background to represent a mirror 426 as one non-limiting example. The input code string 222 can also be mixed. In an example scenario, the code string 222 could be in “Spanglish” where an English-speaking user is familiar with Spanish but cannot remember the term for “left”. The user can verbalize “left mano” (which is received by a microphone as the input device 988 as one example) and will receive relevant generated visualizations and coordinated maps 226, translations 228, records, and visual definitions 230 related to the “left hand” and simultaneously receive the translation 228 as “mano izquierda” (e.g. through the generation module 993 and other components of the physical medium 995 of the system 985). In an alternate example scenario, relevant visualizations and coordinated maps 226, translations 228, records and visual definitions 230 related to the nose may be found by using a “nose emoji” in the code string 222. A code string 222 incorporating “left” and a “nose emoji” would deliver relevant visualizations 226, translations 228, and visual definitions 230 related to the “left nose” whereas searching for just a “nose emoji” would deliver more relevant visualizations 226, translations 228, and visual definitions 230 related to the entire nose. By generating enhanced anatomic site names, the exemplar embodiments of the system 985 enable medical records to be searched for uncoordinated anatomic site descriptors, dissection of the descriptors, and delivery of visualizations, maps, avatars, and records associated with the anatomy of interest.

The example embodiments of the system 985 illustrate the capability of dissecting anatomic site descriptions into components in any language, categorizing those components, and translating those components to other languages while simultaneously applying natural linguistic sequencing, and providing visualizations, anatomic maps, and coded translations (shown in FIG. 2B). FIG. 2C depicts the anatomic site name to code translator 220 dissecting English 240 code strings 222 representative of anatomic site descriptions in semantic language into data blocks shown as chips that are categorized into anatomic description components b. In other words, the human readable input 244 that describes an enhanced anatomic site and dynamic anatomic address is put in the input box 224 and dissected and categorized. FIG. 2D depicts the anatomic site code translator 220 (e.g., shown on the GUI 991) translating an English description 244 of “left (inferior) lateral forehead” into dissected Spanish 242 data blocks shown in a different linguistic sequence from FIG. 2D. When changing the language to Spanish 242, natural linguistic sequencing is shown using a natural language processor as an example of the data processing module 999, where the laterality category is listed after the anatomic site and other automatic sequence changes. The input box 224 can accept (e.g., through the input device 988) code strings, any language (including written and spoken language), symbolic representations, or a mixture of these components and dissect them, categorize and organize them, sequence them, visualize them in all views (with additional enhanced visualization and segmentation), and target them as components of the dynamic anatomic address located in multidimensional anatomic maps and avatars. Additionally, synonyms in any language can be detected, dissected, categorized, looked up, and visualized. In certain embodiments, “pinna” could be searched for by the synonym of “ear”, which would detect English, that it's a synonym, and show the chips, records, and visualizations for “pinna.” In another non-limiting embodiment, synonym detection will recognize “belly button” or “navel” and return the relevant visualizations, translations, records, and visual definitions associated with the “umbilicus” chip. The language is automatically detected as English by the system 985 and the “umbilicus” chip gets loaded into the “anatomic site” field automatically, and if further translated in real time to any coded, linguistic, or symbolic language along with a real time visual preview on standardized diagrams; 3D avatars; or imaging/multimedia that contain a visible “belly button” (e.g., with the record retrieval module 997).

The exemplar embodiments of the system 985 target and isolate anatomic images, maps, points of interest, and regions, to provide a dynamic anatomy library (e.g., stored and communicated with on the medium 995) that includes visualized, coded, linguistic, and/or symbolic descriptions and definitions of anatomy. Additionally, the dynamic anatomy library includes hierarchical, multidimensional maps that can be isolated, segmented, and sub-segmented through visual, coded, linguistic, and symbolic inputs, to output the most relevant visualizations, maps, and images. Sites related to or nearby a point or anatomic site of interest are also shown dynamically in relation to one another with hierarchical selectors 505 that include visual and linguistic, coded, symbolic, and cross-mapped descriptions of anatomy (e.g., with the data processing module 999). The hierarchical selectors 505 (e.g., shown on the GUI 991) allow for simultaneous visual and descriptive travel through a hierarchy, and each option can be used to generate a form or other outputs dynamically that includes the selected visualization and translation. Additionally, for a point of interest, the hierarchical selector shows the point position relative to all overlaying and underlying anatomic sites, along with the enhanced translations.

For a distribution segment, all anatomic sites above and below the currently selected anatomic site, in layer or anatomic hierarchy, are also shown (e.g., on the GUI 991) with translation, borders, colors, patterns, intensities, and surface area calculations (e.g., enabled by the data processing module 999, the generation module 993, and/or the knowledge base module 992). For dynamic, translated form generation, in addition to translated and visualized anatomy data, the forms also automatically include dynamic non-anatomy information, such as demographic information extracted from the patient chart. The forms, whether electronic or paper (e.g. printed to the output device 989), are then processed for a form processing engine (e.g. enabled by the data processing module 999 as one example) and relevant anatomy data, including visualizations, points of interest, distributions, and coordinates, and non-anatomy data are extracted from the dynamic forms, input into the system 985 (e.g. with the input device 988), and processed again into engines for detection, categorization, encoding, translation, and labeling for both the anatomy data and non-anatomy data (e.g. with the data processing module 999). These data apply neural networks to modular engines stored and/or processed in the tangible medium 995 in communication with the processor 986 to generate medical bills, records, visualizations, and labeling protocols, which are all translatable and localizable to any coded, linguistic, or symbolic language, or to geographic region.

Another non-limiting example of where generated isolated visualizations 68 (e.g. with the generation module 993) can be applied is to exports to printable PDF (e.g. the output device 989) for each pin or distribution segment, including a pathology requisition form that generates printable labels that also include targeted, isolated visualizations 68 of the anatomic sites along with a standardized anatomic descriptions that automatically can include laterality, prefixes, suffixes, enhanced directional modifiers, custom descriptors, automatic relationships, patient demographics, symptoms, morphologies, linked photos, linked documents, attachments, diagnoses, diagnoses extensions, billing codes, procedural descriptions, pin or distribution segment or distribution descriptions, tags, codes such as Quick Response (QR) codes, and other anatomy and non-anatomy data. Through the input device 988 (e.g., a camera and/or scanner), multiple QR codes can be scanned at one time for re-creation of an entire requisition form that is generated (e.g., by the generation module 993) at a different point in time and at a different entity, such as the lab, for example. In other words, the form automatically replots all points and fills in information from a single picture or scan of the paper form, and it does so automatically in the contemplated example.

In certain embodiments, a computerized electronic visualization, map, coordinate, and description generation system for creating enhanced, universal anatomic references for an anatomic site, the system configured to: Receive uncoordinated mixed input data correlating to the anatomic site wherein the data may be input by a user or extracted from existing records; detect descriptors of anatomy, modifiers of anatomy, and/or laterality in the inputs; use a dissection and categorization engine to organize the descriptors into digital data blocks; and use the organized data blocks to generate outputs wherein the outputs are translated visualizations, maps, coordinates, and descriptions incorporating the mixed input data that correlates to the anatomic site.

In certain embodiments, the data input by a user is written input. In certain embodiments, the data input by the user is verbal input. In certain embodiments, the data input is linguistic, coded, and/or symbolic. In certain embodiments, the symbolic data can include images, multimedia, and symbolic characters representative of anatomy. In certain embodiments, the outputs are visualizations, avatars, images, videos, vectors, graphics, illustrations, maps, records, linguistic translations, synonyms, labels, symbolic translations, and coded translations. In certain embodiments, the descriptions of anatomy can re-create precise dynamic visualizations and associated metadata on different systems at different time points, and the descriptions can optionally be used as tracking points. In certain embodiments, the re-created dynamic visualizations can be added, amended, and/or modified with associated metadata to create a historical record and timeline at the anatomic site. In certain embodiments, the historical record and timeline can be re-created on visualizations, images, avatars, and multimedia in electronic environments, printed environments, and/or virtual, mixed, or augmented reality environments.

In another embodiment, a computerized electronic visualization, map, coordinate, and description generation system for creating enhanced, universal anatomic references for an anatomic site, the system configured to: receive uncoordinated mixed input data correlating to the anatomic site wherein the data may be input by a user or extracted from existing records; detect descriptors of anatomy, modifiers of anatomy, and/or laterality in the inputs; use a dissection and categorization engine to organize the descriptors into digital data blocks; apply progressive linguistic and visual sub-segmentation to achieve pinpoint precision for the anatomic site and automatic sequencing of inputs into coded and translations that apply natural linguistic sequencing; and use the organized data blocks to generate outputs wherein the outputs are translated visualizations, maps, coordinates, and descriptions incorporating the mixed input data that correlates to the anatomic site.

In certain embodiments, the data input by a user is written input. In certain embodiments, the data input by the user is verbal input. In certain embodiments, the data input is linguistic, coded, and/or symbolic. In certain embodiments, the outputs may include are real-time visualizations, avatars, maps, records, linguistic translations, synonyms, labels, symbolic translations, and coded translations.

In another embodiment, a method of creating enhanced, universal anatomic references for an anatomic site, the method comprising to: receiving mixed input data wherein the mixed input correlates to the anatomic site; detecting descriptors of anatomy, modifiers of anatomy, and/or laterality in the inputs wherein the descriptors can be anatomic or non-anatomic descriptors; categorizing and organizing the descriptors into digital data blocks; and progressively sub-segmenting the inputs to achieve pinpoint precision and automatic sequencing of inputs into coded translations and linguistic translations that apply natural linguistic sequencing.

In certain embodiments, the mixed data may be input by a user or extracted from existing records. In certain embodiments, the mixed input data is linguistic, coded, and/or symbolic. In certain embodiments, the symbolic data can include images, multimedia, and symbolic characters representative of anatomy.

In another embodiment, a method for translating a description of anatomy in any coded, linguistic, or symbolic language to a point within a path, a path segment, a path, a path group, or a coordinated target on multimedia that contains anatomy.an anatomic visualization.

In another embodiment, a system for translating a description of anatomy in any coded, linguistic, or symbolic language to a point within a path, a path segment, a path, a path group, or a coordinated target on multimedia that contains anatomy and anatomic visualization.

In another embodiment, a method for generating anatomic visualizations, targets, maps, avatars, or coordinates from language or code.

In another embodiment, a system for generating anatomic visualizations, targets, maps, avatars, or coordinates from language or code.

(APP03) Traditional solutions used to document anatomic sites with anatomic mapping do not provide targeted isolation, segmentation, and coloring of anatomic images or visualizations. They also do not serve back segmented anatomic maps, or dynamically visualize isolated points of interest relative to site borders in each level of a hierarchy or multiple layers or visualize segmented or complete anatomic sites in a hierarchy with color coding, or isolatable and combinable groups of points of interest. Traditional solutions also do not create dynamic anatomy libraries from record defined and user defined inputs or create dynamic forms from the dynamic anatomy library and non-anatomy data that includes targeted and isolated visualizations of multidimensional anatomy maps, anatomic site translations, and re-creation abilities directly on the forms. Traditional solutions also do not combine anatomy data, non-anatomy information, patient data, country data, coded data, and translation data to create language agnostic, geographic location-optimized translated forms and visualizations. Traditional solutions are also unable to extract anatomy and non-anatomy data from both electronic and digital forms containing anatomy visualizations and use that extracted information in a plurality of engines to re-create dynamic anatomy libraries. Thus, significant manual human effort, time and cognition are needed to use traditional solutions. An example of a traditional paper workflow that is optimized by the exemplar embodied examples of the system 985 is the paper Mohs map used to document skin cancer surgery. Such maps that contain anatomic visualizations must be manually selected from a paper or electronic library, and do not have any relevant data filled in automatically, thus creating a lot of manual steps, handwriting, or typing, and manual record correlation (e.g., looking up pathology report information, which often contains a non-specific anatomic site) that is prone to human error. Further still, such Mohs maps that were printed and documented on paper must be manually scanned back into the patient chart and manually associated with the documented anatomic site, if that is even an option as a category in the electronic health record. The exemplar embodied examples of the system 985 solve what has been pointed out as lacking in traditional solutions with targeted isolation, segmentation, and coloring of anatomic images, anatomic maps, points of interest, and regions of interest into a dynamic anatomy library for point visualization and translation in relation to multidimensional anatomic borders, visualized hierarchical travel and selection, dynamic, translated form generation, and for use in medical record generation and retrieval. In other embodiments, context aware forms are generated (e.g., with the generation module 993) based on location of touch or click or gesture on the GUI 991, with map subsegment, diagram, or group isolation to generate the desired forms and output the generation to the output device 989 and/or GUI 991.

The embodiments illustrated include a system and method for targeted isolation, segmentation, and coloring of anatomic images, anatomic maps, points of interest, and regions of interest for dynamic, translated form generation. For example, the embodied non-limiting example capabilities of the system 985 whereby a description of anatomy that includes an anatomic site, and optionally laterality and other descriptors, targets and isolates diagrams from an anatomic map library (e.g., stored on the medium 995) that fits the anatomy description. A point of interest (e.g., as defined by the input device 988) on a layered, hierarchical map is visualized in relation to the borders of each isolated anatomic site above, below, on the same level along with translated human readable descriptions of the pin position relative to each layer. An anatomic region of interest is also visualized (e.g., on the GUI 991) in relation to sites above, below, and on the same hierarchical or layer level. Targeted isolation of each layer (e.g., with the data processing module 999, the generation module 993, and/or the image interface module 994) provides generated visualization, translation, descriptions, and visual selection points for a visual, translated hierarchical travel method. A dynamic anatomy library combines with other data to generate dynamically filled in and fillable forms with relevant visualizations, codes, translations, and data. Dynamic forms that contain anatomy data are processed through a form processing engine for detection, categorization, encoding, translation, and labeling of the anatomy and non-anatomy data, and those outputs then process through a billing code engine, record generation engine, and image and visualization labeling engine (e.g., engines are enabled by the medium 995 communicating with the processor 986 in the system 985). In certain embodiments, the record retrieval module 997 (e.g. in the tangible medium 995 in communication with the processor 986 and other modules in the medium 995 can create an engine that applies neural networks (enabled by the system 985 (e.g. through modules in the medium 995 in communication with the processor 986)), computer vision, and artificial intelligence to re-create, analyze, categorize, and serve back record defined data to complete the loop-which the system and method describes as application of the coordinated language model 980. In certain embodiments, a visual library of standardized anatomic sites, with automatically added laterality terms and modifier terms and visual subsegmentation is generated in multiple formats (e.g., by the generation module 993 on the GUI 991) such as table or navigable tree (radial, linear, or other), with real-time translation into any coded, linguistic or symbolic language. The library automatically populates relevant visualization links and generates targets for the visualization requested by the user. In certain embodiments, rasterized and vector images representing targeted and targetable, colored areas of anatomy are stored on the physical storage medium 995 as a visual library, and the image and map files are named (e.g. by the data processing module 999), stored (e.g. in the database interface module 996), and retrieved (e.g. by the record retrieval module 997) based on their path anatomic site identifiers, laterality, and other characteristics.

In another non-limiting embodiment, a generative artificial intelligence can apply a combination of the system 985 and components like the coordinated language model 980 to demonstrate skin, health, or cosmetic findings on patients and consumers. For example, video can be input into the system by a user with the input device 988 that contains the user as the subject of the video in shorts and a t-shirt. The user describes, through speech, text, code, or symbol: “show me what this video would look like with psoriasis on the arms and legs.” Since different diagnoses have different morphologies, and that can vary based on environmental conditions, skin type, skin tone, anatomic distribution and other factors, the system is able to account for these variables (e.g., with the knowledge base module 992 in communication with the data processing module 999). The user could alternatively describe, through speech, text, code, or symbol: “show me what this video would look like with guttate psoriasis on the arms and legs.” The user could alternatively describe, through speech, text, code, or symbol: “show me what this video would look like with guttate psoriasis on the arms and legs affecting 4% BSA” wherein BSA means body surface area. The generated psoriasis would appear differently on different skin types. For example, guttate psoriasis as an example appears differently on Fitzpatrick type I skin (lighter skin) than it does on a darker skin type, like Fitzpatrick Type VI. In another example, “muéstrame EK90.1 en este video” generates the same video, but this time from Spanish input and from an ICD-11 code for guttate psoriasis. As one skilled in the art would know, the input could also be reversed, where a patient who has psoriasis asks a question about what they would look like without it; e.g., with treatment with a certain drug as one non-limiting example. Using a vision language model within the system in reverse as a language vision model is also enabled by the system, as the models are omnidirectional to form the coordinated language model 980 engine.

In another embodiment, for cosmetic surgery, before and after expectations can also be set, or compared. In one non-limiting example, a user can ask the system, through the input device 988: “Show me what I would look like after a CO2 laser resurfacing versus a Halo Pro laser.” For some skin types, such as for skin type V, the user would get a response (e.g., through the output device 989) that “your skin type is not a candidate for CO2 laser resurfacing, so results are not shown” and only show the results for the Halo Pro. Also, the system knows that results on the neck are less dramatic than results on the face because settings must be turned down. The generated image (e.g., through the generation module 993) would show realistic results based on different anatomic regions. The system 985 applies textures/patterns, smoothings/overlays/underlays, and other filters containing such as morphologies to the subject based on their request (e.g., with the data processing module 999), aware of skin tone and anatomic site and other extractable health care information or information that could affect the request.

In yet another non-limiting example, through conversational chat bots, visual responses can be shown to questions based on image input and user input (e.g., through the input device 988). Some non-limiting example questions for the AI consultation include: “How will I look during the recovery?”; “Show me what I will look like during my isotretinoin treatment course?”; “How would I look with physician grade skin care?”; “What will I look like if I go out in the sun after CO2 laser during the recovery process?”. Therefore, consultation for a cosmetic procedure was performed through AI with generated responses through the generation module 993.

In another embodiment, using Langer's lines as a guide, and diagnosis data, clinical guidance can be provided through augmented reality goggles, as one example, taking advantage of spatial computing enabled by the system 985. A new or busy clinician “how should this excision be marked?” and the system 985 will know the diagnosis based on the pathology report (e.g., with the record retrieval module 997), and what margin to take (with the knowledge base module 992), and how to orient the excision for the best cosmetic and functional outcome. The system 985 can even mark the patient digitally with augmented reality or physically such as with a marker on a robotic arm (e.g., the output device 989). Continuing this example, an image input (e.g., through the input device 988) would be able to determine anatomic site, orientation, tag location, and appropriate margins (based on current guidelines, e.g., in the knowledge base module 992). A pathology requisition form, including the relevant images and anatomy visualizations could even be generated automatically (e.g., with the generation module 993 and/or the output device 989). The non-limiting example could be generated along with a pathology note and operative note. For example, “biopsy provide melanoma in situ, located on the right superior lateral scapular region, tagged Superior, check margins” along with a procedure note: “biopsy provide melanoma in situ, located on the right superior lateral scapular region, tagged Superior, check margins; “calendar emoji” 2022 Jan. 13, 55 year old Male, whose Date of Birth is “birthday cake emoji” 1966 Oct. 8, with Monk Skin Tone Scale (Monk Skin Tone Scale 02), with Fitzpatrick Skin Type (Fitzpatrick Skin Type 1); Symptoms: Growing; Morphology: Hyperpigmented, Irregular, Asymmetric; Diagnosis: Dx: Melanoma of skin (2C30), melanoma in situ type; Procedure: Excision-Melanoma in situ . . . “as a non-limiting partial example. In certain embodiments, there can be more than one diagnosis associated with an anatomic site and/or representation, and additional diagnoses can have their own set of extension descriptions and/or extension codes. In certain embodiments, additional diagnoses and/or their associated data (e.g., diagnosis extensions) can be added automatically without additional clicks or user input (e.g., enabled by the knowledge base module 992).

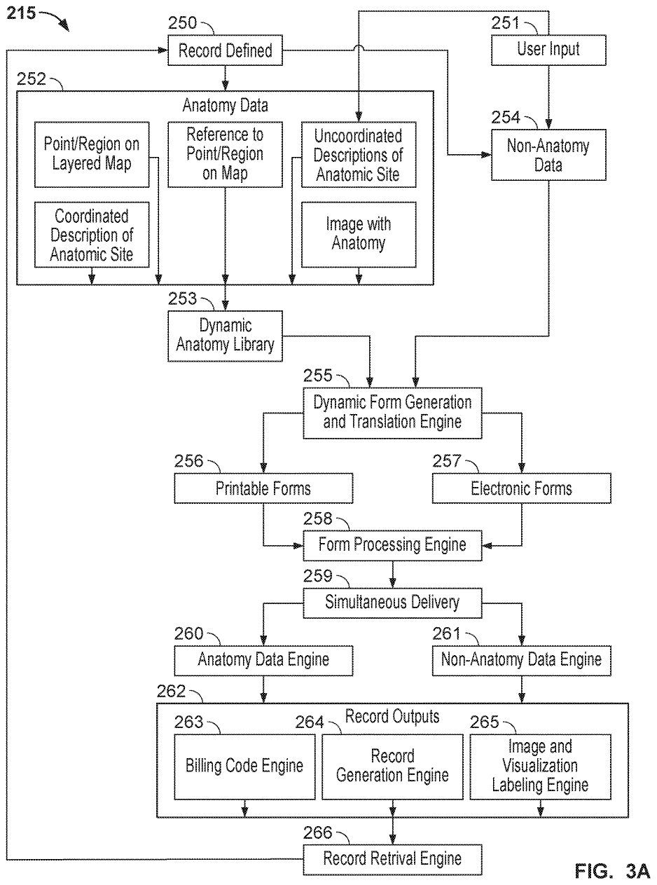

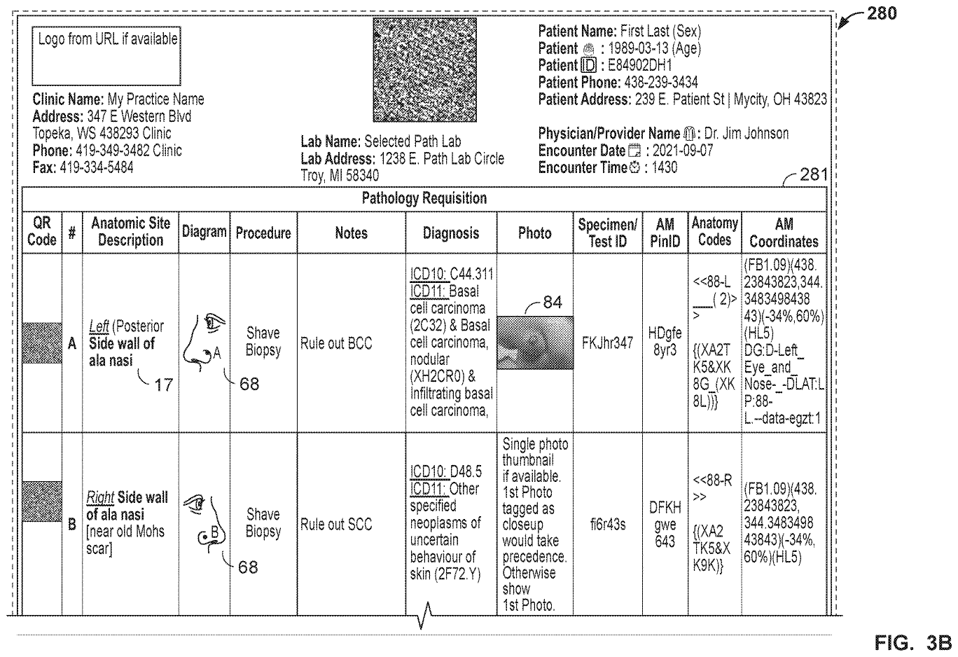

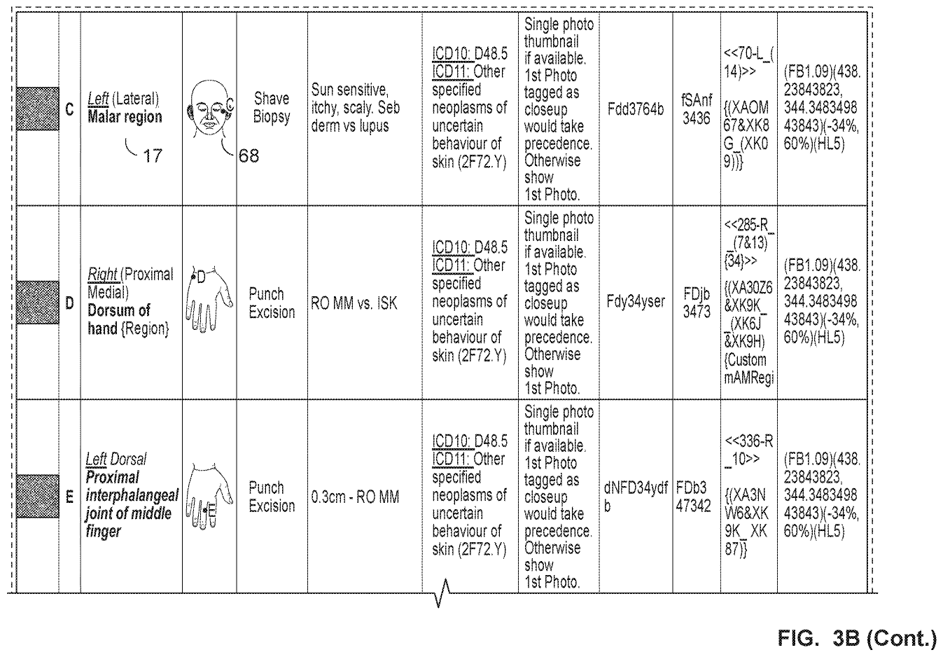

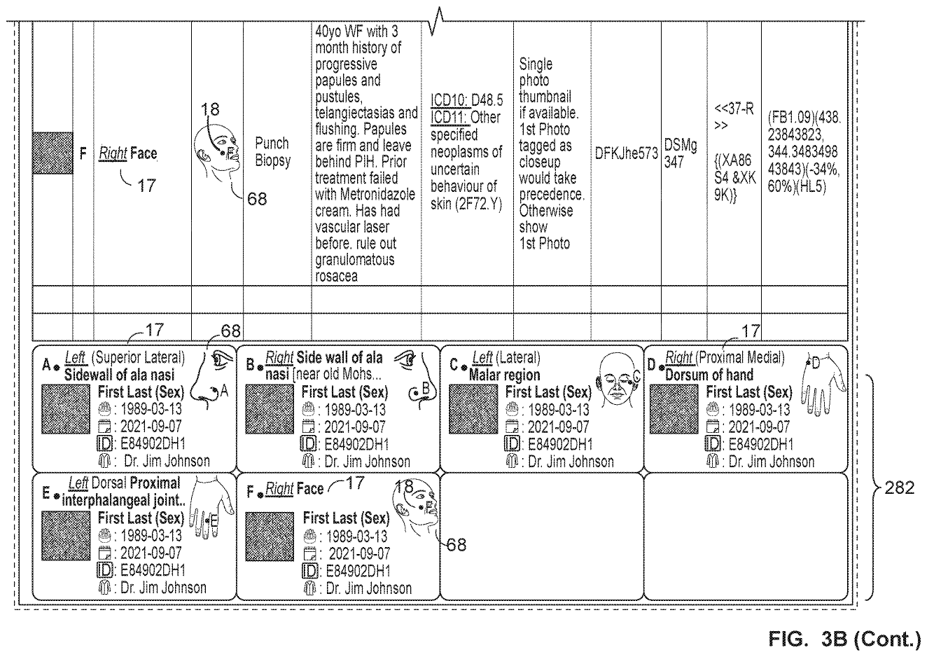

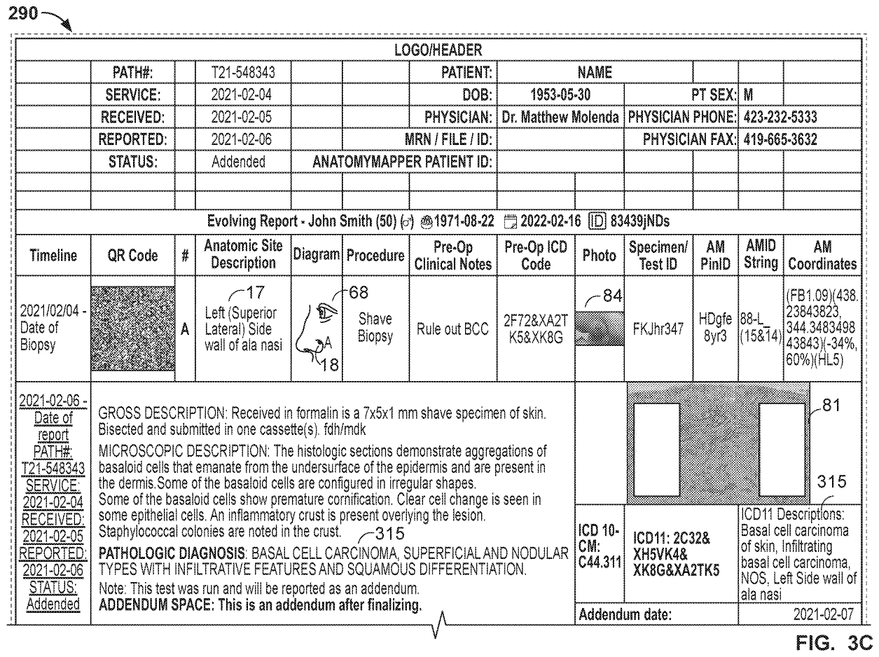

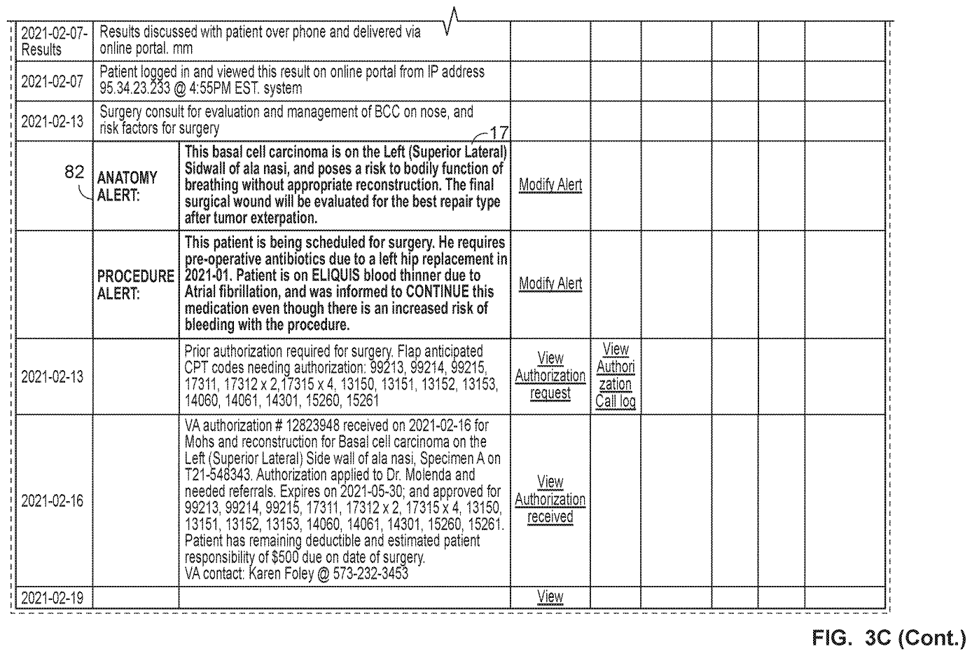

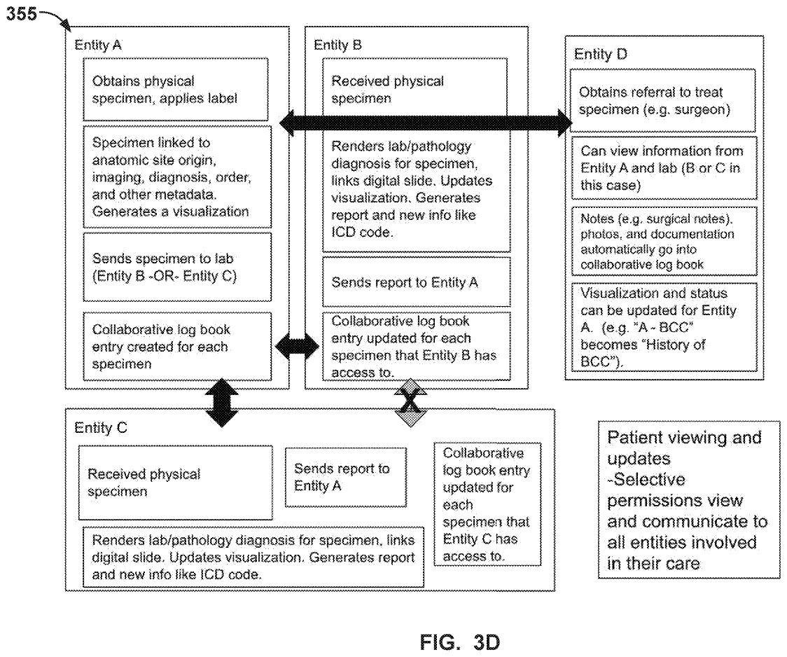

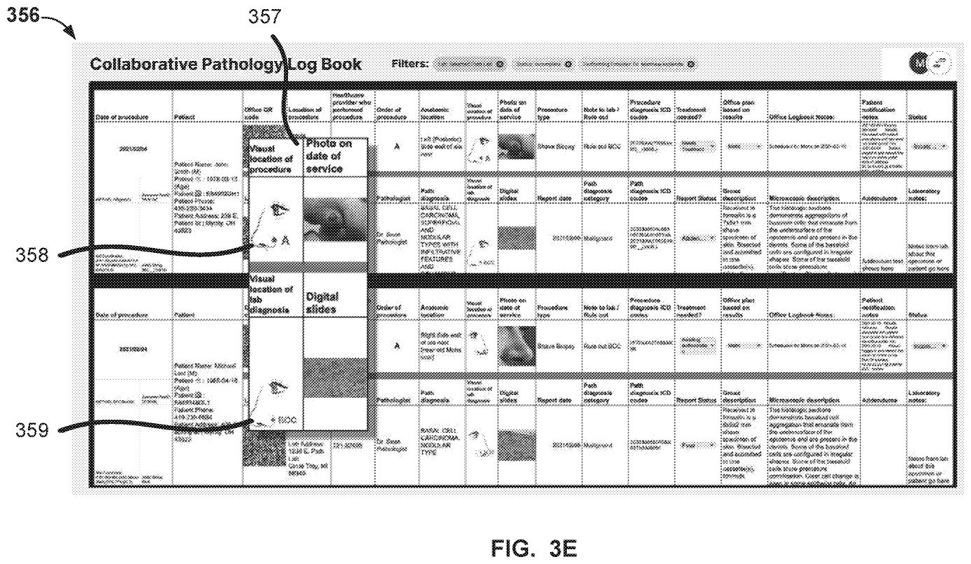

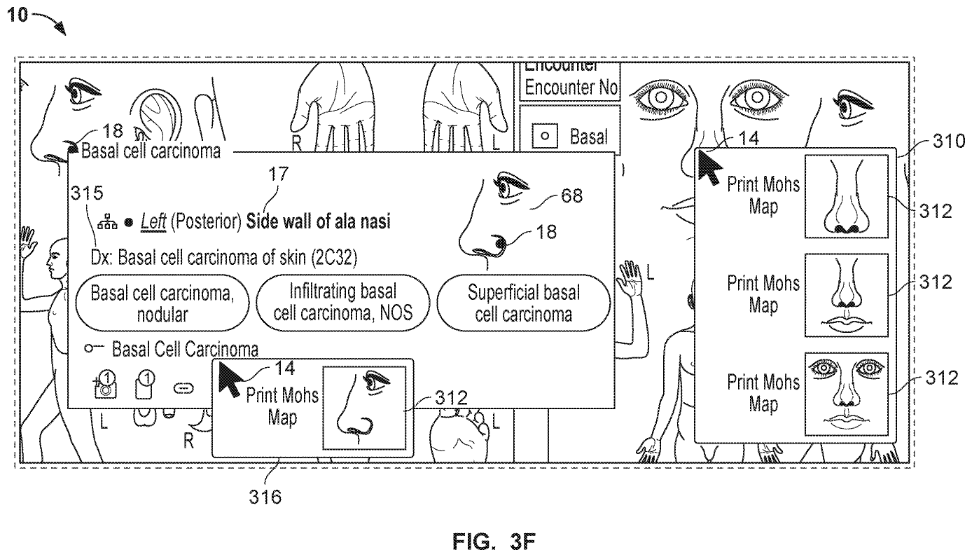

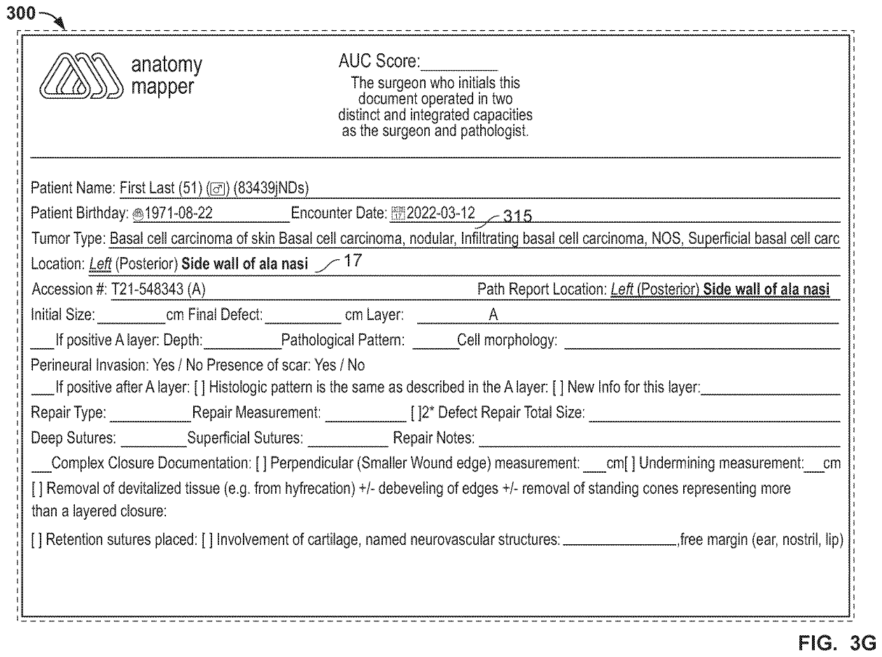

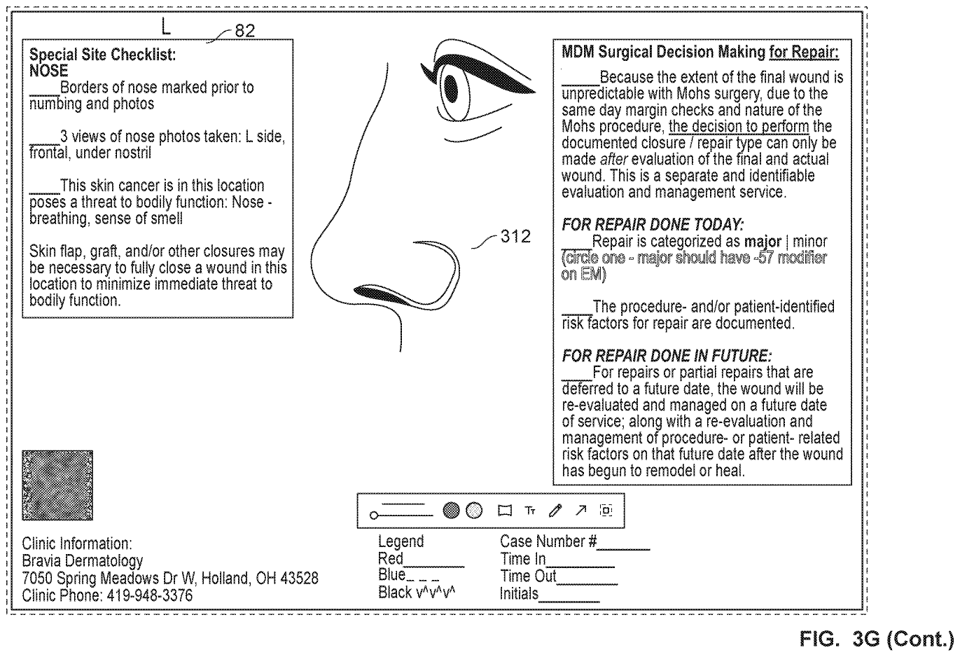

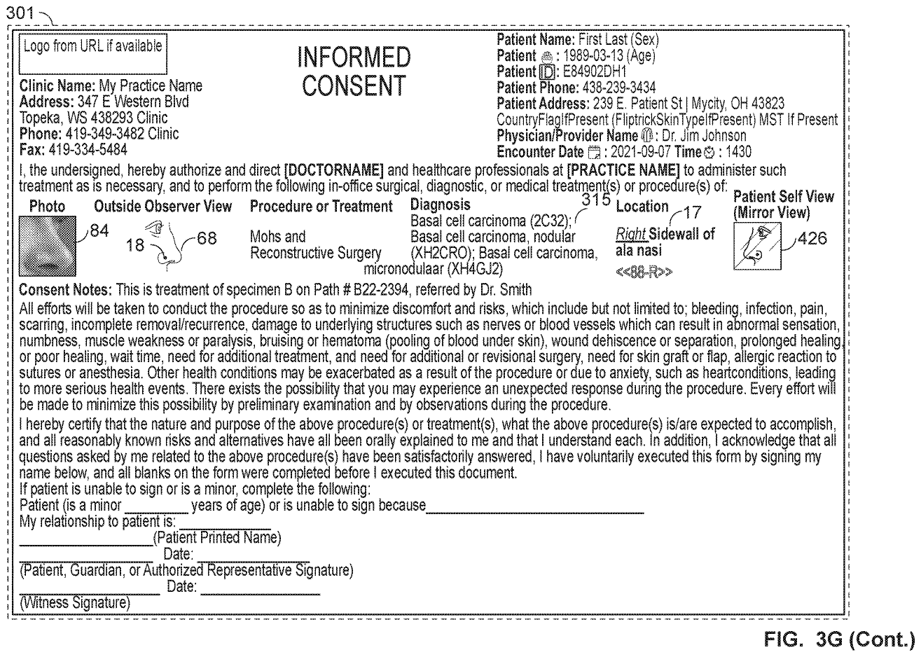

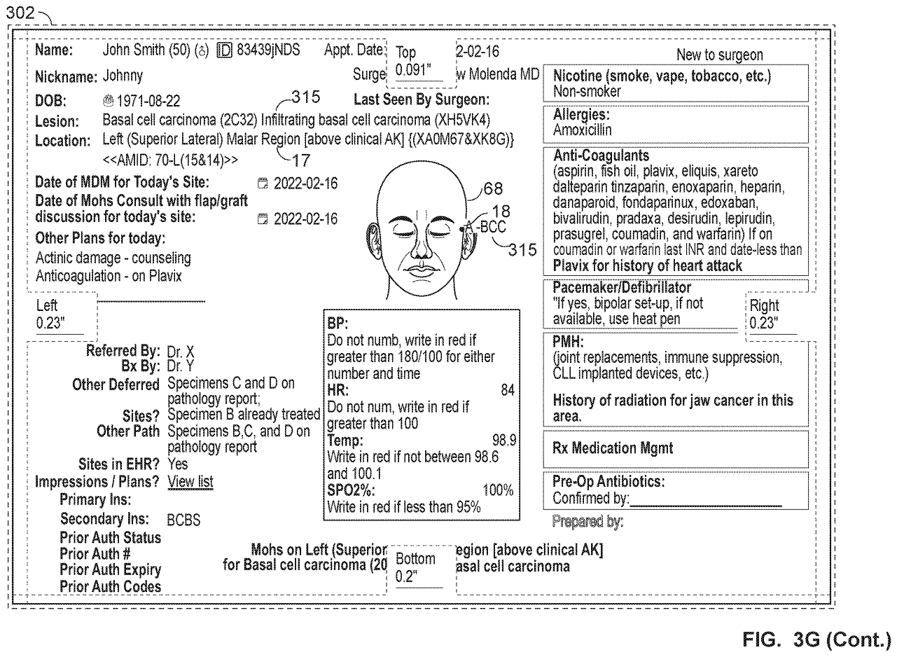

FIG. 3A depicts a flowchart of a method 215 performed and/or enabled by the system 985. Anatomy data 250 in the form of an anatomic site, point, or region of interest is determined from record defined input 261 (e.g., with a computer as the input device 988) or user input 260 (e.g., with a mouse or keyboard as the input device 988 as non-limiting examples). The anatomy data 250 correlates to a point on a layered map in certain embodiments (such as a pin as a point or a distribution segment as a region), reference to point or region on a map (such as a list item in a list of sites with an isolated visual preview 68), description of an anatomic site (such as a code string, linguistic description in any language, or a symbolic description such as an ear emoji to represent the ear), coordinated description of an anatomic site (such as coordinates on a diagram corresponding to a pin or distribution segment), or an image of an anatomy or anatomic site (such as a photograph of the ear with anatomic site detection with aligned overlays and underlays). Any of these inputs creates a dynamic anatomy library of templates 251 (e.g., with the database interface module) with hierarchical, layered, custom-coordinated, corresponding maps which include targeting sites of interest and additional diagrams representing the anatomic site of interest. The dynamic anatomy library visualizations are available in the GUI 991 in unadjusted and mirrored axes, allowing for visualization in outside observer-view and mirror-view (selfie view) 426. The generated hierarchical maps may be complete avatars in two- or three-dimensions, and/or segmented anatomic areas of interest (e.g., enabled by the generation module 993).

Non-anatomy data 258 may also be input in the system 985 through record defined input 261 or user input 260. Non-anatomy data 258 may include data from other records such as electronic health records containing patient demographics and information, encounter demographics and information, diagnosis, diagnosis extensions, patient country, user country, geographic location, patient language, user language, procedure, treatment, symptoms, morphologies, images, multimedia, reports, and other health data, or user input 260 that is either undefined by the record, or added prior to dynamic form generation. It is contemplated that non-anatomy data 258 may include, but is not limited to, measurements, notes, drawings, markups, annotations, custom descriptions, text, patient demographics and information, encounter demographics and information, diagnosis, diagnosis extensions, patient country, user country, geographic location, patient language, user language, procedure, treatment, symptoms, morphologies, images, multimedia, reports, comments, and other health data that one skilled in the art would know. User input 260 can also modify the information prior to dynamic form generation 257. It is contemplated that modifications may include selecting a different dimension (e.g., with a mouse as the input device 988), hierarchy level, axis, description, or diagram from the dynamic anatomy library, or refining the position and description of the point of interest or other manipulation of the input as one skilled in the art would know.

The dynamic anatomy library 251 and the non-anatomy data 258 of the system 985 are translated and transformed into generated dynamic forms with the dynamic form generation and translation engine 257 (e.g., with modules in the medium 995), which include anatomic images, diagrams, maps, and descriptions from the dynamic anatomy library 251, and patient, diagnosis, and encounter information from the non-anatomy data 258. The generated forms may be printable forms 259 or electronic forms 256. A plurality of templates, diagrams, data blocks, maps, records, and known inputs are used to generate each dynamic form, and unknown inputs leave blanks that can be targeted to fill in with record defined input 250 or user input 251. Printable forms 256 are automatically filled in by the dynamic form generation engine 255 with relevant anatomy data 252 and non-anatomy data 254. In certain embodiments, the printable form 256 is a Mohs map, used in micrographic dermatologic surgery, with detectable anatomic diagrams, surgical whiteboards, and checklists that contain visual representations of the anatomic site that will have surgery, along with printed pathology requisition forms and labels that have isolated visual previews of each anatomic site. Printable forms can be printed to the output device 989. Electronic forms 257 include the same data as printed forms 256 but in electronic format. In certain embodiments, the electronic form is a digital consent form capable of accepting electronic inputs from the record retrieval engine 261 (e.g., enabled by the record retrieval module 997) to fill-in blanks on the form, such that it can accept a digital patient signature (e.g., through the input device 988 like a stylus or touch screen). These are only two example forms, there are countless forms used in the medical field and any could be incorporated in this system as one skilled in the art would know.

It is contemplated that both printable forms 256 and electronic forms 257 automatically account for geographic, country, and language considerations. In one exemplary embodiment, complex closure requirements are included on forms generated in the United States and in English because those requirements are only relevant to the current US-based procedural terminology billing codes used in the United States.

It is contemplated that both printable forms 256 and electronic forms 257 also automatically account for anatomy-specific information. In one exemplary embodiment, on a consent form for surgery on the temple, a warning alert about a temporal nerve injury and the consequences of injury (such as the inability to raise the eyebrow) is generated. On the consent form, for example, a mirror or alternate visualization generated by the generation module 993 would enhance patient understanding of the anatomic site on which they are agreeing to surgery. In another exemplary embodiment, on a generated pre-surgery checklist for nasal surgery, photo workflows are included to ensure all views of the nose are photographically captured.

After printable forms 256 and electronic forms 257 are marked up, annotated, or otherwise finalized (e.g. with the input device 988), including but not limited to, filled in blanks and checklists, corrections or edits made, markup on anatomic diagrams representing a map, annotations on anatomic diagrams representing a map, physical labels placed on physical specimen bottles, digital signatures completed, and other data, they are delivered to the form processing engine 258 (e.g. enabled by the data processing module 999). Delivery to the form processing engine 258 may be through electronic or manual means. In one exemplary embodiment, computer vision analysis automatically extracts, categorizes, and digitally documents the information, including precise anatomic names and descriptions correlating with the markup, and delivers the data to the form processing engine 258. In another exemplary embodiment, a QR code representing anatomy and non-anatomy data is scanned and delivers the data, including anatomic visualizations and maps, to the form processing engine 258.

The form processing engine 258 simultaneously delivers data 259 to the anatomy data engine 260 and non-anatomy data engine 261 that categorize, encode, translate, and label the data using neural networks and natural language processing (e.g., with the medium 995 in communication with the processor 986). The two engines generate the record outputs 262 including, the billing code engine 263, the record generation engine 264, and the image and visualization labeling engine 265.

The billing code engine 263 (e.g. enabled by the data processing module 999 and other components of the system 985) uses anatomy data 252 including but not limited to anatomic site, surface area, intensity, nearby structures such as named nerves, and non-anatomy data 254 including but not limited to measurements, counts, diagnosis, diagnosis category, patient country, user country, user region, selected language, procedure types, procedure counts, complexity measurement, and other data. The billing code engine 263 can apply the same data agnostic to language and geography, simultaneously to different billing code sets in different countries such as CPT codes in the USA, and the OPCS Classification of Interventions and Procedures in the United Kingdom.

The record generation engine 264 transforms the anatomy data 262 and non-anatomy data 253 into translatable, dynamic records that includes but is not limited anatomic site listings, anatomic site visualizations, calculations such as surface area and intensity at a time point or at different time points, diagnosis codes, anatomic site codes, procedure codes, descriptions for all the codes, time points, areas, calculations, QR codes, digital bookmarks, file name strings, delimited metadata, PDF records, image records, other file type records, structured language records, unstructured records, database records, encrypted or unencrypted files such as zip files and PDF records, sharable collaborative session records, and other record formats as some non-limiting examples enabled by the system 985. Certain encoding systems, such as ICD-10 for diagnoses, combine anatomy data 262 and non-anatomy data 253 into a single code. The record generation engine 264 delivers both combined data encodings, and separated data encodings simultaneously. Therefore, this translation also works bidirectionally in the system 985, so ICD-11 codes can be converted to ICD-10 and other code sets that include anatomy and non-anatomy data through a translation engine and cross-mapping dataset, and vice versa.

The image and visualization labeling engine 265 detects, visualizes, highlights, translates, and/or labels anatomic sites. In one exemplary embodiment, the image and visualization labeling engine 265 (e.g., enabled by the data processing module 999 and/or the generation module 993) applies labeling in a symbol delimited, order-less string of data that includes alphanumeric and symbolic characters representative of the anatomy and non-anatomy data. An example label could read:

<<“Calendar emoji” to represent the encounter date, e.g. as a delimiter>>20220216<<“left double arrow emoji” to represent first name>>John<<“right double arrow emoji” to represent last name>>Smith<<“birthday cake emoji” to represent birthday>>19780822<<“male emoji” to define that the patient is a male>><<“id emoji” to represent and delimit the medical record number>>83439jNDs<<“LstItem” as a non-symbolic definition to define this example story is about a list item>><<“camera emoji” to define this section of the example story is a photograph >><<“list emoji” to represent the type of list this example story belongs to >>OrderedProcedure<<“half filled circle emoji” to represent the order of the story in a list of stories as one non-limiting example>>A<<“Shave_Biopsy” as a non-emoji example defining the type of procedure involved with the story>><<“head and neck silhouette emoji” to represent this story involves an anatomic site on the head and/or neck as one non-limiting example>><<“speaking emoji” to represent the beginning of a language description for the anatomic site involved in the story>>Malar_region<<“black star emoji” to indicate the beginning of an id number for the anatomic site>>72<<“unfilled star outline emoji” to delimit the beginning of the modified anatomy code strings>>XAOM67-XK9K<<“heart outline emoji” to delimit the beginning of the diagnosis code string>>2F72<<“scale emoji” to delimit the beginning of a test identifier>>6yDgsDcGvs4a<<“target emoji” to delimit the beginning of a pin identifiers such as on a map>>573ns8d74hsD<<“map emoji” to delimit the beginning of an identifier for the map version>>FB1.09<<“left and right arrow emoji” to delimit the x-axis of the story relative to a map or diagram or other anatomy representation>>290.593<<“up and down arrow emoji” to delimit the y-axis of the story relative to a map or diagram or other anatomy representation>>488.343<<“white coat emoji” to delimit the beginning of the physician name relevant to the story>>Matthew_Molenda_MD<<“11102” as a non-symbolic encoding mixed into the store to represent a procedure associated with the story, in this non-limiting example a shave biopsy on the storied anatomic representation e.g. using a CPT code>><<“journal emoji” to delimit a patient history provided for the story>>grew_over_2_weeks<<“tag emoji” to delimit the beginning of a tag string associated with the story>><<“magnifying glass emoji” next to the tag emoji to define this story is tagged as a close-up or magnified view>><<“OK emoji” in the tag string to define this story is tagged as approved to use in research>><<“light bulb” emoji in the tag string to define this story is tagged to have been ascertained under non-polarized lighting conditions, e.g. for a dermoscopy image>><<“ruler emoji” in the tag string to define this story is tagged with a measurement, e.g. such as one directly in the story or the image>>.jpg These emojis described in the embodiments are non-limiting single-character or low-character symbolic representations of data delimiters and/or data definitions, and such symbols do not necessarily need to be emojis (e.g. they can also be represented as linguistic parallels and/or unicode symbols), as one skilled in the art would know. As a practical application, the above example file name, when told with actual symbolic delimiters and/or symbolic definitions, can be condensed (and/or compressed) to fit into less than 256 characters, for example.

The record outputs 262 from these three engines of the system 985 become searchable and modifiable records for the record retrieval engine 266 (e.g., enabled by the record retrieval module 997). In one exemplary embodiment of modification, the symbol delimited example string above is retrieved through a search for images with the diagnosis code “□2F72” and the result is modified by stripping the other content in the symbolic string (e.g., with the record retrieval module 997 and/or the data processing module 999 as non-limiting examples). In another exemplary embodiment of modification, a record including images of anatomic sites linked to a specific diagnosis is modified by removing identifying patient data from the search results, providing a dataset and image set for a research study.

The record retrieval engine 266 is capable of capturing QR codes or anatomy visualizations (e.g. with the input device 988 such as with a camera, scanner, or other capture device) and non-anatomy data on a pathology requisition form at a different time point and using the scanned information as record defined input 250 which can then be fed back into the method 215 enabled by the system 985 to generate new forms and outputs. When the method 215 enabled by the system 985 is looped in this manner and new forms and outputs are generated, the method 215 enabled by the system 985 allows for data linking at different points in time and in different settings on different systems and/or platforms, such as different electronic health records.

Anatomy data 252 can stand on its own in the system 985 without record defined data 250. In one example, images with anatomy such as from text books, online libraries, illustrations, avatars, maps from other sources, rasterized images, vector models, 3D models, different views, alternate rotations, photos, videos, and other multimedia sources can be analyzed (e.g. by the data processing module 999) by artificial intelligence, machine learning, and/or coordinated language model 980 engines to generate new anatomic maps, including multidimensional maps, such as maps that can be applied directly to patient images or videos, to detect anatomic sites and distributions. In certain embodiments, the application can be to live patients in augmented reality using spatial computing, thus identifying anatomic sites of interest, morphologies, distributions, skin tones, diagnoses, and other information directly on the patient blended with the display 987 enabled by the modules in the medium 995 such as the data processing module 999 and/or the image interface module 994. In another embodiment, digital shadow chart information 6213 enabled by the system 985 is shown and filterable directly on the patient.

FIG. 3B depicts an exemplar pathology requisition form 280 generated by the system 985 which includes dynamic anatomy library information and two copies of each isolated visual preview 68, showing the pin position relative to each visual preview (e.g., enabled by the generation module 993). The table section 281 includes the procedures associated with the patient. Each row represents a surgical procedure, and photos and other data are automatically placed into the form. The pre-cut label section 282 on the output device 989 (e.g., specialized paper) has a corresponding summary of each procedure appearing in the table section 281. The first isolated visual preview 68 is on the row that describes the anatomic site, diagnosis, and notes. The second isolated visual preview 68 appears in the label section 282 of the pathology requisition form 280. It is contemplated that the paper size could be adjusted automatically to accommodate users based on their geographic standards. This is just one of a plurality of templates for different forms and outputs that use isolated visual previews 68 of the dynamic anatomy library. It is contemplated that it would be possible to export groups, entire maps, or entire avatars with the relevant rotations. In one exemplary embodiment, physical labels are applied to specimen bottles in this use case, the reproducible isolated visual preview along with the enhanced anatomic description on each label provides a new patient safety enhancement to ensure that bottles are labeled correctly, and the specimens make it into the correct bottles. The reproducible visualization helps reduce medical errors associated with incorrect labeling. In the non-limiting example, a plurality of QR codes is generated (e.g., by the generation module 993), creating workflows that allow for dynamic anatomy library visualization and anatomy data recreation at another site, such as at a separate pathology lab for an evolving report, such as an evolving pathology report linked to the same anatomic site (dynamic anatomic address) over time and in different systems. In certain embodiments, patient demographics, insurance information, encounter information, originating clinic, notes, diagnoses, and other non-anatomy data are added automatically through QR codes (e.g., through an input device 988 that can read the QR code and initiate actions based on the read data through the data processing module 999), simplifying the intake process on the receiving end. Thus, the embodied examples demonstrate the system 985 to precisely reproduce anatomic site names, to generate infinitely scalable vector visualizations and multidimensional maps of anatomy with precise pin locations, to process the non-anatomy information into processed health data. The system 985 enables the processed health data to be compressed into a single code such as a small QR code that fits on a small label (less than one inch needed for the code), and the generation of small isolated visual previews on the same label, that may also be used to recreate or confirm the correct anatomic site. In certain embodiments, the physical label fits on a small skin biopsy specimen bottle. A single QR code recreates the entire encounter on a different day on a different system (such as at a receiving lab), and recreates pins, distributions segments, and visualizations, tags, notes, buckets, test IDs, pin IDs, and other information related to the dynamic anatomic address as non-limiting examples. It is contemplated, each pin or distribution segment in the list can have separate QR codes as well. Dynamic anatomic address recreation enabled by the embodied examples of the system 985 allows for data to be appended, merged, changed (e.g., new pathology diagnosis), visualized, and tracked at different time points. It further is contemplated, the QR codes enabled by the system 985 and its medium 995 are optionally encrypted and can evolve over time.

FIG. 3C depicts an evolving report 290 that is output to the GUI 991 on the Display 987. Dynamic anatomy library visualization, anatomy data, and non-anatomy data recreation at different time points allows for evolving reports that automatically collate, display, and analyze information from different sources, e.g., enabled by the data processing module 999. The QR code evolves along with the report and can be optionally encrypted for storage and interaction on the physical medium 995. Data from different sources, including different clinics, all collate and aggregate together to achieve this “living,” evolving report enabled by the system 985. In the illustrated embodiment, there is a pathology report, a digital microscopic image of the slide, automatic diagnosis encoding and cross-mapping based on anatomic site component of the dynamic anatomy library, anatomy specific alerts, procedure specific alerts, insurance alerts (this case requires authorization) that automatically delivers the possible billing codes for which to seek authorization (based on the anatomic site component of the dynamic anatomic address, and automatic diagnosis category, and country-specific requirements), links to authorizations correspondences, and other information related to the anatomic site and this non-limiting report example (e.g. enabled by the generation module 993, record retrieval module 997, data processing module 999, and/or other modules or components of the system 985). Once surgery occurs, the actual surgical photos automatically become part of this evolving report based on the anatomic site or pin ID components of the dynamic anatomic address. This powerful report collates all relevant health information regarding this anatomic site and diagnosis combination, automatically, over time. This is in contrast to traditional systems that have this information relevant to the anatomic site in fragmented databases, tables, tables, and even systems. Furthermore, the translation engines enable real-time, simultaneous, accurate translation and corresponding visualization for the evolving report in any coded, linguistic, or symbolic language. In one example, a collaborative pathology log book can allow users in different clinics or health systems to collaborate on patient information, having access to only certain fields based on permissions (e.g., stored in the medium 995). Visualizations, images, data, and entry are accessed based on permissions and can be done synchronously or asynchronously. Users do not even need to know the same language, as the coordinated language model 980 and other models will display the correct content for the user based on their language preferences. The collaborative log book may contain visualizations of anatomic sites, with labels that evolve over time and additional, codes that evolve over time, and other data that evolves over time. Clinical photos, digital slides, and other content are also added (e.g., with the input device 988) to the collaborative log book, and can be viewed by the entities that have permission to view them.

FIG. 3D depicts an example of a system for a collaborative log book with different entities having different permissions enabled by the system 985. In the embodiment, Entity A has access to all log book entries because they created the log book entries. Entity B has view access to the data from Entity A for the specimens sent to Entity B from Entity A, and write access to the lab accessed fields for Entity B. Entity C has view access to the data from Entity A for the specimens sent to Entity C from Entity A, and write access to the lab accessed fields for Entity C. Entity B and Entity C do not have any access to any labs that were not sent to them. Expanding this example, if entity B has multiple entities (A1, A2, A3) who send specimens to them, entity B can manage the log book entries for all of these entities in a single unified screen that contains anatomy visualizations, anatomic site descriptions, photos, digital slides, reports, and other relevant information. Entity B can also filter and sort to see Entity A1, A2, A3. Entity D may be a surgeon or another party who gets a referral to perform a specific treatment or procedure, and needs information relevant to the patient. Each entity can manage the visualizations they have access to, and can view information from other entities they have access to based on permission sets determined by the database (e.g., the database interface module 996), knowledge base (e.g., the knowledge base module 992) and authentication systems in place. A similar structure can be used for “anatomy-based chats” where information relevant to the patient, diagnosis, and anatomic site all stick together even when the information is from different entity data sources. Different users within the same organization or entity can also participate in the chat. For example, billing authorizations, prior authorizations for insurance, reception information can be added for the specific anatomic location, diagnosis, treatment, or procedure. The patients themselves can also add photos, notes, and multimedia to the anatomy based chat, such as in a telemedicine follow-up appointment, a wound check, or a lesion check. Anatomy based chats enabled by the system 985 can occur for a pin, a distribution segment, a distribution, a disease, a treatment, a procedure, a dynamic anatomic address, or other. For clarity, there can also be procedure-based chats, diagnosis-based chats, as other non-limiting examples. Permissions management occurs at user level, patient level, and entity levels to manage who has access to add, modify, or view which parts of the chat. Other levels of chat can also be present for more general information, such as a patient-based chat. Anatomy-based permissions management (e.g., as enabled by the database interface module 996), diagnosis-based permissions management, and other permissions management will help facilitate the anatomy-based chats. Another way to think about the chat is a forum or message board. Anatomy based histories, diagnosis-based histories, procedure based histories, and other histories can also use the systems and methods to generate context aware and relevant information a user has access to, even if the information was generated in another system, thus facilitating a collaborative and visual healthcare communication platform that is language agnostic and platform agnostic. The examples are representative of one possible application of the system 985, and unlimited applications are possible with anatomy-based permissions or other health data block based permissions for different users and entities and users within entities.

FIG. 3E is a screenshot of a collaborative log book example. The screenshot 356 is one example of the GUI 991 displayed on the display 987 whereby different users in different entities (business, practices, hospitals, companies, settings) have defined “view” and “edit” permission based on their entity and based on anatomic site and procedure, in the example embodiment. A zoomed in view of a component 357 of the screen shot 356 illustrates the same visualization for the same specific anatomic site, with different information modified by the entity performing the procedure 358 (the “.” and the letter “A” to represent a biopsy in this example) and by the different entity making a pathology diagnosis 359 (the “.” and the letters “BCC” to represent basal cell in this example). The “.” represents a marked anatomic site by a consistent pin point and the visualization is consistent between the diagrams 358 359, but the pin descriptions (the “.” and the letter “A” versus the “.” and the letters “BCC”) have been generated based on the information provided by the different entities. This embodiment can also be described by the system 985 herein where the dynamic anatomic address remains constant for the different entities, but the visualizations 358 359 are generated by information from the different entities, creating different pin colors and pin descriptions in this embodiment. It is contemplated that alerts will be received by different entities when status updates are available relevant to the entity, with the entity performing the biopsy receiving an alert when a pathology diagnosis is available from the other entity, as one non-limiting example.

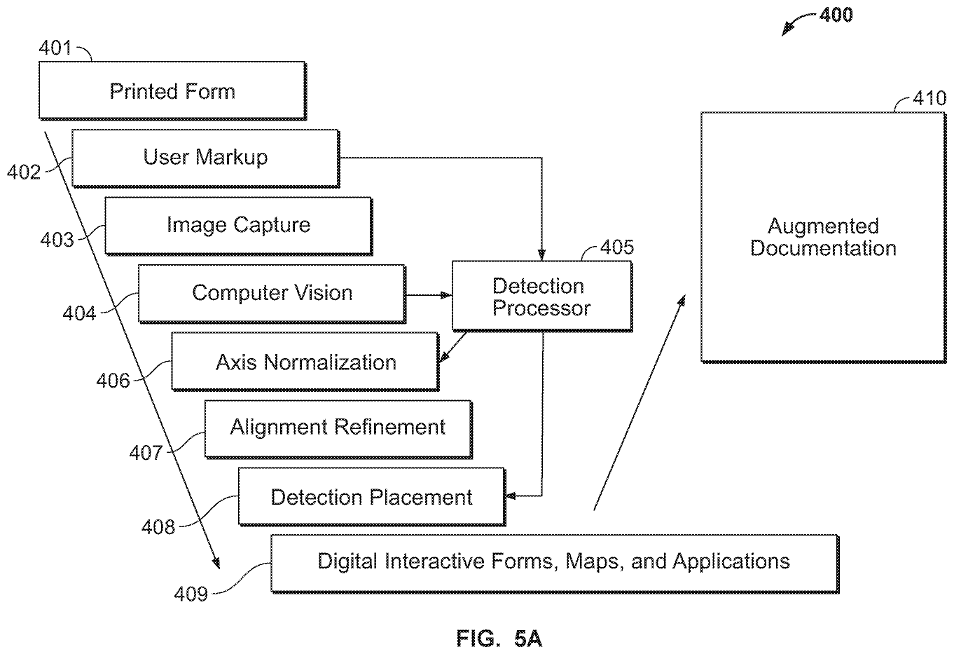

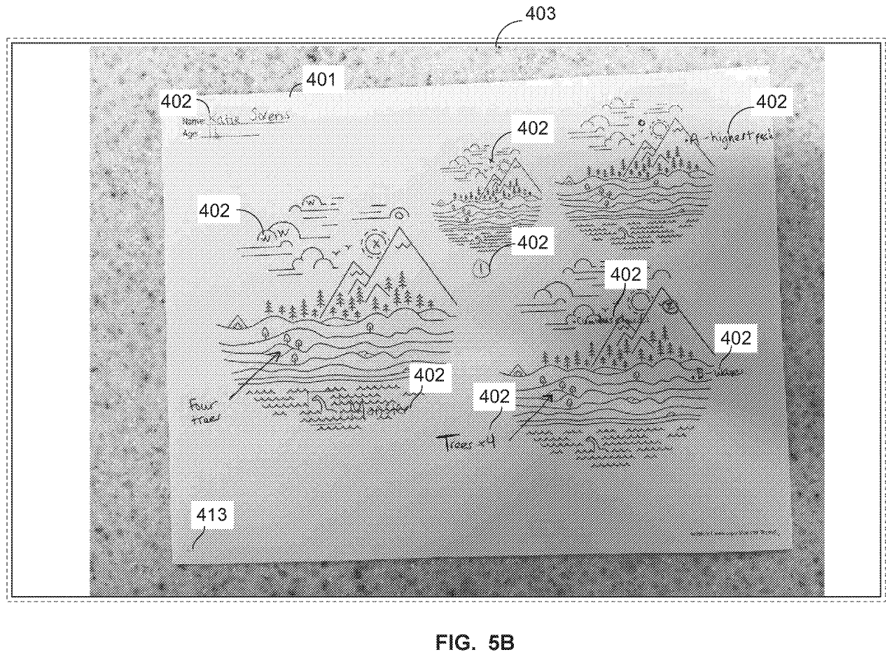

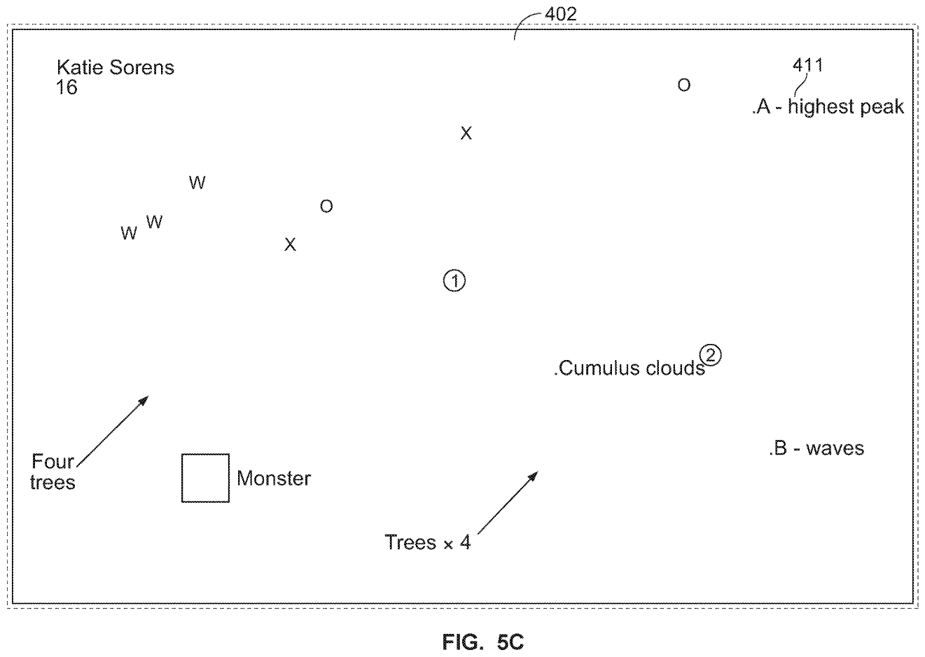

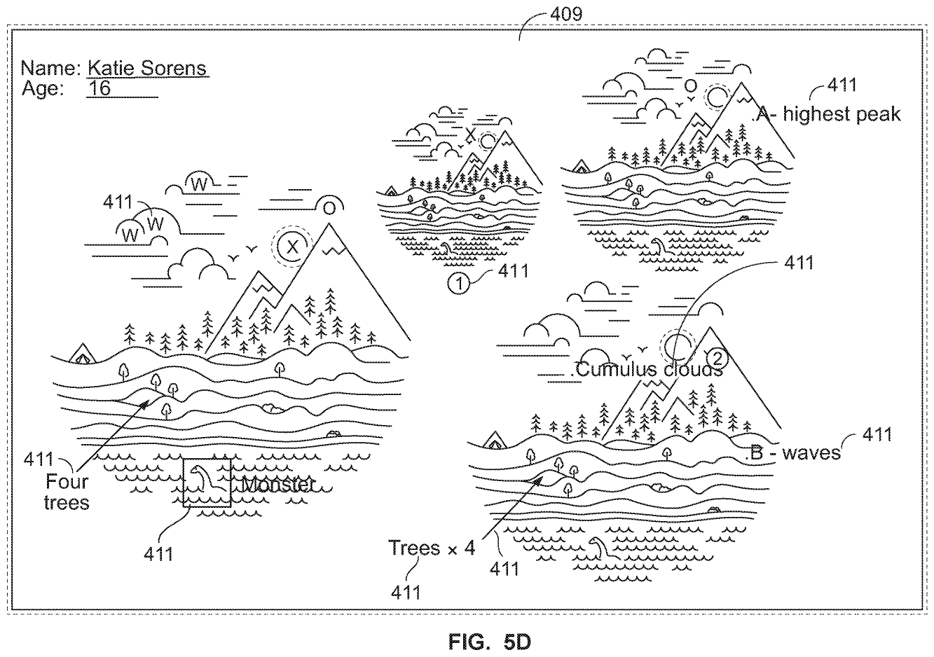

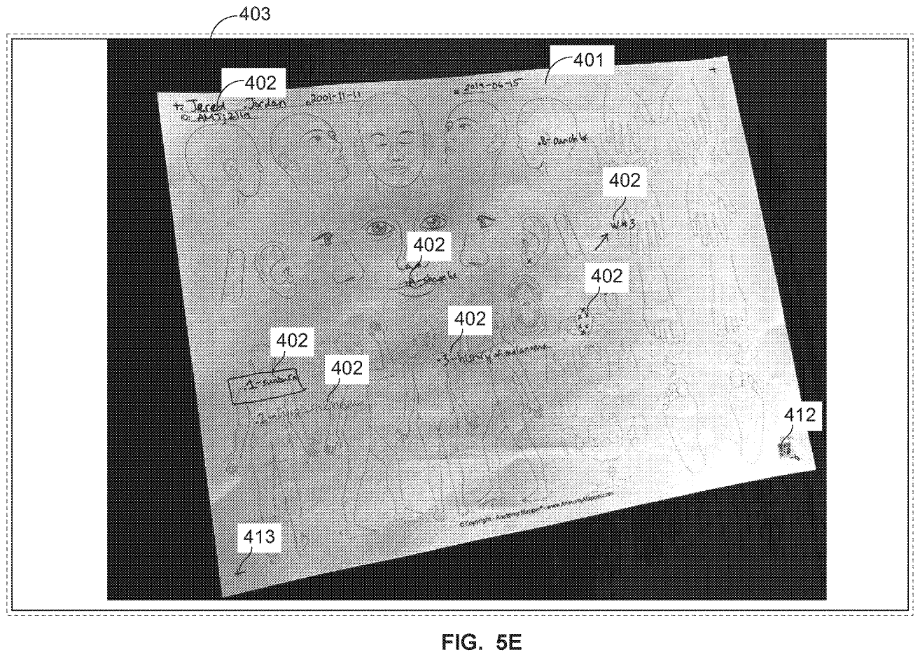

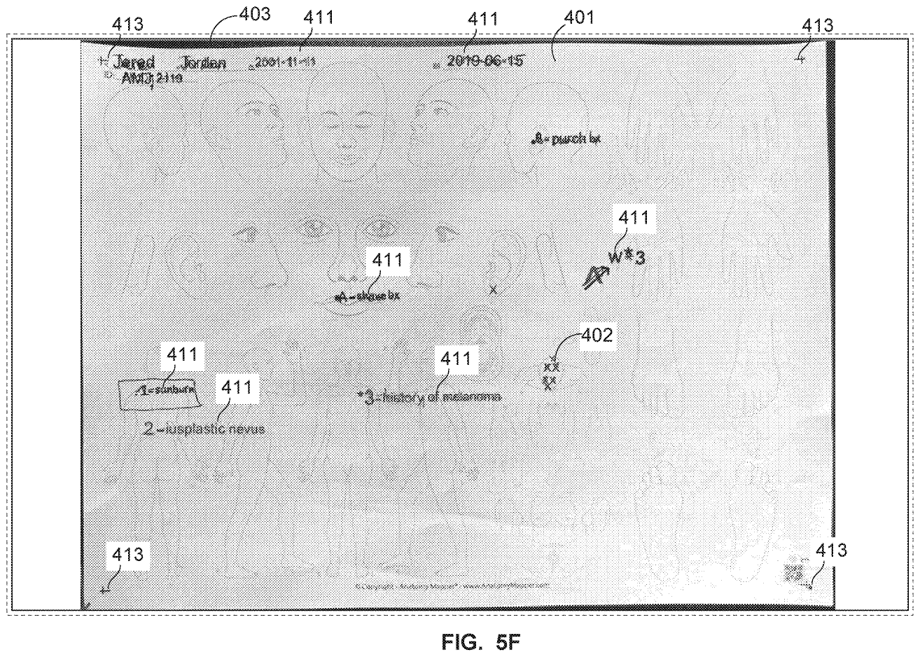

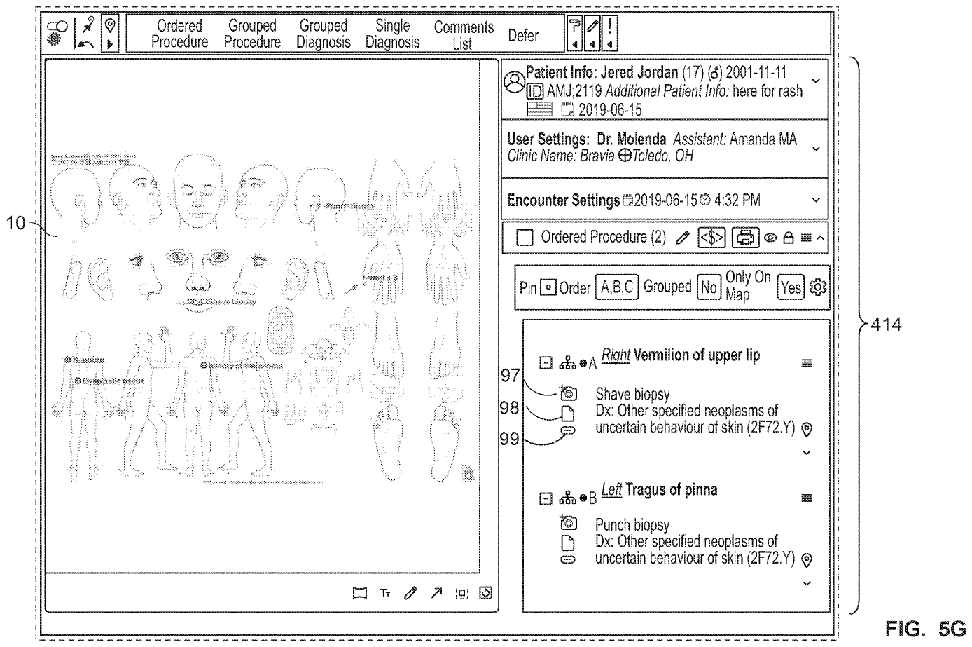

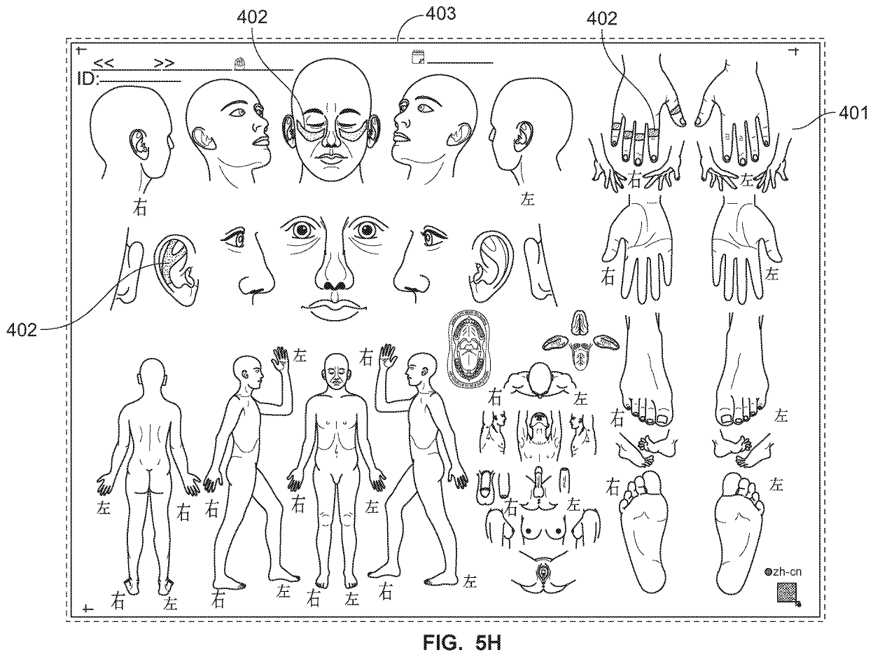

FIG. 3F is an anatomic visualization 10 (e.g., on the GUI 991) which has been automatically replotted based on the basal cell carcinoma shown in the evolving report in FIG. 5 (e.g., with the generation module 993 and/or the data processing module 999), updated with the correct diagnosis and diagnosis extension data blocks. The anatomic site name component 17 remains the same. The pin diagnosis 315 has dynamically changed to represent the pathology diagnosis, rather than the preoperative diagnosis. Context aware menus based on the pinpoint on a layered, multidimensional map are shown automatically to produce segmented form options 316 based on the dynamic anatomy library and the non-anatomy data. In this embodiment, the view of the nose with basal cell carcinoma that was automatically plotted originated from a pathology report from a different lab on a different system and generates a dynamic form with the isolated diagram and map with information from the current user's system. As one skilled in the art would know, Mohs maps documenting cancer mapping are drawn on with manual markup to represent different colors of tissue ink (e.g., with the input device 988), so the pin visualization is intentionally omitted from the form selector 316 and the isolated segmented form selectors 312, but the enhanced linguistic description of the anatomic site is included on the Mohs maps. The form is filled in and marked up electronically or printed and physically marked up. Electronic and physical markup on paper can automatically be detected by a form processing engine and both the anatomy data and non-anatomy data is processed by engines for detection, categorization, encoding, translation, and labeling. Also depicted is a dynamic context menu 310 appears over the nose part of the face diagram to show different isolated segmentations 312 available to include in a dynamic form, in this case, a printed Mohs Map.

FIG. 3G is an exemplar of an automatically generated form 300. In the illustrated embodiment, the form is a Mohs map used in micrographic dermatologic surgery. It is contemplated that the generation module 993 can also be in communication with the system 985 to determine user context (e.g. specialty), the user geographic location, the entity practice locations, the system 985 permissions, etc. as non-limiting examples to generate and/or popular the correct and most useful form data (e.g. with the correct clinic location and laboratory license number (for an entity that has multiple lab locations) listed for the Mohs map in one non-limiting example). The form 300 contains a diagram, map, alerts, and country specific information all generated from the dynamic anatomy library and the non-anatomy data. Patient demographics, encounter demographics, information from the pathology report, diagnosis information, and diagnosis extensions are automatically filled in (e.g., with the data processing module 999 and/or the generation module 993). A QR code (redacted) automatically links this form and other documentation, such as photos during surgery, to the correct dynamic anatomic address.

If this form is printed (e.g., to the output device 989), augmented documentation workflows use computer vision enabled by the system 985 to automatically detect, categorize, digitize, and place handwritten markup, apply it to the map, and attach it to the correct dynamic anatomic address in the correct position in the healthcare and encounter timeline. Anatomic site specific and procedure specific alerts and checklists are shown, with this one being related to the Mohs surgery on the nose. Electronic markup and form filling can also be done. This achieves seamless blending of paper and digital workflows related to surgical documentation, and the QR code provides quick access to add photos to the correct dynamic anatomic address from any device, even one that is not logged in. Also depicted are a consent form 301 and a surgical whiteboard 302 that can be printed, modified, signed, or marked up, and processed through certain embodiments enabled by the system 985, to automatically update and file the electronic records. In certain embodiments, the patient can send photos from their own phone to the dynamic anatomic address. It is contemplated that the same concept can apply to other dynamic anatomic addresses as well, such as in telemedicine workflows enabled by the system 985.

Uses for such dynamic forms generated by the dynamic anatomy library and non-anatomy data (e.g. enabled by the generation module 933) include some non-limiting examples: patient intake; consent forms 301 for procedures with outside observer view, mirror view, and relevant photos and can accept patient signatures digitally or on paper; mapping and documenting treatments and procedures with automatic calculation counting, translation, and coding, cross-linking of records, association with mapped regions of interest, anatomic sites; Mohs maps 300; cosmetic treatment records; automatically translated patient education handouts explaining how to use, and where to use medications (oral, topical, injectable, and other delivery methods); surgical whiteboards 302 that can be printed on paper and/or digital screens (e.g. as the output device 989).

The display 987 (e.g., digital screens) pull up additional metadata associated with the surgery anatomic site in question, provide visual verification that a consent form was signed; include a timeline and photos associated with the anatomic site, path report associated with the surgery, and other health metadata. Automatic display of relevant information or alerts that needs attention in bold, different color like red (e.g., allergies) based on automated non-anatomy data extracted from patient info. As photos, multimedia, and other metadata are added to a patient record and dynamic anatomic address (e.g., with the input device 988), for example with a different device like a phone, they automatically synchronize to the patient record and become visible on the digital whiteboard 302 (e.g., the display 987 that accepts touch and voice inputs through the input device 988) in certain embodiments.

It is contemplated the digital whiteboard 302 also responds to voice commands. Exemplar voice commands include: “Hey Whiteboard, show me the pathology report” with other non-limiting examples detailed above. It is contemplated that the digital whiteboard 302, through voice command or other user input (e.g., touch, mouse, eye tracking in communication with the input device 988), can document additional details and is language agnostic, responding to any language, including mixed languages like mixed Spanish and English language.

It is further contemplated that the digital whiteboard 302 can also speak alerts back to user (e.g., through the output device 989 like a speaker), for a conversational update to the record or treatment plan: “You asked me to send Keflex, and there are 2 interactions and patient has a reported history of allergy to penicillin. Would you like to select an alternative?”

It is further contemplated that the digital whiteboard 302 can load a patient through a QR code from a paper, wrist band, or electronic display, can load the correct patient through patient RFID tracking, facial recognition, or through other verification means (e.g., fingerprint) through the input device 988 to ensure the whiteboard is displaying the correct patient information.

It is further contemplated that the paper or digital whiteboard 302 can be used in a verbal timeout procedure; and automatically log the time out. Commands can be interpreted in any linguistic language, and document on the correct dynamic form version and correct anatomic location and visualization as enabled by the system 985.

FIG. 3G is also an exemplar of an automatically generated paper form 300 as an example from the output device 989. In the illustrated embodiment, the form is a Mohs map used in micrographic dermatologic surgery. The form 300 contains a diagram, map, alerts, and country specific information all generated from the dynamic anatomy library and the non-anatomy data (e.g., as enabled by the generation module 993). Patient demographics, encounter demographics, information from the pathology report, diagnosis information, and diagnosis extensions are automatically filled in. A QR code (redacted) automatically links this form and other documentation, such as photos during surgery, to the correct dynamic anatomic address.

In certain embodiments, a computer-implemented method for generating dynamic forms for medical records comprising: inputting, using the processor 986 in communication with stored data and/or engines, anatomy data relating to a medical record stored in the tangible medium 995; creating a dynamic anatomy library of templates wherein the library of templates comprises a plurality of anatomic diagrams, images, and/or hierarchical, layered, custom-coordinated, corresponding maps which include targeting sites of interest and additional visualizations representing anatomic site of interest; wherein the anatomic site of interest is isolatable on a layered, hierarchical map and/or visualizable in relation to the borders of each isolated anatomic site above, below, and on the same level, along with translated human readable descriptions of the anatomic site of interest position relative to each layer; inputting, using the processor 986, non-anatomy data relating to a medical record; translating the dynamic anatomy library of templates and the non-anatomy data with the dynamic form generation and translation engine to generate dynamic forms wherein the dynamic forms may include anatomic images, diagrams, maps, encoding, descriptions, translations, and/or labeling anatomic sites as well as patient, diagnosis, and/or encounter information; transforming the dynamic forms with targeted isolation, segmentation, and/or coloring of anatomic images, anatomic maps, points of interest, and/or regions of interest; wherein the targeted isolation, segmentation, and/or coloring of each layer of the hierarchical map provides simultaneous visualization, translation, descriptions, and visual selection points for a visual, translated hierarchical travel and/or selection method; optionally interacting with the dynamic forms through verbal or text based conversation in any coded, linguistic, or symbolic language.

In certain embodiments, the anatomy data is user input and/or record input. In certain embodiments, the anatomy data is a point on a layered map, reference to point or region on a map, description of the anatomic site, coordinated description of the anatomic site, uncoordinated description of the anatomic site, coded description of the anatomic site, and/or an image of the anatomic site. In certain embodiments, the dynamic anatomy templates are available in unadjusted and mirrored axes. In certain embodiments, the hierarchical, layered, custom-coordinated, corresponding maps are complete avatars. In certain embodiments, the hierarchical, layered, custom-coordinated, corresponding maps are segmented and/or isolated anatomic areas of interest. In certain embodiments, the anatomic site of interest is a pin and/or an area. In certain embodiments, the non-anatomy data is user input and/or record input. In certain embodiments, the non-anatomy data is patient demographics and information, encounter demographics and information, diagnosis, diagnosis extensions, patient country, user country, geographic location, patient language, user language, procedure, treatment, symptoms, morphologies, images, multimedia, reports, and/or other health data. In certain embodiments, the dynamic forms contain the inherent ability to re-create, translate, cross-map, plot, map, visualize, and/or encode anatomy data and non-anatomy data. In certain embodiments, a QR code less than one inch in size can recreate anatomy data and non-anatomy data. In certain embodiments, the dynamic forms are printable forms. In certain embodiments, the printable forms are automatically filled in by the dynamic form generation and translation engine with relevant data. In certain embodiments, the dynamic forms are supplemented with detectable anatomic diagrams, labels, surgical whiteboards, logs, reports, notes, and checklists that contain visual representations of the anatomic site. In certain embodiments, the dynamic forms are electronic forms. In certain embodiments, the dynamic forms automatically account for geographic, country, and language considerations. In certain embodiments, the targeted isolation provides visualization, translation, descriptions, and visual selection points for a visual, translated hierarchical travel and/or selection method.

In another embodiment, a computerized electronic system for generating dynamic forms for medical records configured to: receive anatomy data input; create a dynamic anatomy library of templates wherein the library of templates comprises a plurality of anatomic diagrams, images, and/or hierarchical, layered, custom-coordinated, corresponding maps which may include targeting sites of interest and additional visualizations representing anatomic site of interest; receive non-anatomy data input; translate and transform the dynamic anatomy library of templates and the non-anatomy data with the dynamic form generation and translation engine to generate dynamic forms wherein the dynamic forms include anatomic images, diagrams, maps, avatars, and descriptions as well as patient, diagnosis, and/or encounter information; display the generated dynamic form on a graphical user interface 991 wherein the generated dynamic form compiles anatomy data and non-anatomy data for the anatomic site of interest into a comprehensive dynamic record.

In certain embodiments, the display shows a visualization of anatomic sites and site segments relative to different layers and hierarchical levels, with simultaneously translated descriptions. In certain embodiments, the visualization depicts the anatomic point of interest in different layers and hierarchical levels, with simultaneously translated descriptions. In certain embodiments, the dynamic forms are interactive with verbal or text-based conversation in any coded, linguistic, or symbolic language.

In another embodiment, a method for displaying information related to the same anatomic site at different time points on the same anatomy visualization, wherein the order, pin-type, color, pattern, intensity, label, description, or linked information such as diagnosis evolves while the anatomy visualization remains constant.

In another embodiment, a system for displaying information related to the same anatomic site at different time points on the same anatomy visualization, wherein the order, pin-type, color, pattern, intensity, label, description, or linked information such as diagnosis evolves while the anatomy visualization remains constant.

In another embodiment, a system for generating a list of anatomic sites based on interaction with a map, avatar, image, or multimedia containing anatomy wherein: each generated list item contains a name description and anatomic site information for the selected anatomic site and the correlating isolated anatomic site visualization; optionally displaying a combined visualization for all anatomic sites in the list, visualization area, or grouping; optionally displaying all available anatomic site descriptions and visualizations under or above or around a selected point along with different visual borders displayed for each visualization; optionally automatically associating additional information with the selected anatomic site including order, order style, surface area, intensity, measurement, calculation, diagnosis, photos, attachments, links, notes, and morphologies; optionally translating the anatomic site information, visualizations, and additional information into any coded, linguistic, or symbolic language.

In another embodiment, a method for generating a list of anatomic sites based on interaction with a map, avatar, image, or multimedia containing anatomy wherein: each generated list item contains a name description and anatomic site information for the selected anatomic site and the correlating isolated anatomic site visualization; optionally displaying a combined visualization for all anatomic sites in the list, visualization area, or grouping; optionally displaying all available anatomic site descriptions and visualizations under or above or around a selected point along with different visual borders displayed for each visualization; optionally automatically associating additional information with the selected anatomic site including order, order style, surface area, intensity, measurement, calculation, diagnosis, photos, attachments, links, notes, and morphologies; optionally translating the anatomic site information, visualizations, and additional information into any coded, linguistic, or symbolic language.

(APP04) The embodiments illustrated include non-limiting examples enabling the system 985 capabilities for data collation, retrieval, organization, analysis, and summarization of health data by utilizing order-agnostic symbol delimited data linked to standardized symbols, such as emojis or unicode characters. A file name and metadata building system and method uses a data-blocking engine (e.g., enabled by the data processing module 999 and/or other modules in the medium 995) with low-character-count, symbolic delimiters automatically applied to each data field. The data blocks are orderless and structureless, with no header requirements. Similar to physical construction blocks, the digital data blocks can be constructed, built upon, deconstructed, rearranged, or modified. The data blocks build digital foundations in the tangible medium 995 on which artificial intelligence (AI) and/or machine learning can gather, collate, modify, serve, and generate language agnostic, database agnostic, and platform agnostic data for individual patients or for populations (e.g. enabled by the data processing module 999, the generation module 993, the record retrieval module 997, and/or other components of the system 985), such as in a research search engine that retrieves automatically de-identified datasets of data tagged as “OK” to use in research, among other tags, solving privacy concerns at the same time.

Traditionally, across the world different documentation systems and Electronic Health Records (EHRs) with different data and database structures are used. No uniform system is in place. While different standards like Health Level 7 (HL7) have been proposed, complex bridges among systems are required and interoperability among systems remains an elusive and unfulfilled promise, especially in the United States. In the US, different organizations, hospitals, and practices can utilize an EHR of their choosing, thus creating further fragmentation of the country's health data. And, while EHRs may try to integrate existing standards, existing standards have challenges like legacy support of outdated database structures and fields, different naming conventions, forward- and backward-compatibility, and poor image support.

Another problem with existing standards is the complexity of applying standards. Published standards are most useful when they are automatically applied, easy to use, and require medical knowledge and health literacy in only limited circumstances. Existing published standards also typically address patient demographics, facility demographics, and encounter demographics separately from diagnoses, procedures, treatments, and anatomic sites or regions; with no single method to link or unlink all of these standards together. Still further, different data headers in different countries presents meaningful challenges when performing multi-national research—where just one data point has multiple different data headers—for example “date of birth” may be abbreviated as “DOB” or “BDAY” in a US record, and as “FDN” for “fecha de nacimiento” in a Spanish system, and something else in another language, thus creating a challenge for analysis that must be manually overcome. Certain non-limiting embodiment examples enable the system 985 to solve this by associating the date of birth with a “birthday cake emoji” directly in the filename, digital bookmark within a note or record, folder name that stores the information, and file metadata (e.g., with the database interface module 996 and/or other components of the tangible medium 995). The “birthday cake emoji” in the embodiments may appear differently in different countries and operating systems, but through encodings such as unicode are distinguishable across the world, platforms, and languages. In certain embodiments, generative AI can generate (e.g., enabled by the generation module 993) symbolically delimited and defined names with symbolic delimited and defined information as file names, digital bookmarks, and file metadata. One such example of a symbolically delimited and defined bookmark is one used to find and label the exact slice, view, and angle of a CT scan or MRI that is relevant to the current context. The aforementioned non-limiting example bookmark can be stored, targeted, and interacted with in the tangible medium 995.

Categorizing and linking health data into standardized and symbol-delimited data blocks (e.g., in a medium 995) presents a unique opportunity to automatically collate, display, store, modify, and analyze health information with AI assistance as a practical application. The symbol-delimited and symbol-defined data blocks can be stored in whole, or in part, into existing systems as one practical application, and/or become part of the file names themselves or the individual file's metadata regardless of the existing system's database structure or available fields. For data block storage, the two most common non-proprietary file formats in healthcare records are documents saved (these can be stored on the medium 995) in Portable Document Format (.PDF) and images saved with compression in Joint Photographic Experts Group (.JPEG) format. These file formats already support additional metadata, like Exchangeable Image File Format (EXIF) in .JPEG. Since these ubiquitous file types can be stored either inside or outside of electronic medical records (e.g., as determined by the database interface module 996), they are more accessible and platform agnostic than medical images stored with other proprietary data types, and therefore easier to store metadata within regardless of the user's operating system or record system.

Automatic AI-assisted collation and aggregation of data blocks, especially in the context of blending anatomy and non-anatomy health data through multiple interconnected neural networks (e.g., enabled by the system 985 (e.g., through modules in the medium 995 in communication with the processor 986), allows for innumerable practical application opportunities in clinical practice and in research. In the clinical setting, this saves countless hours of searching through different medical databases and tabs to find relevant information for a single patient. Applying data block aggregation and automatic de-identification to research (e.g., enabled by the data processing module 999) creates paradigm shifts in research capabilities. The exemplar embodied illustrations apply to surface and deep anatomy; and apply to aggregation of non-anatomy associated diagnoses and other data as well. Automatically linking records, images, and reports related to a basal cell carcinoma on the “left ala nasi” with a “nose emoji” for example, create a single-character symbol-defined filter point within the patient record to easily find all records dealing with the nose (e.g., enabled by the record retrieval module 997). Adding a laterality symbolic character for “left” plus the “nose emoji” creates an even more powerful filter point that only brings up records related to the left nose. Combining a large number of these filter points creates targeted data and/or record retrieval; and creating these filter points across many patients, with the “nose emoji” for example, allows for retrieval of records related to the nose across populations. It can be contemplated that coupling a “nose emoji” data block with a diagnosis code block, like 2C32 for basal cell carcinoma from the International Classification of Disease, 11th revision (ICD-11), that the search engine included in the embodiments illustrated could collate information about all patients in a record system who have basal cell carcinoma on their nose, including photos, records, and reports-which dramatically simplifies manual collation to create automatic data sets for epidemiology research thus enabling a practical application of the system 985.

A research search engine (a “reSearch engine”) is also illustrated by the embodiments, which for individual patients can retrieve specific health data from a plurality of databases, file formats, and bookmarks (e.g. enabled by the record retrieval module 997); and for populations can retrieve automatically de-identified datasets of data tagged as “OK” to use in research among other tags, solving privacy concerns at the same time.

Automatic anatomy categorization linked to other categorizations (such as country, region, sex, age, race, diagnosis, diagnosis group, procedure, procedure group, and other health data as non-limiting examples) can happen simultaneously into various and unlimited categories with data blocks. For example, just for anatomy in certain embodiments, a point of interest on the mid left lower anterior thoracic region can be categorized into a symbolic emoji group of “tree emoji” “left arrow emoji”; a US-based CPT coding group of “trunk arms or legs”; a descriptive group of “milk line”, and ICD-11 hierarchy including “anterior thoracic region”, “chest wall”, “thorax”, “upper trunk,” and “trunk” a cross-mapping group of NYU number 217; a SNOMED CT group of 264242009 (with its own hierarchical structure); and other categorizations that can be automatically applied to additional steps, such as assigning a country- or region-specific billing code based on the appropriate categorization or assigning a tracking ID to join the categorizations into research applications, triage applications, file naming conventions, search engines, or other practical applications. Photos and other attachments can automatically be added to these categorizations and defined anatomic sites, and the metadata can be combined from the encounter, the anatomic site, and the additional metadata. For example, some cameras (e.g., enabled as the input device 988) use geographic GPS coordinates in (Exchangeable Image File Format (EXIF)) a photo's metadata to document the location of a photo. In certain embodiments, defined anatomic sites and the encounter data blocks are automatically combined with the geographic GPS data from the photos, and the data blocks can further be used to triage wartime injuries to the most appropriate medical outpost. It is contemplated that burns, percentage body surface area, gunshot wounds, chemical injuries are examples of critical data points that need to be communicated quickly, perhaps initially even over radio communication with low data bandwidth methods. In a triage example, like in a military operation (even where operatives speak different languages), injuries are documented and communicated in any coded, linguistic, or symbolic language and automatically triaged based on anatomic sites of involvement, body surface area, injury type, injury intensity, injury count, injury distribution, and geographic GPS (geographic global positioning system) location (e.g., enabled by the data processing module 997). In certain embodiments, the anatomic site encoding system is even customizable and encryptable, so even if intercepted, the coded communications would need a decoder to make sense of it. The embodied visual and language-agnostic exemplar approach also has practical applications in both healthcare and military use cases. Certain embodiments enabled by the system 985 for tracking anatomic sites and metadata associated with the anatomic sites for individuals and populations have similar relevance. For example, in mass casualty events or wartime injuries, easily applied standardized labeling of injury locations and categorizations (e.g., severity, type) can assist in tactical and triage decisions (e.g., burns vs. penetrating injuries might be routed differently, based on injury type, injury severity, or anatomic location). Reporting could automatically summarize current data in real time, resulting in rapid turnaround incident management, as one non-limiting practical application example. Mapping skin findings/lesions/scars, tattoos, dental findings, Xray findings, implanted devices, and other unique features on soldiers at registration (e.g., enabled by the medium 995) is another use case in biometric identification for deep fake mimics/body doubles or for body identification in mass casualty events. Mapping of known features on military enemies and world leaders can also assist in biometric identification and deep fake detection, e.g., videos and multimedia that mimic the voice and face of another person that are hard for a human to determine are fake. It can also be used to detect timing of a video. For example, if a world leader has a subtle new lump or cyst on the left superior lateral malar cheek, and a deep fake video is created without this feature, it would be flagged as suspicious for being fake or created in the past. In other words, the anatomic map features can determine temporal accuracy of the content as a practical application (e.g., enabled by the data processing module 999 and/or the knowledge base module 992). Mapping and cataloging of moles, tattoos, birthmarks, fingerprints, dental records, implants, and other features linked to anatomy provides even greater detail in identity verification. This has practical applications in prisoner or criminal tracking. Also, one skilled in the art can appreciate the practical application in John Doe/Jane Doe verification of unknown bodies enabled by the system 985. In another example, mapping physical impacts on mannequins on standardized anatomic sites can provide military and safety data for analysis. Mapping occurs on a dummy, mannequin or any physical recreation of all or portion of human anatomy in one example, with sensors within and around the anatomic area to detect impact, temperature, and other features of external forces. This along with spatial computing could help identify armor deficiencies and weaknesses as one practical application example. In another example, self-reporting photos, videos, and multimedia attached to anatomic sites and into secure, encrypted and time-stamped packages are enabled by the system 985, and the self-attachment can present anatomy in the perspective that makes sense to the user (e.g., selfie view). A practical application the system 985 is with victims of violence or sexual assault, who may not seek care right away and may not photograph their injuries due to fear of their assailant finding photos and videos on their phones (e.g., the input device 988). The example embodiment ensures accuracy and privacy while preserving evidentiary value. This embodied application system 985 allows users to self-document their findings such as injuries over time as symptoms arise, progress and resolve. They do not have to store their images in their camera roll. Encrypted device local storage (e.g., enabled by the database interface module 996), emailed encrypted attachments, and encrypted cloud options can help alleviate victim concern, and the application icon enabled by the system 985 can be a decoy icon for maximum privacy.

Certain embodiments enabled by the system 985 apply a vocabulary builder and a site naming sequence configuration through artificial neural networks and/or other databases and/or information networks and/or language models and/or vision models to break down anatomic site descriptions into data blocks including site name, laterality, prefixes, suffixes, enhanced modifiers to describe direction, custom descriptions and triangulations, automatic relationship descriptions with magnitude modifiers, code sequences, translations, synonyms, groupings, symbolic references, cross-mappings, and other metadata as non-limiting examples (e.g. enabled by the data processing module 999). In other words, these are some of the “anatomic components” or data blocks of the anatomic site name, and the embodiments illustrated can arrange these in customized ways based on user preference or language. An example of this rearrangement is with natural linguistic sequencing applied through natural language processing (e.g., enabled by the data processing module 999) to show the anatomic site name first in Spanish; followed by laterality (“left hand” in English is most naturally “mano izquierda” in Spanish (which sequentially translates to “hand left”). Certain embodiments can also detect, translate, and visualize combinations of coded, linguistic, and/or symbolic inputs like “Spenglish” “left mano” for left hand could automatically be translated to the correct linguistic and coded language for mano izquierda, and thus the correct symbolic language outputs into delimiters or standalone symbols, aka “symbol-defined data blocks.” Through natural language processing, laterality and other modifier terms can also be added within the anatomic site terms. For example, “Left (Superior) Crura of antihelix” with the laterality “left” and modifier “superior” and site term “Crura of antihelix” could be presented (e.g., displayed on the GUI 991) as the “Superior aspect of the Crura of the left antihelix.” Expanding this further, the hierarchical terms can also be added with one example, substituting the synonym term “ear” for “pinna”, being “Superior aspect of the Crura of the left antihelix of the left ear.” One skilled in the art would know there are numerous combinations of language and modifiers that can confer the same meaning on a physical embodiment, location on a person, or location on a diagram. As another single example, applying different semantic order and modifiers to the anatomic site descriptions can enhance human understanding, for example, with natural language processing to transform the “Left Crus of antihelix” and enhance the term to read as “Superior crus of left anithelix.” Slang symbols and semantics/language can also be mixed in, for example, in certain embodiments where a patient reports a concern of “itching” on their “izquierdo” “peach emoji”, at which point certain embodiments enabled by the system 985 coordinate the concern to be “itching on the left buttock.” In other words, if someone has limited anatomy knowledge of one linguistic spoken language, they could mix languages to achieve desired translations, coordinates, visualizations, categorizations, and other data as a practical application. Language inputs can also be verbal or spoken and translated into a standardized anatomic site name and visualization. Automatic language modification also applies language specific considerations, such as changing laterality endings for masculine vs. feminine terms (izquierdo vs izquierda); or to display considerations for languages that read from right-to-left, such as Hebrew and Arabic. This, combined with applying neural networks to data block translations of the GUI 991 symptoms, morphologies, durations, numbers/alphabets, descriptions, diagnoses, diagnoses extensions, tags, visualizations, legends, and all other components of the embodied application system 985 engine allow of the embodiments illustrated for automatic, enhanced translation of the entire medical encounter, with or without anatomy visualizations and images. In certain embodiments, the entire medical record can be translated, visualized, and mapped into any coded, linguistic, or symbolic language and categorization. For example, in an embodiment that applies a coordinated language model 980 to existing healthcare records, such as in a practical application of a migration from one electronic health record to another, wherein the records contain varying descriptions of anatomy with varying descriptions of accuracy, precision, specificity, and reproducibility, the detected health data is translated, processed and plotted (e.g. into processed health data, e.g. by the data processing module 999) automatically on maps, avatars, and images; and the plotting can be refined, merged, deleted, or modified by the user of the system 985; and described, encoded, translated, and categorized in any coded, symbolic, or linguistic language.

The anatomic site, standardized anatomy codes, names, and symbols, dynamic anatomy codes and visualizations, targets, patient data, diagnosis data, encounter data, tags, and other data can be used to generate (e.g. enabled by the generation module 993) language-agnostic file naming, grouping, and exporting function with optional universal symbolic low-character-count delimiters to automatically write a language agnostic story about files, documentation blocks, bookmarks within patient charts, labeled specimens, photos (and other multimedia), attachments, links, and other metadata (e.g. enabled by the medium 995). This symbolic delimited and defined data (termed as “data blocks” in the embodiments illustrated) can be truncated in a file name, encrypted into static or evolving QR codes (or other codes with or without encryption), stored in exported file metadata, exported to a database or file wrapper (such as a Digital Imaging and Communications in Medicine (DICOM) wrapper), filtered, searched, de-identified, encoded, and tagged (e.g. enabled by the data processing module 999). In other words, the data blocks are combined and separated with meaningful delimiters and/or definitions, such as symbolic delimiters and/or definitions like universally translatable emojis, into an order-independent, structureless, meaningful story (e.g., by the data processing module 999 and/or the generation module 993). Reiterating this, in certain embodiments, the system 995 writes a “novel” about the file using data blocks that does not have to fit into an EHR or other defined data structure (since the system is database independent and agnostic, and data structure independent), and include that “novel” in the filenames, file metadata, bookmarks, folder names, or all of the preceding; or include that novel as bookmarks within other records, thus automatically creating a filter and data target point. A reSearch engine, used for research on individual patients or populations, can retrieve identifiable or scrubbed (de-identified) health data based on the data blocks. In another application AI collates these data blocks to put together a history or timeline for an individual patient related to a specific anatomic region of interest, by using a single anatomic site or category, or a group of anatomic sites or categories, or other non-anatomy data blocks. Using a coordinated language mode 980 type model, a proximity-based history based on visual input or mixed inputs can be generated with automatic context awareness and boundaries or custom anatomic boundaries.

FIG. 4A depicts an omnidirectional data model 330 enabled by the system 985 where uncoordinated 332 and coordinated 334 anatomy data 336, non-anatomy data 338, geographic data 342, tags 344, and data buckets 340 containing photos, attachments, and links enable the capabilities 350 of the data block engine 352. For anatomy 336, an uncoordinated data 332 example would be a linguistic description like “right ear.” Coordinated data 334 examples would be the position of a pin on a visual anatomic map, or selection of an anatomic region of interest on an image that has an anatomic map. A membership and category 346 example would be the “right ear” belonging to the “head and neck” in a hierarchical relationship, and the “auditory system” in a functional system. Data buckets 340 overlap with anatomy data 336, tags 344, non-anatomy data 338, and communicate with geographic 342 and linguistic language 348, and contain data such as photographs, attachments such as pdf reports, and links such as hyperlinks to a specific bookmark in a medical record. A geographic coordinated data 342 example includes the geographic GPS coordinates of a photograph captured by a GPS enabled phone with a camera that stores the GPS data in the photograph. In this embodiment, tags 344 are used to supply data block tags to the data block engine 352, with an example being “OK” tag symbolized by the “OK emoji” signifying that the patient has approved to allow their data to be used in research. It is contemplated that the data block engine 352 has practical application capabilities 350 in record generation, retrieval, deidentification, translation to any coded, linguistic or symbolic language, sequencing timed data into timelines, form generation, visualization of healthcare data (such as re-creating a point on an anatomic map to show the location of a disease, or selecting a region of interest on an anatomic map to search the research engine), tracking of records and health data including monitoring of populations, and filtering and scrubbing structureless, orderless health data to deliver the required results. The data blocks flow in all directions, making the system 985 omnidirectional. It is contemplated that all of the data are connected to a neural network (e.g., enabled by modules in the medium 995) that can be used by AI and/or machine learning.

FIG. 4B depicts the screenshot of FIG. 1N with portions translated to Chinese. This non-limiting embodiment has been simultaneously and real-time translated to Chinese 244 in all areas except select manual inputs still in English 240. This embodiment also shows a symbolically delimited and symbolically defined filename 249 for the image 247 in a data bucket (e.g., enabled by the database interface module 996) belonging to this pin at this dynamic anatomic address. The symbolically delimited and defined filename 249 tells a language agnostic story about the pin by using symbols, and order does not matter because of the symbolic delimitation. The symbolic story telling file name can also be included with the metadata of the exported or saved file (e.g., saving enabled by the medium 995), based on user preference. The exemplar photo 247 in this embodiment belongs to the dynamic anatomic address and pin and is editable and able to be marked up (e.g., enabled by the input device 988). Additionally, the anatomic site of interest has been detected on the image 555. Additionally, the same photos or attachments or links or forms can simultaneously belong to other dynamic anatomic addresses, such as the other * pin (obscured in this screenshot by the translated photo modal 248) and exist in multiple buckets and dynamic anatomic addresses simultaneously. Some useful examples of multimedia have multiple dynamic anatomic addresses are illustrated by the photo in this figure. On the map example of the GUI 991, there is a shave biopsy to rule out melanoma (. A, red); and an inflamed seborrheic keratosis (*, brown) treated with cryosurgery right above it. the recent surgical scar above that can also have details automatically pulled about that surgery based on its data blocks derived from its dynamic anatomic address (not shown) and other data blocks, into a shadow chart 6213 or timeline view, for example. This photo 247 can belong to all three dynamic anatomic addresses and have differentiated, and the same data blocks related to the anatomic locations, in the ongoing example simultaneously, because it is relevant to each. It is contemplated that timeline views, data collation, anatomic region filtering, communication, translation, and future documentation into the correct buckets are all achieved through the dynamic anatomic addressing and data blocking models applied by the described teachings enabled the system 985 with modular components in the medium 995 to facilitate engines and artificial neural networks (e.g., enabled in the system 985) working together. Also shown in this embodiment is a translated dropdown that allows for conversion of dynamic anatomic address component of a pin to be converted to a translated distribution segment that is visually represented on the map (not shown), and has other differences such as diagnosis, but maintain its pin position through an invisible anchor pin, and bucket contents. It is contemplated that some of the data blocks remain the same (like anatomy data blocks), while other data blocks (like non-anatomy data blocks such as diagnosis), would change or evolve under such circumstances, and as one example. The aforementioned example modification of the data blocks would be enabled by the system 985 components (e.g., the tangible medium 995).

FIG. 4C is a screenshot showing options to customize the shown data with options to automatically and dynamically order, categorize, and show data 270 like coded translations, symbolic categorizations of anatomy and health data, and symbolic delimiters and symbolic definitions related to data blocks 274 (e.g., enabled by the input device 988 in communication with the GUI 991 through the medium 995). The naming sequence can individually toggle data blocks 272 related to anatomic site name components, code translations and options, optional separators, and symbolic categorizations and symbolic definitions such as emoji groups in this non-limiting exemplar. As shown in FIG. 4C the images and attachments can be customized 274 to a desired configuration in the system 985 as well. The images and attachment of certain embodied examples of the system 985 use symbolic delimiters (such as a “birthday cake emoji” for date of birth) and symbolic definitions or categorizations (such as patient sex) automatically, allowing for automatic storytelling, data aggregation, and data filtering through filenames, file metadata, bookmarks, links, file wrappers, and other digital repositories for data and metadata (e.g. as enabled by the database interface module 996 as one example). Importantly, it is contemplated that anatomic site and dynamic anatomic address component data blocks can be represented simultaneously in multiple ways in the story (e.g. enabled by the knowledge base module 992 and/or other modules of the medium 995), including but not limited to emoji groups as a symbolic category or definition, code strings, linguistic description and categories, separated laterality and site name components, test ID, pin ID, pin coordinates, pin angles and deviations, pin relationships, pin level of hierarchy, pin organ system, pin anatomy system, site relationships, site segmentations, site level of hierarchy, site organ system, site anatomy system, and other representations.

FIG. 4D is an embodiment shows an example file name 246 for a photo 247 that is named with symbolic delimiters, health data including anatomic site data, and symbolic definitions. Within the filename (e.g., stored on the medium 995), unique symbolic characters (emojis and unicode characters) construct a rich story about the photo and automatically link different data concepts together into data blocks such as encounter demographics, patient demographics, anatomic sites, diagnoses, and even free text symptoms. Even more data blocks can be saved into the actual file metadata or into different sections of a progress note or report, for example. It is contemplated that file names may have size limitations (such as 256 characters), the data blocks may be truncated in the filename and simultaneously placed into metadata fields for the file which have larger data storage capabilities (e.g., enabled by the medium 995). It is also contemplated that data blocks can document changes and histories over time, such as when some data blocks change at different time points. Just from the exemplar filename 246, which has written a novel or rich story about the file, including details about its dynamic anatomic address, it is contemplated that even a human reader could ascertain practical information about the file from the file name. Each symbolic delimited and symbolic defined component of the non-limiting exemplar file name 246 is broken down in a table 245. It is contemplated that even more data blocks can be stored within the file metadata or within file wrappers, such as DICOM wrappers. It is contemplated that symbolic delimiters and symbolic definitions can optionally use standard character-based delimiters and text abbreviations or descriptions of the symbols for legacy systems that do not support all the unicode and emoji symbols in other certain embodiments, so they may be used in parallel with legacy datasets. There are a multitude of practical application benefits in storing data blocks as described, including but not limited to: (1) platform agnosticism: single- or low-character count categorizations that are stored in the filename and/or file metadata, or within sections or progress notes or reports, allowing this concept to work regardless of the electronic health record system or database in use; (2) order agnosticism: the order of the data blocks does not matter; (3) allowing for structureless and orderless data; (3) no database is required (e.g. stored in a repository on the medium 995); (4) language agnosticism: standardized symbolic delimiters and symbolic definitions confer meaning regardless of language, and construct a human readable story just with the data blocks; (5) modifiable: as additional information becomes available, additional data blocks can be added to existing records without actually altering the integrity of the record. For example, in the exemplar, the diagnosis might have been neoplasm of uncertain behavior (2F72.Y) at the time of the photo; but after biopsy it was determined to be basal cell carcinoma, nodular type (2C32-XH2CR0); (6) data collation, aggregation and de-identification: select data blocks can be searched for, collated and/or aggregated, and automatically de-identified on an as-needed basis simply by removing the health data blocks that contain Protected Health Information (PHI) (e.g. enabled by the data processing module 999 before outputting newly named files to the generation module 993 that then go to the GUI 991 and the output device 989). For example, for compliance with PHI and Health Insurance Portability and Accountability Act (HIPAA); (7) uniquely targetable: traditionally, it is exceedingly rare to see emojis and unicode symbols in medical records or their metadata, so their use in the method set forth by this disclosure would mitigate any legacy issues while simultaneously unlocking new data frontiers. If found visually distracting, the data blocks and symbols can also be targeted to hide from the user's view, serving as invisible data blocks or “bookmarks” within a progress note, for example. Unique targeting also enables future search, collation, and aggregation capabilities.

It is contemplated that from a research perspective, formulaic searches (e.g., enabled by the data processing module 999, the record retrieval module 997, and/or other components of the system 985) through health data blocks can enable filtered, de-identified, aggregated research data that has been pre-approved for use in research (e.g., tagged as “OK” to indicate patient approval). Granular tagging and data block application of dynamic anatomic addresses, or individual photos or files, can address patient privacy concerns automatically by only allowing appropriately tagged content into research search results. Standardized and symbol-delimited health data blocks create a foundation for a research search engine (a “reSearch engine”) for healthcare research data. The included formulaic search example searches, aggregates, and delivers de-identified photos (camera-symbol for “camera”) for all male patients (mars-symbol for “male”) with basal cell carcinoma (2C32) on the nose (nose-symbol for “nose”), between ages 30-40 (calculated as age at time of encounter, by encounter date minus birthdate) by searching and modifying the health metadata blocks that have been tagged as “OK to use in research” (tag- or ok-symbol for “tag” and symbol for “OK”). It is contemplated that the order of the formulaic query does not matter (e.g., as enabled by the record retrieval module 997 and/or the data processing module 999). It is further contemplated that the files are automatically scrubbed of identifiable patient information and delivered to the researcher with relabeled data blocks file naming protocols of the system 985. Standardized and symbol-delimited and symbol defined data blocks represent a new frontier in medicine and research and are the key to innumerable new clinical and research capabilities. Applying health data block labeling and tagging to health information creates foundations for AI-assisted collation, retrieval, organization, and summarization of health data and records. For a single patient, multiple diagnoses and treatments can be linked simultaneously to a dynamic anatomic address and different anatomic regions and dynamic anatomic addresses through their data block components. It can be contemplated that machine learning and AI generate a history about a region of interest by using health data blocks including blocks from dynamic anatomic addresses, diagnoses, treatments, and dates in an area of interest. To restate, it can be contemplated that just like physical blocks, the digital health data blocks stored on the medium 995 can be combined, deconstructed, and built upon, and exist simultaneously in unlimited dynamic anatomic addresses and in unlimited block data structures, including a block chain. It can further be contemplated that a context aware, automatically generated history includes direct links to relevant photos or other imaging (like X-rays, ultrasounds, etc.), documents, forms, reports, and data collated from and made possible by the data blocks.

In certain embodiments, the system 985 uses encounter data, patient data, anatomy data 252, procedure data 254, image data 1006, extracted data, consent form data 254, and other data to automatically tag images, reports, and multimedia as non-limiting examples. For example, if a patient has signed a photography consent form (e.g., with the input device 988) allowing the clinic to post their de-identified photos online or in a research study or repository, photos captures under the signed consent policy can automatically be tagged as “OK to use in research and to publish my images.” Automated tagging, as a practical application in this example, solves privacy issues for images and mitigates user forgetfulness to appropriately tag images for research. In another embodiment, photographs associated with the genitals or breasts are automatically tagged as “Sensitive.” And in another embodiment, photographs of the face or photographs containing a tattoo or scar, are automatically be tagged as “Identifiable” because they contain potentially identifiable patient features. Photos with such tags could be processed manually or automatically with de-identification or obfuscation methods (such as blurring, pixelating, or redacting select areas) by the processor 986.

FIG. 4E depicts a screenshot illustrating a selected anatomic site 18 at the left dorsal proximal interphalangeal joint of little finger, coordinated anatomy data 16 is shown in correspondence with a color-coded legend 12; and symbolic definitions for the anatomic site group 20 are shown for each dimension, level and layer in an English embodiment at the end of each anatomic site description 19. It is contemplated that the symbolic delimited and symbolic defined emoji categorization captures all of anatomic site data in a granular way simultaneously regardless of language or code set through a cross-mapping data set, neural networks, and data block engine enabled by the system 985.

FIGS. 4F and 4G depicts non-limiting exemplar emoji searches 65 (e.g., enabled by the input device 988 in communication with the GUI 991 and the processor 986 to execute a search in the medium 995) and application examples 66 with their unicode backup. While emoji characters may render differently on different systems, they are backed up by unicode and confer the same meaning nearly universally. Some emojis may even display differently in different countries based on inherent emoji localization features. It is contemplated that the “birthday cake emoji” may display differently in Japan than in the US; and the “birthday cake emoji” is closer to a global universal symbol for “date of birth” than “DOB.” Furthering this example, in Spanish, “fecha de nacimiento” means “date of birth” or “birthday” and may be abbreviated as FDN in a record system, thus it is contemplated that these different data headers in different languages create the need for manual data cross-mapping in multi-national research on health data as a practical application. The system 985 solves this non-limiting example issue with a unified symbolic definition stored on the medium 995.

FIG. 4H depicts an exemplar single AI, context aware, automatically generated history (e.g., enabled by the record retrieval module 997, the generation module 993, other modules, and/or components of the system 985) includes direct links to relevant photos or other imaging (like X-rays, ultrasounds, etc.), documents, forms, reports, and health data. The underlined sections 64 shown in the illustrated embodiment represent hyperlinks directly to the relevant notes, reports, photos, and other health record information. AI and machine learning generated single patient history in English collated, organized, and presented from data blocks in the selected anatomic site. It is contemplated that data are organized in a timeline based on the selected anatomic site 52, and account for regional anatomic sites as well (with information delivered for the left cheek, which is automatically included in the specialty context and the anatomy context) (e.g., enabled by modules and/or components of the system 985). It is contemplated that automatic cross-links are generated to procedure summaries, photos, results, prescriptions, and other health data associated with the dynamic anatomic addresses and data blocks, represented as blue hyperlinks 64 in the figure. It is further contemplated that progression, transformation, recurrence, growth, resolution, and other changes can be tracked, documented, and analyzed automatically because of the platform that includes the dynamic anatomic addressing method and the data blocking engine. It is further contemplated that symbolic delimitations and symbolic definitions and symbolic categorization in all systems form artificial neural networks in the system 985 that enable language agnostic, order agnostic, platform agnostic, modifiable, and targetable results, collation, and aggregation.

It is contemplated that an example of a more global result, with deep anatomic sites (not shown), includes applying the dynamic anatomic address and data blocks, and the data block engine of the embodied systems and methods, to answer global questions that affect superficial, deep, or systems-based anatomy as a practical application. For example, a standardized distribution tracking output that automatically segments the lungs in layered or three-dimensional space tracks how a respiratory virus affects different areas of the lungs with fibrosis, inflammation, hemorrhage, and other morphology features identified on medical imaging or biopsies. Taking this non-limiting example further, the inflammatory profile is linked to different dynamic anatomic address areas of the lung where fluid samples were taken, providing dynamic collated answers to questions like: Is the inflammatory response different in the lower lung versus the upper lung? Does the inflammatory profile of the different lung areas change over time during a disease course? How does drug X affect the inflammatory profiles in different areas of the lung? Does drug X alter progression to pulmonary fibrosis? Is the left or right lung more likely to progress to fibrosis? The system 985 is configured to communicate with the data processing module 999, the record retrieval module 997, other modules of the tangible medium 995, and/or other components of the system 985 to answer these questions.

In one embodiment, a method to apply symbolic delimiters and symbolic definitions to blocks of data, which may be order-agnostic, language-agnostic, and standardized.

In another embodiment, a search engine for symbol delimited and symbol defined data blocks that includes formulaic search capabilities, and data generation, retrieval, de-identification, translation, sequencing, organization, form generation, visualization, tracking, filtering, scrubbing, collation, aggregation, de-identification, tagging, translation, application, mapping, cross-mapping.

In another embodiment, a data block engine for symbol delimited and symbol defined data blocks that includes formulaic search capabilities, and capabilities in data generation, retrieval, de-identification, calculation, translation, sequencing, encoding, organization, form generation, visualization, tracking, filtering, scrubbing, collation, aggregation, de-identification, tagging, translation, application, mapping, cross-mapping.

In another embodiment, a data block engine for translation of mixed coded, linguistic, and symbolic data into other coded, linguistic, and symbolic language.

In another embodiment, a data block engine for translation of coded, linguistic, and symbolic data into visualizations and maps, including but not limited to anatomic visualizations, anatomic maps, geographic maps, and timelines.

In another embodiment, a data block engine for translation of medical records.

In another embodiment, a data block engine that can construct, deconstruct, build upon, collate, aggregate, reorder, group, isolate, encode, translate, and target blocks of data.

In another embodiment, a data block engine that uses customizable blocks for secure or military applications.

In another embodiment, a system that applies artificial intelligence, machine learning, neural networks, and natural language processing to blocks of health data to automatically summarize, filter, collate, organize, calculate, encode, translate, and generate health information relevant to a desired context.

In certain embodiments, the desired context is based on medical specialty or user preferences. In certain embodiments, the desired context is based on anatomic location, anatomic region, or anatomic distribution based on coordinated or data-based relationships. In certain embodiments, the desired context is based on diagnostic information, procedural information, treatment information, or other non-anatomic health information; and optionally automatically summarizes, filters, collates, and organizes health information relevant to a desired context and procedural or treatment information.

In another embodiment, a method that applies artificial intelligence, machine learning, neural networks, and natural language processing to blocks of health data to automatically summarize, filter, collate, organize, calculate, encode, translate, and generate health information relevant to a desired context. In certain embodiments, the desired context is based on medical specialty or user preferences. In certain embodiments, the desired context is based on anatomic location, anatomic region, or anatomic distribution based on coordinated or data-based relationships. In certain embodiments, the desired context is based on diagnostic information, procedural information, treatment information, or other non-anatomic health information. In certain embodiments, the symbolic delimiters and symbolic definitions have linguistic parallels in any coded or linguistic language.

(APP05) Traditionally, documentation in healthcare can be on paper records, electronic records, or often both. Many records systems do not have a way to automatically document anatomic site names, and the ones that do may have three-dimensional models that require rotation and manipulation, or multiple screens to click through, thus creating challenges with documentation efficiency. Associating the correct diagnosis, treatment, or plan with the correct anatomic site label also requires medical knowledge and knowledge on how to navigate the electronic health record input system. Medical documentation is easier, more time-efficient, and requires less human training hours when performed on paper but having related documentation in different formats makes having a consolidated record difficult. Additionally, traditional methods of converting from one form to the other, lose valuable characteristics and features of the record type and/or require dual entry and transcription from paper to electronic records. For example, scanning a paper record into digital format does not necessarily create a digital file with the same degree of functionality and information as one originally created as a digital file. However, it is not always convenient or possible or preferred to create a digital record, particularly when treating patients. Internet connectivity issues and server issues also arise, necessitating backup paper charts. Furthermore, many workflows and industries still rely on paper documentation and markup on diagrams, with an exemplar workflow being Mohs micrographic surgery that almost universally documents Mohs maps on printed forms. Other embodiments that commonly output to printable forms include pathology requisition forms that include physical pieces of paper and labels that travel along with physical specimens removed from a patient; and pathology report forms that are commonly communicated by fax in the United States, or printed out for a patient when delivering results. By creating the system 985 capable of accurately detecting the handwritten annotations, coloring, and markup on a paper form and categorizing, aligning and converting them to digital information, an electronic record that is already correctly input from the paper form can be further augmented with photos, attachments, links, and additional electronic information.

Optical character recognition, computer vision (CV), and handwriting recognition technologies are well established. The embodiments illustrated combine and improve upon these technologies to apply detections to automatically create a digital medical record that precisely aligns detected data (e.g., from data enabled by the input device 988) to digital forms and multidimensional anatomic maps on the GUI 991 that are stored on and interacted with on the medium 995. The embodiments illustrated creates interactive health data points on digital forms that contain not only the detections, but meaningful health data such as diagnostic and procedural information that becomes interactive.

The embodiments illustrated includes a method that applies computer vision to detect annotation, coloring, and markup performed on paper forms (e.g. enabled as the input device 988) that may include diagrams, typed language, fields, codes such as QR codes, orientation markers, images, workflow initiators (such as checkboxes), form information such as a version number, and language indicators, all of which are also detected. Certain non-limiting embodiments automatically detect, categorize, align, and convert the detections to digital annotation (e.g., with the data processing module 999), coloring, markup, and coordinates. The detected annotation, coloring (including intensity, shades, and patterns), and markup includes labels, pins, areas, regions, characters, symbols, shapes, drawings, text, handwriting, codes such as QR codes. In certain embodiments, encircling, lassoing, highlighting, emboxing, drawing, pointing, encasing, and/or otherwise indicating selection, intensity, origin, destination, and/or other features can be enabled by the input device 988. The detections are all digitized and refined in alignment with coordinate normalization. Workflow initiators like checkboxes (e.g., on the physical paper output enabled by the output device 989), when checked, may trigger a digital event like a refill on a medication. An electronic record may be started or appended with information detected, aligned, and placed from the paper form, and the electronic record can then be modified or augmented with additional details. In certain embodiments, adding photographs to biopsy sites that are already mapped, labeled with correct anatomic site descriptions, and ordered with a diagnosis in place from markup on a paper map is enabled by the system 985. Certain embodiments of the system 985 can be described as “Augmented Documentation.” The digital copy of the paper form can be as simple as overlaying the detections on a digital copy, or as complicated as applying neural networks (e.g. as enabled by modules in the medium 995 such as the data processing module 999) for dropping pins, distribution segments, and health data onto multidimensional anatomic maps to automatically document anatomic locations, anatomic distributions, medical procedures, and diagnoses with automatic application of the documentation to calculate the correct code sets based on country and language. The digital documentation enabled by the medium 992 can then be modified or augmented with additional details, photos or other multimedia, and attachments linked directly to the anatomic site, diagnostic, or other record elements (e.g., enabled by the input device 988). In one embodiment, the shadow chart 6213 information containing past, present, and future information and multimedia is overlaid directly on the patient in augmented reality, mixed reality, or virtual reality using spatial computing and an augmented vision device such as glasses, contact lenses, a headset, or goggles as non-limiting examples, thus providing easily retrievable data about findings associated with the patient. Expanding on this embodiment, using a gesture to point to an anatomic location or region on a patient (or on self) can visualize a timeline of information associated with that location or region, with one example pointing to the right hip where the patient has a history of hip fracture, x-rays, scans, hip replacement, and hip physical therapy notes.

As one non-limiting example, relative position calculation and automatic relationship descriptions (e.g. enabled by the data processing module, generation module 993, and/or other components of the system 985) between two or more points by comparing variable combinations of their descriptions, individual axes and coordinate plane positions for each point, overall image or avatar or spatial axes (global coordinates) for each point in two-, three-, and four-dimensional space (over time). Through overlays, underlays, and combinations of multi-dimensional custom-coordinated, custom-axes planes, and calculations of deviations from centers and proximities to neighboring and underlying defined landmarks and data-based relationships; even points on separate diagrams or images or avatars can be compared with automatic relationship descriptions between them. In the described example, output or relationship descriptions are generated (e.g., enabled by the generation module 993) in human readable and machine-readable formats, in any coded, linguistic, or symbolic language.

It is contemplated that certain embodiments of the system 985 have practical applications in other industries as well. For example, an architect meeting with a client could annotate a blueprint with the client's requested changes and be able to later convert those notes into a digital record that could even be manipulatable in electronic form to directly edit the blueprint. Another application could be a civil engineer assessing infrastructure for deterioration and repairs in the field taking notes and marking representation maps or photographs of the site would be able to create an electronic record and later be able to update that record when repaired or to continue to track deterioration. In another embodiment, any combination of certain embodiments of the system 985, with paper or digital forms can be used to track abuse victim or accident victim injuries over time, with proper terminology, timestamping, documentation, categorization, encryption and docketing; and the outputs could communicate and interface with legal platforms such as one used by a personal injury attorney, as an example.

FIG. 5A depicts an example flowchart of the augmented document method and/or process 400 enabled by the system 985 wherein a printed form 401 and the user markup 402 (e.g., enabled by the input device 988) are converted into digital interactive forms, data, and maps 409 and ultimately augmented documentation 410 enabled by the system 985. A printed form 401 can include diagrams, typed language, fields, blank spaces, codes such as QR codes or bar codes, orientation markers, images, workflow initiators (such as checkboxes), form information such as a version number, language indicators, labels such as those associated with images, or any other contemplated information depicted in paper form. User markup 402 can include annotation, coloring (including intensity, shades, and patterns), and markup (such as labels, pins, areas, regions, characters, symbols, shapes, drawings, text, handwriting, and codes such as QR codes and bar codes placed on with a sticker, such as a patient label as one non-limiting exemplar). The system 985 takes in the user markup 402 from the printed form 401 through image capture 403 or alternately directly to a detection processor 405 as an example of the data processing module 999. In certain embodiments, using an electronic pen with coordinate detection on a specialized form to read the user markup 402 would be directly interpreted by a detection processor 405 as an example of the data processing module 999. In another embodiment, taking a digital photograph or scanning the paper form to capture the user markup 402 would be using image capture 403.

The image capture 403 (e.g., enabled by the input device 988) would be processed by computer vision 404 (e.g., enabled by the system 985). In this step, the captured image is automatically rotated, cropped, perspective warped, and rid of any artifacts (e.g., shadows on an image taken with a photo camera). Computer vision 404 also detects the form-determined information like diagrams, typed language (e.g., forms that are populated with name, date of birth, and demographic information already), filled in fields, empty fields, codes such as QR codes, orientation markers, images, workflow initiators (e.g., checkboxes), form information such as a version number, language indicators, and labels such as those associated with images (e.g., laterality labels in one form version that contains anatomic maps). Computer vision 404 of the system 985 would further detect user markup 402 and their coordinates, colors, intensities, and properties. The detections, or regions of interest (ROIs) determined in the computer vision 404 stage move on to the detection processor 405 as an example of the data processing module 999. Here the ROI information is categorized, organized, grouped, collated, and refined based on the context and language of the printed form 401 (for example, who the user is and what their preferences are; or what specialization is the form, for example dermatology versus dentistry). In the illustrated embodiment, orientation markers on the printed form 401 serve as defined axis points for the form, which allow for axis normalization 406 (e.g., enabled by the data processing module 999). This allows the system to automatically account for size variation in paper forms as well as different printable margins and zoom settings, ultimately eliminating user and printer errors in the printing process. In certain embodiments, the corners of the form may contain hash marks that not only serve as orientation markers, but also serve as coordinate definitions used for axis normalization 406. The ROI information is then processed for alignment refinement 407 where detected borders of form-printed content, such as a line art drawing, are used by the system 985 to even more precisely place the detections into the correct locations in forms and in particular on digital maps, diagrams, and images.

Once the ROI information has been normalized and refined, detection placement 408 on the digital interactive forms, maps, records, and applications 409 occurs. The ROI information is reconciled and placed into the correct spots on a digital version of the printed form (e.g., enabled by the output device 989) or record (e.g., enabled by the generation module 993 and/or database interface module 996). In certain embodiments, the digital information from the paper form may be applied to multiple digital forms and records simultaneously, thus also creating a propagation point for the generation of new paper forms, such as a Mohs surgery form for a patient needing a second surgical layer as one non-limiting exemplar. To restate, the digital information allows for propagation and auto-filled generation of new forms, or for opening the correct area of an application automatically. In certain embodiments, the digital interactive forms, maps, and applications 409 can automatically open the correct patient chart in an electronic health record, document the detections into various forms and maps within the application, and be ready for interactivity and additional augmented input from the user (e.g., enabled by the input device 988). These processes all combine into a practical application of the augmented documentation 410 workflows enabled by the system 985. In certain embodiments, a pin is automatically placed on a multi-dimensional anatomy map to represent a procedure, such as a shave biopsy, and has the correct diagnosis, procedure type, order in a list, procedure description, category, color, map placement, anatomic description, billing code, patient information, and visual preview, and the application is ready to associate clinical photographs with the pin, or the application is ready print (e.g. enabled by the output device 989) a new generated form (e.g. enabled by the generation module 993) from the digital material like a pathology requisition form. In another embodiment, a paper Mohs map used in micrographic dermatologic surgery. Multiple paper maps are used to mark areas of removed tissue for skin cancer removal and photographs are taken (e.g., enabled by the input device 988) before, during, and after the surgery. The paper Mohs maps processed in the system become a digital interactive form or map 409 capable of accepting the digital photos (e.g., enabled by the database interface module 996) and creating augmented documentation of the procedure.

FIG. 5B depicts an exemplar image capture 403 (photo) of a printed form 401 with multiple handwritten annotations as the user markup 402 as non-limiting examples of an input device 988. User markup 402 may include different colors representing shapes, characters (alone or clustered), labels, arrows, pinpoints, pin orders, shading, coloring, and other markup. Using image capture 403 or detection processor 405 of the system 985 (e.g., as non-limiting examples of the input device 988 and/or the data processing module 999), the system 985 detects the user markup 402. FIG. 5C is a representation of the detected user markup 402 from the printed form in FIG. 5B. FIG. 5D depicts the digital interactive map 409 generated after the user markup is processed through the system. The user markup has been converted to digital markup 411 with precise coordinates related to the document corners and refined by the images and placed onto a digital version of the printed form, here a digital interactive map 409.

FIG. 5E is another exemplar of an image capture 403 (photo, e.g., enabled by the input device 988) of a printed form 401 depicting an anatomic map with handwritten annotations user markup 402. In this embodiment, an English anatomic map is printed on letter sized paper. It is noted that the image capture 403 in this exemplar is at an angle and has a distorted perspective, and it is contemplated that computer vision 404 will rotate, crop, and perspective warp this image. Using computer vision 404 enabled by the system 985 conversion of the handwritten annotations to digital markup and categorization occurs. User markup 402 includes patient demographic information like the patient's name and date of birth, which was handwritten in. (These paper maps can be preprinted with patient demographics, which can also be detected in certain embodiments).

FIG. 5F shows the digital markup 411 is overlaid on the image capture 403 of the printed form 401. The digital detections are appropriately and automatically applied to the correct diagnostic, procedural, map, diagram, drawing, coordinates, and demographic input areas of the application, applying the extracted digital documentation to the session which synchronizes to other session workflows enabled by the system 985, such as automatic billing code calculation, diagnosis categorization, and more. The map contains orientation corners 413 and a QR code 412 containing map information (e.g., enabled by the input device 988), including map language which is used by computer vision as the default interpretation language unless otherwise specified by the user. Paper size and orientation corners 413 are detected (e.g., enabled by the image interface module 994 and/or the data processing module 999) for automatic map alignment, rotation, cropping, perspective warping, and detections, and other processing. Alignments are simultaneously refined further to the map version that was automatically detected.

In FIG. 5G, this information is automatically applied to anatomic visualization 10, or digital map enabled by the system 985, with automatic documentation 414 of correct procedures, diagnoses, anatomic sites, notes, patient demographics, and billing codes (e.g., enabled by the data processing module 999 and/or the generation module 993 as non-limiting examples). The documentation can now be modified, enhanced, or augmented by attaching photos, attachments, forms and other data (e.g., enabled by the input device 988) directly to the dynamic anatomic addresses, thus creating augmented documentation. It is contemplated that additional forms can be propagated and generated from the electronic record (e.g., enabled by the record retrieval module 997 and/or the generation module 993 as non-limiting examples).

Augmented documentation allows for attaching photos, attachments, forms and other data directly to the dynamic anatomic addresses. Additionally, data blocks can be changed, modified, rearranged, or added as non-limiting examples (e.g., enabled by the data processing module 999). Other workflows, such as label printing with isolated visual previews and other dynamic anatomic address information, become instantly available (e.g., enabled by the output device 989). Furthermore, the session, data, and visualizations are still translatable by the system 985 to any coded, linguistic, or symbolic language.

FIGS. 5H, 5I, and 5J depict another exemplar conversion of printed form 401 to digital information enabled by the system 985. The printed form 401 in FIG. 5H is a Chinese version of an anatomic map. Again, user markup 402 is depicted, this time depicting distributions of anatomy for different diagnoses represented by different manually shaded in colors on the paper form 401. FIG. 5I is the generated electronic record of the paper form with detected color, area, intensity, and anatomic distribution in Chinese. In this exemplar, each color represented a diagnosis, and the anatomic distribution is reported automatically along with the diagnosis (e.g., enabled by the generation module 993 and/or the knowledge base module 992). The exemplar depicts automatic conversion of detections to digital map, including appropriate selection and coloring of hierarchical anatomic site components of dynamic anatomic addresses, visualizations, diagnostic categories, and anatomic groupings. Additionally, it is contemplated that surface area calculations, intensities, and overlaps are detected and applied by the system 985 (e.g., enabled by the data processing module 999). It is further contemplated that augmented documentation allows for attaching photos, attachments, forms, and other data directly to the documented dynamic anatomic addresses. Additionally, data blocks and the diagnosis can be changed, modified, rearranged, or added.

FIG. 5J shows the automatic English translation enabled by the system 985 of the generated electronic version in FIG. 5I. It is contemplated that paper form markup can occur in one language, and that the digital information can automatically be translated and applied to an electronic record in another language.

In certain embodiments, a method of creating an improved electronic healthcare record, said method comprising: converting a paper form to digital images through an image capture device; receiving the digital images on a computing device; employing computer vision, on the computing device to analyze the digital images to detect information on the paper form; detecting healthcare record information on the paper form to convert it to digital information; detecting non-medical information on the paper form to capture context and language characteristics of the paper form; categorizing the converted healthcare record information according to the captured context and language characteristics; organizing the categorized healthcare record information according to the captured context and language characteristics; processing the organized information to refine alignment and determine precise locations for the organized information; placing the processed information on a digital interactive record representative of the paper record to create a digital version of the paper record; and displaying the digital interactive record.

In certain embodiments, the image capture device directly analyzes the digital images to detect information and places the detected information on a digital interactive record representative of the paper record. In certain embodiments, the paper form has orientation markers to serve as defined axis points for the form and allows for axis normalization. In certain embodiments, the paper form has a QR code or detectable text containing information to define the context and language characteristics of the paper form. In certain embodiments, the method further comprising augmenting the digital interactive record with additional data. In certain embodiments, the additional data is attached directly to a dynamic anatomic address and becomes instantly available to users. In certain embodiments, the detection of shapes, characters, labels, markup, symbols, and colors serve to differentiate and categorize the detections. In certain embodiments, the information is translatable to any coded, linguistic, or symbolic language.

In another embodiment, a computerized electronic healthcare record management system for improved consolidation of medical data from varying types of healthcare records, the system configured to: convert at least one paper form with markings wherein the markings represent healthcare record information through an image capture device; receive images of the paper form; interpret the received images using computer vision wherein the markings on the paper form are digitized to an electronic form; using the digitized data, create or append to a digital interactive record representative of the paper record to create or append to a digital version of the paper record; augment the digital interactive record with additional information wherein the additional information is already digital; and display the augmented record on a graphical user interface 991 wherein the resulting augmented record is a combination of records with different original formats.

In certain embodiments, the image capture device directly analyzes the digital images to detect information and places the detected information on a digital interactive record representative of the paper record. In certain embodiments, the additional information is attached directly to a dynamic anatomic address and becomes instantly available to users. In certain embodiments, the interpretation of received images is context aware. In certain embodiments, the interpretation of shapes, characters, labels, markup, symbols, and colors on the received images serve to differentiate and categorize the detections. In certain embodiments, the information is translatable to any coded, linguistic, or symbolic language.

In another embodiment, a method for generating language to describe anatomy from computer vision detected anatomy, as a vision language model.

In another embodiment, a system for generating language to describe anatomy from computer vision detected anatomy, as a vision language model.

(APP06) With electronic medical records being ubiquitous, the number of clicks, taps, searches, separate databases, and windows necessitated with the existing systems has bogged down healthcare with inefficient practices. The complexity of information needing to be collated continues to expand; and often the user has to keep open a separate window/application for notes, or keep paper handwritten notes as they sift through the records trying to piece together what happened in the past, what the past studies showed, why the patient is present for a visit and what needs to be addressed at the visit, and what is already scheduled to be addressed at future appointments. Then, to update that information, separate windows and applications must be updated manually, with no single way to update information across systems. The manual collation, presentation, and updating of information takes various trained staff people to perform these processes correctly, and it takes significant human capital to perform these tasks. Training gaps, education gaps, and inconsistencies in workflows also compound to increase human error and omission in performing the current systems' manual tasks of collating and updating to the patient record. The embodiments of the system 985 illustrated include a practical application to reduce the time spent by medical assistants, scribes, and healthcare workers on manual collation and updates of healthcare data by automatically collating, displaying, and allowing interaction with past, present, and future health information in different applications, systems, and formats. Manual collation and addition of data can also occur in a unified shadow chart method and/or process enabled by the system 985 that blends paper and digital data. In one embodiment, a coordinated language model 980 engine is formed by any combination of language models, vision-language models, language-vision models, visualizations, maps, avatars, multimedia, code sets, cross-maps, language, semantics, translation, user inputs, and detections. As one non-limiting example, in the United States, physicians may look to hiring virtual scribes from overseas or AI-assisted scribing due to local staffing shortages; but the virtual scribes are unable to accurately, precisely, and reproducibly label anatomy as reliably and quickly as a person in the room examining a patient with a paper representation of anatomy in front of them. And other AI-scribes are currently limited to listening to human healthcare conversations and documenting text descriptions of the verbal inputs without accurate generation of visual representations of anatomy. Listening to human conversations without generating accurate and/or modifiable and/or targetable visualizations is not significantly better than free-text descriptions that have been traditionally used in healthcare documentation. The paper representation of anatomy and health data on the shadow chart paper is available when the internet is down or offline. With augmented documentation workflows exemplified herein, the paper representations of anatomy also enable asynchronous paper and electronic documentation and enhanced efficiency in both documentation workflow.

The embodiments illustrated of the system 985 use health data in digital shadow charts, paper shadow charts, or both to create a comprehensive record and to seamlessly blend paper and electronic documentation. The exemplar embodied shadow charts automatically collate past, current, and future healthcare data that are automatically linked to anatomic maps and healthcare metadata for visualization, modification, augmentation, and automation of healthcare record generation, and retrieval. Shadow charts (e.g., enabled by the generation module 993, record retrieval module 997, and/or the output device 989 in certain non-limiting embodiments) collate and display information from dynamic anatomic addresses, data blocks and metadata blocks on an anatomic map translated to user language, with recreated anatomic sites and health data. Automated workflows such as prescription refills, suggested diagnostic tests, flag areas of concern for follow up, etc. are included on the shadow chart. Digital markup and workflow initiation on a digital shadow chart is done in real time (e.g., enabled by the input device 988), and physical markup on a paper shadow chart may be done in real time with certain devices like a pen that tracks position on paper or after the fact with a photographic or scanned capture of the marked-up paper shadow chart. Computer vision enabled by the system 985 can interpret the markups and account for any changes and the content can then be synchronized in the record in the plurality of appropriate locations, databases, and systems for the data. The user can be alerted to any discrepancies upon after-the-fact synchronization, and is automatically prompted to reconcile them. To prevent clutter in the shadow chart (e.g., enabled by the GUI 991), points of interest can be retired, selectively turned off, filtered, or selectively served based on manual interaction (e.g., enabled by the input device 988), user preferences or automatically using user or organizational settings.

The shadow chart can have features from past historical visits, deferred diagnosis or treatments or issues needing follow-up or more information, or other issues related to the patient. Additionally, it can have information about past encounters (like history, medication list, prior prescriptions, prior medical or cosmetic treatments or recommendations, prior purchases), about the current encounter (like name, date of birth, medical record number, date of service, appointment demographics, patient demographics, doctor/hospital demographics, insurance demographics, financial info), future encounters (like future appointments already scheduled, collated from a variety of databases and information systems, e.g. enabled by the record retrieval module 997) or suggestions for future appointments, treatments, products or services (e.g. enabled by the record retrieval module 997 and/or other components of the system 985). The examples included in the aforementioned sentence are a non-limiting list, and it is contemplated that one skilled in the art would know other examples.

The shadow chart can contain anatomic maps and images representing relevant past, current, and future information from the patient records which can be printed on paper in the user's preferred color scheme, including but not limited to grayscale, black and white, bluescale, color, a defined color palette for color coding, in a combination of monochrome and color, or in any other contemplated color scheme as one skilled in the art would know. Historical information (e.g., diagnoses, treatments, follow-ups needed) that are associated with an anatomic site will be automatically made visible on an anatomic map representing their locations (e.g., enabled by the GUI 991).

The anatomic maps on the shadow chart can be derived from anatomic site names and coordinates in two- or three dimensions, or in four-dimensions by showing changes over time (e.g., enabled by the data processing module 999, the generation module 993, the record retrieval module 997, and/or other components enabled by the tangible medium 995 and/or the system 985). These can then be presented to the user in any dimension, as three-dimensional maps with the appropriate views or two-dimensional maps that can be printed containing the relevant information, or can be interacted with in a digital shadow chart (like rotating a three-dimensional model representing a time point or blended time points, e.g., enabled by the input device 988).

The shadow chart can automatically place different historical contexts into different sequences. In one example, a skin cancer history of past treated skin cancers could be shown in sequence 1, 2, 3 . . . while a list of skin cancers that are deferred and still needing treatment could be shown in 01, 02, 03 . . . order, while a list of prior biopsies that the user needs to deliver results on can be generated and presented on the GUI 991 as a, b, c . . . , while the list of new ordered procedures created for that encounter can be delivered as A, B, C; while still other items can be sequenced based on dates, order of reports, order of entry, or other user sequence preferences that may be desired as enabled by the system 985.

Different shapes can have different meanings on the shadow chart and marking on the shape could digitally load standardized templates (e.g., enabled by the knowledge base module 992) and documentation for the patient. For example, a square could be a checkbox associated with an anatomic site with the results listed next to it, indicating that when the user places a checkmark in the box, the system automatically documents that they provided the patient their results, answered their questions, and finished with that task.

It is contemplated that a legend for each generated sequence (e.g., enabled by the generation module 993) could provide more information available for both the digital and paper shadow chart. The digital shadow chart could pull up and display additional information, such as past photos, reports, and other multimedia associated with the charted items on a shadow chart (e.g., enabled by the record retrieval module 997 in communication with the GUI 991). Based on user preference or selection, the additional information associated with the shadow chart could be easily retrieved for viewing or printing (e.g., enabled by the output device 989). A paper shadow chart that contains mapped and charted items can also be modified electronically, before, during, or after an encounter with a patient; or before, during, or after printing as one practical application example.

A shadow chart can have various protocols loaded for certain commonly performed tasks/procedures. For example, for an annual physical, the map may be pre-populated with pins to locations where a part of the examination is to occur. Each pin can be customized to accept the data relevant for the particular examination task, or in a paper version, provide an area to accept the relevant data. Protocols and/or the data within can be toggled on/off (e.g., enabled by the input device 988 in communication with the GUI 991) to be visible on the digital shadow chart.

A paper shadow chart, aimed at consolidating and collating context-aware information about the visit, can be marked up in a different color (e.g., with the input device 988). For example, on a black and white printout of the shadow chart that contains a grayscale anatomic map, a user can create a markup in blue pen, erasable red pen, colored pencil, marker, colored stamps, or other forms of markup as one skilled in the art would know. The color or stroke-width differential or handwriting detection from the markup can be used to detect changes and additions to the shadow chart. A digital photo or scan of the shadow chart (e.g., enabled by the input device 988) automatically can generate a digital version with the updated, added, or deleted information (e.g., enabled by the generation module 993). The information can further be augmented with additional material such as with live capture of photo, multimedia, or attachments; association of prior photos, multimedia, or attachments; and/or addition/modification/deletion/moving/re-categorizing of information) (e.g., Enabled by the input device 988). Those changes and additions can then be applied across the various data systems (electronic health record, scheduling system, practice management system, billing system) enabled by the system 985.

The shadow chart enabled by the system 985 can filter records to show relevant past diagnoses and treatments. As one non-limiting example, the shadow chart enabled by the system 985 can pull all instances of past precancerous actinic keratoses that were documented as treated and show them on an anatomic map with dates of treatment, photos or multimedia, path reports showing it was a biopsy proven at one point in time, and other clinical data. The shadow chart can visually help determine if a lesion that is concerning for squamous cell carcinoma is a new lesion, or if it has progressed from a prior pre-cancerous lesion, as one non-limiting practical application of the system 985. This is just one of a plurality of practical application examples relevant to diagnoses and treatments, and it is contemplated that filtering can be applied to other health data as one skilled in the art would know.

In certain embodiments, the user could also choose to permanently alert the user of an important history with options like “Always show on shadow charts” (e.g., enabled by the input device 988 and/or the GUI 991). Conversely, if a matter is resolved permanently the user could omit the history from future shadow charts “Resolved-hide from future shadow charts.”

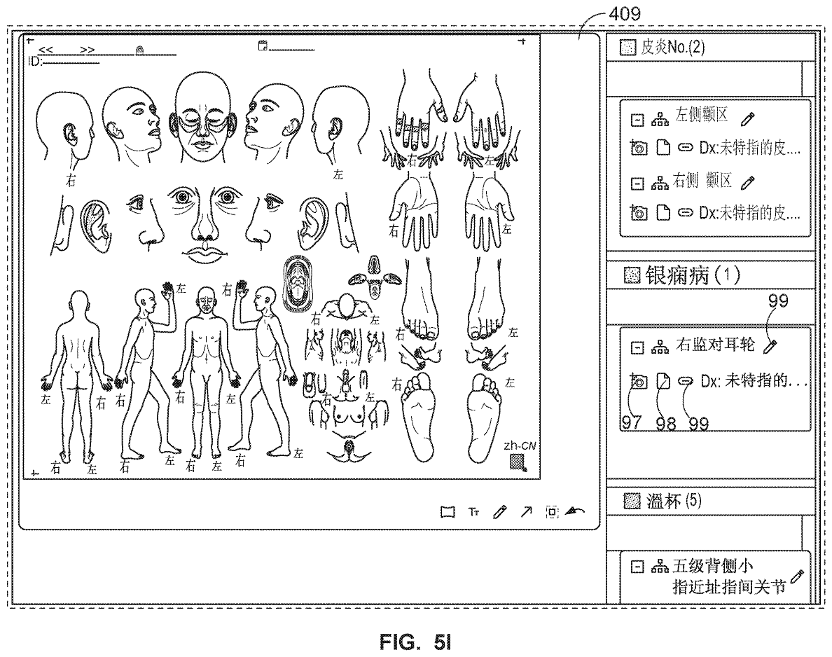

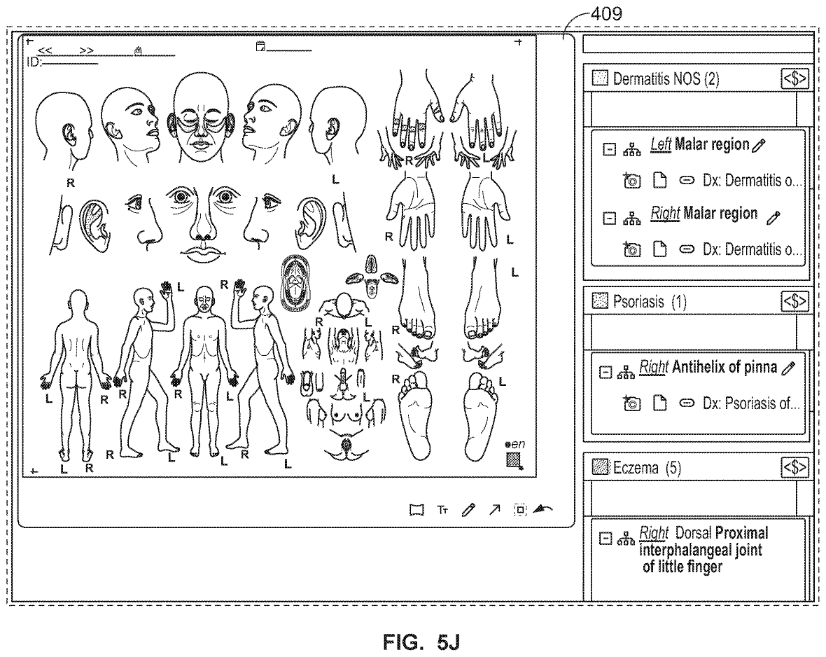

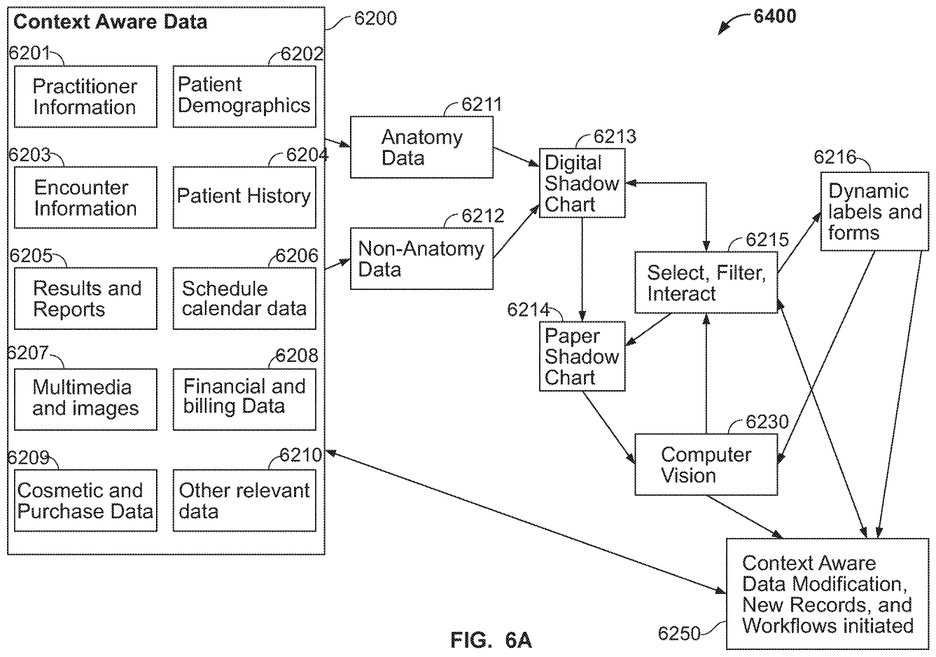

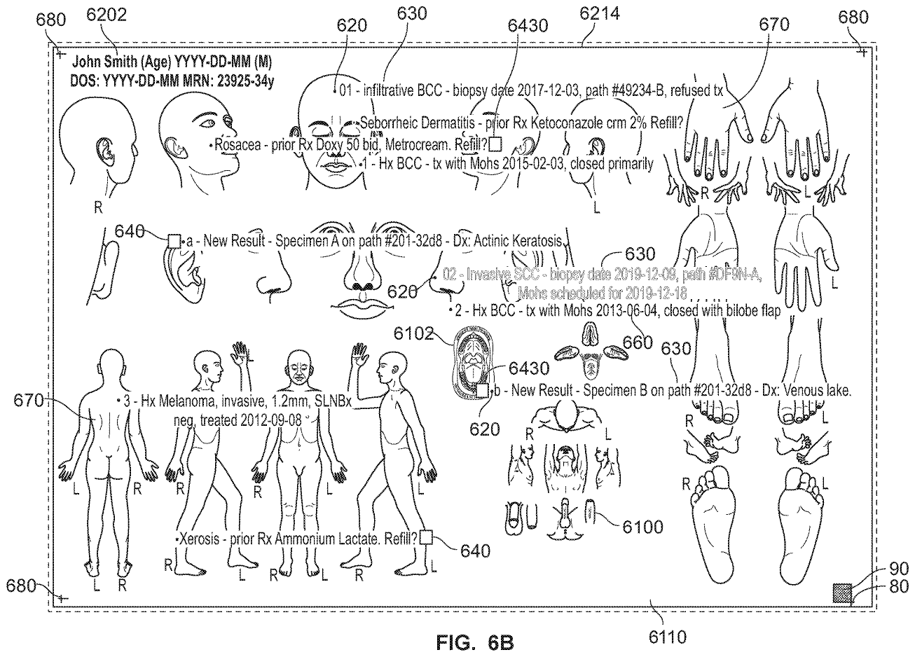

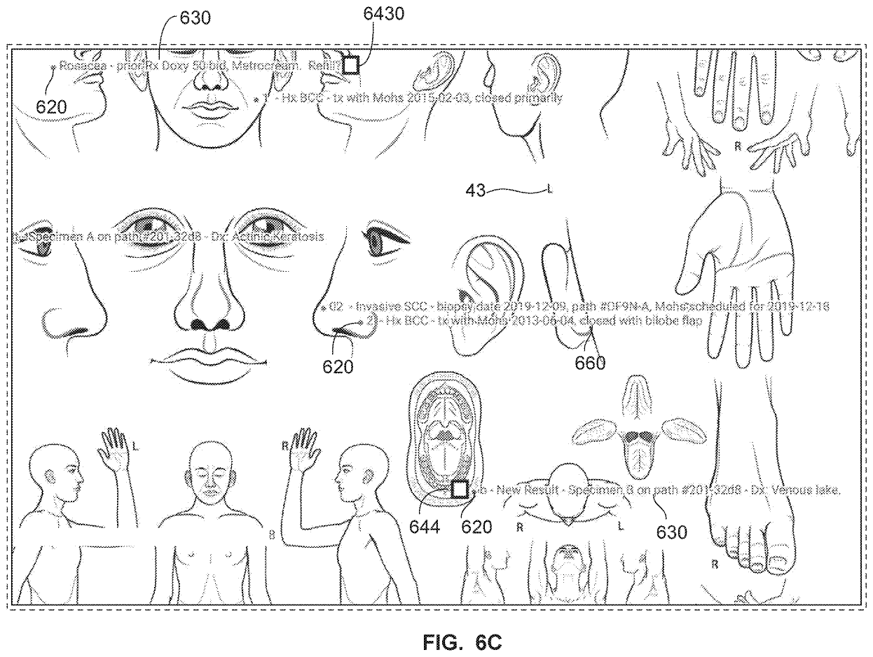

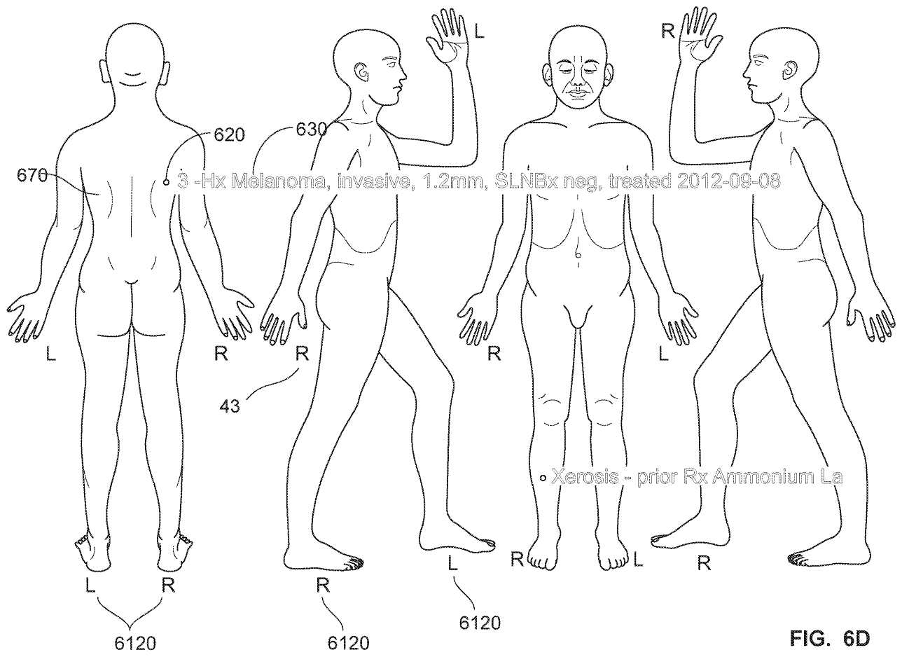

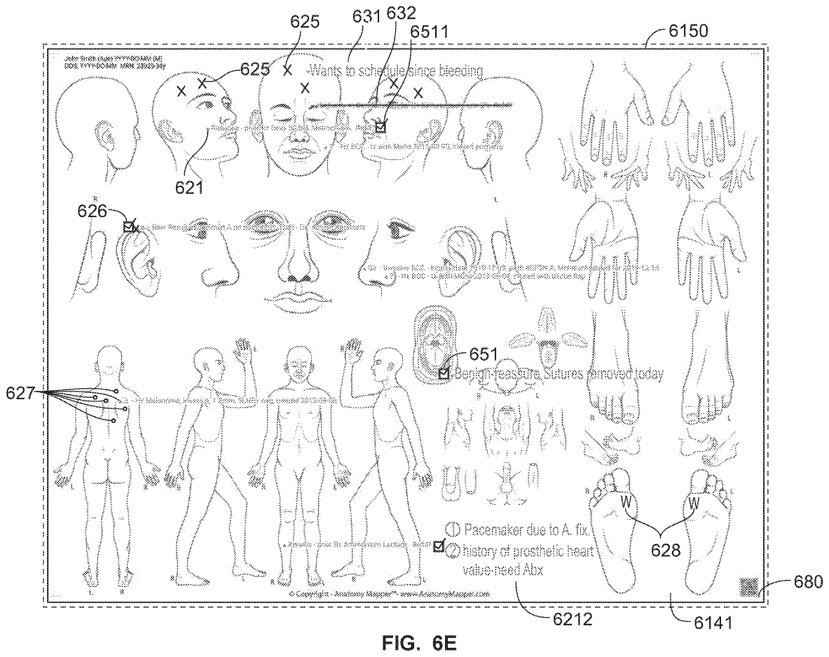

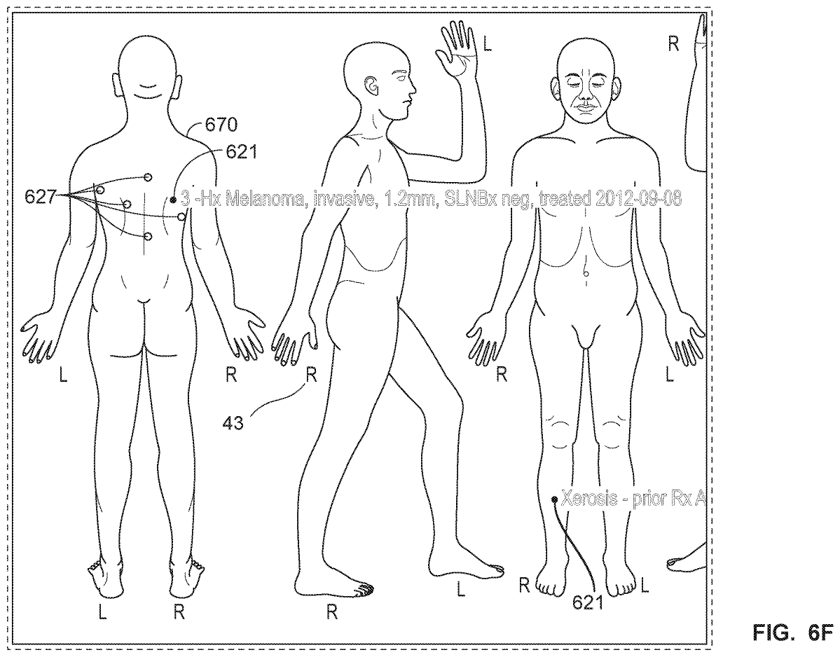

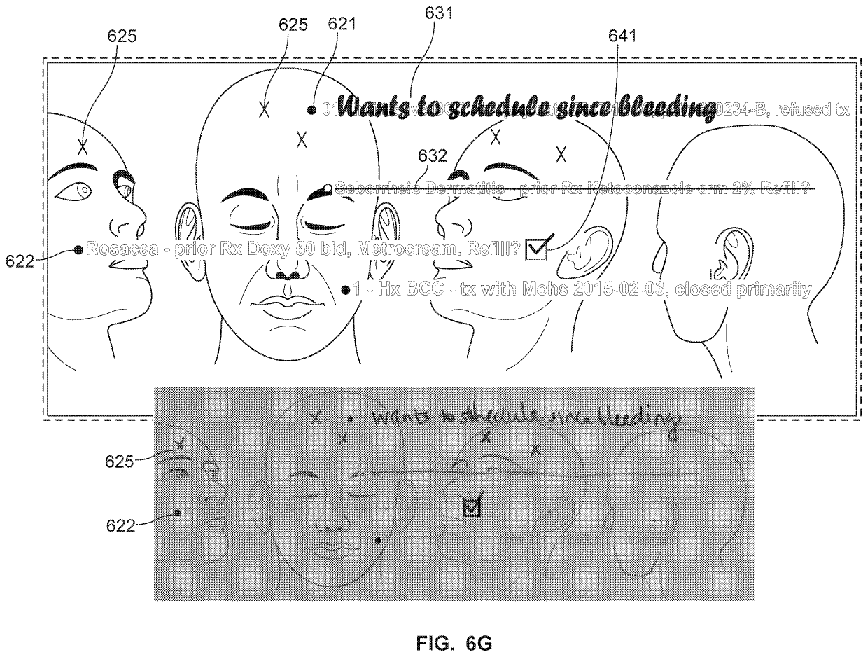

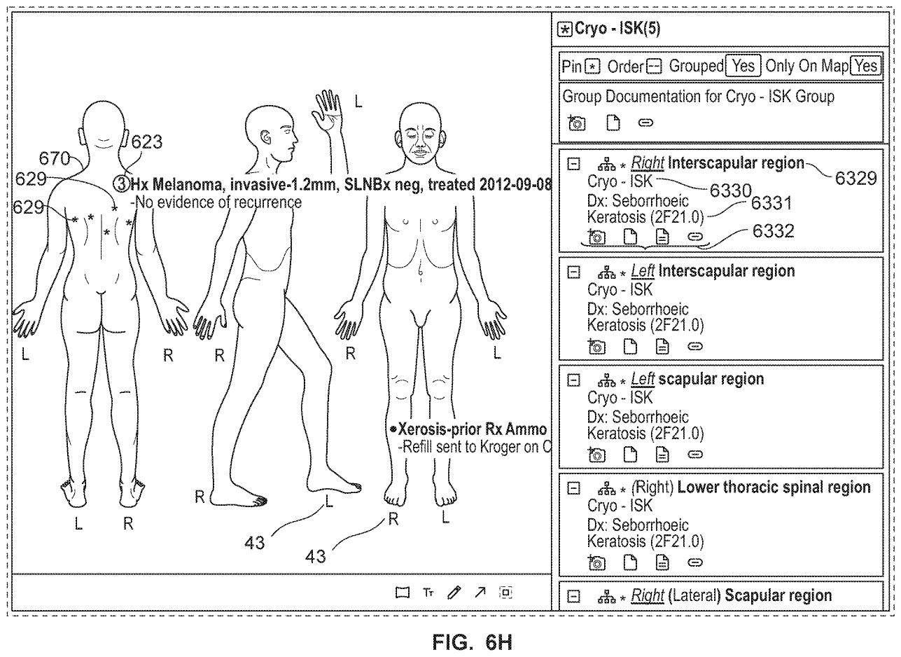

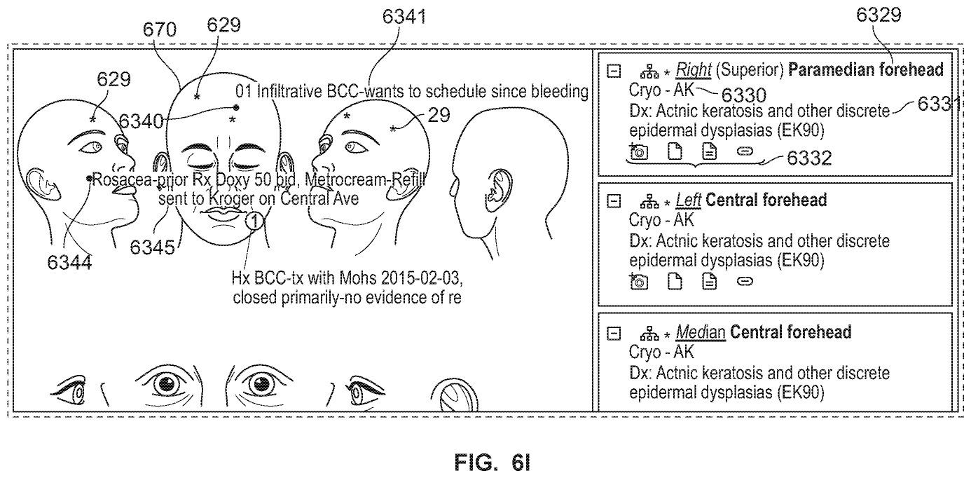

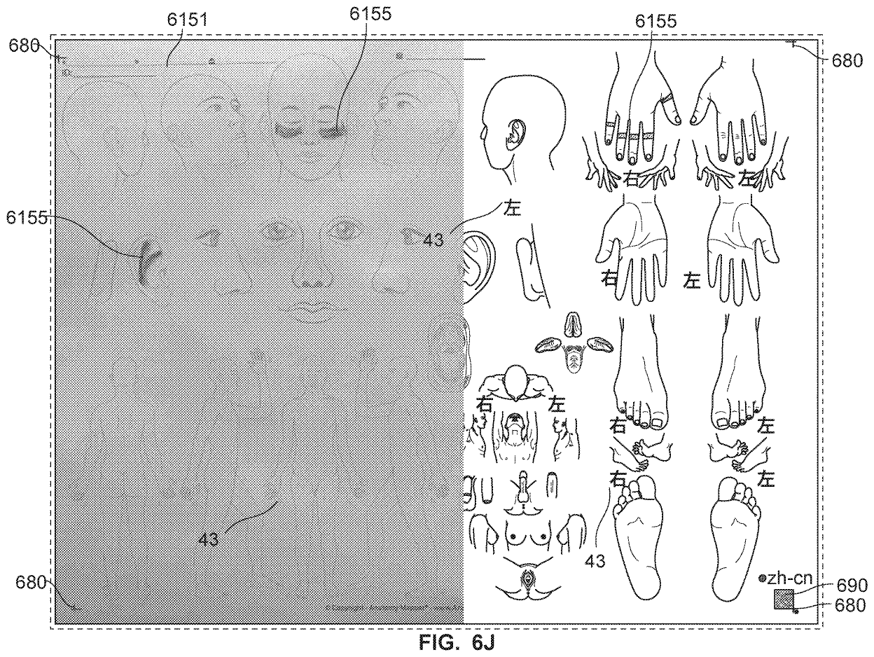

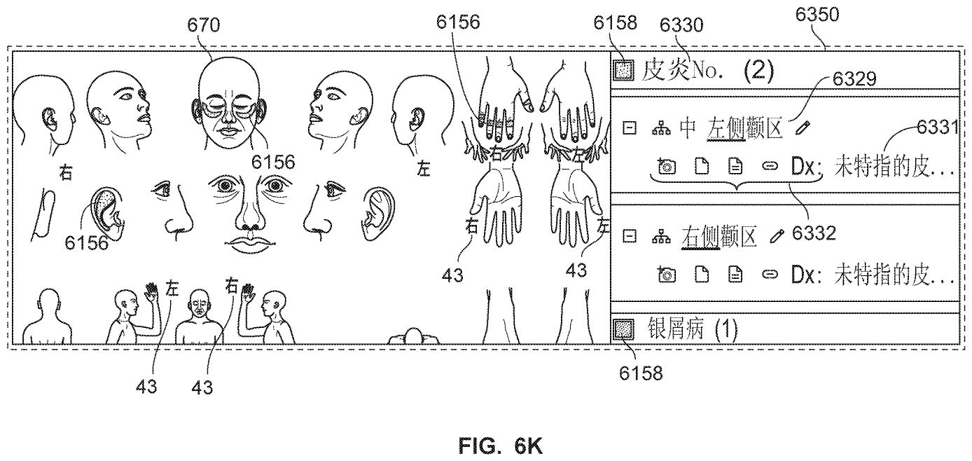

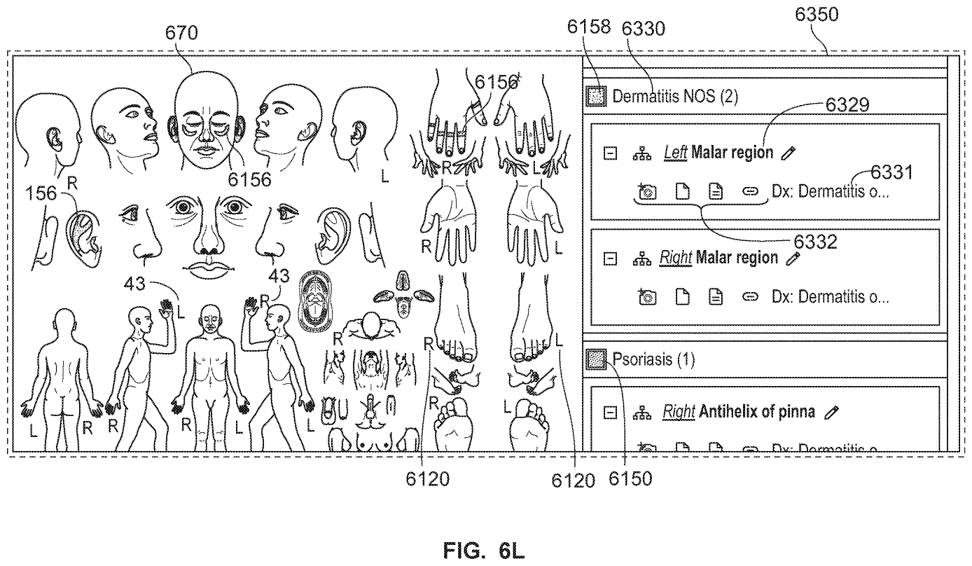

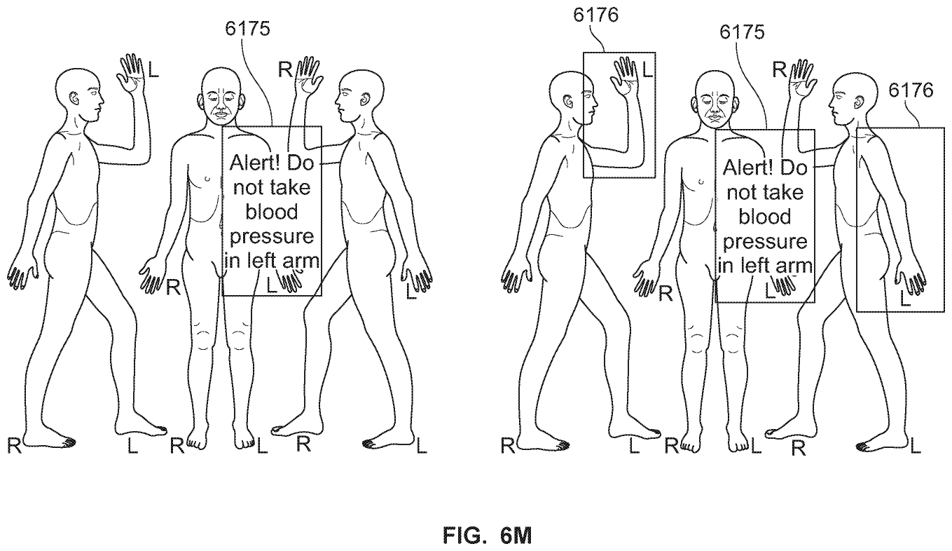

FIG. 6A is a flowchart illustrating the information management in a method and/or process 6400 enabled by the system 985, specifically how digital and paper shadow charts interact with past, present, and future data to create a comprehensive record as a practical application. Context aware data 6200 a user wishes to incorporate into an electronic medical record or into shadow charts includes any data relevant to the medical record and can be anatomy data 6211 and/or non-anatomy data 6212. The anatomy data 6211 and non-anatomy data 6212 are placed into appropriate places into a digital shadow chart 6213. The digital shadow chart 6213 can then be directly printed into a paper shadow chart 6214 (e.g., enabled by the generation module 993 in communication with the output device 988) or modified in electronic form with different selections, filters, and interaction modifiers 6215. Examples of filters could include, but are not limited to, diagnoses and history relevant to the practitioner, specialty, procedures, morphologies, symptoms, treatment recommendations, or anatomic sites of interest (e.g., enabled by modules on the medium 995, such as the data processing module 999, in communication with the processor 986). Once modified an updated paper shadow chart 6214 may be printed (e.g., enabled by the generation module 993 in communication with the output device 989). Alternatively, other dynamically created paper labels and forms 6216 could be created and printed. Paper shadow charts 6214 and dynamic forms and labels 6216 may be scanned or captured with a camera through image capture and processed through computer vision 6220. Paper shadow charts 6214 may contain anatomy visualizations and areas of non-anatomy data that can be marked up, filled in, labeled, annotated, colored, and/or drawn on. The image capture (e.g. enabled by the input device 988) of the paper shadow chart 6214 processed through computer vision 6220 can remove artifacts such as physical shadows or pixilation or blurs, detect orientation and alignment, detect anatomic sites and their descriptions and relationships, normalize axes, and detect, categorize, and place the compiled data 6250 (e.g. processed health data, enabled by the system 985) into the correct digital locations in the electronic record. The compiled data 6250 may be context aware data modification, new records, and/or initiated workflows.

In certain embodiments, a paper shadow chart 6214 (e.g., enabled by the output device 989, later serving as the input device 988) could be a pathology requisition form that contains a list of biopsy sites with anatomic site descriptions and isolated visual previews of the anatomic sites that also includes physical label printing which can be placed onto physical biopsy specimens. Such labels may contain anatomy information, patient information, and anatomy visualizations and may recreate a generated digital anatomic map and points.

In another embodiment, a template could be a paper Mohs map (e.g., enabled by the output device 989, later enabled by the input device 988 as one non-limiting example) that contains anatomy data, anatomy visualizations, non-anatomy data, and blanks to be filled in. After image capture, the chart is processed and categorized with computer vision 6220 and the compiled data 6250 is placed into the electronic record which is now modified based on the context aware data.

The electronic record components can then be further modified through selection, filters, and interaction modifiers 6215 (e.g., enabled by the input device 988). This process creates “augmented documentation” workflows enabled by the system 985. In certain embodiments, a paper shadow chart 6214 is scanned and processed with computer vision 6220 and digital photographs are linked to the anatomic sites documented on the paper shadow chart 6214 thereby augmenting the electronic record and the compiled data 6250 is modified based on the context aware data.

In another embodiment, a shadow chart is processed with computer vision 220 by the system 985 which detects the chart language as English, the patient sex as male, the documented patient encounter date, and the practice specialty as dermatology. These detections created the compiled data 6250 which updates the electronic record with the context aware data modification. In yet another non-limiting embodiment of data modification, a dermatology diagnosis is resolved and the compiled data 6250 triggers the removal of the diagnosis from the dermatology record modifying the record in light of the new context. In yet another embodiment, a paper shadow chart 6214 is scanned and processed with computer vision 6220 (e.g., enabled by the system 985) which detects a new patient encounter, an encounter date, and annotations and markups on specific anatomic locations and the compiled data 6250 generates a new record from the detections. In yet another embodiment, a checkbox is checked on the paper shadow chart 6214 indicating a medication refill is necessary. The paper shadow chart 6214 is scanned (e.g., enabled by the input device 988) and computer vision 6220 detects the checked box and initiates the workflow 250 to automatically refill the medication and document the refill on the electronic record. It is further contemplated that the compiled data 6250 can also modify or be modified by the data in the context aware data 6200 (e.g., enabled by the data processing module 999).

It is contemplated that context aware data 6200 in communication with the medium 995 and/or the system 985 may be comprised of: (i) practitioner information 6201; (ii) patient demographics 6202; (iii) encounter information 6203; (iv) patient history 6204; (v) results and reports 6205; (vi) schedule calendar data 6206; (vii) multimedia and images 6207; (viii) financial and billing data 6208; (ix) cosmetic and purchase data 6209; and (x) other relevant data 6210 not described elsewhere as non-limiting examples, as one skilled in the art would know. Practitioner information 6201 examples include clinic name, clinic location, practice specialty, physician or healthcare provider data, practice type such as surgery or medical, and other information as one skilled in the art would know. This allows for granular data filtering by the system 985. In certain embodiments, a physician can filter to only see (e.g., enabled by the GUI 991) shadow charts with content relevant to their specialty or only records generated by them.

Patient demographics 6202 includes but is not limited to patient name, nickname, date of birth, patient address, patient identifiers like medical record number and/or social security number and/or license number, married name, emergency contacts, e-mail address, patient portal information, power of attorney, acceptance of policies, credit card on file status, insurance information, patient specific notes, and other information as one skilled in the art would know.

Encounter info 6203 contains information about the reason for appointment, what was or is being or will be addressed at an appointment, who referred the patient for this encounter, encounter date, encounter time, encounter location (place of service), encounter location type (outpatient, inpatient, telemedicine), and other information as one skilled in the art would know. Further encounter information 203 may be for past, present, or future encounters.

Patient history 6204 includes but is not limited to past diagnoses, past symptoms, past medications, active medications, past treatments, allergies, family history, past surgeries, anatomic location associated with any of the history data components, and other information as one skilled in the art would know.

Results and reports 6205 includes but is not limited to pathology reports, blood tests, imaging reports, and statuses about the reports. For example, if a patient has been counseled on the result or if treatment has been performed or needs to be performed. Reports may also be related to imaging studies like radiographic studies including x-rays, CT scans, MRIs, ultrasounds, PET scans, and their variants.

Schedule calendar data 6206 includes but is not limited to information about past, present, and future appointments, results, reports, progress notes, treatment summaries, images, and other data. It is contemplated that temporal data can be specific to date, time, and time zone or a combination of these, and that the temporal data can be arranged into a plurality of timeline views.

Multimedia and images 6207 include, but is not limited to, photos, images, videos, diagrams, and other multimedia that contain anatomic sites. The sites can be labeled as part of the multimedia or image metadata, or detected from the images and multimedia. The multimedia and images 6207 may also be from image related studies like radiographic studies including x-rays, CT scans, MRIs, ultrasounds, PET scans, and their variants. Additionally, it is contemplated that the shadow chart can directly link to the view of interest, such as a tumor in a CT scan at the correct zoom level and slice level.

Financial and billing data 6208 includes but is not limited to billing codes relevant to a particular country or region, insurance information, eligibility checks, deductibles, coinsurance, patient balances, copay status and amount, credit history, credit card on file status, and other financial and billing data relevant to the delivery and payment for health care that one skilled in the art would know.

Cosmetic and purchase data 6209 includes information about cosmetic or self-pay treatments such as laser surgery, cosmetic surgery, skin tag removal, retail purchases such as skin care, filler, botulinum toxin, and other injectable cosmetic treatment mapping, and other data that one skilled in the art would know. It is contemplated that treatment recommendations may include visual and descriptive regimen maps that explain “what products to use where” on the body with color coding on different anatomic distributions and may be for cosmetics and/or prescription, over-the-counter, and non-topical recommendations. In certain embodiments, cosmetic and purchase data 6209 for different settings for laser treatment could be mapped on a shadow chart. It is further contemplated that the information can be recreated or modified within the shadow chart. It is further contemplated that automatic treatment recommendations based on timing can also be applied, for example that a patient's botulinum toxin treatment should have worn off by now and the patient is due for another treatment, and the number of units used in the last treatment can be shown on the shadow chart. A plurality of templates related to cosmetic treatment and follow up is possible as one skilled in the art would know.

Anatomy data 6211 includes but is not limited to visualizations, diagrams, dynamic anatomic addresses that are trackable multidimensional locations through space (spatially relative to two-dimensional or three-dimensional anatomy) and time (fourth-dimensional anatomy), coordinates, site descriptions, hierarchical anatomy relationships, coordinated anatomy elements, uncoordinated anatomy elements, anatomy modifiers, coded descriptions of anatomy, linguistic descriptions of anatomy, symbolic descriptions of anatomy, mixed descriptions of anatomy, custom descriptions of anatomy, relational data between different anatomic sites, photographs of anatomy, maps of anatomy, localized or migrating findings (such as symptoms or morphology), anatomy specific recommendations (such as what topical treatments to use where on body), imaging containing anatomy, reports containing anatomy, notes containing anatomy, multimedia containing anatomy, and other anatomy data as one skilled in the art would know. Anatomy data 6211 can be used to create collation points for records from various sources and types on a shadow chart. In certain embodiments, a “broken bone emoji” could be placed on the location of a fracture, and x-rays and imaging and their reports from different time points would automatically collate into that emoji in a digital shadow chart 6213. Anatomy data 6211 is also data that is associated with anatomic sites, anatomic distributions, anatomic distribution intensity, visualizations, points on maps, photographs and imaging of anatomic sites, reports and records linked to anatomic sites, prescriptions and recommendations linked to anatomic sites, appointments and/or plans linked to anatomic sites, dynamic anatomy addresses that track anatomic sites, and other anatomy data. An anatomic site might be a “right knee” in a patient with a history of an artificial knee replacement as one non-limiting example. An anatomic distribution example enabled by the system 985 is the “face, back, and chest” for a diagnosis of acne with the acne intensity being worst on the face followed by the back followed by mild involvement on the chest (face>back>>chest).

Non-anatomy data 6212 includes but is not limited to patient demographic information, non-localized findings, diagnoses, and conditions such as hypertension or fatigue, encounter and schedule demographic information, billing and financial information, non-anatomic information associated with records containing anatomy data, blood tests and other non-anatomy data. It is contemplated that blood tests can also be part of anatomy data when dealing with organ systems and functional systems, such as the hematopoietic system, and when describing the anatomy of blood cells under the microscope such as macroblastic or microblastic features of red blood cells. It is further contemplated that microscopic and dermatoscopic anatomy and morphology can also belong to both anatomy data and non-anatomy data. Additional non-anatomy data would also fall into this category as one skilled in the art would know.

It is contemplated that the examples provided to describe this figure are some of a vast plurality of templates for data, shadow charts, and interactions, as one skilled in the art would know.

FIG. 6B is an exemplar shadow chart 6213 enabled by the system 985 that contains past, present, and future information. In this embodiment, the shadow chart shows shadow pin descriptions 630 that are context aware diagnoses and data in light gray text (e.g., enabled by the record retrieval module 997). Representative demographic information 610, including patient and encounter demographic information such as the patient name, date of birth, medical record number, and date of service, is shown on top left of the paper shadow chart 6214. There are shadow pins 620 for visualizations of skin cancers that still require treatment located at their anatomic locations. In this embodiment the sequence begins at “01” and the shadow pin descriptions 630 indicate the diagnosis, the visualized anatomic location, and future information regarding the future appointment for Mohs surgery. Each shadow pin 620 is on a specific anatomic site, and has a description 630 of past, present, and future information associated with it. In this embodiment, the shadow chart 213 shows shadow pin descriptions 630 that are context aware diagnoses and data in light gray text. The shadow pin description 630 for the first visualization in the sequence reads “01-Infiltrative BCC-biopsy date 2017 Dec. 3, path #49234-B, refused tx” which tells the reader the following clinically relevant information about this shadow pin: (i) that it is in the list of skin cancers still needing treatment, and its position in the list based on the “01” designation, (ii) a visualization of the anatomic location based on the shadow pin 620 position, (iii) a diagnosis “Infiltrative BCC,” (iv) the procedure date for the original biopsy “biopsy date 2017 Dec. 3,” (v) the pathology result number “49234” and order on the pathology report “B”, and (vi) that the patient has not had it treated and is refusing treatment “refused tx.” It is contemplated that a reproducible and trackable anatomic site description accompanies each shadow pin 620, and that relevant information can be displayed from a plurality of databases and from a plurality of display types such as different pin types, different shadow pin description types, and other customizable information displays. It is further contemplated that colors, including differential colors (such as pin fill or pin drop being a different color than a pin label, or two different distributions painted that are different colors), can help to organize visualizations and inputs and categories of both shadow and new documentation (e.g., enabled by the system 985 and components such as the input device 988). It is also contemplated that shadow pins that are light in color, such as on paper, can be filled in with a darker color to indicate to add the documentation to the present electronic chart, and that such addition will happen automatically with computer vision detection. Shadow pins 620 that are selected on a digital shadow cart can also be edited, updated, moved, merged, hidden, deleted, converted, or expanded. Thus, it is contemplated that a digital shadow chart 6213 has significantly more information readily available by tapping or hovering over the shadow pin 620 or pin label 630 to for example pull up a timeline of information associated with the anatomic site or pin, or with other user interface interactions that one skilled in the art would know.

FIG. 6C shows a magnified portion of the shadow chart 6213 of FIG. 6B. A shadow pin description 630 for the second visualization in the “01, 02, 03” sequence reads “02-Invasive SCC-biopsy date 2019 Dec. 9, path #DF9N-A, Mohs scheduled for 2019 Dec. 18.” From this context aware shadow pin 620 and pin description 630 we know the anatomic location in a visualization, the diagnosis and details relative to the diagnosis like the biopsy date, the pathology report information, as well as future information about a scheduled appointment. It is contemplated that one skilled in the art would recognize that this represents data about the future, specifically appointment data and data on what is to be treated in the future, and that other future data could be added on a shadow chart as well.

FIGS. 6B and 6C show workflow initiators 640 depicted at the end of shadow pin descriptions 630 as well as directly next to shadow pins 620. Workflow initiators 640 relevant to a diagnosis can be used on both paper and digital shadow charts in certain embodiments. On certain embodiments of paper shadow charts, the workflow is initiated after computer vision detection of a check in the box. On digital shadow charts, the workflow is initiated upon checking the digital checkbox. In the embodiment depicted in FIG. 6C, a workflow initiated by a workflow initiator 6430 is a new documentation update for a diagnosis that includes a list of medications associated with the diagnosis where a refill of a medication is automatically sent or queued to the patient's preferred pharmacy, and all of the documentation related to that workflow is automatically documented in the electronic health record in the relevant sections (diagnosis, progress note, refills, medication lists, etc.). Also depicted in FIG. 6C, the workflow initiator 644 that appears directly next to or otherwise related to the shadow pin (instead of after the description like workflow initiator) provides visual differentiation and indicates that they are different workflows. In certain embodiments, checking the workflow initiator 644 (e.g. enabled by the input device 988) that appears directly next to the shadow pin could document that results for a test were discussed with the patient, and the diagnosis, treatment plan, counseling templates and other templates could be automatically input into the patient's electronic health record and associated with the reproducible anatomic location as determined by the shadow pin 620.

It is contemplated that shadow pins 620 may have different sequences and labeling types enabled by the system 985 to indicate different list memberships. It is further contemplated that color grouping, pin type changes, and order type changes can help further visually differentiate shadow information (e.g., A, B, C is different than 1, 2, 3 is different than 01, 02, 03 is different than a, b, c is different than i, ii, iii). As shown in FIG. 3, a shadow pin description for a shadow pin with a different sequence 660 reads as “2-Hx BCC-tx with Mohs 2013 Jun. 4, closed with bilobe flap.” The list sequence type, shown here as “2” in a “1, 2, 3” type sequence, is different from the previously discussed “01, 02” sequence, and indicates historical past treatment information in this non-limiting exemplar.

It is contemplated that a shadow chart, whether paper or digital, can also accept new inputs, for example, on an unmarked diagram and anatomic map 670 as well as on diagrams and maps that already contain shadow information (e.g., enabled by the input device 988). New inputs could be writing or coloring annotations and markup on paper, or digitally placed annotations and markup on a digital shadow chart.

Orientation and axis normalization markers 680 are automatically included on printed shadow charts 6214 and included with any diagram or map or form field. These normalization markers 680 allow forms and diagrams to be printed in any size and on any size of paper (e.g. enabled by the output device 989) and that computer vision enabled by the system 985 can properly detect and place or update detections into the correct digital entry points on forms, on maps (on “land” in a geographic context or corollary) and on whitespace between diagrams when there are multiple (in the “sea” in a geographic context or corollary).

Each shadow form chart can have a coded indicator 690 about the form, such as a QR code, that provides details of the form version, form language, form context, user preferences (e.g., how to interpret the markup and annotation), map and diagram properties, digital fields the form maps to, patient and demographic information, and other information that one skilled in the art would know.

It is contemplated that a shadow chart enabled by the system 985 can be automatically modified based on patient characteristics. In the non-limiting example, male anatomy 6100 is shown (e.g., enabled by the GUI 991), but female anatomy has been filtered out to create whitespace 610 on the shadow chart. It is contemplated that the whitespace 610 could allow for non-anatomy information to be placed on a paper shadow chart. It is further contemplated that the whitespace 610 can be available on a digital shadow chart when consistent workflows and visualizations are desirable. It is further contemplated that the whitespace 610 and that illustrated patient characteristics assist the computer vision in determining and verifying the patient characteristics, such as patient sex or whether diagnoses or findings related to oral anatomy are included in the form context. In the present exemplar, the oral anatomy 6102 patient characteristic has been included in the shadow chart with an automatically included workflow initiator 6430 and shadow pin 620 and context aware pin description 630.

It is contemplated that shadow charts enabled by the system 985 can have meaningful laterality abbreviations and symbols, depicted here in FIGS. 6B and 6D. Context, language, and perspective aware laterality labels 43 are shown with the “R” indicating the right side of the patient in an outside observer perspective, and the “L” indicating the left side of a patient in an outside observer perspective. Such laterality markers 43 in the illustrated embodiment show the diagrams from an outside observer perspective. It is contemplated that the lateralities and modifiers (such as those describing directional modifiers like superior, lateral, medial, inferior) of anatomy can be selectively reversed, reflected, or rotated into a mirror view 426 or “selfie view” enabled by the system 985 to accommodate a shadow chart that the patient can more easily perform self-documentation and self-updates on, in paper, digital, or both formats. In other words, the “L” would be replaced with “R” and the “R” would be replaced with “L” in English, on select views where such mirroring makes sense. It is further contemplated that all views do not have to be switched simultaneously, and they can be selectively or individually switched in perspective; and both perspectives can be shown simultaneously in different practical applications of the system 985. It is further contemplated that such labels could be language aware (e.g., enabled by the knowledge base module 992 and/or the generation module 993). For a user preferring Spanish, for example, the laterality labels would automatically display as “D” for derecho instead of “R”, and “I” for izquierdo instead of “L.”

It is contemplated that diagrams and maps on shadow charts can contain automatic past, present, and future interaction points, but can also simultaneously serve as present interaction areas for markup and annotation, including drawings and different color schemes, legends, and dictionaries based on context and user and organizational preferences.

It is contemplated that filters and user interaction prior to paper shadow chart printing could modify what is shown on the shadow chart. It is further contemplated that digital shadow charts can dynamically apply filters, view changes, and display additional information, tools, markup, and annotations; and that one skilled in the art would understand additional applications for this technology.

FIG. 6C shows a workflow initiator 643 at the end of a pin description 630 to send a refill on a medication. The workflow initiator 643 is currently unchecked. A user could initiate the medication refill by checking the box. Checking the box on a digital shadow chart would automatically refill the medication and document the refill on the electronic record. Alternately, if checked on a paper shadow chart, computer vision could detect the checkmark on the image capture and automatically refill the medication and document the refill on the electronic record. An alternate workflow initiator 644 shown before a pin 620 and pin description 630 represents a different type of workflow for automatically documenting and tracking counseling on a new pathology result in electronic health record.

FIG. 6D shows a portion of an anatomic diagram 670 does not contain any shadow pins or shadow documentation such as distribution mapping. The same anatomic diagram, representing the back of the body, contains a shadow pin 620 and a context aware description 630. In digital format (e.g., enabled by the GUI 991), the shadow chart has interactive components (e.g., enabled by the input device 988) associated with the pins, pin descriptions, anatomic sites, labels, distribution segments, shapes, and other digital components that a user can use to update the electronic medical record with relevant information. When a shadow chart or form is generated and printed (e.g., enabled by the generation module 993 and/or the output device 989), the shadow documentation can be interacted with, and the portions of anatomic diagrams that do not contain shadow pins or shadow documentation can be marked up and annotated. In certain embodiments, once the annotated shadow chart is scanned, computer vision enabled by the system 985 detects the annotations from the captured image and those detections are used to update the electronic record.

FIG. 6E is an exemplar captured image 6150 (e.g., enabled by the input device 988) of an annotated and marked up paper shadow chart, containing artifacts from capture like perspective warp artifacts and a light-created shadow artifact 6141. These undesired artifacts can be accounted for and ultimately resolved when the captured image 6150 is analyzed with computer vision. The paper orientation and normalization of the axis is also accomplished as the computer vision can detect the normalization markers 680 and the resulting digitized version will have perspective correction, adjusted rotation, and unwanted artifacts removed. The present exemplar contains numerous new markups, annotations, shadings, workflow initiations, and modifications. A shadow pin has been updated with a new pin “x” 626 which in this context aware example means “cryosurgery” was performed in the anatomic location for a premalignant lesion called an actinic keratosis. There are new pin locations 625 on the paper shadow chart as well as shaded in shadow pins 621. The same type of pin can be shaded with different order and list types, exemplifying that different documentation and workflow types can be performed by the same shadow pin type. Information about a pin can be added by writing over the pin description 631 in one exemplified workflow—in the illustrated embodiment, “01—Infiltrative BCC-biopsy date 2017 Dec. 3, path #49234-B, refused tx” has been updated with handwriting to say “wants to schedule since bleeding” indicating that the patient now wishes to treat the skin cancer because of symptoms of bleeding, and prompting interaction with future appointment scheduling workflows. Additionally, a diagnosis has been crossed out 632 to remove or archive the information from the record. It is contemplated that different inks, pigments, shapes, or patterns could additionally be used to initiate different workflows and processes. Checked checkbox 6511 on the printed paper shadow chart will initiate and complete workflows enabled by the system 985 to refill medication in the electronic record after image capture and analysis with computer vision. Checked checkbox 651, initiates the workflow of automatically documenting and tracking counseling on a new pathology result (e.g., enabled by the data processing module 999, the database interface module 996, and/or other components of the system 985). Specifically, documenting counseling, treatment recommendations, and next steps for a “venous lake” diagnosis for the patient, with the anatomic location of the venous lake being consistently documented at the shaded in shadow pin that previously read as “New Result-Specimen B on path #201-32d8-Dx: Venous lake” and the updated additional pin description reading: “Benign-reassurance. Sutures removed today” thus automatically documenting manual descriptions of the healthcare interventions performed at that anatomic location.

The non-limiting example paper shadow chart 6150 also contains new markup and annotations. New markup annotation 627 indicated with “o” in this non-limiting example means that “cryosurgery was performed to inflamed seborrheic keratoses at the marked locations,” whereas new markup annotation 628 indicated with “w” in this example means that “cryosurgery was performed to warts at the marked locations.” It is contemplated that different detectable properties like characters, shapes, labels, orders, pins, symbols, colors, intensities, patterns, shading, lines, and markers can be interpreted through a plurality of templates based on context and preferences and settings. It is further contemplated that detections on a paper shadow chart can be categorized and displayed based on their detected properties, so an “x” and “o” and “w” on the paper shadow chart may use the same pin, but different colors on the digital shadow chart (such as a snowflake or star or asterisk meaning cryosurgery, but different colors could indicate different diagnoses and diagnosis categories).

Non-anatomic information 6212 can also be added to a shadow chart enabled by the system 985 in any detected or patient characteristic-generated whitespace, such as adding diagnoses not associated with the anatomic site or automatically added through specialty context (e.g., health data in void space 930 like shown in FIG. 9D). In FIG. 6E, non-anatomic information 6212 is annotated in a previous whitespace, the handwritten diagnoses of “(1) Pacemaker due to A. fib” and “(2) history of prosthetic heart valve-needs Abx” prompt documentation and workflow processes relevant to the context such as alerts “bipolar or heat cautery should be used due to presence of pacemaker” or “patient is allergic to amoxicillin, and has a prosthetic heart valve requiring antibiotics prior to skin surgery, prescribe azithromycin prior to surgery?”. It is contemplated that such alerts and prompts are possible because of detections on the shadow chart and represent one of a plurality of templates and possible workflows.

FIG. 6F is a magnified view of the lower left portion of the capture from FIG. 6E. New annotations 627 and re-documented annotations 621 are depicted. Re-documented annotations 621, indicated by filling in existing shadow pins, denotes an update to the existing record at that location and automatically documents context aware documentation into the electronic chart. In certain embodiments, this could be no evidence of reoccurrence for melanoma or refill of a prescription. It is contemplated this automatic documentation and workflow and process completion is context aware based on settings, preferences, specialty, language, country, and other characteristics. The new annotations 627, indicated by the character “o,” denotes new healthcare data. In certain embodiments, the “o” may be interpreted as procedures performed on specific diagnoses at the indicated locations such as “cryosurgery to inflamed seborrheic keratosis” as a non-limiting example.

FIG. 6G is a magnified view of the upper central portion of the capture from FIG. 6E and there is an example of an image captured (e.g., enabled by the input device 988) in this non-limiting embodiment. New annotations 625, updated documentation 631, removal or archiving of documentation 632, redocumentation 621, and an initiated workflow 641 are depicted. In the exemplar, updated documentation 631 for a shadow pin description is provided with annotations including a filled in shadow pin 621 and written words over the existing shadow pin description. Shadow documentation may be removed or archived 632 through an annotation that crosses out the existing shadow pin description. As indicated above, annotations to indicate re-documentation may be done by filling in existing shadow pins to denote an update to the existing record at that location. In certain embodiments, a diagnosis for rosacea may be re-documented for a patient experiencing an outbreak. Further, a checked workflow initiator could automatically refill (or queue for refill) the prescription for treating the outbreak. In certain embodiments, the filled in shadow pin 622 and accompanying checked workflow initiator 641 redocuments the patient's rosacea and orders a refill for Doxycycline 50 mg twice daily, and Metrocream once daily. It is contemplated that automatic documentation and workflow and process completion enabled by the system 985 is context aware based on settings, preferences, specialty, language, country, and other characteristics as one skilled in the art would know.

FIG. 6H is a screenshot of a portion of an annotated paper shadow chart converted to a digital record by computer vision enabled by the system 985. The digital record has new pins 629. The new pins 629 were extracted, categorized, and plotted based on the computer vision detections of the handwritten annotations 627 in FIG. 6F. The history of the record for this location began as an anatomic diagram 670 lacking any documentation in FIG. 6B which was annotated by hand on a paper shadow chart depicted by new annotations 627 in FIG. 6F and is now shown with new pins 629 in the electronic record. In the illustrated embodiment, asterisks represent cryosurgery to inflamed seborrheic keratosis (category: benign) in this single example in an area of a diagram that did not have shadow documentation in the original shadow chart in FIG. 6B. Shown in a side panel 6350, each new digital pin 629 has a corresponding anatomic site description 6329, pin description 6330, a diagnosis 6331, and other associated data 6332 (such as photographs, attachments, and links, e.g. as enabled by the input device 988). It is contemplated that the pin description 6330 can optionally be shown or hidden on the anatomic diagram 670 by the system 985 to avoid crowding (e.g., enabled by the GUI 991).

Re-documented shadow pin 623 and accompanying pin description 633 are generated from the filled in shadow pin 621 in FIG. 6F and are automatically updated to include context aware information. In the illustrated embodiment, the redocumented shadow pin 623 and accompanying pin description includes the label “No evidence of recurrence” for a history of melanoma that was examined and documented during the patient encounter. It is contemplated that the documentation points enabled by the system 985 can be modified, moved, deleted, augmented with more information such as photographs, attachments, and links.

FIG. 6I is a screenshot of a portion of an annotated paper shadow chart converted to a digital record by computer vision enabled by the system 985. The digital record has new pins 629 and re-documented pins for infiltrative BCC 6340, rosacea 6344, Hx BCC 6348 diagnoses. The new pins and re-documented pins were extracted, categorized, and plotted based on the computer vision detections of the handwritten annotations in FIG. 6G. In the illustrated embodiment, the history of the record for re-documented infiltrative BCC 6340 began as an anatomic diagram with existing documentation including a shadow pin 620 and shadow pin description 630 in FIG. 6B. The location was annotated by hand with a filled in shadow pin 621 and handwritten text 631 over the existing documentation as depicted in FIG. 6G and is now shown with a re-documented pin 6340 and accompanying updated shadow pin description 6341 (e.g., enabled by the input device 988 and/or other components of the system 985). As another example of the record tracking information enabled by the system 985 is illustrated in the non-limiting embodiment, the history of the record for re-documented rosacea 6344 began as an anatomic diagram with existing documentation including a shadow pin and shadow pin description that was annotated by hand with a filled in shadow pin 622 and checked workflow initiator 641 over the existing documentation as depicted in FIG. 6G and is now shown with a re-documented pin 6344 and accompanying updated shadow pin description 6345.

The new pins 629 appear as asterisks in certain embodiments and have corresponding anatomic site descriptions 6329, pin descriptions 6330, a diagnosis 6331, and other associated data 332 (such as photographs, attachments, and links) shown in a side panel 6350 on the screen. It is contemplated that the pin description 6330 can optionally be shown or hidden on the anatomic diagram 670 by the system 985 to avoid crowding. It is contemplated that different colors can further be used to enhance the tracking and recording of information on the shadow charts.

FIG. 6J is an example of distribution coloring 6155 with different colors and intensities on a paper shadow chart 6213. The example is split in half between a digital photograph of the markup and a line art representation for illustrative purposes to enable the teachings herein. The picture of the shadow chart (e.g., enabled by the input device 988) additionally has orientation and axis normalization markers 680 for enhanced alignment of the shadow documentation relative to the diagrams. Context data such as language for the shadow chart can be determined by detected areas like a QR code 690. This shadow chart enabled by the system 985 also has blank fields 6151 for patient demographics indicating that it could be used as a backup chart without shadow information, useful in times where internet access is limited or not available such as during an internet outage or in remote military operations. The shadow chart could later be scanned (e.g., enabled by the input device 988), at which time computer vision enabled by the system 985 will detect information in the blanks and create a digital record.

The non-limiting example shows shaded annotations in various colors 6155 at anatomic distribution sites. It is contemplated that different colors would indicate different relevant information allowing computer vision enabled by the system 985 to detect and interpret the color variations. Context awareness allows for these detections to be categorized and documented correctly into the electronic medical record by the system 985. In certain embodiments, blue could correlate to a diagnosis of “dermatitis”, green could correlate to a diagnosis of “lupus”, and red could correlate to a diagnosis of “psoriasis” (e.g., enabled by the knowledge base module 992, the generation module 993, the data processing module 999, and/or other components of the system 985). Computer vision would automatically color the distributions on digital records in their correct colors, associate diagnoses, and group the anatomic sites into consolidated named distributions when possible.

FIG. 6K depicts a screenshot digital record of FIG. 6J converted by the system 985. The detected distributions 6156 appear in the same colors as the shaded annotations 6155 in FIG. 6J. The side panel 6350 includes the corresponding anatomic site descriptions 6329, pin descriptions 6330, diagnoses 6331, and other associated data 6332, shown here in Chinese which indicates the QR Code 690 in FIG. 6J provided language context for the chart as Chinese. It is contemplated that these anatomic site descriptions in this example are automatically combinable to be “bilateral malar region” or “butterfly rash” in their Chinese translations as one skilled in the art would know. The side panel 6350 further organizes the generated information by color-coded distribution with a color indicator 6158 matching the color on the anatomic diagram 670. Further, the laterality labels 43 from the outside observer perspective of the shadow chart are shown in Chinese in this non-limiting embodiment.

FIG. 6L shows the converted digital record from FIG. 6K automatically translated to English with the laterality labels 43 and side panel 6350 information, including anatomic site descriptions 6329, pin descriptions 6330, diagnoses 6331, and other associated data 6332, all shown in English. It is contemplated that these anatomic site descriptions in this example are automatically combinable to be “bilateral malar region” or “butterfly rash” as one skilled in the art would know. It is further contemplated that these detections, descriptions, diagnoses, colors, photos, attachments, and links are automatically translatable to any coded, linguistic, or symbolic language.

FIG. 6M depicts a screenshot that illustrates a visual alert 6175 on the shadow chart that displays anatomy specific warnings enabled by the system 985. It is contemplated that the visual alert 6175 can optionally be highlighted on alternate views 6176 and angles of the affected anatomy, in different perspectives (e.g., enabled by the generation module 993 and/or the GUI 991). Such alerts can appear on digital shadow charts, paper shadow charts, both, or neither based on context awareness. For example, if the medical assistant who normally takes blood pressure during the rooming process has the electronic health record open, the alert can be shown to them during the vitals capture stage of the encounter (e.g., enabled by modules in the medium 995 in communication with the processor 986 of the system 985). A smart and connected blood pressure cuff could also verbally alert the assistant in this non-limiting example (e.g., enabled by the input device 988 and/or the output device 989).

In one embodiment, a method for interacting with electronic records, the method comprising: creating a shadow chart or shadow form by compiling data into a plurality of templates; interacting with a shadow chart or shadow form wherein the shadow chart or shadow form has at least one anatomic diagram and data relevant to a particular patient; translating interactions into new or modified context aware data; generating and/or updating electronic records with the data compiled from the translated data; generating and/or updating descriptions for anatomic sites and for context aware data on the shadow chart based on the translated data; and creating updated shadow charts or shadow forms with the translated data and associated descriptions.

In certain embodiment, the shadow chart or shadow form is in digital form, physical form, or both, and interactions are in digital form, physical form, or both. In certain embodiments, the method further comprising modifying the translated data and generated and/or updated descriptions through selection, filters, and interactions modifiers. In certain embodiments, the method is a loop so that new or updated relevant data is incorporated into the records. In certain embodiments, the data includes anatomy information, patient information, anatomy visualizations, and/or other context aware data. In certain embodiments, select diagrams can be hidden or modified when relevant or irrelevant to a particular context. In certain embodiments, the physical shadow chart has a code that supplies additional information about the shadow chart when the digital image is processed with computer vision. In certain embodiments, the data includes past, present, and/or future practitioner information, patient demographics, encounter information, patient history, results and reports, schedule calendar data, multimedia and images, financial and billing data, cosmetic and purchase data, and/or other relevant data. In certain embodiments, the data includes anatomical and non-anatomical data. In certain embodiments, the shadow chart or shadow form has various anatomic diagrams in various perspectives.

In another embodiment, a system for creating and updating electronic records, the system comprising: a central information management system operable to store a multitude of records; context aware data wherein the context aware data is relevant to a desired record contained in the central information management system; a shadow chart or shadow form wherein the shadow chart or shadow form includes relevant data; a shadow chart or shadow form that can be interacted with; at least one computer processor 986 configured to at least: translate interactions with a shadow chart or shadow form into detected data; process the detected data; categorize the detected data; generate and/or translate new or updated records, shadow charts, and/or shadow forms.

In certain embodiments, the shadow chart or shadow form is in digital form, physical form, or both, and interactions are in digital form, physical form, or both. In certain embodiments, the generated and/or translated data includes anatomy information, patient information, and/or anatomy visualizations. In certain embodiments, the generated and/or translated data are created through selection, filters, and interactions modifiers. In certain embodiments, select diagrams can be hidden or modified when relevant or irrelevant to a particular context. In certain embodiments, the physical shadow chart has a code that supplies additional information about the shadow chart when the digital image is processed with computer vision. In certain embodiments, the whitespace and documentation space on a physical shadow chart or physical shadow form validates and applies context awareness. In certain embodiments, the physical shadow chart interactions are captured, processed, and applied in a context aware manner. In certain embodiments, the data includes past, present, and future practitioner information, patient demographics, encounter information, patient history, results and reports, schedule calendar data, multimedia and images, financial and billing data, cosmetic and purchase data, and/or other relevant data. In certain embodiments, anatomy specific alerts are visualized in a shadow chart.

(APP07) Traditionally, accurate description and categorization of morphologies, and of skin type and skin tone, and of anatomic sites and distributions of involvement is a manual and disjointed process that requires human cognition, human intelligence, and human analysis. Broad categories of skin type and skin tone exist to attempt to simplify human analysis and automated detections can throw off computerized analysis because skin color can vary in images based on environmental factors (e.g., lighting conditions), type of device (e.g., image-processing occurs on smartphone differently than it does on a Digital Single Lens Reflex (DSLR) camera), and anatomic location (e.g., sun exposed areas may be less accurate representation of patient's actual skin type and skin tone compared to less sun exposed anatomic locations).

The system 985 of the embodiments illustrated detects various aspects of multimedia like images, videos, illustrations, avatars, 3D captures, and captures over time that contain anatomy and describable features or findings, e.g., morphologies, which can be counted, measured, categorized, and area-calculated (e.g., enabled by the data processing module 999). Other data may be present as well, such as user input patient data (e.g., enabled by the input device 988) or patient data available within the image capture or image analysis applications. The patient data may include, for non-limiting example, details on the diagnosis, patient age, patient race, patient sex, and other characteristics. Environmental data can also be extrapolated automatically from GPS coordinates of the image capture, multimedia metadata such as EXIF, detection of lighting conditions, weather in that geographic location based on time and date of image, whether the image was captured indoors vs. outdoors, etc. (e.g., enabled by the input device 988 and/or data processing module 999). Additionally, aberrations, distortions, and artifacts can be detected in the images and removed by the system 985 from the analysis. For example, correcting washout of a shiny papule caused by a camera flash or removing a reflection off an oily skin surface from a flash or overhead light. The various detections and categorizations are enabled by the system 985 applying computer vision, data extraction, and manual annotation and refinement. The anatomy, counts, calculations, measurements, surface area, dynamic anatomic addresses, morphology, patient data, and skin type and skin tone can also be transformed by an anatomy visualization engine (e.g., enabled by the data processing module 999 and/or other components in the system 985). In addition to anatomic sites, physically marked locations on an image, such as those marked with a permanent or ink-based marker on the patient before photographing, as one example, can also be automatically plotted, ordered, and associated with the correct metadata (e.g., enabled by the data processing module 999 and/or other components in the system 985). For example, 3 lesions with ink dots around them in a blue marker on skin type 2, with lesions labeled in marker as A, B, and C are detected, plotted, labeled, described, mapped and ordered. All the data can be transformed, analyzed, and translated through neural networks of the system 985 and other information systems (e.g., enabled by the database interface module 996) that have omnidirectional communication capabilities, and can apply machine learning, artificial intelligence, and augmented intelligence. In certain embodiments, the data from the neural networks are processed through an analysis engine to deliver automatic diagnosis and automatic skin subtyping and subtoning, which take into account various data points like anatomic locations of images, age, patient data, environmental conditions, and detected skin type and tone features to deliver subcategories of skin type and skin tone. In one example, spatial computing is utilized to detect, categorize, describe, locate, calculate such data points through application of a coordinated language model 980 and other vision-language 1101, language-vision 1102 and language models 1103.

The embodiments illustrated automatically generate descriptions, such as anatomic site/distribution, lesion or rash morphology (e.g., erythematous, scaly, etc.), measurements, surface area calculations, skin type categorization (e.g., Fitzpatrick scale), or severity (e.g. enabled by the generation module 993) on patient images or multimedia (e.g. enabled by the input device 988), with synchronous real time visualization on corollary avatars or diagrams and real time synchronous automatic translation and categorization in any coded, linguistic, symbolic language in multidimensional, multi-axised (multiple axes, multi-axial with custom rotations and scalings in the different axes) spaces. Additional non-limiting examples include automatic detection of optimal excision or repair orientation for tissue removals and closures (flaps, grafts, primary closures) based on anatomic site; Automatic detection of distribution in dermatomes; Automatic detection of tissue laxity, tension vectors, and optimal orientation for surgical excisions and repairs (e.g. enabled by the knowledge base module 992); Automatic anatomic distribution detection (e.g. seborrheic, photo distributed) and categorization or morphology features to render suggested diagnosis; and applying Langer's lines and dermatomes automatically to images, videos, augmented reality, avatars, and diagrams, and other multimedia (e.g. enabled by the input device 988), while simultaneously describing anatomy in real time in any coded, linguistic, or symbolic language.

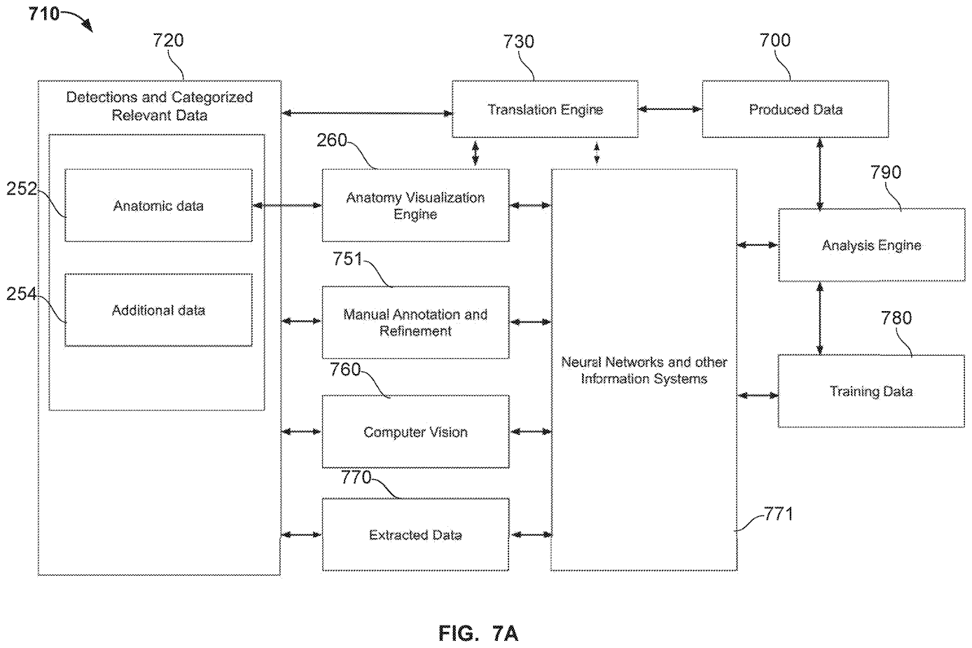

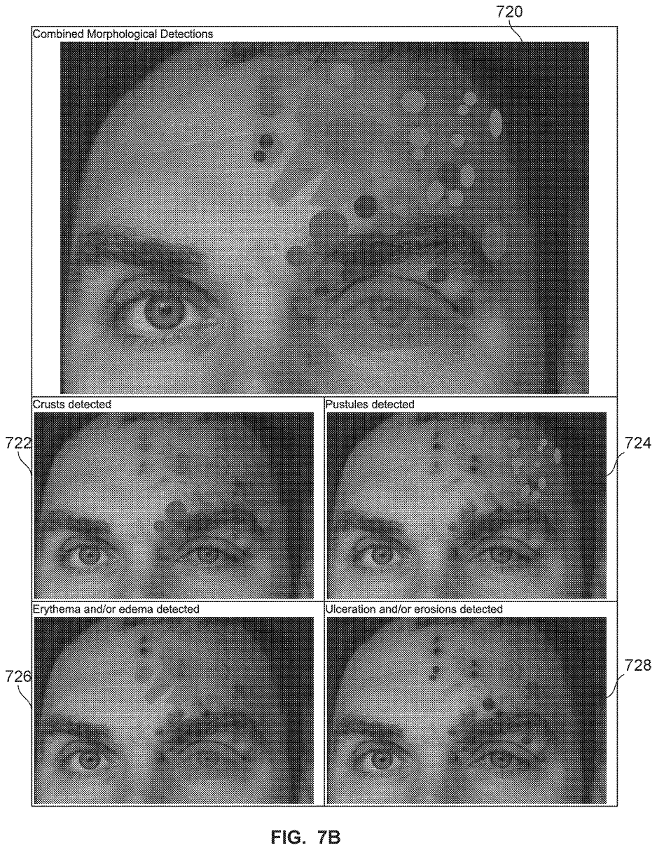

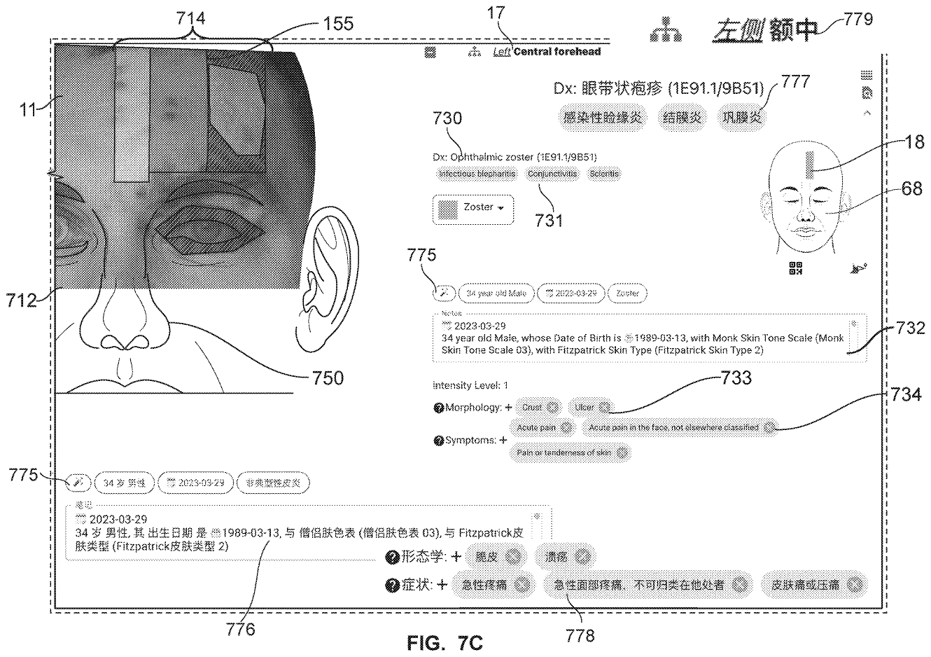

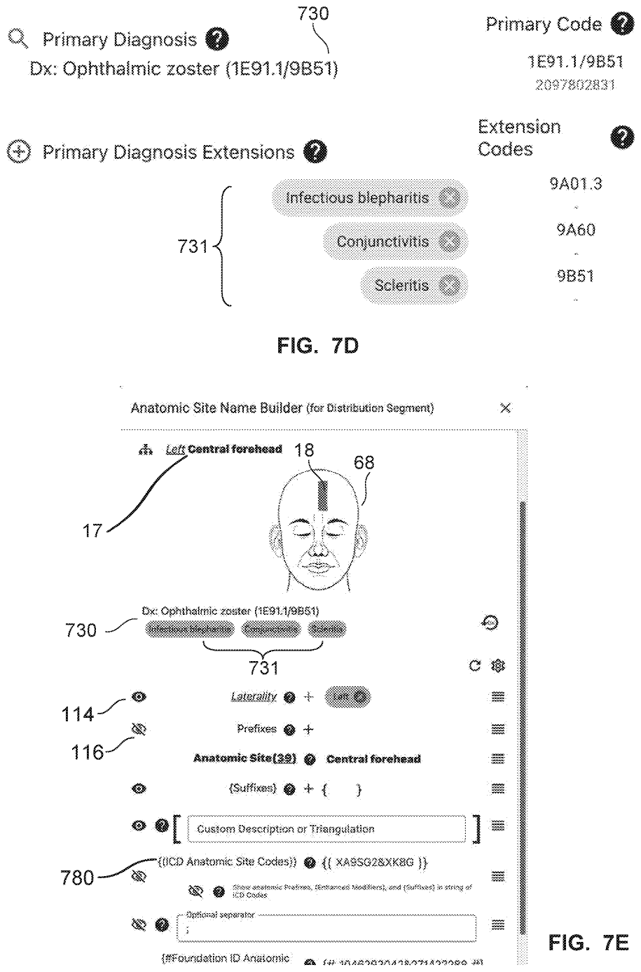

FIG. 7A is a simplified block diagram of a process and/or method 710 enabled by the system 985 relevant to anatomy and morphology. The method 710 enabled by the system 985 includes relevant data 720 detected and categorized from an image with computer vision, a translation engine 730, an anatomy visualization engine 260, manual annotation and refinement 751, computer vision 760, extracted data 770, neural networks 771, training data 780 and an analysis engine 790. The method 710 enabled by the system 985 utilizes computer vision to detect and categorize relevant data 720 including anatomy, morphology, patient data, skin type and skin tone, environment data, and aberrations, distortions and other artifacts (e.g., enabled by the knowledge base module 992, the data-processing module 999, and/or other components of the system 985). Part of the relevant data 720 detected and categorized is anatomic data 252, such as anatomic sites, anatomic site segments, anatomic distributions, counts, measurements, surface area, dynamic anatomic addresses, which is automatically communicated with the anatomy visualization engine 260 (e.g., enabled by the data processing module 999). Relevant data 720 also includes additional data 254, such as morphology, patient data, and skin type and skin tone, which may optionally be automatically communicated with the anatomic data 252 to the anatomy visualization engine 260. It is contemplated that morphology, patient data, skin type and skin tone can be detected from patient records, images, video, and other multimedia, and can vary in description, category, and other data and calculations based on anatomic site, measurements, sex, gender, and other patient data. Other relevant data 720 includes environmental data and aberrations, distortions, and other artifacts. The method 710 enabled by the system 985 provides for omnidirectional communication between the detected and categorized relevant data 720, translation engine 730, anatomy visualization engine 260, manual annotation and refinement 751, computer vision 760, extracted data 770, and neural networks 771. It is contemplated that additional neural network mapping enabled by the system 985 could allow for additional communication connections within the system 985. Other network and information systems may be substituted for neural networks as one skilled in the art would know.

The neural networks 772 generate training data 780 for artificial intelligence and machine learning. Neural networks 771 and the deduced findings from the training data 780 are applied in an analysis engine 790 to categorize, summarize, calculate, predict, diagnose, and generate produced data 700 (e.g., processed health data enabled by the system 985) such as reports, automated detections and/or categorizations, automated diagnosis, automated skin subtyping and subtoning, and translations.

FIG. 10D shows a patient photo 11 that when analyzed by the system 10 has generated (e.g. enabled by the generation module 993) a produced anatomic map FIG. 10E that contains detected anatomic distribution, morphologies, counts, and surface area used to determine a diagnosis from the analyzed patient photo 11 (e.g. received from the input device 988 and enabled by the data processing module 999 as one non-limiting example). Multidimensional and hierarchical painting 155 applying components of dynamic anatomic addresses is done on the patient image and is performed automatically with computer vision and artificial intelligence in this embodiment of the system 985. Different subsegments can be labeled with different average findings as well. In this embodiment, the system 985 detects automatically that this is zoster that involves the eye based on: anatomic distribution in a dermatome, anatomic location of specific lesions within the distribution, different lesional morphology at the same time (crusts, pustules, vesicles, papules), morphology of eye and eyelids (swollen eyelid, red eye, on one side). Lesional counts are automatically obtained within different subsegments (e.g., enabled by the data processing module 999 and/or the image interface module 995 as non-limiting examples), and automatically categorized based on morphology detections as well. This differentiates the diagnosis from smallpox with a high degree of confidence, as one non-limiting example, based on its anatomic distribution and the mixed lesional morphology within the anatomic distribution and dynamic anatomic address, as smallpox would have all lesional morphology in the same stage, such as they would all be crusts rather than have a mixture of pustules, crusts, vesicles, and papules (e.g. as enabled by the knowledge base module 992). From here, the system 985 initiates generation of automatic treatment recommendations and management pathways, such as prescription acyclovir, prednisone, referral to ophthalmology for ocular involvement and workflow and management pathways are initiated automatically by the computerized algorithm stored and processed in the medium 995. Additional data the system 985 may detect in the photo 11 could include hair color, eye color, skin type, and skin tone. Detecting these features as properties, along with anatomic location of the detections, and environmental and other conditions, can be combined to give a skin type and skin tone subtype through skin typing and toning algorithms and scales in certain embodiments of the system 985 (e.g., enabled by the data processing module 999). It is contemplated that the systems, methods and algorithms of the present non-limiting examples are applicable to multimedia containing deep anatomy or microscopic anatomy such as x-rays, CT scans, and even digital microscopic slides (e.g., enabled by the input device 988 and/or the image interface module 994). For example, detecting the morphology of cells and cell groups and micro-anatomic location of cells in the basal layer could describe the cells and their melanin content to skin tone and subtone based on the location and concentration of the melanin pigment in the epidermis. The same detections are applicable to dermoscopy (e.g., enabled by a dermatoscope as the input device 988), accounting for the location of pigment in magnified images, under different lighting conditions like polarization, and the detected anatomic locations and distributions within the lesion itself and relative to the body location.

Further, the system 985 automatically detects, categorizes, counts, and calculates surface area of morphological findings on different anatomic segments in a definable anatomic distribution. The morphological detections can be highlighted in different colors, opacities, shapes, sizes, and angles to distinguish different detections. FIG. 7B depicts an example of this, based on the photo 11 from FIG. 10D and FIG. 10E, highlighting different categories of detections (e.g., enabled on the GUI 991). In this embodiment, there are four morphological categories detected. The shapes, sizes, and opacities determine relative surface area, intensity, counts, measurements, and other calculations for each detection, and they can overlap. They are illustrated in FIG. 7B in a combined morphology 720 and individually in a crust morphology 722, pustule morphology 724, erythema and/or edema morphology 726, and ulceration and/or erosion depiction 728. Overlapping detections can apply mathematical algorithms of the system 985 stored in the medium 995 in communication with the processor 986 to subtract, add, multiply, divide, square, square root, or otherwise calculate relationships. It is contemplated that unlimited categories and segmentations can be applied based on user preference, sensitivity, specificity, and other parameters and settings. It is further contemplated that the morphological detection data can be combined with the anatomic detection data to assist with downstream functions such as automated diagnosis, translation, description, encoding, billing, prescription recommendations, and other functions.

FIG. 7C further shows detections from the photo 11 in FIG. 1ZD with a combined anatomic map image 712 and summary of the detections 713. Each detected anatomic location is highlighted on a portion of the anatomic map 712 with multidimensional and hierarchical painting 714 combined into a visualization. The detection summary 713 includes a diagnosis 730 (labeled here with the English description and automatically encoded ICD code “1E91.1/9B52”), diagnosis extensions 731 (infectious blepharitis, conjunctivitis, scleritis), patient note 732 written by a “automatic note writer” 775 (including patient skin tone and patient skin type), relevant morphologies 733, and symptoms 734. In this embodiment, the patient note 732 includes information with the date, patient age, date of birth, skin tone, skin type, and other detected and known information and symptoms 734 include acute pain, acute pain in the face, and pain and tenderness of skin. It is contemplated that the anatomic site detections enabled by the system 985 can be scaled, grouped, moved, combined, added, subtracted, divided, multiplied, area calculated, aligned to better fit actual patient or avatar-based multimedia. It is further contemplated that groups, for example, can have a group description, calculation, scaling, movement, or other property. An example of a group description in English for the illustrated embodiment would be “left forehead”. The “automatic note writer” uses data stored and extracted from a patient encounter (e.g. enabled by the database interface module 996) or history (e.g. enabled by the record retrieval module 997), a database that hosts a plurality of templates, wherein the templates contain variables, tokens, targets, coordinates, and visualizations, and the processor 986 and a coordinated language model 980 engine to generate information about a particular anatomic site or group of sites. A non-limiting list of example variables and tokens and targets includes: System Variables: Patient form info: Patient_FirstName; Patient_LastName; Patient_FullName; Patient_Prefix_Mr; Patient_Pronoun_he; Patient_Pronoun_his; Patient_Pronoun_him (automatically inserting correct one based on patient sex or gender in preceding 4 examples); Patient_Sex; Patient_MRN; Patient_ID; Patient_AdditionalInfo; Patient_Birthday; Patient_Age_at_encounter (based on encounter date); Patient_Age_today (based on system date); Patient_Country; Patient_Fitzpatrick_Skin_type; Patient_Monk_Skin_tone; User/Clinic/Entity form info: Encounter_Clinic_Name; Encounter_Clinic_Address; Encounter_Clinic_City; Encounter_Clinic_StateOrProvince Encounter_Clinic_Fax; Encounter_Clinic_Website; Encounter_Clinic_AdditionalInfo; Encounter_Doctor_Provider_Name; Encounter_Assistant_Names; Encounter form info; Encounter_date; Encounter_time; Appointment_time; Encounter_notes; -Listinfo; ListGroup_PrimaryDiagnosis; ListGroup_PrimaryDiagnosisExtensions; ListGroup_AlternateDiagnosis; ListGroup_AlternateDiagnosisExtensions; ListGroup_Morphologies; ListGroup_Symptoms; ListGroup_FirstPhotoThumbnail; ListGroup_FirstPhotoNotes; ListGroup_FirstPhotoTags; ListGroup_AllPhotoThumbnails; ListGroup_AllPhotoNotes; ListGroup_AllPhotoTags; ListItem_AnatomySiteName; ListItem_AnatomyCodeStringICD; ListItem_AnatomyCodeStringAMID; ListItem_AnatomyCodeStringFoundationID; ListItem_AnatomyEmojiCodeString; ListItem_Anatomy_Coordinates; ListItem_Anatomy_Deviation; ListItem_Anatomy_Laterality; ListItem_Anatomy_NYUNumber; ListItem_Anatomy_VisualPreview; ListItem_Anatomy_VisualPreview_Mirrored; ListItem_Anatomy_VisualPreview_with_Borders (each VisualPreview may have defined sizes like xsmall, small, medium, large wherein the size of the diagram changes but the size of the pin and pin description remains constant in one example); ListItem_PinDescription; ListItem_Tool_selection; ListItem_Measurements; ListItem_PrimaryDiagnosis; ListItem_PrimaryDiagnosisExtensions; ListItem_AlternateDiagnosis; ListItem_AlternateDiagnosisExtensions; ListItem_Morphologies; ListItem_Symptoms; ListItem_FirstPhotoThumbnail; ListItem_FirstPhotoNotes; ListItem_FirstPhotoTags; ListItem_FirstPhotoSymboliclFile_name; ListItem_AllPhotoThumbnails; ListItem_AllPhotoNotes; ListItem_AllPhotoTags; ListItem_AllPhotoSymbolicFile_names.

In certain embodiments, a new automatic note can be written by the system 985 at different time points (e.g., encounter dates, time of entry, etc.) as more health data becomes available, as a practical application of the system. Each new note can represent a “chapter” in the evolving story and/or novel written about the health data to generate (e.g., enabled by the generation module 993) a historical record to be stored on the medium 995. The historical record can be analyzed, rewritten, updated, moved, organized, collated, and/or otherwise modified by the system 985 as non-limiting examples. In certain embodiments, the historical record is stored within its own file and/or file system on the medium 985, such as an SVG file or EXIF metadata as non-limiting examples.

The system 985 automatically encodes diagnoses and diagnosis extensions (e.g., enabled by the knowledge base module 992). FIG. 7D shows the primary diagnosis 730 and diagnosis extensions 731 from FIG. 7C. In this embodiment, the primary diagnosis 730 is ophthalmic zoster (with accompanying ICD code “1E91.1/9B52”). The diagnosis extensions 731 automatically encoded are infectious blepharitis (9A01.3), conjunctivitis (9A60), and scleritis (9B51). It is contemplated that one skilled in the art would know that this is just one example for illustrative purposes, and there are unlimited examples of encodings, links, and cross-maps that will evolve over time.

The system 985 analyzes the relevant data to determine skin tone and skin type. The system 985 utilizes a skin color shading gradient that combines Fitzpatrick skin type (I-VI) and Monk Skin Tone scale (01-10) and allows for custom skin typing and skin toning by allowing application of simultaneous scales or subscales (e.g., enabled by the knowledge base module 992 in communication with the data processing module 999 as one non-limiting example). Additionally, color blending is used to combine multiple scales HEX, RGB (red, green, blue), Monk skin tone, Fitzpatrick skin type, or other color scale values for a more realistic match. The system 985 also adjusts for lighting, flash, contour, shadow, and other image metadata for greater accuracy. The system's color scale (e.g., enabled by the tangible medium 995) can be used for research stratification and when combined with other anatomic features, such as hair color, eye color, facial feature shapes, and body shapes, provide for ethnicity and race prediction. It is contemplated in a commercial implementation the system's color scale could be used to apply a custom blend of makeup recommendations and pigments, as practical applications of the system 985.

The translation engine of the system 985 (e.g., enabled by the knowledge base module 992 and/or data processing module 999) allows for all languages and encodings to be supported, including cross-maps to other terminology sets such as SNOMED-CT and Foundational Model of Anatomy and the “NYU” numbering system (e.g., enabled by the database interface module 996). FIG. 7C also shows Chinese translations for the anatomic site name in the isolated visual preview, a note written by artificial intelligence with automatic note writer 775 function that combines visualizations, patient information, health information, and detections to automatically write a note in any language, diagnosis and diagnosis extensions 776, and morphology and symptoms and coordinate the information with a coordinated language model 980 engine and/or type model enabled by the system 985. Further, the system applies natural language processing (e.g., enabled by the data processing module 999) to anatomic site descriptions 17 and other data in this embodiment. The automatic note writer function 775 applies artificial intelligence, coordinated language model 980 engines, detections, and inputs to generate text-based and visual summaries of the anatomy, health data, patient data morphologies, and diagnoses. The visualizations can be isolated 68 or combined, and are callable through tokens or text expanding macros or inputs or templates, thus giving the user control over how the visualizations are presented based on their preferences. As one example, “@isolatedvisualpreview@” is a non-limiting example token that pulls and generates the isolated visualization into the medical note (e.g., enabled by the generation module 993). The visualizations can be presented differently to different users in a context aware manner or based on user preferences.

The engines of the system 985 also allows for translation to linguistic languages, coded languages and symbolic languages. FIG. 7E shows a screenshot for the selected “left central forehead” of cross-mappings to coded 780 and symbolic languages. It is contemplated that one skilled in the art would know that this is just one example for illustrative purposes, and there are unlimited examples of encodings, links, and cross-maps that will evolve over time. Also depicted are an isolated anatomic distribution segment representing an anatomic site 18 shown on an isolated visual preview 68 with the correlating anatomic site name 17. The diagnosis 730 and diagnosis extensions 731 are also shown (e.g., enabled by the GUI 991). Anatomic sites are broken down and dissected into their components with the engines in the system 985, and the laterality is visible 114 while prefixes are toggled with visibility off 116 and empty.

In one embodiment, a computer vision method for detecting, extracting, and categorizing anatomic findings and morphologies from multimedia, the method comprising; receiving one or more multimedia of an anatomic site, anatomic site segment, or anatomic group; identifying findings and/or points of interest and/or areas of interest on the multimedia; optionally describing the anatomic site, anatomic site segment, or anatomic group; analyzing the findings; optionally detecting counts, proximities, relationships, and surface areas of regions of interest and areas of interest; optionally categorizing morphologic names and classifications of findings and/or points of interest and/or areas of interest; optionally annotating an anatomic map and/or avatar and/or multimedia with the categorized findings and/or points of interest and/or areas of interest wherein the annotations are made at the corresponding anatomic site, anatomic site segment, or anatomic group of the anatomic map and/or avatar; and generating a summary of the categorized skin findings.

In certain embodiments, the multimedia comprises an illustration, avatar, map, photograph, file, or video that contains anatomy and a detectable finding. In certain embodiments, the analysis of the findings related to the skin and/or anatomy refines the skin type and tone accounting for patient characteristics. In certain embodiments, the analysis of the findings related to the skin and/or anatomy refines the skin type and tone accounting for environmental conditions. In certain embodiments, the generated summary is translatable in any coded, linguistic, or symbolic language. In certain embodiments, the areas of interest marked with ink before image capture are detected, described, labeled, mapped, and ordered.

In another embodiment, a computer vision system for detecting, extracting, and categorizing anatomic morphologies from multimedia, the system comprising; the processor 986 in communication with the data processing module 999 in the tangible medium 995 for the identification of findings and detection of counts, proximities, relationships, and surface areas of the findings in an image wherein an output of said processor 986 categorizes morphologic names and classifications of the findings; a multimedia of an anatomic site, anatomic site segment, or anatomic group; a display wherein a user can view an anatomic location representative of the multimedia; a record generation engine being configured to fill a set of fields with the specific patient information; and a non-transitory computer readable medium, storing machine executable instructions executable by the processor 986, the machine executable instructions configured to: receive one or more multimedia of an anatomic site, anatomic site segment, or anatomic group; optionally identify findings on the anatomic site, anatomic site segment, or anatomic group; optionally analyze and/or describe distribution of the findings; optionally detect counts, proximities, relationships, and surface areas of findings; categorize morphologic names and classifications of findings; optionally annotate an anatomic map and/or avatar and/or multimedia with the categorized findings wherein the annotations are made at the corresponding anatomic site, anatomic site segment, or anatomic group of the anatomic map and/or avatar and/or multimedia; optionally categorize skin type, skin tone, and custom skin subtype and skin subtone; and optionally generate a summary of the categorized findings.

In certain embodiments, the multimedia comprises an illustration, avatar, map, photograph, file, and video that contains anatomy or a detectable finding. In certain embodiments, analysis of the findings related to the skin and/or anatomy refines the skin type and tone accounting for patient characteristics. In certain embodiments, the analysis of the findings related to the skin and/or anatomy refines the skin type and tone accounting for environmental conditions. In certain embodiments, the generated summary is translatable in any coded, linguistic, or symbolic language. In certain embodiments, the annotated anatomic maps and/or avatars and/or multimedia are reintroduced to the system as a training model for machine learning, artificial intelligence, and deep learning. In certain embodiments, the areas of interest marked with ink before image capture are detected, described, labeled, mapped, and ordered.

In another embodiment, a system to generate representative visualization from any combination of linguistic, coded, or symbolic descriptions of any combination of anatomy, morphology, patient characteristics, procedural data, skin type, and skin tone, the system configured to: generate visualizations of diseases, diagnoses, procedures, and health findings wherein the visualizations may be images, videos, avatars, maps, diagrams, illustrations, or photos.

(APP08) Traditionally available anatomy lexicons and ontologies are disjointed with no way to establish relationships among the different anatomic entities. Anatomic collections and their relationships have practical applications in healthcare such as defining anatomic distributions or defining very precise anatomic structures that fit into a certain billing category. For example, in the United States based Current Procedural Terminology (CPT) coding set maintained by the American Medical Association (AMA), a shave biopsy procedure of the eyelid has a different procedural code than a shave biopsy procedure on the eyelid margin. The laterality of the procedure code does not matter. However, a diagnosis code from the international classification of disease (ICD) for a basal cell carcinoma (BCC) is different depending on if the BCC is on the left or right eyelid, and it combines the anatomic concepts of the eyelid and eyelid margins but separates laterality. Through using different range categorizations or different collections of the anatomy enabled by the system 985 for different encodings and visualizations, novel data relationships are established to perform different coding tasks in tandem, but keeping the data related, thus forming a component of a coordinated language model 980 engine and/or type model.

Taking this further, collections of ICD anatomy codes with specific inclusions and exclusions can be used to create anatomic distribution descriptions and relationships. For example, anatomic sites in the “milk line” might include the breasts, nipples, and areas on the abdomen which are commonly used to describe a diagnosis of an accessory nipple (extra nipple) (e.g., enabled by the knowledge base module 992).

Language detection of diagnosis types and automatic encoding is enabled by the system 985 with or without anatomy data. An example of the description “basal cell carcinoma, nodular and superficial type”, would automatically encode as 2C32&XH2CR0&XH5NL6 in the embodiments illustrated using ICD-11 encoding. This same example in Spanish, “Carcinoma basocelular de piel, tipo superficial y nodular” would automatically encode to 2C32&XH2CR0&XH5NL6 in the embodiments illustrated as non-limiting examples (e.g., enabled by the knowledge base module 992 and/or the data processing module 999). The embodiments illustrated enabled by the system 985 apply relevant language through natural language processing and removes redundant language (e.g., enabled by the data processing module 999). Taking this example further, “Basal cell carcinoma, superficial and nodular type, on the Left (Superior) Lateral forehead” would become the encoded string “2C32&XH2CR0&XH5NL6&XA1Z38&XK9K& (XK5N).” The order of the language and the order of the encoding does not matter. It is contemplated that in certain embodiments, the language or the code string could be converted to a generated visualization on an avatar or map (e.g., enabled by the generation module 993). It is contemplated that as another example, the description or the code sequence could be highlighted on a patient image, video, or other multimedia. It is further contemplated that as another example, procedures could automatically be described, encoded, tracked, or linked to anatomic sites based on a range categorization and procedure type and automatic billing could occur based on these relationships. As one non-limiting example, “2C32&XH2CR0&XH5NL6&XA1Z38&XK9K&(XK5N)&11312” adds the “11312” to the encoded string which is an example of a CPT code published by the American Medical Association (e.g., enabled by the knowledge base module 992) that correlates with a shave removal biopsy on the face, 1.1-2 cm in size. Based on the range categorization enabled by the system 985, this code is automatically generated based on the procedure description and characteristics (e.g., measurement and anatomic site in this case). Another non-limiting example of an encoded string with an appendage encoding might be “2C32&XH2CR0&XH5NL6&XA1Z38&XK9K&(XK5N)&17311&13132”. The range categorization in this example allows for selection of the correct procedure coding for the Mohs surgery (e.g., “17311” instead of “17313” for the body) and for the closure (e.g., “13132” which is a closure code for a repair on the forehead). Through language, code, symbols, images, text, visualizations, ranges, and categorizations, automatic descriptions and encodings are enabled by the system 985 and practical applications are illustrated by the embodiments in the teachings herein. In addition to creating automatic descriptions and encodings based on existing coding and classification systems, custom encodings for procedures and data points are also contemplated that can be cross-referenced to existing or newly developed coding and classification systems, with proprietary codesets (e.g., enabled by the knowledge base module 992) being used with requisite licensing. The embodiments illustrated generate custom encoded strings incorporating different concepts, data, procedures, visualizations, language, and descriptions. It is contemplated the system would serve the proprietary counterparts to those who are licensed to receive them (e.g., enabled by the knowledge base module 992 and/or other components of the system 985). It is contemplated that additional coding sets could be incorporated with the embodiments illustrated, for example, the OPCS Classification of Interventions and Procedures coding system published by the National Health Service for the United Kingdom. Detection of the appropriate code sets to assist with automatic encoding, translation, and visualization relies on detected data (like relevant country), license status, range categorizations related to the code sets, anatomy, procedure, diagnosis, visualizations, and other data (e.g., enabled by the knowledge base module 992 in communication with the data processing module 999 as one non-limiting example). Table 1 contains non-limiting example codes and descriptions of the codes that are copyrighted to the AMA, included for illustrative purposes only. The descriptions of the code contain specific anatomic sites. Ranges of anatomy in the exemplar embodied system 982 that allow interaction with this codeset, and ranges that allow access to custom encodings for procedures, interact with each other and other health data such as measurements or depths in the embodiments illustrated to generate the correct and desired outputs (e.g., enabled by the generation module 993).

TABLE 1

| | |
|---|---|
| 11312 | Shave removal of a single lesion face, ears, eyelids, nose, |
| | lips, mucous membrane 1.1 cm-2.0 cm |
| 17311 | Mohs surgery on Head, Neck, Hands, Feet, Genitalia, or any |
| | location with surgery directly involving muscle, cartilage, |
| | bone, tendon, major nerves, or vessels, First Stage, up to |
| | 5 tissue blocks |
| 13132 | Complex primary closure forehead, cheeks, chin, mouth, neck, |
| | axillae, genitalia, hands and/or feet; 2.6 cm to 7.5 cm |

Range types in the embodiments illustrated enabled by the system 985 are contemplated to be any combination of alphanumeric and numerical indices, collections of codes or language (or language components or a large language model), symbols, or image ranges, or coordinates such as those on maps, avatars, visualizations, or multimedia. As an example, ranges and collections of anatomy can be used to explain, visualize, and describe the locations or distributions on which to use a topical medication. In certain embodiments, “Under breasts and in groin folds” for a prescription for ketoconazole 2% cream for a diagnosis of intertrigo can generate encoded, translated, and visualized outputs based on the language description and range of anatomy it includes based on language and visualization. An emoji or symbolic description based on the ranges of anatomy can also be generated such as “down arrow emoji” and “bikini emoji” or symbols to signify that the medication should be used under breasts and in groin folds, as one example (e.g., enabled by the generation module 993).

In certain non-limiting embodiments of the system 985 that take advantage of range categorization, regimen maps can be saved and retrieved later for modification (eliminate or add products), progress (patient reported response), status updates, workflow automation (e.g., refills). Regimen maps that are generated by the system to describe and visualize “what to use where” (e.g., on a label in “mirror view” as described in the teachings herein) and correlating descriptions can be automatically translated to any coded or linguistic language. Active ingredients and alternatives can also be listed based on the recipient's language and country/location. For example, the same product might be known by a different name in Arabic. Or the same product might not be available in the Middle East, but a similar product could be available in the recipient's country. Certain embodiments enabled by the system 985 allow patients in one country to communicate through photos and text with physicians/professionals in another country in a language agnostic manner. For example, telemedicine consults and recommendations can automatically be performed, with a treatment regimen mapped to the patient images or an avatar, and the regimen can be automatically translated (e.g., with the generation module 993 in communication with the knowledge base module 992 as one non-limiting example) to the recipient's language and/or to a symbolic language such as Emojis. Product categorization can vary based on country and local regulations as well. For example, Tretinoin 0.05% cream or Hydroquinone 4% cream are prescriptions available at a pharmacy in the US; but they are available over the counter in Mexico. In certain embodiments, products and recommendations are categorized on where/how to acquire based on the recipient's location. Real-time syncing with local/online vendors, stores, in-office inventory, and pharmacy inventory can also provide patients with expected availability and pricing. Manufacturers may automatically link savings cards, coupons, or other promotions by storing them in the medium 995 and displaying them on the GUI 991 as one non-limiting example. Continuing this example, when in-office inventory is low on office-dispensed products, automatic re-ordering workflows can be initiated by the system 985. Warnings such as allergic reactions and expected side effects can be automatically applied to each recommendation in the regimen (e.g., enabled by the knowledge base module 992 and/or the record retrieval module 997). Patients often receive multiple recommendations, sometimes with 10 or more products listed, and can understandably get confused on “What to use where” and “When.” In certain embodiments, visual hierarchical mapping with anatomic site or site group descriptions can help to color code this information in an easily digestible out format for the patient, in any language (e.g., enabled by the output device 989). Since some products may be used in multiple anatomic sites/groups, we can show this potentially confusing information with a color coded map/image and also automatically broken down into different categories when generating the output (e.g., enabled by the generation module 993 and/or the output device 989) such as: Order/Frequency; By Product; By Area; By Condition. Additionally, for products in a multi-step regimen or to be used in a specific area, certain embodiments automatically prints physical labels that the patient can apply to their products. Labels might include product name, order/sequence information (like AM2 indicating this is step 2 in the morning), location(s) information, warnings, and other metadata about the product. The generated labels themselves can contain a highlighted, isolated or combined visual preview on an anatomic avatar, or the patient's image, showing exactly where to apply the product(s) or use the product(s). This physical label example shows the product, where to use it, what order to use it in, what it is used for, notes, and lists other areas of use. Since, in certain embodiments, generated physical labels are intended to be placed on topical product bottles, during the generation by the generation module 993, the labels can reflect the printed visualization to appear similar to what the patient will see when they look in the mirror. Symbol containing generated labels, such as those with emojis as one example, can also describe how to use the medication. (For example, a pill would automatically show a mouth from the hierarchical visualization with a cup of water); “Take with food” could show “food emoji”. “Take with fatty food” would include the “cheese” emoji. “Avoid dairy within 2 hours of this medication” for 2 hrs shown as “no emoji” and “cheese emoji” and there could be a symbolic representation of a clock showing the passage of 2 hours, in certain embodiments. While the paper workflow generates a digestible report that can be handed to the patient; the electronic regimen can be saved into the patient's chart (e.g., enabled by the database interface module 996) for tracking the regimen over time and initiation of other workflows. The electronic regimen stored on the medium 995 can evolve over time, with input from the patient and the professional (e.g., on separate or the same devices as the input device 988 in certain embodiments), in their preferred language. For example, in an electronic evolving regimen: the patient could give feedback on a product, report a side effect from a product, initiate a refill request for a product, find up to date manufacturer's coupons for a product, view product recall information, ask their professional about the product, report stopping the product, report starting a new product, or other electronic tasks (e.g. enabled by the input device 988 in communication with the GUI 991 and/or other components of the system 985). Automatic alerts could be sent to the patient and physician if there is a product recall.

An example of how the same anatomic sites might have different interactive ranges and categories is illustrated from several different datasets in the embodiments illustrated proprietary database, neural networks, and information systems. In certain embodiments of a range in the system 985, the nail plate of the thumb is represented by AMID 316; Parent 313; Hierarchical level 7; emoji group “hand emoji”; ICD code XA5V24; English description “Nail plate of thumb.” In another embodiment, the collection for “nail_plate” includes the ranges “316; 332; 348; 364; 380; 454; 470; 486; 502; 518” which includes the 316 identifiers with English semantic description “Nail plate of thumb” in a separate interactive collection. Still another separate range collection example, called the “cpt_nail_unit” includes 308-316; 324-332; 340-348; 356-364; 372-380; 446-454; 462-470; 478-486; 494-502; 510-518. The range collections of the embodiments illustrated are used to determine how the anatomic site interacts with engines related to billing, diagnosis, summarization, visualization, translation, and encoding of data. There are limitless examples of how these interactive ranges waterfall into one another and interact in an omnidirectional manner as enabled by the system 985.

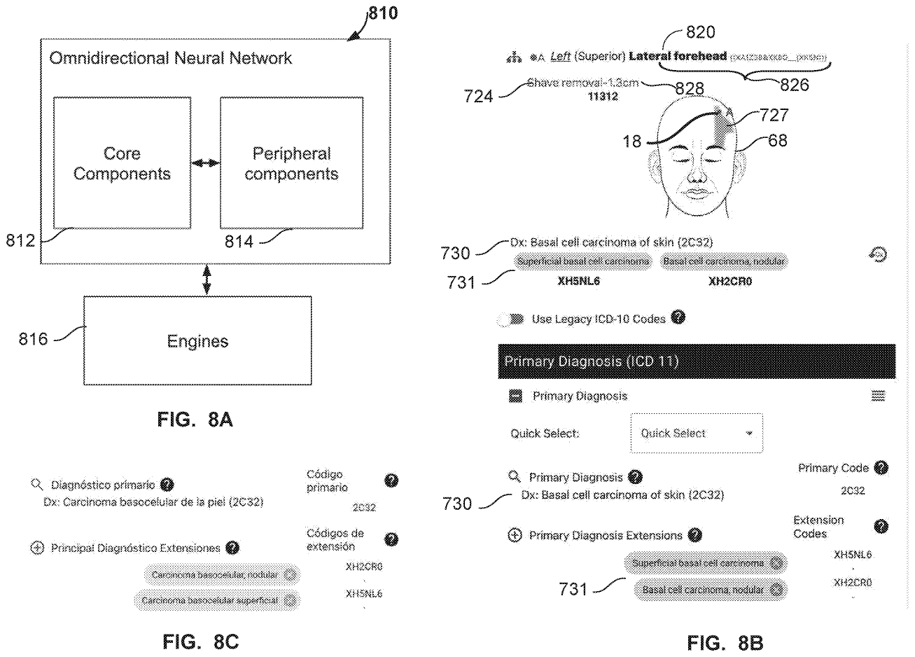

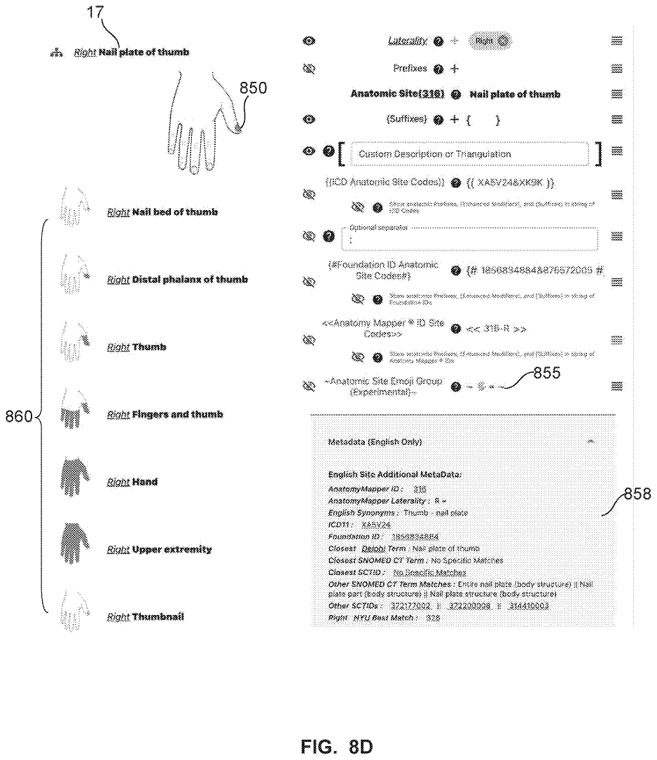

FIG. 8A illustrates a simplified block diagram of an omnidirectional neural network 810 enabled by the system 985 for ranges, collections, and categorizations of system 985 data. In certain embodiments, the system 985 data includes core components 812 and peripheral components 814. In certain embodiments, the omnidirectional neural network is configured for health data. Core components 812 include data such as anatomy, diagnosis, procedures, and other health data. Peripheral components 814 include data such as indices, codes, points, paths, maps, coordinates, cross-maps, descriptions, visualizations, and semantic and linguistic elements. The core components 812 within the omnidirectional neural network are organized in ranges, collections, and categorizations that enable omnidirectional connections allowing the various core components 812 to interact with each other in automated and new ways. Additionally, the peripheral components 814 are organized in their own unique ranges, collections, and categorizations and can communicate with one or more core components simultaneously forming the neural network in the system 985. The neural network 810 is able to determine the appropriate health data for the necessary engines 816, such as a billing engine, diagnosis engine, summarization engine, visualization engine, translation engine, or encoding engine (e.g., enabled by the data processing module 999).

In certain embodiments, indices in a range of anatomy plus semantic elements like a laterality of “right” linked to a diagnosis of actinic keratosis and a procedure of photodynamic therapy that uses a medical supply of aminolevulinic acid are automatically categorized, translated and encoded with any linguistic, symbolic, or coded language and also cross-mapped to other health data such as the National Drug Code for the aminolevulinic acid (e.g. enabled by modules on the medium 995 and/or components of the system 985). In certain embodiments, the omnidirectional neural network enabled by the system 985 uses ranges, collections, and categorizations to determine that the anatomy ranges and semantic components do not matter for the billing engine but do matter to the summarization and visualization engines. In another non-limiting embodiment, cryosurgery is performed for a diagnosis of warts on the penis and perigenital region. Other health data determines the patient is located in the United States and two different CPT billing codes (17110 and 54056) would be determined by the neural network enabled by the system 985 based on the anatomic range categorization of the warts being within the system's proprietary ranges of 233-245 and 246-248. In the current state of the art, manual selection of two different procedure types is necessary to document cryosurgery of warts in different billable anatomic areas, and therefore the user must have knowledge of these billing rules to correctly document these procedures in the United States. Since documentation may be delegated to less trained staff, charge capture, revenue capture, and appropriate billing may not be accurate, thus the system 985 improves the current state of the art and has practical application.

The range categorizations or relevance can be the same or different based on other health data, such as the OPCS procedure coding in the United Kingdom, which may treat the system's 985 ranges of 233-248 the same. Diagnosis data may also be presented in ranges, collections, and categorizations. For example, actinic keratosis may be categorized as pre-malignant, whereas the wart may be categorized as benign. Various subtypes of warts, such as verruca planae, verruca vulgaris, verruca plantaris, condyloma can all be categorized into a benign collection, with ranges or severity, and allowed on certain anatomic ranges. For example, verruca plantaris might only be allowed on the anatomy ranges consistent with the plantar feet (e.g., enabled by the knowledge base module 992 and/or other components of the system 985). Current systems use templates to determine correct codings, whereas certain embodiments of the system 985 use ranges, collections and categorizations that interact with one another to generate the outputs (e.g., enabled by the generation module).

Another non-limiting example of other data, which does not necessarily need to be health data, affecting the outputs would be country of the patient. Since different countries may use different versions (e.g., International Classification of Diseases, 10th revision versus 11th revision (ICD-10 vs. ICD-11)) of codesets or different codesets all together for diagnosis (e.g., enabled by the knowledge base module 992), an omnidirectional neural network can generate and display the appropriate encodings based on that data (e.g., enabled by the generation module 993 and/or the GUI 991).

Traditionally, these concepts exist in independent databases and data structures that may link one or two of the concepts together. However, ranging, grouping, categorizing, and collecting ranges of different concepts and codes in certain embodiments of the system 985 allow for enhanced accuracy and precision of description, language, and encoding in large language models 1103, language-vision models 1102, vision-language models 1101, and/or coordinated language model 980 engines. The coordinated language model 980 engine of the system 985 in certain embodiments is optimized with enhanced precision and accuracy through structured core and peripheral components that interact with one another. Omnidirectional neural networks and other information systems (such as relational databases (e.g., enabled by the database interface module 996)), along with interactivity of the ranges and collections, and the collections formed by certain embodiments of the system 985 enable machine learning and artificial intelligence to accurately bill, diagnose, summarize, visualize, encode, and translate data in a platform-agnostic, language-agnostic, codeset-agnostic manner. Additionally, the ranges, categorizations, and collections of certain embodiments of the system 985 are applicable to vision-language models 1101 and language-vision models 1102. That is, computer vision enabled by the system 985 can be applied to images, avatars, maps, videos, and diagrams to generate language-based and code-based descriptions of anatomy and other health data such as procedures and diagnoses; and the language-based descriptions of anatomy, procedures, diagnoses, and other health data (e.g. enabled by the input device 988) can be applied to generate visualizations, images, avatars, maps, videos, forms, records, and/or diagrams (e.g. enabled by the generation module 993).

FIG. 8B shows a screenshot of certain embodiments of the system 985 that use interactive range categorizations and data collections to correctly describe and encode anatomy 820, diagnosis 730, and procedure 824. Based on the coordinate range (e.g. point within the defined path in the illustrated embodiment) 18, anatomic site range (e.g. path name within a database or hierarchy of anatomy in the illustrated embodiment) 826, visualization range (e.g. anatomic site path relative to a diagram or map in the illustrated embodiment) 827, diagnosis range (e.g. skin cancer 730 and subtypes 731 in the illustrated embodiment), procedure range (e.g. shave removal associated with the forehead in the illustrated embodiment) 824, and other metadata (e.g. measurement of 1.3 cm in the illustrated embodiment) 828, certain embodiments of the system 985 are automatically able to describe (with natural language processing) and visualize a “Shave removal procedure of a Basal cell carcinoma, superficial and nodular type measuring 1.3 cm on the forehead” it as Left (Superior) Lateral and encode 2C32&XH5NL6&XH2CR0&XA1Z38&XK9K&(XK5N)&11312 applying two completely different code sets: ICD-11 and CPT in the illustrated embodiment. Changing one of the core components would change or persist other components depending on the change because the range categories can dynamically be re-processed through the different engines in certain embodiments of the system 985. It is contemplated that anatomic site ranges 826 may also be, as non-limiting examples enabled by the system 985, any combination of ranges or single range from coordinates on a map; point coordinate within a map relative to a map element or path; a point coordinate within a map relative to other map elements or the entire map; path coordinates relative to other paths, maps, diagrams, images, avatars; language in a hierarchy or database; encodings in a hierarchy or database; symbols within a hierarchy or database; and/or ids or indices in a hierarchy or database.

FIG. 8C is an example of a diagnosis and diagnosis extensions shown in Spanish with full translations and encodings before natural language processing enabled the system 985. Redundant language is removed by certain embodiments of the system 985 through natural language processing for enhanced human readability and understanding. Certain embodiments of the system 985 remove redundant language from coded descriptions and combines concepts into human and machine-readable descriptions. For example, the diagnosis and extensions in the Spanish exemplar of this figure would be condensed through natural language processing to read “Carcinoma basocelular de piel, tipo superficial y nodular” and would automatically encode to 2C32&XH2CR0&XH5NL6. In other words, the “Carcinoma basocelular” prefix is removed for two out of the three instances because it is describing the same diagnosis. The 2C32 diagnosis can be categorized in numerous ranges and collections as well, such as being a cutaneous carcinoma, a sun-induced carcinoma, and a carcinoma with a high cure rate. It is contemplated that exclusionary categories also may exist, such as “exclude this carcinoma from life insurance considerations” or “exclude this carcinoma from typical internal anatomy coding (e.g., provide a special warning that usually this would not be encoded with a deep anatomy term)” or “exclude this carcinoma from organs without an epidermis.”

FIG. 1S shows a screenshot of a visualization of an embodiment of ranges of anatomy that are linked to diagnosis and treatment recommendations. Anatomy areas of interest 17 are shaded differently to easily distinguish. In a color embodiment, the shading may be any color or pattern, opacity, or blended color or patterns. Ranges of anatomy are described with English language in this embodiment, but can be described in any coded, linguistic, or symbolic language.

Another embodiment of anatomic site ranges broken up into distribution segments is translated into Spanish with natural language processing, and linked to a diagnosis by certain embodiments of the system 985 described herein (e.g., enabled by the data processing module 999). Translation into other languages such as Chinese, Arabic, Punjabi, Greek, Ukrainian, Hebrew and any other linguistic language that uses non-roman characters, right-to-left instead of left-to-right language, or other alphabets and numbering systems applies to certain embodiments of the system 985 described herein. Language specific considerations are also accounted for by certain embodiments of the system 985, such as masculine versus feminine terms for laterality in Spanish (e.g., enabled by the knowledge base module 992). Additionally, the system 985 embodied herein can update, evolve, and improve when new data and translations become available, such as new or updated translations from other data sources. Such evolution can occur with machine learning, deep learning, neural network, or information systems improvements through automated means enabled by modules in the medium 995 and/or other components of the system 985.

FIG. 8D shows a screenshot of the visual ranges 860 of anatomy under a given point or site 850 which has an invisible pin associated with it in this embodiment as part of a hierarchical painting and selection application enabled by the system 985. The visual definition of the anatomic site 850 is shown along with the term 17. The visual ranges 860 show the alternative anatomic site descriptions along with the visualizations and are shown relative to the right hand in this embodiment. Additional contemplated certain embodiments of visualizations include but are not limited to bilateral nail plates of thumb, all nail plates of right hand, bilateral visualization of any of the shown sites (both sides), bilateral visualization of both upper extremities, and visualization of all extremities (e.g., enabled by the GUI 991). Certain embodiments can also mirror or otherwise alter the axes of visualization generated by the system 985 (e.g., enabled by the generation module 993). The possibilities are endless and build upon anatomic site ranges, categorizations, and collections along with other data categorizations such as laterality. Additionally, in the example embodiments, an anatomic site emoji 855 is assigned and it is contemplated different categories exist for different anatomy concepts, such as the “arm emoji” being used when the upper extremity is selected instead of the “hand emoji” as used to categorize the other terms in the illustrated embodiment. The illustrated embodiment also uses range categorization and collections to determine cross-mapping relationships 858 to other terminology sets like ICD-11, and custom proprietary encoding to enable the downstream engines to automatically bill, diagnose, summarize, visualize, translate, and encode. While this figure illustrates anatomy examples, examples of non-anatomic visualizations might be photos, videos, or descriptions of procedures; procedure metadata like measurements and counts, and descriptions of morphology generated by computer vision enabled by the system 985 or by the user (e.g. enabled by the input device 988) plus descriptions of symptoms to render a diagnosis with the diagnosis engine (e.g. enabled by the knowledge base module 992 and/or data processing module 999).

In another example embodiment of collections of surface anatomy under a cursor or point of interest (e.g., enabled by the input device 988), the anatomy can be filtered based on desired characteristics. For example, sometimes less granular collections may be desirable based on the practical application use case, so filtering by collection, range, or category allows for the desired interactivity and minimizes distractions when precision is less critical, such as in distribution mapping rather than pin-point mapping. In one example, the color coded legend can correlate with the filter for visualization of the filter (e.g., enabled by data processing module 999, the generation module 993, and/or the GUI 991 as non-limiting examples). Filters can be applied dynamically, on demand, or hard-coded for maximum benefit to the use case.

In another embodiment, a method to group anatomic sites into different interactive data comprising ranges, categories, relationships, visualizations, and collections.

In certain embodiments, the data interacts with diagnosis ranges, categories, relationships, visualizations, and collections. In certain embodiments, the data interacts with procedure ranges, categories, relationships, visualization, and collections. In certain embodiments, the data interacts with other data in ranges, categories, relationships, visualization, and collections. In certain embodiments, the interaction of the data forms an omnidirectional neural network that interacts with modules in the system 985 comprising engines for automatic billing, diagnosis, summarization, visualization, translation, and/or encoding. In certain embodiments, the data can be translated into any coded, linguistic, or symbolic language. In certain embodiments, the data can be filtered and/or belong to more than one categorization simultaneously. In certain embodiments, the translations of proprietary codesets, language, and data can be custom encoded and only served to those who are licensed.

In another embodiment, a system to group anatomic sites into different interactive data comprising ranges, categories, relationships, visualizations, and collections.

In certain embodiments, the data interacts with diagnosis ranges, categories, relationships, visualizations, and collections. In certain embodiments, the data interacts with procedure ranges, categories, relationships, visualization, and collections. In certain embodiments, the data interacts with other data in ranges, categories, relationships, visualization, and collections. In certain embodiments, the interaction of the data forms an omnidirectional neural network that interacts with the non-limiting embodied application system 985 described in the teachings herein which can include engines for automatic billing, diagnosis, summarization, visualization, translation, and/or encoding. In certain embodiments, the data can be translated into any coded, linguistic, or symbolic language. In certain embodiments, the data can be filtered and/or belong to more than one categorization simultaneously. In certain embodiments, the translations of proprietary codesets, language, and data can be custom encoded and only served to those who are licensed.

(APP09) Traditionally, areas of interest on an anatomic map, avatar, image, or video, such as pins, points, segments, and regions, may be highlighted but such highlighting reflects manually entered and static data. Additionally, the areas of interest in traditional technology exist as static (non-dynamic) points in two-dimensional or three-dimensional space with no ability to precisely and reproducibly describe, isolate, group, relate, order, target, translate, transform, merge, categorize, convert, or modify the area of interest as it changes through space and time, e.g., with spatial computing. The embodiments of the system 985 illustrated place dynamic, isolated, transformable, translatable, and targetable areas of interest onto anatomic maps, avatars, images, and videos and onto unmapped void spaces.

Each area of interest exists as its own dynamic “data island” represented by a visualization, description, optional position on multidimensional maps and void spaces, associated features and properties. A generated visualization (e.g. enabled by the generation module 993 and/or the GUI 991) associated with an area of interest may include a pin, pin type, pin order, pin description, coordinates relative to a map, coordinates relative to a map segment, coordinates relative to other areas of interest, relational information, void space information, attached data, cross-linked data, diagnostic information, morphological information, symptoms, historical health information (e.g. enabled by the record retrieval module 997), and other data as one skilled in the art would know. Each area of interest in the embodiments illustrated is describable, isolatable, groupable, relatable, orderable, targetable, translatable, transformable, mergeable, categorizable, convertible, and modifiable.

Certain embodiments of the system 985 are illustrated by means of an example describing two areas of interest represented as “pins” on a diagram on a map element. The areas of interest as pins and text can be shown or hidden on the map (e.g., made invisible or visible) (e.g. enabled by the GUI 991); related to one another; isolated from one another; grouped to each other; associated with diagnoses, morphologies, symptoms, tags, detections, attachments, multimedia, procedures, encodings, and other health data; the same or different colors; individually targeted (e.g. for modification, association, categorization, or isolated visualization or finding its location on a map); group targeted; reordered; reproduced in a new session; automatically encoded with anatomy, diagnostic, procedural, and other encodings; automatically translated into any coded, symbolic, or linguistic language; described with optional enhancements such as those describing the direction of the pin relative to the anatomic sites above and below the pin; and moved, translated or transformed dependently or independently in relation to coordinates, axes, data-based positions, organ systems, functional systems, cross-mappings, synonyms, slang, symbols, emojis, diagnosis, category, data memberships, and other data as non-limiting examples as one skilled in the art would know. The system 985 is a substantial improvement on traditional systems that use static areas of interest, which have issues with re-ordering, re-categorizing, and segregating different data components. For example, in traditional electronic health records (EHRs), if a procedure's anatomic location, order, or type is changed (such as a biopsy order or type, e.g., enabled by the input device 988), the associated photos, documentation, notes, and other content must also be deleted and re-documented. For example, with existing EHRs, if four biopsies are recommended with areas of interest being: A-shave biopsy; B-shave removal; C-punch biopsy; and D-punch excision, but the patient refuses to have the procedure on locations A and C, the system would require the documentation to start over-causing frustration and potential for errors and data loss in the documentation process. In the embodiments illustrated of the system 985, the areas of interest could be re-categorized and B would become A, and D would become B. Further continuing this example, if the procedure type for original D (now B) was initially entered incorrectly, and is actually a shave removal, the system of the embodiments illustrated could change the targeted data point in the area of interest file without risking data loss or time-consuming manual tasks of re-association of data. Existing solutions also do not have dynamic areas of interest that can be associated with changing or evolving optional anatomy data, changing procedural and diagnostic data, photos, images, videos, morphologies, visualizations, symptoms, and other data at different time points. In certain embodiments, pin-based and invisible-pin-based documentation occurs simultaneously, and the pins serve different functions, have different graphical representations (e.g., enabled by the GUI 991), and can travel through multidimensional space while maintaining relationships with each other, its contained data, and with other anatomic sites (e.g., enabled by the medium 995 and/or other components of the system 985).

In certain embodiments, the area of interest (AOI) represented by a pin is a separate scalable vector graphic (SVG) that has its own coordinate system (e.g., enabled by the data processing module 999 and/or the database interface module 993) and structure that is transposed on a map that itself contains mapped and unmapped regions. The AOI contains its own coordinate systems and structures; thereby allowing the AOI to exist by itself and also co-exist in relation to unlimited coordinate systems under, above, around, and related to it and in relation to data-driven relationships and connections enabled by the system 985. The AOI size, scale, rotation, angle, and topology can also be variables to relationships, surface areas, intensities, and other data. It is contemplated that an AOI can also belong to multiple neighboring anatomic sites defined by paths simultaneously and is represented by a drawn polygon, curve, path or shape (e.g., enabled by the medium 995). It is also contemplated that an AOI can have several non-obvious but practical invisible connections to other AOIs or other health data, such as through data-driven relationships or invisible anatomic map layers to other organ systems or functional systems (e.g., enabled by the system 985).

In another non-limiting embodiment, the AOI is a hidden and invisible SVG file that is nested into a multidimensional map with coordinates associated with at least one dimension of the map. In this embodiment, the AOI can be thought of as an “invisible pin” that can travel through map elements and void space of the system 985; and in order to allow for precise and visual travel, this “invisible pin” can temporarily be made visible (e.g., enabled by the GUI 991) during the travel process. In the embodiment, the dimension and anatomic site in which the pin resides can be color, patterned or highlighted providing a visualization of its current dynamic anatomic address.

AOIs have their own coordinate system and exist independently from a map, allowing the AOIs to travel through two-dimensional, three-dimensional, and multi-dimensional space and time on a map and to travel through void, unmapped space. It is contemplated that within the coordinate system, various data components stored in the medium 995 can be organized and selectively shown. For example, the description of the AOI can change over time and by different users, with each description containing its own targetable metadata (e.g., an audit log or history stored directly within the AOI enabled by the system 985, rather than on a separate database, as non-limiting practical applications) stored on the medium 995. AOIs can also exist in unmapped void space to visualize dynamic health information which does not have associated map or anatomy data. AOIs also have their own data sets associated with them related to patient demographic information, images, multimedia, reports, diagnoses, procedures, codes, descriptions, translations, and other information as demonstrated in the figures. AOIs are also movable, mergeable, modifiable, reproducible, searchable, and relatable based on the patient information, diagnosis information, procedure information, health information, positional information, data-based information with or without a map-based coordinate and/or anatomic visualization, as non-limiting examples. Additionally, AOI-coordinate systems can interact with map-based coordinate systems that are above, below, neighboring, or nearby the AOI coordinate system, and the AOI-coordinate systems can be independently targeted and modified, enabled by the system 985.

Targeted, dynamically changeable AOIs that have order and can be grouped, segmented, and tracked as separate islands (e.g., files or databases, e.g., enabled by the database interface module 996) of information that can interact with map and void space information have practical applications beyond anatomic mapping. In a geographic context for a military application, for example, it is contemplated that the land could be mapped in greater detail than the sea, and the sea is correlated with the void space as previously described. It is contemplated that the AOIs could correlate with military troops that contain their own properties and personnel files and skillsets, and these can be ordered, re-ordered, and tracked dynamically through space and time. Continuing this example, the AOI could be an asset like a land/sea vehicle or aircraft carrier; an AOI could be stealthily assigned to a region with an invisible AOI that has separate permissions and classification statuses; time-sensitive decisions can reorder assets in real time; and this vehicle or aircraft carrier acts as its own independent filesystem and database that has its own coordinate system to navigates the interiors and compartments within the vehicle or carrier; personnel inside the vehicle or carrier interact with the vehicle or carrier's coordinate system to determine their position within the vehicle or carrier; and personnel inside the vehicle or carrier have anatomic maps with their own anatomic coordinate systems associated with their personal health file. The AOI is its own file, database, or “data island” but it interacts with available mapping and tracking data.

In other certain embodiments like in a sports context, it is contemplated that the AOIs could be sports players on a map of a sports field. A group of AOIs would make up a team. The statistics on the AOI change dynamically over time as the AOI is tracked on a field map. It is contemplated that wearable or portable technology such as watches, glasses, mobile phones, or other devices can serve as a dynamic AOI or segment of a dynamic AOI, and interact with map data and void spaces, whether those maps be anatomic, geographic, or situation specific. Another example would be asset tracking where each AOI is an asset with its own bucket of information that interacts with an office map, schedule, and other data. As another example, if an AOI is a particular medical laser asset that has usage logs, patient treatment logs, scheduled patients for use, scheduled physicians who are using the asset, physician training logs and certificates for those qualified to operate the asset, payment logs, before and after photos, operator manuals, warranty information, service representative contact information, patient instruction sets, special electrical plug-in requirements, special warnings (e.g., no use in room with windows), setting recommendations for different parts of the body and different skin types and skin tones, safety notices like particular eye safety goggles needed with different wavelength and optical density settings, and other information relevant to the asset. Having all of the asset AOI information in the contemplated example interact with an office map dynamically joins relevant information to one place and interacts with specific rooms on the office map that can support the AOI, such as those with special electrical requirements. The teachings herein demonstrate non-limiting practical applications of the system 985.

Dynamic AOIs can also be thought of as buckets or files that contain segments of information (e.g., enabled by a tangible storage medium 995). Segments and files can belong to multiple buckets or dynamic AOIs simultaneously. They can be structured or unstructured, and serve as their own database, file system, and coordinate system. It is contemplated that each AOI can be its own database (e.g., enabled by the database interface module 996) that interacts with two-dimensional maps, three-dimensional maps, multidimensional maps, map elements, and void spaces enabled by the system 985. It is further contemplated that dynamic AOIs may also be translated in whole or in part into any coded, linguistic, symbolic, or multimedia based language. For example, a visualization SVG file for the AOI for a biopsy contains information that interacts with a map based on its position on the map and may also contain procedure information, coded information, and order information directly in the AOI file. The AOI may be translated to Chinese, in part or in whole, as one example, while the map data and data-based relationships remain in English. Procedure codes such as the Current Procedural Terminology (CPT) codes may be calculated and generated based on combining AOI data and map data, as another example (e.g., enabled by the data processing module 999, the knowledge base module 992, and/or the generation module 993). AOI position on a map combined with AOI diagnosis code data may modify the diagnosis code and anatomy coded result. The description of position of the AOI over the map can be translated to Chinese, remain in English, or shown and interacted with in any coded, linguistic, symbolic, or multimedia (such as images) based language. Still other benefits and advantages of the embodiments will become apparent to those skilled in the art to which it pertains upon a reading and understanding of the teachings herein.

In FIG. 9A the representative dynamic Areas Of Interest are biopsies as non-limiting examples enabled by the system 985. The AOIs are represented in the embodiment as “pins” on a multidimensional anatomic map diagram 910 enabled by the medium 992 and/or other components of the system 985. The general anatomic location of the pins can be described, in English, as the “left central cheek.” The first pin 912 is described and appears with a text label 13 as “A-Shave biopsy-r/o BCC” and the second pin 914 is described and appears with a text label 13 as “B-Punch biopsy-r/o MM” as non-limiting examples (e.g., enabled by the GUI 991). The pins and text labels can be shown or hidden on the map (e.g., made invisible or visible) through use of the visibility toggle 503 (e.g. enabled by the input device 988 in communication with the GUI 991); related to one another through automatically assigned relationships 54 (e.g. enabled by the data processing module 999); isolated from one another shown as isolated visual previews 68 in the illustrated embodiment (e.g. enabled by the GUI 991); grouped to each other; associated with diagnoses, morphologies, symptoms, tags, detections, photos 97, attachments 98 (e.g. enabled by the input device 988), multimedia, procedures, encodings, and other health data; the same or different colors; individually targeted (e.g., for modification, association, categorization, or isolated visualization or finding its location on a map); group targeted; reordered (e.g., A becomes B and B becomes A); reproduced in a new session (e.g. enabled by the record retrieval module 997); automatically encoded with anatomy, diagnostic, and other encodings; automatically translated into any coded, symbolic, or linguistic language (e.g. enabled by the knowledge base module 992, generation module 993, data processing module 999, and/or other components of the system 985); described with optional enhancements such as those describing the direction of the pin relative to the anatomic sites above and below the pin; and moved, translated or transformed dependently or independently in relation to coordinates, axes, data-based positions, organ systems, functional systems, cross-mappings, synonyms, diagnosis, category, data memberships, and other data as one skilled in the art would know.

In this embodiment enabled by the system 985, the first pin 912 is its own dynamic image file (e.g., an SVG file) and includes targetable data elements 918 (e.g., enabled by the tangible medium 995). These example targetable data elements 918 in the illustrated embodiment include but are not limited to: a point in 2D, 3D, or multidimensional space (e.g., the center of first pin 912); an associated procedure (e.g., “shave biopsy” for the first pin 912); color, pin type, order type, order, and grouping; associated diagnosis; associated data buckets for images/multimedia, links, forms, health data, patient info; pin and point properties, cross-mappings, and metadata (e.g. enabled by the tangible medium 995); pin descriptions; automatic relationships; targeting and unique IDs; isolated pin visualization combined with anatomy visualization; and visibility with the pin being visible. The data elements 918 are all dynamically linked to a separate AOI file (e.g., a system within the system 985), in this non-limiting case the SVG file, that has its own coordinate system and an anchoring point that interacts with mapped and unmapped (void space) regions on an anatomic map in the illustrated embodiment. In certain embodiments, shading applies an additive color or pattern sequence to show the different anatomy map elements 12, which have different dimensions, to show which sites the first pin 912 and second pin 914 belong to simultaneously. Here the first pin 912 and the second pin 914 exist simultaneously in the medium 995 on the left central cheek, left cheek, face, head, and head and neck in this embodiment. The AOIs also exist simultaneously in deep anatomy (e.g., enabled by the medium 995) that is not visible in the illustrated embodiment (e.g., enabled by the GUI 991), such as the fat pads and muscles underlying the first pin 912 and the second pin 914. Automatic relationships 54 (e.g., enabled by the data processing module 999) are also shown, specifically here “A (this pin) is Medial and Superior from B” and “B (this pin) is Lateral and Inferior from A.” The individual AOIs are associated with data that is optionally dependent and relatable to the map enabled by the system 985.

FIG. 9B shows an anatomic map 910 on the left that a user would see (e.g., enabled by the GUI 991) with the same pins from FIG. 9A. This anatomic map has an additional AOI associated with the same general anatomic location but the point associated with the AOI is invisible to the user on the map showing pins 910 while the color shading in the illustrated embodiment has been dynamically targeted to visually represent an anatomic site, the left central cheek, in the illustrated embodiment 910 (e.g., enabled by the GUI 991). The targetable properties 918 for this distribution segment may include in a non-limiting list: point in 2D, 3D, or multidimensional space; associated procedure; color, pattern, grouping; associated diagnosis; associated data buckets for images/multimedia attachments, links, forms, health data, patient info; pin descriptions (visible only when pin is also visible); automatic relationships; targeting, unique IDs; Isolated pin visualization combined with anatomy visualization (when pin is made visible in the current embodiment); Invisible (on left of figure, made visible on right of figure and also made pins A and B invisible on the rightmost portion of the embodiment as one example). In certain embodiments, the invisible AOI 925 can be made visible (e.g., enabled by the input device 988) as shown in the second anatomic map 926 shown on the right for reference and graphically depicted as an anchor (e.g., enabled by the GUI). Making the invisible AOI 925 temporarily made visible enables move workflows to relocate the anchor to different coordinates. The invisible AOI 925 can also keep the same coordinates and move through different map dimensions with a hierarchical selector 505 (e.g., enabled by the input device 988 and/or the GUI 991 as non-limiting examples); in which case it would synchronize map coloration to the visualizations on the map and isolated visual preview with a new selection point of anatomy. The invisible AOI 925 is also its own dynamic image file and includes targetable data elements 915. As with visible AOIs, data elements are all dynamically linked to a separate AOI file that has its own coordinate system and an anchoring point that interacts with mapped and unmapped (void space) regions on an anatomic map. The invisible AOI 925 also exists simultaneously on the left central cheek, left cheek, face, head, and head and neck, (e.g., enabled by the medium 995) but is shown with only the left central cheek shading in this embodiment.

FIG. 9C shows an anatomic map 10 with AOIs from FIG. 9A reproduced at a different point in time with dynamic changes to pin properties, descriptions, diagnosis, color, categorization, and other properties (e.g., enabled by the system 985). The first pin 12 received a pathological diagnosis of Basal Cell Carcinoma (e.g., enabled by the input device 988), so the AOI was kept in a constant map position and the properties were dynamically updated at a different time point. The second pin 14 received a pathological diagnosis of Melanoma, so the AOI was kept in a constant map position and the properties were dynamically updated at a different time point. Each AOI can be changed dynamically therefore these two AOIs can be grouped into a “skin cancer” group as one example. Additionally, if first pin 12 was accidentally stamped on the wrong anatomic site, such as the wrong side and it should have been stamped on the “right central cheek”, the entire AOI can be moved or refined (e.g. enabled by the input device 988) without data loss or corruption, and only dynamically affecting the position of the AOI and the generated anatomic description of its position (e.g. enabled by the generation module 993). In other words, all documentation can follow the AOI, and is dynamically changeable with or without map interaction enabled by the system 985.

In another embodiment, the AOI is its own file or data island with its own coordinate system and properties that dynamically interact with underlying, overlying, and nearby coordinate systems, file systems, and map elements. In such an embodiment, the AOI has a nested file containing its own coordinate system and properties (thus forming a system, within a system, e.g., enabled by the system 985 and its components such as the tangible medium 995 in communication with the processor 986). The different files can have associated orders, properties, and segmented structured data. The coordinate systems are relatable to map elements, void spaces, and to other AOIs. It is contemplated that each AOI can have nested AOIs that relate to and interact with self, other AOIs, map elements on maps and avatars, and void spaces enabled by the system 985. It is further contemplated that each AOI can act as its own database or file system, which can interact with itself, other AOIs, map elements on maps and avatars, and void spaces (e.g., enabled by the database interface module 996).

In the system 985 and as a non-limiting example, components of the separate file and AOI can be modified (e.g., enabled by the medium 995), such as a component of a pin description as shown on the map. It is contemplated that other properties are also modifiable, such as color, pin type, pin size, surface area, and position of file. The AOI interacts with itself, with nearby AOIs, with its group, and with underlying map elements and void spaces enabled by the system 985.

FIG. 9D shows an anatomic map 10 with a void space AOI 30 that is in a void unmapped space enabled by the system 985, represented here by an encapsulated sequence (1) pin type and not located on an anatomic site. In this example, the diagnosis of “Essential hypertension” is not associated with a specific anatomic site or on a mapped area. The AOI still has its own file structure including buckets to store health information 32, such as photos, attachments, links (e.g., enabled by the input device 988), cross-links, forms, diagnosis, order, pin type, color, groupings, and other properties. Digital and other assets enabled by the system 985 can belong to, or be copied to, or be removed from one of many buckets simultaneously. In certain embodiments, a single photo may have multiple detected anatomic sites and AOIs in it. Another non-limiting example would be a pathology report or a progress note generated by the system 985 that has multiple sites on it (e.g., enabled by the generation module 993). The report belongs to multiple data buckets (e.g., enabled by the tangible medium 995) simultaneously, and continuing this example, each component in the report can belong to separate data buckets or groups of data buckets and be targeted independently from the other components (or dependently targeted when desired).

AOIs located in void spaces enabled by the system 985 can be moved or assigned to mapped locations (e.g., enabled by the input device 988). FIG. 9E shows the void space AOI 30 from FIG. 9D moved to a mapped location. The mapped AOI 34 in FIG. 9E has been assigned to a specific location and all data associated 32 with the AOI 34 has been moved from the void space to a mapped area. The AOI 34 interacts with the anatomic map 10 to generate a description of the anatomic location (e.g., enabled by the generation module 993), here indicating the anatomic location at which the blood pressure measurement was obtained in the illustrated embodiment: the left upper arm.

AOIs can be reordered (e.g., enabled by the input device 988) while maintaining the data associated with the respective AOI. FIG. 9F shows the areas of interest from FIG. 9A reordered enabled by the system 985. The procedure type, diagnosis, descriptions, and associated map positions remained constant. Only the order was dynamically targeted and changed. The first pin 912 is now “B” and the second pin 914 is now “A” but the text labels remain the same as depicted in FIG. 9A. The automatic relationships 54 (e.g., enabled by the data processing module 999) were also automatically described and updated. While in FIG. 9A, the relationships were described as “A (this pin) is Medial and Superior from B” and “B (this pin) is Lateral and Inferior from A.”, in this Figure, the relationships are described as “A (this pin) is Lateral and Inferior from B” and “B (this pin) is Medial and Superior from A.” The flexible system 985 and/or method like the embodied examples prevents data loss and minimizes confusion and the need to restart documentation.

Sometimes selecting a pin or moving a pin, such as on a touch screen (e.g., enabled by the input device 988), can be a challenge at different zoom levels and screen orientations. Some non-limiting examples of how certain embodiments of the system 985 presented herein improve usability include: differential zooming on the map (the pins stay the same size but appear to be getting further apart when zooming in for example); touch-point halos around the pins such as during a select workflow (e.g. enabled by the GUI 991 and/or the input device 988) to improve usability as shown around a selectable AOI 925 in certain embodiments, as exemplified in FIG. 9B. Another example to improve usability during move workflows, which work with touch screen and mouse input by default, is by adding a joystick and mini-map navigation (e.g., on a touchscreen enabled by the input device 988). The joystick may be digital on the screen or a physical device. Other input devices may also be used. A minimap makes larger touch points on a zoomed in area of the map, especially useful on the GUI 991 on mobile phones, to improve the touchscreen experience as one example.

Certain embodiments of the system 985 allow for axial mirroring, rotation, reflection, and scaling (e.g., enabled by the GUI 991 and/or the data processing module 999); so, the pins could be shown in mirror view as one example, in isolation or in combination with other pins and findings. A reflected view is useful when a patient is looking at their anatomy in the mirror or on a “selfie” camera, as a single example of many. The AOIs maintain their correct positioning even when the anatomic maps and visualizations are reflected such that even the descriptions describing the relationships between pins remain accurate. When shown a reflected view 426 the automatic relationships 54 would still display true: “A (this pin) is Lateral and Inferior from B” and “B (this pin) is Medial and Superior from A.” as one non-limiting example.

AOIs may also be presented in translated versions as well, into any coded, linguistic, or symbolic language (e.g., enabled by the knowledge base module 992 in communication with the data processing module 999 as a non-limiting example). Some components may remain in the language or input type they were initially entered in with, depending on user preference and as an example. Roman characters (e.g., A, B, C) can remain to show order such as in a collaborative session with multilingual participants (e.g., enabled by the medium 995, the output device 989, and/or other components of the system 985), but the remaining text can be dynamically targeted and updated, while maintaining the pin positions on the anatomic maps and hierarchical painting and health metadata relationships. Natural language processing, also describable as a natural semantic sequence, can be applied to targeted or all components of certain embodiments of the system 985 (e.g., enabled by the data processing module 999).

In one embodiment, a computer implemented system for defining an area of interest compromising: the processor for hosting a defined map or image of containing at least one area of interest wherein the defined map or image has a coordinate system; an area of interest wherein the area of interest has a coordinate system, an associated data set, dynamic properties, and at least one tracking point; wherein the coordinate system of the area of interest is independent from the coordinate system of the defined map or image; and a non-transitory computer readable medium, storing machine executable instructions executable by the processor, the machine executable instructions configured to receive input wherein the input identifies a location or region on the map or image to associate with the area of interest.

In certain embodiments, the area of interest can be nested into file systems and databases, interact with file systems and databases, and contain their own file systems and databases. In certain embodiments, the area of interest is movable, mergeable, modifiable, reproducible, searchable, and relatable based on the associated coordinate system and data set. In certain embodiments, the area of interest exists simultaneously in multiple dimensions of the map or image. In certain embodiments, the tracking points are related to diagnosis, diagnosis category, procedure, procedure counts, measurements, and calculatable and analyzable metadata. In certain embodiments, the area of interest is located within an unmapped void space while maintaining its own coordinate system, data, and dynamic properties. In certain embodiments, the data sets relate to patient demographic information, images, multimedia, reports, diagnoses, procedures, codes, descriptions, translations, and other medical information. In certain embodiments, the dynamic properties are modifiable. In certain embodiments, the area of interest coordinate system can be independently targeted and modified. In certain embodiments, the coordinate systems can interact with each other. In certain embodiments, the system comprises at least two areas of interest wherein one area of interest is not visible and wherein said invisible area of interest can select, relate, and modify visible map areas, define intensity and define overlap on the map.

In one embodiment, a system of a coordinated language model engine, comprised of at least three of the following components: a database that contains linguistic terms for anatomy; a database that contains linguistic terms for anatomy; laterality, prefixes, suffixes, directional modifiers, groupings, hierarchical relationships, relationships, collections, morphologies, cross-mappings, diagnoses, symptoms, procedures, tags, slang, synonyms, or translations; a database that contains encoded information anatomy, laterality, prefixes, suffixes, directional modifiers, groupings, hierarchical relationships, relationships, collections, morphologies, cross-mappings, diagnoses, symptoms, procedures, tags, slang, synonyms, or translations; a database that contains symbolic information anatomy, laterality, prefixes, suffixes, directional modifiers, groupings, hierarchical relationships, relationships, collections, morphologies, cross-mappings, diagnoses, symptoms, procedures, tags, slang, synonyms, or translations; a map defined by two or more defined, coordinated paths of different sizes wherein the paths are automatically relatable to one another through directional planes; a map defined by two or more defined, coordinated paths of different sizes wherein the paths are automatically relatable to one another through custom axes; a map defined by two or more defined, coordinated paths of different sizes wherein the paths are automatically linguistically segmented and described with enhanced directional modifier terms; multimedia input including a visualization, image, illustration, avatar, photograph, or video that contains anatomy; verbal, written, selected, typed, extracted, symbolic, or detected input that describes at least one of the following: anatomy, modifiers of anatomy, morphology, symptoms, modifiers of health data, or other symbol-delimited or symbol-defined health data; vision-language models, language-vision models, or language models that can be used alone or in combination to describe, relate, track, target, collate, organize, visualize, display, translate, anatomy, morphology, symptoms, treatment regimens, or health-related findings; multimedia output including a visualization, image, illustration, avatar, photograph, or video that displays anatomy in isolation, combination, or relation to self, other anatomy, or other areas of interest; alerts related to anatomic site or health information associated with an anatomic site such as a procedure or treatment recommendation; generative capabilities to create targeted visualizations, anatomic maps, anatomic descriptions from mixed visual, language, coordinated, or uncoordinated inputs that contain anatomy.

In another embodiment, a system for a search engine to improve healthcare data usability, the system configured to: utilize at least one of and any combination of language models, language-vision models, vision-language models, or coordinated language model type models and/or engines; find, collate, modify, organize, relate, aggregate, visualize, target, analyze, calculate, summarize, communicate, encode, translate, map, cross-map, track, or display health information wherein the health information is related to anatomy data or related to non-anatomy data.

In another embodiment, a computer enabled method for identifying, visualizing, describing, relating, linking, and recording data performed medical procedure at a specific anatomic location comprising: displaying a graphical user interface 991 with a multimediagraphic representing the anatomical region of a patient and/or an input prompt; identifying at least one specific input coordinate based on the defined anatomic location on the displayed graphic graphical user interface 991 representative of a location on the patient; generating at least one visualization output or translation output of the identified input defined anatomic location wherein the generated output is displayable, describable, relatable, translatable, and sequenceable with any combination of linguistic, coded, or symbolic terms (or data) related to the anatomic location; inputting a patient specific data comprising of at least one of a selected procedure, diagnosis, morphology, treatment recommendation, multimedia, symptom, or finding on the patient at said input defined anatomic location; retrieving from a knowledge base storing a plurality of templates, each comprising a set of fields or anatomic visualizations, with a first template of the plurality of templates having a different set of fields than a second template of the plurality of templates wherein said templates each identify a unique event or location attribute; selecting an associated one of the plurality of templates according to each of the selected event and the location on the described or displayed or representative anatomical region; populating the set of fields associated with the selected template, at least one of the set of fields comprising translated and character-based description or a visualization describing the selected event and the location on the described or displayed representative anatomical region; filling at least one of the set of fields with data related to the procedure, diagnosis, morphology, treatment recommendation, multimedia (including a photograph, video, diagram, attachment, or link), symptom, finding, patient data, clinic data, encounter data, photographs, links, attachments, videos, diagrams, additional multimedia items, or other patient specific data wherein the multimedia has maps overlaid or underlaid with defined and relatable map data; first multimedia item associated with the anatomical region of the patient taken prior, during or after the selected procedure and additional multimedia items associated with the anatomical region of the patient wherein the multimedia items have maps overlaid or underlaid with defined, translated, encoded, and optionally related terminology; linking the selected templates wherein said inputs become a combined record; generating a digital or printable label for the combined record listing at least the anatomical region, a name of the patient, a visualization or multimedia, and a medical record number; formatting the combined record into a database record suitable for an electronic health records database associated with the system; and filling at least one of the set of fields with a multimedia item associated with the anatomical region and the selected procedure wherein the multimedia items have maps overlaid or underlaid with defined, translated, encoded, and optionally related terminology; formatting the combined record into a database record suitable for an electronic health records database; generating a complete patient history of the anatomical region wherein said specific coordinate defined or input defined anatomic locations within the anatomic region are defined according to multiple custom axes, direction planes, and data-based relationships allowing for each patient specific input from the user to act as its own frame of reference to neighboring, underlying and overlying anatomic regions, based on user identified or created events; and storing the complete patient history associated with the defined anatomical location.

In certain embodiments, the displayed generated output is a visualization in isolation (that also contains non-anatomy data such as order, color, procedure, diagnosis, morphology); a visualization in relation to other anatomic locations; a map or avatar with layers, custom axes, and direction planes; with a pin or color-coded highlighting in relation to the identified anatomic site or visualization; in association with other data; In certain embodiments, the method further comprising: processing a received image of a product label associated with the selected procedure; and extracting at least one of the set of fields associated with the selected template from the received image.

In one embodiment, a method for generating a medical record, comprises: rendering, on a display, an anatomic representation; receiving an input, from an input device, having health data, wherein the input is text-based, visual-based, audio-based, and/or based on an interaction with the anatomic representation; processing the input to generate processed health data, the processed health data including a procedure, a diagnosis, a name of an anatomic site, and/or a description of an anatomic site; rendering, on the display, a marked anatomic site on the anatomic representation that is based on the processed health data, or an isolated anatomic representation having a marked anatomic site that is based on the processed health data; selectively rendering, on the display, a mirror image of the anatomic representation with the marked anatomic site or the isolated anatomic representation with the marked anatomic site based on a user preference; selecting one of a plurality of templates, each of the templates having one or more fields; populating one of the fields with the anatomic representation with the marked anatomic site or the isolated anatomic representation with the marked anatomic site to generate a populated selected template; and generating a health record including the populated selected template.

In certain embodiments, the anatomic representation includes a plurality of predefined anatomic sites. In certain embodiments, one of the predefined anatomic sites is located on and associated with an anatomic region with one or more subregions. In certain embodiments, the interaction with the anatomic representation includes generating a preview of the anatomic region with the one or more subregions associated with the one of the predefined anatomic sites. In certain embodiments, the input is a scanned image of a physical print of the anatomic representation with markups, wherein the markups include the health data. In certain embodiments, the physical print of the anatomic representation with markups includes orientation markers and processing the input includes detecting orientation markers and normalizing the axis based on the orientation markers. In certain embodiments, processing the input is performed using a language model, a vision-language model, and/or a language-vision model. In certain embodiments, the marked anatomic site is associated with the processed data. In certain embodiments, the description of the anatomic site includes a relationship between the marked anatomic site and another anatomic site, wherein the relationship includes a distance between the marked anatomic site and the another anatomic site, a spatial relationship between the marked anatomic site and the another anatomic site, and/or a data-based relationship between the marked anatomic site and the another anatomic site. In certain embodiments, populating one of the fields includes populating one or more additional fields of the fields with the processed health data. In certain embodiments, the processing an input representing an anatomic site automatically includes translatable elements with non-limiting examples being laterality, prefixes, suffixes, relationships, cross-mappings, groups, synonyms, and other alternative descriptions in any linguistic, coded, and/or symbolic language. In certain embodiments, processing the input includes detecting a nontechnical term for the processed health data and converting the nontechnical term into a technical term. In certain embodiments, processing the input includes detecting a plurality of languages and/or encodings and/or symbolic representations and/or coordinates on a visualization and translating the plurality of languages and/or detections into the processed health data. In certain embodiments, the input includes a coded input and processing the input includes decoding the coded input into the processed health data. In certain embodiments, different linguistic languages process the processed health data differently applying natural language processing. In certain embodiments, generating the health record includes formatting the health record into a database record suitable for an electronic health record database.

In one embodiment, a system for generating a medical record, comprises: a processer; a medium in communication with the processor 986, wherein the medium is tangible, non-transitory, and computer readable; processer-executable instructions stored on the medium 995, the processor-executable instructions (e.g. in the data processing module 999) defining a mapping platform including a data processing module 999, a knowledge base module 992, and a generation module 993; a display 987 in communication with the medium 995; and an input device 988 in communication with the medium 995 and display 987; wherein the mapping platform is configured to: render, using the processor 986, an anatomic representation of a human on the display, receive, from the input device, an input having health data, wherein the input is text-based, visual-based, audio-based, and/or based on an interaction with the anatomic representation, process, using the data processing module, the input to generate processed health data, the processed health data including a procedure, a diagnosis, a name of an anatomic site, and/or a description of an anatomic site, render, using the processor 986, on the display, a marked anatomic site on the anatomic representation that is based on the processed health data, or an isolated anatomic representation having a marked anatomic site that is based on the processed health data, selectively rendering, using the processer, a mirror image of the anatomic representation with the marked anatomic site or the isolated anatomic representation with the marked anatomic site on the display based on a user preference; selecting one of a plurality of templates, from the knowledge base module, each of the templates having one or more fields; populating one of the fields, using the generation module, with the anatomic representation with the marked anatomic site or the isolated anatomic representation with the marked anatomic site to generate a populated selected template; and generating, using the generation module, a health record including the populated selected template.

In certain embodiments, the system further comprises a printer as an output device 989 configured to print a physical health record. In certain embodiments, the input device 988 includes an image capturing device configured to scan a physical representation of the anatomic representation with markups, wherein the markups include the health data. In certain embodiments, the processor 986 and the medium are located on one or more servers. In certain embodiments, the processor 986, the medium 995, and the display 987 are located on a mobile phone, a tablet, a laptop, a computer, and/or an electronic device. In one embodiment, a tangible, non-transitory, and computer-readable medium having processer-executable instructions stored thereon that when executed by a processor causes a method for generating a medical record, comprises: rendering, on a display, an anatomic representation; receiving an input, from an input device, having health data, wherein the input is text-based, visual-based, audio-based, and/or based on an interaction with the anatomic representation; processing the input to generate processed health data, the processed health data including a procedure, a diagnosis, a name of an anatomic site, and/or a description of an anatomic site; rendering, on the display, a marked anatomic site on the anatomic representation that is based on the processed health data, or an isolated anatomic representation having a marked anatomic site that is based on the processed health data; selectively rendering, on the display, a mirror image of the anatomic representation with the marked anatomic site or the isolated anatomic representation with the marked anatomic site based on a user preference; selecting one of a plurality of templates, each of the templates having one or more fields; populating one of the fields with the anatomic representation with the marked anatomic site or the isolated anatomic representation with the marked anatomic site to generate a populated selected template; and generating a health record including the populated selected template.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simply to describe and illustrate examples of the invention and are not intended to limit the scope of the invention or its protection, which is to be given the full breadth of the appended claims and any and all equivalents thereof.

What is claimed is:

1. A computer system comprising:
(a) a processor; and
(b) a non-transitory computer-readable medium storing instructions that, when executed by the processor, cause the processor to:
(i) receive an unstructured spoken input describing healthcare information;
(ii) extract, from the unstructured spoken input, an anatomic descriptor to generate an extracted anatomic descriptor;
(iii) coordinate the extracted anatomic descriptor to the anatomic location or distribution;
(iv) associate the anatomic location or distribution with a record target selected from:
(A) a text target related to a healthcare record;
(B) a code target related to a healthcare record;
(C) a document target related to a healthcare record;
(D) stored healthcare information associated with the extracted anatomic descriptor; or
(E) a location or distribution on or relative to an anatomic representation; and
(v) generate an output comprising healthcare information associated with the anatomic location or distribution.

2. The system of claim 1, wherein the unstructured spoken input comprises a conversational query regarding previously stored, newly generated, or prospective healthcare information.

3. The system of claim 1, wherein generating the output further comprises calculating a quantitative measure or relationship associated with the anatomic location or distribution.

4. The system of claim 1, wherein extracting comprises resolving a synonym of the anatomic descriptor.

5. The system of claim 1, wherein coordinating comprises resolving laterality information, modifier information, quantitative information, relational information, spatial information, or sequential information included in the unstructured spoken input.

6. The system of claim 1, wherein coordinating comprises disambiguating between multiple anatomic locations or distributions based on contextual information or sequence information in the unstructured spoken input.

7. The system of claim 1, wherein the document target comprises a pathology report, operative report, lab report, imaging report, billing report, clinical note, procedural note, surgical note, consultation report, discharge summary, patient instructions, label, document section, or other healthcare document.

8. The system of claim 1, wherein multiple anatomic descriptors are extracted from the unstructured spoken input.

9. The system of claim 1, wherein the healthcare information comprises diagnosis information, procedure information, morphology information, symptom information, schedule information, sequence information, or combinations thereof.

10. The system of claim 1, wherein the output comprises a visualization, target, or description associated with the anatomic location or distribution.

11. A computer system comprising:
(a) a processor; and
(b) a non-transitory computer-readable medium storing instructions that cause the processor to:
(i) receive a spoken input comprising an anatomic site reference and associated healthcare data;
(ii) extract and categorize the anatomic site reference and the associated healthcare data to generate categorized healthcare data;
(iii) coordinate the anatomic site reference to an anatomic location or a distribution to generate a coordinated anatomic location or distribution;
(iv) populate an electronic or printable healthcare form with the categorized healthcare data to generate a populated healthcare form; and
(v) generate a healthcare record.

12. The system of claim 11, wherein the healthcare form comprises a pathology requisition form, billing form, surgical map, scheduling form, pathology report, lab report, patient instruction form, label, or other healthcare form.

13. The system of claim 11, wherein populating the healthcare form further comprises calculating a quantitative measure or relationship associated with the coordinated anatomic location or distribution.

14. The system of claim 11, wherein the populated healthcare form is printable.

15. The system of claim 11, wherein the populated healthcare form is transmitted to another computing system.

16. The system of claim 11, wherein the system enables user refinement prior to storage.

17. A computer-implemented method comprising:
(a) receiving a spoken input including a non-standard anatomic term;
(b) extracting the non-standard anatomic term;
(c) coordinating the non-standard anatomic term to an anatomic descriptor corresponding to an anatomic location or distribution;
(d) accessing a stored healthcare information associated with the anatomic descriptor; and
(e) generating a response comprising the stored healthcare information.

18. The method of claim 17, wherein coordinating comprises resolving a synonym to the anatomic descriptor.

19. The method of claim 17, wherein the response includes a visualization of the anatomic location or distribution.

20. The method of claim 17, wherein response is transmitted to a remote user device.
